(19) United States
(12) Patent Application Publication
(10) Pub. No.: US 2026/0035697 A1
(43) Pub. Date: Feb. 5, 2026

(54) Compositions and Methods for Lactate Dehydrogenase (LDHA) Gene Editing

(71) Applicant: Intellia Therapeutics, Inc., Cambridge, MA (US)

(72) Inventors: Zachary William Dymek, Cambridge, MA (US); Shobu Odate, Arlington, MA (US); Anette Huebner, Somerville, MA (US); Srijani Sridhar, Cambridge, MA (US); Bradley Andrew Murray, Boston, MA (US); Walter Strapps, Dedham, MA (US)

(73) Assignee: Intellia Therapeutics, Inc., Cambridge, MA (US)

(21) Appl. No.: 19/210,426

(22) Filed: May 16, 2025

Related U.S. Application Data
(63) Continuation of application No. 17/212,901, filed on Mar. 25, 2021, now abandoned.
Application No. 17/212,901 is a continuation of application No. PCT/US2019/053423, filed on Sep. 27, 2019.
(60) Provisional application No. 62/738,956, filed on Sep. 28, 2018.
(60) Provisional application No. 62/834,334, filed on Apr. 15, 2019.
(60) Provisional application No. 62/841,740, filed on May 1, 2019.

(51) Int. Cl. C12N 15/113; A61P 13/02; C12N 9/22

(52) U.S. Cl. CPC C12N 15/113; A61P 13/02; C12N 9/22; C12N 2310/315; C12N 2310/321; C12N 2310/322; C12N 2310/531

(57) ABSTRACT

Compositions and methods for editing, e.g., introducing double-stranded breaks, within the LDHA gene are provided. Compositions and methods for treating subjects having hyperoxaluria are provided.

This application is a Continuation of U.S. patent application Ser. No. 17/212,901, filed Mar. 25, 2021, which is a Continuation application of International Application No. PCT/US2019/053423, filed Sep. 27, 2019, which claims the benefit of priority of U.S. Provisional Patent Application No. 62/738,956, filed Sep. 28, 2018, U.S. Provisional Patent Application No. 62/834,334, filed Apr. 15, 2019, and U.S. Provisional Patent Application No. 62/841,740, filed May 1, 2019, the contents of each of which are incorporated by reference for their entirety for all purposes.

### REFERENCE TO ELECTRONIC SEQUENCE LISTING

The application contains a Sequence Listing which has been submitted electronically in .XML format and is hereby incorporated by reference in its entirety. Said .XML copy, created on May 14, 2025, is named “01155-0025-01US.xml” and is 2,746,716 bytes in size. The sequence listing contained in this .XML file is part of the specification and is hereby incorporated by reference herein in its entirety.

Oxalate, normally eliminated in urine as waste by the kidneys, is elevated in subjects with hyperoxaluria. There are several types of hyperoxaluria, including primary hyperoxaluria, oxalosis, enteric hyperoxaluria, and hyperoxaluria related to eating high-oxalate foods. Excess oxalate can combine with calcium to form calcium oxalate in the kidney and other organs. Deposits of calcium oxalate can produce widespread deposition of calcium oxalate (nephrocalcinosis) or formation of kidney and bladder stones (urolithiasis) and lead to kidney damage. Common kidney complications in hyperoxaluria include blood in the urine (hematuria), urinary tract infections, kidney damage, and end-stage renal disease (ESRD). Over time, kidneys in patients with hyperoxaluria may begin to fail, and levels of oxalate may rise in the blood. Deposition of oxalate in tissues throughout the body, e.g., systemic oxalosis, may occur due to high blood levels of oxalate and can lead to complications in at least bone, heart, skin, and eye. Kidney failure can occur at any age, including in children, especially in subjects with hyperoxaluria. Renal dialysis or dual kidney/liver organ transplant as the only treatment options.

Primary hyperoxaluria (PH) is a rare genetic disorder effecting subjects of all ages from infants to elderly. PH includes three subtypes involving genetic defects that alter the expression of three distinct proteins. PH1 involves alanine-glyoxylate aminotransferase, or AGT/AGT1. PH2 involves glyoxylate/hydroxypyruvate reductase, or GR/HPR, and PH3 involves 4-hydroxy-2-oxoglutarate aldolase, or HOGA. In PH1, mutations are found in the enzyme alanine glyoxylate aminotransferase (AGT or AGT1) that is encoded by the AGXT gene. Normally, AGT converts glyoxylate into glycine in liver peroxisomes. In patients with PH1, mutant AGT is unable to break down glyoxylate, and levels of glyoxylate and its metabolite oxalate increase. Humans cannot oxidize oxalate, and high levels of oxalate in subjects with PH1 cause hyperoxaluria.

To determine whether a subject has hyperoxaluria, a 24-hour urine may be collected and the oxalate, glycolate, and other organic acid levels are measured. Genetic testing or liver biopsy can be performed for a definitive diagnosis of genetic forms of hyperoxaluria. See, e.g., Cochat P et al., (2012) Nephrol Dial Transplant 5:1729-36. In normal healthy subjects the 24-hour urine oxalate and glycolate levels are less than 45 mg/day but in hyperoxaluria patients, levels of urinary oxalate greater than 100 mg/day are typical. See, e.g., Cochat P. (2013). N Engl J Med 369:649-658.

Plasma glycolate levels in normal subjects are typically 4-8 micromolar but in hyperoxaluria patients glycolate levels can range widely and are elevated in ⅔rds of hyperoxaluria subjects. See, e.g., Marangella, M et al. (1992) J. Urol. 148:986-989. While most patients with genetic forms of hyperoxaluria are now diagnosed through genetic testing, a 24-hour urine test is the primary method used to follow hyperoxaluria subjects for treatment responses. Id.

Lactate dehydrogenase (LDH) is an enzyme found in nearly every cell that regulates both the homeostasis of lactate and pyruvate, and of glyoxylate and oxalate metabolism. LDH is comprised of 4 polypeptides that form a tetramer. Five isozymes of LDH differing in their subunit composition and tissue distribution have been identified. The two most common forms of LDH are the muscle (M) form encoded by the LDHA gene, and the heart (H) form encoded by LDHB gene. In the perioxisome of liver cells, LDH is the key enzyme responsible for converting glyoxalate to oxalate which is then secreted into the plasma and excreted by the kidneys. Lai et al. (2018) Mol Ther. 26(8):1983-1995.

An increase in oxalate production results in the precipitation of calcium oxalate crystals in the kidneys and renal disease. As hyperoxaluria progresses, oxalate is deposited in all tissues. Subjects with hereditary lactate dehydrogenase M-subunit deficiency do not display impaired liver function or a liver-specific phenotype suggesting that inhibiting or diminishing the amount of hepatic lactate dehydrogenase (LDH) expression, the proposed key enzyme responsible for converting glyoxylate to oxalate, may prevent the accumulation of oxalate in subjects with hyperoxaluria without adverse effects due to loss of the lactate dehydrogenase M-subunit. This hypothesis was tested in genetically engineered murine models of hyperoxaluria, and a murine model in which hyperoxaluria is chemically induced with ethylene glycol (EG). See, Kanno, T et al. (1988) Clin. Chim. Acta 173, 89-98; Takahashi, Y et al. (1995) Intern. Med. 34, 326-329; and Tsujino, S et al. (1994) Ann. Neurol. 36, 661-665.

As LDH is key in the final step of oxalate production, LDHA siRNA directed to hepatocytes via conjugation with N-acetylgalactosamine (GalNAc) residues was used to mediate LDHA silencing in mouse models of hyperoxaluria. See, Lai et al. (2018) Mol Ther. 26(8):1983-1995. Treatment of mice with this LDHA siRNA resulted in a reduction of hepatic LDH and efficient oxalate reduction and prevented calcium oxalate crystal deposition in both genetically engineered mouse models of hyperoxaluria and in chemically induced hyperoxaluria mouse models. Id. Suppression of hepatic LDH in mice did not result in acute elevation of circulating liver enzymes, lactate acidosis, or exertional myopathy.

The idea of treating patients with hyperoxaluria by inhibition of LDHA is further supported by the LDHA siRNA treatment of both non-human primates and humanized chimeric mice in which the liver is comprised of up to 80% human hepatocytes. Id.

Accordingly, the following embodiments are provided. In some embodiments, the disclosure provides compositions and methods using a guide RNA with an RNA-guided DNA binding agent such as the CRISPR/Cas system to substantially reduce or knockout expression of the LDHA gene, thereby substantially reducing or eliminating the production of LDH, thereby reducing urinary oxalate and increasing serum glycolate. The substantial reduction or elimination of the production of LDH through alteration of the LDHA gene can be a long-term or permanent treatment for hyperoxaluria.

### SUMMARY

The following embodiments are provided.

Embodiment 01 A method of inducing a double-stranded break (DSB) or single-stranded break (SSB) within the LDHA gene, comprising delivering a composition to a cell, wherein the composition comprises:
a. a guide RNA comprising
   i. a guide sequence selected from SEQ ID NOs:1-84 and 100-192; or
   ii. at least 17, 18, 19, or 20 contiguous nucleotides of a sequence selected from SEQ ID NOs:1-84 and 100-192; or
   iii. a guide sequence that is at least 99%, 98%, 97%, 96%, 95%, 94%, 93%, 92%, 91%, or 90% identical to a sequence selected from SEQ ID NOs:1-84 and 100-192; or
   iv. a guide sequence comprising any one of SEQ ID NOs: 1, 5, 7, 8, 14, 23, 27, 32, 45, 48, 62, 66, 68, 70, 73, 75, 76, 77, 78, and 80; or
   v. a guide sequence comprising any one of SEQ ID No: 1, 5, 7, 8, 14, 23, 27, 32, 45, and 48; or
   vi. a guide sequence comprising any one of SEQ ID NOs: 1, 5, 7, 8, 14, 23, 25, 27, 32, 45, 48, 62, 66, 68, 70, 73, 75, 76, 77, 78, 80, 103, 109, 123, 133, 149, 153, 156, and 184; or
   vii. a guide sequence comprising any one of SEQ ID NOs: 1, 5, 7, 8, 14, 23, 25, 27, 32, 45, 48, 103, and 123; and optionally
b. an RNA-guided DNA binding agent or a nucleic acid encoding an RNA-guided DNA binding agent.
   Embodiment 02 A method of reducing the expression of the LDHA gene comprising delivering a composition to a cell, wherein the composition comprises:
a. a guide RNA comprising
   i. a guide sequence selected from SEQ ID NOs:1-84 and 100-192; or
   ii. at least 17, 18, 19, or 20 contiguous nucleotides of a sequence selected from SEQ ID NOs:1-84 and 100-192; or
   iii. a guide sequence that is at least 99%, 98%, 97%, 96%, 95%, 94%, 93%, 92%, 91%, or 90% identical to a sequence selected from SEQ ID NOs:1-84 and 100-192; or
   iv. a guide sequence comprising any one of SEQ ID NOs: 1, 5, 7, 8, 14, 23, 27, 32, 45, 48, 62, 66, 68, 70, 73, 75, 76, 77, 78, and 80; or
   v. a guide sequence comprising any one of SEQ ID No: 1, 5, 7, 8, 14, 23, 27, 32, 45, and 48; or
   vi. a guide sequence comprising any one of SEQ ID NOs: 1, 5, 7, 8, 14, 23, 25, 27, 32, 45, 48, 62, 66, 68, 70, 73, 75, 76, 77, 78, 80, 103, 109, 123, 133, 149, 153, 156, and 184; or
   vii. a guide sequence comprising any one of SEQ ID NOs: 1, 5, 7, 8, 14, 23, 25, 27, 32, 45, 48, 103, and 123; and optionally
b. an RNA-guided DNA binding agent or a nucleic acid encoding an RNA-guided DNA binding agent.
   Embodiment 03 A method of treating or preventing hyperoxaluria comprising administering a composition to a subject in need thereof, wherein the composition comprises:
a. a guide RNA comprising
   i. a guide sequence selected from SEQ ID NOs:1-84 and 100-192; or
   ii. at least 17, 18, 19, or 20 contiguous nucleotides of a sequence selected from SEQ ID NOs:1-84 and 100-192; or
   iii. a guide sequence that is at least 99%, 98%, 97%, 96%, 95%, 94%, 93%, 92%, 91%, or 90% identical to a sequence selected from SEQ ID NOs:1-84 and 100-192; or
   iv. a guide sequence comprising any one of SEQ ID NOs: 1, 5, 7, 8, 14, 23, 27, 32, 45, 48, 62, 66, 68, 70, 73, 75, 76, 77, 78, and 80; or
   v. a guide sequence comprising any one of SEQ ID No: 1, 5, 7, 8, 14, 23, 27, 32, 45, and 48; or
   vi. a guide sequence comprising any one of SEQ ID NOs: 1, 5, 7, 8, 14, 23, 25, 27, 32, 45, 48, 62, 66, 68, 70, 73, 75, 76, 77, 78, 80, 103, 109, 123, 133, 149, 153, 156, and 184; or
   vii. a guide sequence comprising any one of SEQ ID NOs: 1, 5, 7, 8, 14, 23, 25, 27, 32, 45, 48, 103, and 123; and optionally
b. an RNA-guided DNA binding agent or nucleic acid encoding an RNA-guided DNA binding agent, thereby treating or preventing hyperoxaluria.
   Embodiment 04 A method of treating or preventing end stage renal disease (ESRD) caused by hyperoxaluria comprising administering a composition to a subject in need thereof, wherein the composition comprises:
a. a guide RNA comprising
   i. a guide sequence selected from SEQ ID NOs:1-84 and 100-192; or
   ii. at least 17, 18, 19, or 20 contiguous nucleotides of a sequence selected from SEQ ID NOs:1-84 and 100-192; or
   iii. a guide sequence that is at least 99%, 98%, 97%, 96%, 95%, 94%, 93%, 92%, 91%, or 90% identical to a sequence selected from SEQ ID NOs:1-84 and 100-192; or
   iv. a guide sequence comprising any one of SEQ ID NOs: 1, 5, 7, 8, 14, 23, 27, 32, 45, 48, 62, 66, 68, 70, 73, 75, 76, 77, 78, and 80; or
   v. a guide sequence comprising any one of SEQ ID NOs: 1, 5, 7, 8, 14, 23, 27, 32, 45, and 48; or
   vi. a guide sequence comprising any one of SEQ ID NOs: 1, 5, 7, 8, 14, 23, 25, 27, 32, 45, 48, 62, 66, 68, 70, 73, 75, 76, 77, 78, 80, 103, 109, 123, 133, 149, 153, 156, and 184; or
   vii. a guide sequence comprising any one of SEQ ID NOs: 1, 5, 7, 8, 14, 23, 25, 27, 32, 45, 48, 103, and 123; and optionally
b. an RNA-guided DNA binding agent or nucleic acid encoding an RNA-guided DNA binding agent, thereby treating or preventing (ESRD) caused by hyperoxaluria.
   Embodiment 05 A method of treating or preventing any one of calcium oxalate production and deposition, primary hyperoxaluria (including PH1, PH2, and PH3), oxalosis, hematuria, enteric hyperoxaluria, hyperoxaluria related to eating high-oxalate foods; and delaying or ameliorating the need for kidney or liver transplant comprising administering a composition to a subject in need thereof, wherein the composition comprises:
a. a guide RNA comprising
   i. a guide sequence selected from SEQ ID NOs:1-84 and 100-192; or
   ii. at least 17, 18, 19, or 20 contiguous nucleotides of a sequence selected from SEQ ID NOs:1-84 and 100-192; or
   iii. a guide sequence that is at least 99%, 98%, 97%, 96%, 95%, 94%, 93%, 92%, 91%, or 90% identical to a sequence selected from SEQ ID NOs:1-84 and 100-192; or
   iv. a guide sequence comprising any one of SEQ ID NOs: 1, 5, 7, 8, 14, 23, 27, 32, 45, 48, 62, 66, 68, 70, 73, 75, 76, 77, 78, and 80; or
   v. a guide sequence comprising any one of SEQ ID No: 1, 5, 7, 8, 14, 23, 27, 32, 45, and 48; or
   vi. a guide sequence comprising any one of SEQ ID NOs: 1, 5, 7, 8, 14, 23, 25, 27, 32, 45, 48, 62, 66, 68, 70, 73, 75, 76, 77, 78, 80, 103, 109, 123, 133, 149, 153, 156, and 184; or
   vii. a guide sequence comprising any one of SEQ ID NOs: 1, 5, 7, 8, 14, 23, 25, 27, 32, 45, 48, 103, and 123; and optionally
b. an RNA-guided DNA binding agent or nucleic acid encoding an RNA-guided DNA binding agent, thereby treating or preventing any one of calcium oxalate production and deposition, primary hyperoxaluria, oxalosis, hematuria, and delaying or ameliorating the need for kidney or liver transplant.
   Embodiment 06 A method of increasing serum glycolate concentration, comprising administering a composition to a subject in need thereof, wherein the composition comprises:
a. a guide RNA comprising
   i. a guide sequence selected from SEQ ID NOs:1-84 and 100-192; or
   ii. at least 17, 18, 19, or 20 contiguous nucleotides of a sequence selected from SEQ ID NOs:1-84 and 100-192; or
   iii. a guide sequence that is at least 99%, 98%, 97%, 96%, 95%, 94%, 93%, 92%, 91%, or 90% identical to a sequence selected from SEQ ID NOs:1-84 and 100-192; or
   iv. a guide sequence comprising any one of SEQ ID NOs: 1, 5, 7, 8, 14, 23, 27, 32, 45, 48, 62, 66, 68, 70, 73, 75, 76, 77, 78, and 80; or
   v. a guide sequence comprising any one of SEQ ID No: 1, 5, 7, 8, 14, 23, 27, 32, 45, and 48; or
   vi. a guide sequence comprising any one of SEQ ID NOs: 1, 5, 7, 8, 14, 23, 25, 27, 32, 45, 48, 62, 66, 68, 70, 73, 75, 76, 77, 78, 80, 103, 109, 123, 133, 149, 153, 156, and 184; or
   vii. a guide sequence comprising any one of SEQ ID NOs: 1, 5, 7, 8, 14, 23, 25, 27, 32, 45, 48, 103, and 123; and optionally
b. an RNA-guided DNA binding agent or nucleic acid encoding an RNA-guided DNA binding agent, thereby increasing serum glycolate concentration.
   Embodiment 07 A method for reducing oxylate in urine in a subject, comprising administering a composition to a subject in need thereof, wherein the composition comprises:
a. a guide RNA comprising
   i. a guide sequence selected from SEQ ID NOs:1-84 and 100-192; or
   ii. at least 17, 18, 19, or 20 contiguous nucleotides of a sequence selected from SEQ ID NOs:1-84 and 100-192; or
   iii. a guide sequence that is at least 99%, 98%, 97%, 96%, 95%, 94%, 93%, 92%, 91%, or 90% identical to a sequence selected from SEQ ID NOs:1-84 and 100-192; or
   iv. a guide sequence comprising any one of SEQ ID NOs: 1, 5, 7, 8, 14, 23, 27, 32, 45, 48, 62, 66, 68, 70, 73, 75, 76, 77, 78, and 80; or
   v. a guide sequence comprising any one of SEQ ID No: 1, 5, 7, 8, 14, 23, 27, 32, 45, and 48; or
   vi. a guide sequence comprising any one of SEQ ID NOs: 1, 5, 7, 8, 14, 23, 25, 27, 32, 45, 48, 62, 66, 68, 70, 73, 75, 76, 77, 78, 80, 103, 109, 123, 133, 149, 153, 156, and 184; or
   vii. a guide sequence comprising any one of SEQ ID NOs: 1, 5, 7, 8, 14, 23, 25, 27, 32, 45, 48, 103, and 123; and optionally
b. an RNA-guided DNA binding agent or nucleic acid encoding an RNA-guided DNA binding agent, thereby reducing oxalate in the urine of a subject.
   Embodiment 08 The method of any one of the preceding embodiments, wherein an RNA-guided DNA binding agent or nucleic acid encoding an RNA-guided DNA binding agent is administered.
   Embodiment 09 A composition comprising:
a. a guide RNA comprising
   i. a guide sequence selected from SEQ ID NOs:1-84 and 100-192; or
   ii. at least 17, 18, 19, or 20 contiguous nucleotides of a sequence selected from SEQ ID NOs:1-84 and 100-192; or
   iii. a guide sequence that is at least 99%, 98%, 97%, 96%, 95%, 94%, 93%, 92%, 91%, or 90% identical to a sequence selected from SEQ ID NOs:1-84 and 100-192; or
   iv. a guide sequence comprising any one of SEQ ID NOs: 1, 5, 7, 8, 14, 23, 27, 32, 45, 48, 62, 66, 68, 70, 73, 75, 76, 77, 78, and 80 or
   v. a guide sequence comprising any one of SEQ ID No: 1, 5, 7, 8, 14, 23, 27, 32, 45, and 48; or
   vi. a guide sequence comprising any one of SEQ ID NOs: 1, 5, 7, 8, 14, 23, 25, 27, 32, 45, 48, 62, 66, 68, 70, 73, 75, 76, 77, 78, 80, 103, 109, 123, 133, 149, 153, 156, and 184; or
   vii. a guide sequence comprising any one of SEQ ID NOs: 1, 5, 7, 8, 14, 23, 25, 27, 32, 45, 48, 103, and 123; and optionally
b. an RNA-guided DNA binding agent or nucleic acid encoding an RNA-guided DNA binding agent.
   Embodiment 10 A composition comprising a short-single guide RNA (short-sgRNA), comprising:
a. a guide sequence comprising:
   i. any one of the guide sequences selected from SEQ ID NOs:1-84 and 100-192; or
   ii. at least 17, 18, 19, or 20 contiguous nucleotides of any one of the guide sequences selected from SEQ ID NOs:1-84 and 100-192; or
   iii. at least 99%, 98%, 97%, 96%, 95%, 94%, 93%, 92%, 91%, or 90% identical to a sequence selected from SEQ ID NOs:1-84 and 100-192; or
   iv. any one of SEQ ID NOs: 1, 5, 7, 8, 14, 23, 27, 32, 45, 48, 62, 66, 68, 70, 73, 75, 76, 77, 78, and 80; or
   v. any one of SEQ ID No: 1, 5, 7, 8, 14, 23, 27, 32, 45, and 48; or
   vi. a guide sequence comprising any one of SEQ ID NOs: 1, 5, 7, 8, 14, 23, 25, 27, 32, 45, 48, 62, 66, 68, 70, 73, 75, 76, 77, 78, 80, 103, 109, 123, 133, 149, 153, 156, and 184; or
   vii. a guide sequence comprising any one of SEQ ID NOs: 1, 5, 7, 8, 14, 23, 25, 27, 32, 45, 48, 103, and 123; and
b. a conserved portion of an sgRNA comprising a hairpin region, wherein the hairpin region lacks at least 5-10 nucleotides and optionally wherein the short-sgRNA comprises one or more of a 5′ end modification and a 3′ end modification.
   Embodiment 11 The composition of embodiment 10, comprising the sequence of SEQ ID NO: 202.
   Embodiment 12 The composition of embodiment 10 or embodiment 11, comprising a 5′ end modification.
   Embodiment 13 The composition of any one of embodiments 10-12, wherein the short-sgRNA comprises a 3′ end modification.
   Embodiment 14 The composition of any one of embodiments 10-13, wherein the short-sgRNA comprises a 5′ end modification and a 3′ end modification.
   Embodiment 15 The composition of any one of embodiments 10-14, wherein the short-sgRNA comprises a 3′ tail.
   Embodiment 16 The composition of embodiment 15, wherein the 3′ tail comprises 1, 2, 3, 4, 5, 6, 7, 8, 9, or 10 nucleotides.
   Embodiment 17 The composition of embodiment 15, wherein the 3′ tail comprises about 1-2, 1-3, 1-4, 1-5, 1-7, 1-10, at least 1-2, at least 1-3, at least 1-4, at least 1-5, at least 1-7, or at least 1-10 nucleotides.
   Embodiment 18 The composition of any one of embodiments 10-17, wherein the short-sgRNA does not comprise a 3′ tail.
   Embodiment 19 The composition of any one of embodiments 10-18, comprising a modification in the hairpin region.
   Embodiment 20 The composition of any one of embodiments 10-19, comprising a 3′ end modification, and a modification in the hairpin region.
   Embodiment 21 The composition of any one of embodiments 10-20, comprising a 3′ end modification, a modification in the hairpin region, and a 5′ end modification.
   Embodiment 22 The composition of any one of embodiments 10-21, comprising a 5′ end modification, and a modification in the hairpin region.
   Embodiment 23 The composition of any one of embodiments 10-22, wherein the hairpin region lacks at least 5 consecutive nucleotides.
   Embodiment 24 The composition of any one of embodiments 10-23, wherein the at least 5-10 lacking nucleotides:
a. are within hairpin 1;
b. are within hairpin 1 and the “N” between hairpin 1 and hairpin 2;
c. are within hairpin 1 and the two nucleotides immediately 3′ of hairpin 1;
d. include at least a portion of hairpin 1;
e. are within hairpin 2;
f. include at least a portion of hairpin 2;
g. are within hairpin 1 and hairpin 2;
h. include at least a portion of hairpin 1 and include the “N” between hairpin 1 and hairpin 2;
i. include at least a portion of hairpin 2 and include the “N” between hairpin 1 and hairpin 2;
j. include at least a portion of hairpin 1, include the “N” between hairpin 1 and hairpin 2, and include at least a portion of hairpin 2;
k. are within hairpin 1 or hairpin 2, optionally including the “N” between hairpin 1 and hairpin 2;
l. are consecutive;
m. are consecutive and include the “N” between hairpin 1 and hairpin 2;
n. are consecutive and span at least a portion of hairpin 1 and a portion of hairpin 2;
o. are consecutive and span at least a portion of hairpin 1 and the “N” between hairpin 1 and hairpin 2;
p. are consecutive and span at least a portion of hairpin 1 and two nucleotides immediately 3′ of hairpin 1;
q. consist of 5-10 nucleotides;
r. consist of 6-10 nucleotides;
s. consist of 5-10 consecutive nucleotides;
t. consist of 6-10 consecutive nucleotides; or
u. consist of nucleotides 54-58 of SEQ ID NO: 400.
   Embodiment 25 The composition of any one of embodiments 10-24, comprising a conserved portion of an sgRNA comprising a nexus region, wherein the nexus region lacks at least one nucleotide.
   Embodiment 26 The composition of embodiment 25, wherein the nucleotides lacking in the nexus region comprise any one or more of:
a. at least 2, 3, 4, 5, 6, 7, 8, 9, or 10 nucleotides in the nexus region;
b. at least or exactly 1-2 nucleotides, 1-3 nucleotides, 1-4 nucleotides, 1-5 nucleotides, 1-6 nucleotides, 1-10 nucleotides, or 1-15 nucleotides in the nexus region; and
c. each nucleotide in the nexus region.
   Embodiment 27 A composition comprising a modified single guide RNA (sgRNA) comprising
a. a guide sequence comprising:
   i. any one of the guide sequences selected from SEQ ID NOs:1-84 and 100-192; or
   ii. at least 17, 18, 19, or 20 contiguous nucleotides of any one of the guide sequences selected from SEQ ID NOs:1-84 and 100-192; or
   iii. at least 99%, 98%, 97%, 96%, 95%, 94%, 93%, 92%, 91%, or 90% identical to a sequence selected from SEQ ID NOs:1-84 and 100-192; or
   iv. any one of SEQ ID NOs: 1, 5, 7, 8, 14, 23, 27, 32, 45, 48, 62, 66, 68, 70, 73, 75, 76, 77, 78, and 80; or
   v. any one of SEQ ID No: 1, 5, 7, 8, 14, 23, 27, 32, 45, and 48; or
   vi. any one of SEQ ID NOs: 1, 5, 7, 8, 14, 23, 25, 27, 32, 45, 48, 62, 66, 68, 70, 73, 75, 76, 77, 78, 80, 103, 109, 123, 133, 149, 153, 156, and 184; or
   vii. any one of SEQ ID NOs: 1, 5, 7, 8, 14, 23, 25, 27, 32, 45, 48, 103, and 123; and further comprising
b. one or more modifications selected from:
   1. a YA modification at one or more guide region YA sites;
   2. a YA modification at one or more conserved region YA sites;
   3. a YA modification at one or more guide region YA sites and at one or more conserved region YA sites;
   4. i) a YA modification at two or more guide region YA sites;
      ii) a YA modification at one or more of conserved region YA sites 2, 3, 4, and 10; and
      iii) a YA modification at one or more of conserved region YA sites 1 and 8; or
   5. i) a YA modification at one or more guide region YA sites, wherein the guide region YA site is at or after nucleotide 8 from the 5′ end of the 5′ terminus;
      ii) a YA modification at one or more of conserved region YA sites 2, 3, 4, and 10; and optionally;
      iii) a YA modification at one or more of conserved region YA sites 1 and 8; or
   6. i) a YA modification at one or more guide region YA sites, wherein the guide region YA site is within 13 nucleotides of the 3′ terminal nucleotide of the guide region;
      ii) a YA modification at one or more of conserved region YA sites 2, 3, 4, and 10; and
      iii) a YA modification at one or more of conserved region YA sites 1 and 8; or
   7. i) a 5′ end modification and a 3′ end modification;
      ii) a YA modification at one or more of conserved region YA sites 2, 3, 4, and 10; and
      iii) a YA modification at one or more of conserved region YA sites 1 and 8; or
   8. i) a YA modification at a guide region YA site, wherein the modification of the guide region YA site comprises a modification that at least one nucleotide located 5′ of the guide region YA site does not comprise;
      ii) a YA modification at one or more of conserved region YA sites 2, 3, 4, and 10; and
      iii) a YA modification at one or more of conserved region YA sites 1 and 8; or
   9. i) a YA modification at one or more of conserved region YA sites 2, 3, 4, and 10; and
      ii) a YA modification at conserved region YA sites 1 and 8; or
   10. i) a YA modification at one or more guide region YA sites, wherein the YA site is at or after nucleotide 8 from the 5′ terminus;
      ii) a YA modification at one or more of conserved region YA sites 2, 3, 4, and 10; and
      iii) a modification at one or more of H1-1 and H2-1; or
   11. i) a YA modification at one or more of conserved region YA sites 2, 3, 4, and 10;
      ii) a YA modification at one or more of conserved region YA sites 1, 5, 6, 7, 8, and 9; and
      iii) a modification at one or more of H1-1 and H2-1; or
   12. i) a modification, such as a YA modification, at one or more nucleotides located at or after nucleotide 6 from the 5′ terminus;
      ii) a YA modification at one or more guide sequence YA sites;
      iii) a modification at one or more of B3, B4, and B5, wherein B6 does not comprise a 2′-OMe modification or comprises a modification other than 2′-OMe;
      iv) a modification at LS10, wherein LS10 comprises a modification other than 2′-fluoro; and/or
      v) a modification at N2, N3, N4, N5, N6, N7, N10, or N11; and wherein at least one of the following is true:
         i. a YA modification at one or more guide region YA sites;
         ii. a YA modification at one or more conserved region YA sites;
         iii. a YA modification at one or more guide region YA sites and at one or more conserved region YA sites;
         iv. at least one of nucleotides 8-11, 13, 14, 17, or 18 from the 5′ end of the 5′ terminus does not comprise a 2′-fluoro modification;
         v. at least one of nucleotides 6-10 from the 5′ end of the 5′ terminus does not comprise a phosphorothioate linkage;
         vi. at least one of B2, B3, B4, or B5 does not comprise a 2′-OMe modification;
         vii. at least one of LS1, LS8, or LS10 does not comprise a 2′-OMe modification;
         viii. at least one of N2, N3, N4, N5, N6, N7, N10, N11, N16, or N17 does not comprise a 2′-OMe modification;
         ix. H1-1 comprises a modification;
         x. H2-1 comprises a modification; or
         xi. at least one of H1-2, H1-3, H1-4, H1-5, H1-6, H1-7, H1-8, H1-9, H1-10, H2-1, H2-2, H2-3, H2-4, H2-5, H2-6, H2-7, H2-8, H2-9, H2-10, H2-11, H2-12, H2-13, H2-14, or H2-15 does not comprise a phosphorothioate linkage.
            Embodiment 28 The composition of embodiment 27, comprising SEQ ID NO: 450.
            Embodiment 29 The composition of any one of embodiments 9-28, for use in inducing a double-stranded break (DSB) or single-stranded break (SSB) within the LDHA gene in a cell or subject.
            Embodiment 30 The composition of any one of embodiments 9-28, for use in reducing the expression of the LDHA gene in a cell or subject.
            Embodiment 31 The composition of any one of embodiments 9-28, for use in treating or preventing hyperoxaluria in a subject.
            Embodiment 32 The composition of any one of embodiments 9-28, for use in increasing serum and/or plasma glycolate concentration in a subject.
            Embodiment 33 The composition of any one of embodiments 9-28, for use in reducing urinary oxalate concentration in a subject.
            Embodiment 34 The composition of any one of embodiments 9-28, for use in treating or preventing oxalate production, calcium oxalate deposition in organs, primary hyperoxaluria, oxalosis, including systemic oxalosis, hematuria, end stage renal disease (ESRD) and/or delaying or ameliorating the need for kidney or liver transplant.
            Embodiment 35 The method of any of embodiments 1-8, further comprising:
a. inducing a double-stranded break (DSB) within the LDHA gene in a cell or subject;
b. reducing the expression of the LDHA gene in a cell or subject;
c. treating or preventing hyperoxaluria in a subject;
d. treating or preventing primary hyperoxaluria in a subject;
e. treating or preventing PH1, PH2, and/or PH3 in a subject;
f. treating or preventing enteric hyperoxaluria in a subject;
g. treating or preventing hyperoxaluria related to eating high-oxalate foods in a subject;
h. increasing serum and/or plasma glycolate concentration in a subject;
i. reducing urinary oxalate concentration in a subject;
j. reducing oxalate production;
k. reducing calcium oxalate deposition in organs;
l. reducing hyperoxaluria;
m. treating or preventing oxalosis, including systemic oxalosis;
n. treating or preventing hematuria;
o. preventing end stage renal disease (ESRD); and/or
p. delaying or ameliorating the need for kidney or liver transplant.
   Embodiment 36 The method or composition for use of any one of embodiments 1-8 or 29-35, wherein the composition increases serum and/or plasma glycolate levels.
   Embodiment 37 The method or composition for use of any one of embodiments 1-8 or 29-35, wherein the composition results in editing of the LDHA gene.
   Embodiment 38 The method or composition for use of embodiment 37, wherein the editing is calculated as a percentage of the population that is edited (percent editing).
   Embodiment 39 The method or composition for use of embodiment 38, wherein the percent editing is between 30 and 99% of the population.
   Embodiment 40 The method or composition for use of embodiment 38, wherein the percent editing is between 30 and 35%, 35 and 40%, 40 and 45%, 45 and 50%, 50 and 55%, 55 and 60%, 60 and 65%, 65 and 70%, 70 and 75%, 75 and 80%, 80 and 85%, 85 and 90%, 90 and 95%, or 95 and 99% of the population.
   Embodiment 41 The method or composition for use of any one of embodiments 1-8 or 29-35, wherein the composition reduces urinary oxalate concentration.
   Embodiment 42 The method or composition for use of embodiment 41, wherein a reduction in urinary oxalate results in decreased kidney stones and/or calcium oxalate deposition in the kidney, liver, bladder, heart, skin or eye.
   Embodiment 43 The method or composition of any one of the preceding embodiments, wherein the guide sequence is selected from
a. SEQ ID NOs:1-84 and 100-192;
b. SEQ ID NOs: 1, 5, 7, 8, 14, 23, 27, 32, 45, 48, 62, 66, 68, 70, 73, 75, 76, 77, 78, and 80;
c. SEQ ID NOs: 1, 5, 7, 8, 14, 23, 27, 32, 45, and 48;
d. SEQ ID NOs: 1, 5, 7, 8, 14, 23, 25, 27, 32, 45, 48, 62, 66, 68, 70, 73, 75, 76, 77, 78, 80, 103, 109, 123, 133, 149, 153, 156, and 184; and
e. SEQ ID NOs: 1, 5, 7, 8, 14, 23, 25, 27, 32, 45, 48, 103, and 123.
   Embodiment 44 The method or composition of any one of the preceding embodiments, wherein the composition comprises an sgRNA comprising
a. any one of SEQ ID NOs: 1001, 1005, 1007, 1008, 1014, 1023, 1027, 1032, 1045, 1048, 1063, 1067, 1069, 1071, 1074, 1076, 1077, 1078, 1079, and 1081; or
b. any one of SEQ ID NOs: 2001, 2005, 2007, 2008, 2014, 2023, 2027, 2032, 2045, 2048, 2063, 2067, 2069, 2071, 2074, 2076, 2077, 2078, 2079, and 2081; or
c. a guide sequence selected from SEQ ID NOs: 1, 5, 7, 8, 14, 23, 27, 32, 45, 48, 62, 66, 68, 70, 73, 75, 76, 77, 78, and 80; or
d. a guide sequence selected from SEQ ID NOs: 1, 5, 7, 8, 14, 23, 27, 32, 45, and 48;
e. a guide sequence selected from SEQ ID NOs: 1, 5, 7, 8, 14, 23, 25, 27, 32, 45, 48, 62, 66, 68, 70, 73, 75, 76, 77, 78, 80, 103, 109, 123, 133, 149, 153, 156, and 184; and
f. a guide sequence selected from SEQ ID NOs: 1, 5, 7, 8, 14, 23, 25, 27, 32, 45, 48, 103, and 123.
   Embodiment 45 The method or composition of any one of the preceding embodiments, wherein the target sequence is in any one of exons 1-8 of the human LDHA gene.
   Embodiment 46 The method or composition of embodiment 45, wherein the target sequence is in exon 1 or 2 of the human LDHA gene.
   Embodiment 47 The method or composition of embodiment 45, wherein the target sequence is in exon 3 of the human LDHA gene.
   Embodiment 48 The method or composition of embodiment 45, wherein the target sequence is in exon 4 of the human LDHA gene.
   Embodiment 49 The method or composition of embodiment 45, wherein the target sequence is in exon 5 or 6 of the human LDHA gene.
   Embodiment 50 The method or composition of embodiment 45, wherein the target sequence is in exon 7 or 8 of the human LDHA gene.
   Embodiment 51 The method or composition of any one of embodiments 1-50, wherein the guide sequence is complementary to a target sequence in the positive strand of LDHA.
   Embodiment 52 The method or composition of any one of embodiments 1-50, wherein the guide sequence is complementary to a target sequence in the negative strand of LDHA.
   Embodiment 53 The method or composition of any one of embodiments 1-50, wherein the first guide sequence is complementary to a first target sequence in the positive strand of the LDHA gene, and wherein the composition further comprises a second guide sequence that is complementary to a second target sequence in the negative strand of the LDHA gene.
   Embodiment 54 The method or composition of any one of the preceding embodiments, wherein the guide RNA comprises a guide sequence selected from any one of SEQ ID NOs 1-84 and 100-192 and further comprises a nucleotide sequence of SEQ ID NO: 200, wherein the nucleotides of SEQ ID NO: 200 follow the guide sequence at its 3′ end.
   Embodiment 55 The method or composition of any one of the preceding embodiments, wherein the guide RNA comprises a guide sequence selected from any one of SEQ ID NOs 1-84 and 100-192 and further comprises a nucleotide sequence of SEQ ID NO: 201, SEQ ID NO: 202, SEQ ID NO: 203, or any one of SEQ ID NO: 400-450 wherein the nucleotides of SEQ ID NO: 201, SEQ ID NO: 202, or SEQ ID NO: 203 follow the guide sequence at its 3′ end.
   Embodiment 56 The method or composition of any one of the preceding embodiments, wherein the guide RNA is a single guide (sgRNA).
   Embodiment 57 The method or composition of embodiment 56, wherein the sgRNA comprises a guide sequence comprising any one of SEQ ID NOs: 1001, 1005, 1007, 1008, 1014, 1023, 1027, 1032, 1045, 1048, 1063, 1067, 1069, 1071, 1074, 1076, 1077, 1078, 1079, and 1081.
   Embodiment 58 The method or composition of embodiment 56, wherein the sgRNA comprises any one of SEQ ID NOs: 1001, 1005, 1007, 1008, 1014, 1023, 1027, 1032, 1045, 1048, 1063, 1067, 1069, 1071, 1074, 1076, 1077, 1078, 1079, and 1081, or modified versions thereof, optionally wherein the modified versions comprise SEQ ID NOs: 2001, 2005, 2007, 2008, 2014, 2023, 2027, 2032, 2045, 2048, 2063, 2067, 2069, 2071, 2074, 2076, 2077, 2078, 2079, and 2081.
   Embodiment 59 The method or composition of any one of the preceding embodiments, wherein the guide RNA is modified according to the pattern of SEQ ID NO: 300, wherein the N's are collectively any one of the guide sequences of Table 1 (SEQ ID NOs 1-84 and 100-192).
   Embodiment 60 The method or composition of embodiment 59, wherein each N in SEQ ID NO: 300 is any natural or non-natural nucleotide, wherein the N's form the guide sequence, and the guide sequence targets Cas9 to the LDHA gene.
   Embodiment 61 The method or composition of any one of the preceding embodiments, wherein the sgRNA comprises any one of the guide sequences of SEQ ID NOs:1-84 and 100-192 and the nucleotides of SEQ ID NO: 201, SEQ ID NO: 202, or SEQ ID NO: 203.
   Embodiment 62 The method or composition of any one of embodiments 56-61, wherein the sgRNA comprises a guide sequence that is at least 99%, 98%, 97%, 96%, 95%, 94%, 93%, 92%, 91%, or 90% identical to a sequence selected from SEQ ID NOs: 1-84 and 100-192.
   Embodiment 63 The method or composition of embodiment 62, wherein the sgRNA comprises a sequence selected from SEQ ID NOs: 1, 5, 7, 8, 14, 23, 27, 32, 45, 48, 62, 66, 68, 70, 73, 75, 76, 77, 78, 80, 1001, 1005, 1007, 1008, 1014, 1023, 1027, 1032, 1045, 1048, 1063, 1067, 1069, 1071, 1074, 1076, 1077, 1078, 1079, 1081, 2001, 2005, 2007, 2008, 2014, 2023, 2027, 2032, 2045, 2048, 2063, 2067, 2069, 2071, 2074, 2076, 2077, 2078, 2079, and 2081.
   Embodiment 64 The method or composition of any one of the preceding embodiments, wherein the guide RNA comprises at least one modification.
   Embodiment 65 The method or composition of embodiment 64, wherein the at least one modification includes a 2′-O-methyl (2′-O-Me) modified nucleotide.
   Embodiment 66 The method or composition of embodiment 64 or 65, comprising a phosphorothioate (PS) bond between nucleotides.
   Embodiment 67 The method or composition of any one of embodiments 64-66, comprising a 2′-fluoro (2′-F) modified nucleotide.
   Embodiment 68 The method or composition of any one of embodiments 64-67, comprising a modification at one or more of the first five nucleotides at the 5′ end of the guide RNA.
   Embodiment 69 The method or composition of any one of embodiments 64-68, comprising a modification at one or more of the last five nucleotides at the 3′ end of the guide RNA.
   Embodiment 70 The method or composition of any one of embodiments 64-69, comprising a PS bond between the first four nucleotides of the guide RNA.
   Embodiment 71 The method or composition of any one of embodiments 64-70, comprising a PS bond between the last four nucleotides of the guide RNA.
   Embodiment 72 The method or composition of any one of embodiments 64-71, comprising a 2′-O-Me modified nucleotide at the first three nucleotides at the 5′ end of the guide RNA.
   Embodiment 73 The method or composition of any one of embodiments 64-72, comprising a 2′-O-Me modified nucleotide at the last three nucleotides at the 3′ end of the guide RNA.
   Embodiment 74 The method or composition of any one of embodiments 64-73, wherein the guide RNA comprises the modified nucleotides of SEQ ID NO: 300.
   Embodiment 75 The method or composition of any one of embodiments 1-74, wherein the composition further comprises a pharmaceutically acceptable excipient.
   Embodiment 76 The method or composition of any one of embodiments 1-75, wherein the guide RNA is associated with a lipid nanoparticle (LNP).
   Embodiment 77 The method or composition of embodiment 76, wherein the LNP comprises a cationic lipid.
   Embodiment 78 The method or composition of embodiment 77, wherein the cationic lipid is (9Z,12Z)-3-((4,4-bis(octyloxy)butanoyl)oxy)-2-((((3-(diethylamino)propoxy)carbonyl)oxy)methyl)propyl octadeca-9,12-dienoate, also called 3-((4,4-bis(octyloxy)butanoyl)oxy)-2-((((3-(diethylamino)propoxy)carbonyl)oxy)methyl)propyl (9Z,12Z)-octadeca-9,12-dienoate.
   Embodiment 79 The method or composition of any one of embodiments 76-78, wherein the LNP comprises a neutral lipid.
   Embodiment 80 The method or composition of embodiment 79, wherein the neutral lipid is DSPC.
   Embodiment 81 The method or composition of any one of embodiments 76-80, wherein the LNP comprises a helper lipid.
   Embodiment 82 The method or composition of embodiment 81, wherein the helper lipid is cholesterol.
   Embodiment 83 The method or composition of any one of embodiments 76-82, wherein the LNP comprises a stealth lipid.
   Embodiment 84 The method or composition of embodiment 83, wherein the stealth lipid is PEG2k-DMG.
   Embodiment 85 The method or composition of any one of the preceding embodiments, wherein the composition further comprises an RNA-guided DNA binding agent.
   Embodiment 86 The method or composition of any one of the preceding embodiments, wherein the composition further comprises an mRNA that encodes an RNA-guided DNA binding agent.
   Embodiment 87 The method or composition of embodiment 85 or 86, wherein the RNA-guided DNA binding agent is Cas9.
   Embodiment 88 The method or composition of any one of the preceding embodiments, wherein the composition is a pharmaceutical formulation and further comprises a pharmaceutically acceptable carrier.
   Embodiment 89 The method or composition of any one of embodiments 1-88, wherein the sequence selected from SEQ ID NOs:1-84 and 100-192 is SEQ ID NO: 1.
   Embodiment 90 The method or composition of any one of embodiments 1-88, wherein the sequence selected from SEQ ID NOs:1-84 and 100-192 is SEQ ID NO: 2.
   Embodiment 91 The method or composition of any one of embodiments 1-88, wherein the sequence selected from SEQ ID NOs:1-84 and 100-192 is SEQ ID NO: 3.
   Embodiment 92 The method or composition of any one of embodiments 1-88, wherein the sequence selected from SEQ ID NOs:1-84 and 100-192 is SEQ ID NO: 4.
   Embodiment 93 The method or composition of any one of embodiments 1-88, wherein the sequence selected from SEQ ID NOs:1-84 and 100-192 is SEQ ID NO: 5.
   Embodiment 94 The method or composition of any one of embodiments 1-88, wherein the sequence selected from SEQ ID NOs:1-84 and 100-192 is SEQ ID NO: 6.
   Embodiment 95 The method or composition of any one of embodiments 1-88, wherein the sequence selected from SEQ ID NOs:1-84 and 100-192 is SEQ ID NO: 7.
   Embodiment 96 The method or composition of any one of embodiments 1-88, wherein the sequence selected from SEQ ID NOs:1-84 and 100-192 is SEQ ID NO: 8.
   Embodiment 97 The method or composition of any one of embodiments 1-88, wherein the sequence selected from SEQ ID NOs:1-84 and 100-192 is SEQ ID NO: 9.
   Embodiment 98 The method or composition of any one of embodiments 1-88, wherein the sequence selected from SEQ ID NOs: 1-84 and 100-192 is SEQ ID NO: 10.
   Embodiment 99 The method or composition of any one of embodiments 1-88, wherein the sequence selected from SEQ ID NOs:1-84 and 100-192 is SEQ ID NO: 11.
   Embodiment 100 The method or composition of any one of embodiments 1-88, wherein the sequence selected from SEQ ID NOs:1-84 and 100-192 is SEQ ID NO: 12.
   Embodiment 101 The method or composition of any one of embodiments 1-88, wherein the sequence selected from SEQ ID NOs:1-84 and 100-192 is SEQ ID NO: 13.
   Embodiment 102 The method or composition of any one of embodiments 1-88, wherein the sequence selected from SEQ ID NOs:1-84 and 100-192 is SEQ ID NO: 14.
   Embodiment 103 The method or composition of any one of embodiments 1-88, wherein the sequence selected from SEQ ID NOs:1-84 and 100-192 is SEQ ID NO: 15.
   Embodiment 104 The method or composition of any one of embodiments 1-88, wherein the sequence selected from SEQ ID NOs:1-84 and 100-192 is SEQ ID NO: 16.
   Embodiment 105 The method or composition of any one of embodiments 1-88, wherein the sequence selected from SEQ ID NOs:1-84 and 100-192 is SEQ ID NO: 17.
   Embodiment 106 The method or composition of any one of embodiments 1-88, wherein the sequence selected from SEQ ID NOs:1-84 and 100-192 is SEQ ID NO: 18.
   Embodiment 107 The method or composition of any one of embodiments 1-88, wherein the sequence selected from SEQ ID NOs:1-84 and 100-192 is SEQ ID NO: 19.
   Embodiment 108 The method or composition of any one of embodiments 1-88, wherein the sequence selected from SEQ ID NOs:1-84 and 100-192 is SEQ ID NO: 20.
   Embodiment 109 The method or composition of any one of embodiments 1-88, wherein the sequence selected from SEQ ID NOs:1-84 and 100-192 is SEQ ID NO: 21.
   Embodiment 110 The method or composition of any one of embodiments 1-88, wherein the sequence selected from SEQ ID NOs:1-84 and 100-192 is SEQ ID NO: 22.
   Embodiment 111 The method or composition of any one of embodiments 1-88, wherein the sequence selected from SEQ ID NOs:1-84 and 100-192 is SEQ ID NO: 23.
   Embodiment 112 The method or composition of any one of embodiments 1-88, wherein the sequence selected from SEQ ID NOs:1-84 and 100-192 is SEQ ID NO: 24.
   Embodiment 113 The method or composition of any one of embodiments 1-88, wherein the sequence selected from SEQ ID NOs:1-84 and 100-192 is SEQ ID NO: 25.
   Embodiment 114 The method or composition of any one of embodiments 1-88, wherein the sequence selected from SEQ ID NOs:1-84 and 100-192 is SEQ ID NO: 26.
   Embodiment 115 The method or composition of any one of embodiments 1-88, wherein the sequence selected from SEQ ID NOs:1-84 and 100-192 is SEQ ID NO: 27.
   Embodiment 116 The method or composition of any one of embodiments 1-88, wherein the sequence selected from SEQ ID NOs:1-84 and 100-192 is SEQ ID NO: 28.
   Embodiment 117 The method or composition of any one of embodiments 1-88, wherein the sequence selected from SEQ ID NOs:1-84 and 100-192 is SEQ ID NO: 29.
   Embodiment 118 The method or composition of any one of embodiments 1-88, wherein the sequence selected from SEQ ID NOs:1-84 and 100-192 is SEQ ID NO: 30.
   Embodiment 119 The method or composition of any one of embodiments 1-88, wherein the sequence selected from SEQ ID NOs:1-84 and 100-192 is SEQ ID NO: 31.
   Embodiment 120 The method or composition of any one of embodiments 1-88, wherein the sequence selected from SEQ ID NOs:1-84 and 100-192 is SEQ ID NO: 32.
   Embodiment 121 The method or composition of any one of embodiments 1-88, wherein the sequence selected from SEQ ID NOs:1-84 and 100-192 is SEQ ID NO: 33.
   Embodiment 122 The method or composition of any one of embodiments 1-88, wherein the sequence selected from SEQ ID NOs:1-84 and 100-192 is SEQ ID NO: 34.
   Embodiment 123 The method or composition of any one of embodiments 1-88, wherein the sequence selected from SEQ ID NOs:1-84 and 100-192 is SEQ ID NO: 35.
   Embodiment 124 The method or composition of any one of embodiments 1-88, wherein the sequence selected from SEQ ID NOs:1-84 and 100-192 is SEQ ID NO: 36.
   Embodiment 125 The method or composition of any one of embodiments 1-88, wherein the sequence selected from SEQ ID NOs:1-84 and 100-192 is SEQ ID NO: 37.
   Embodiment 126 The method or composition of any one of embodiments 1-88, wherein the sequence selected from SEQ ID NOs:1-84 and 100-192 is SEQ ID NO: 38.
   Embodiment 127 The method or composition of any one of embodiments 1-88, wherein the sequence selected from SEQ ID NOs:1-84 and 100-192 is SEQ ID NO: 39.
   Embodiment 128 The method or composition of any one of embodiments 1-88, wherein the sequence selected from SEQ ID NOs:1-84 and 100-192 is SEQ ID NO: 40.
   Embodiment 129 The method or composition of any one of embodiments 1-88, wherein the sequence selected from SEQ ID NOs:1-84 and 100-192 is SEQ ID NO: 41.
   Embodiment 130 The method or composition of any one of embodiments 1-88, wherein the sequence selected from SEQ ID NOs:1-84 and 100-192 is SEQ ID NO: 42.
   Embodiment 131 The method or composition of any one of embodiments 1-88, wherein the sequence selected from SEQ ID NOs:1-84 and 100-192 is SEQ ID NO: 43.
   Embodiment 132 The method or composition of any one of embodiments 1-88, wherein the sequence selected from SEQ ID NOs:1-84 and 100-192 is SEQ ID NO: 44.
   Embodiment 133 The method or composition of any one of embodiments 1-88, wherein the sequence selected from SEQ ID NOs:1-84 and 100-192 is SEQ ID NO: 45.
   Embodiment 134 The method or composition of any one of embodiments 1-88, wherein the sequence selected from SEQ ID NOs:1-84 and 100-192 is SEQ ID NO: 46.
   Embodiment 135 The method or composition of any one of embodiments 1-88, wherein the sequence selected from SEQ ID NOs:1-84 and 100-192 is SEQ ID NO: 47.
   Embodiment 136 The method or composition of any one of embodiments 1-88, wherein the sequence selected from SEQ ID NOs:1-84 and 100-192 is SEQ ID NO: 48.
   Embodiment 137 The method or composition of any one of embodiments 1-88, wherein the sequence selected from SEQ ID NOs:1-84 and 100-192 is SEQ ID NO: 49.
   Embodiment 138 The method or composition of any one of embodiments 1-88, wherein the sequence selected from SEQ ID NOs:1-84 and 100-192 is SEQ ID NO: 50.
   Embodiment 139 The method or composition of any one of embodiments 1-88, wherein the sequence selected from SEQ ID NOs:1-84 and 100-192 is SEQ ID NO: 51.
   Embodiment 140 The method or composition of any one of embodiments 1-88, wherein the sequence selected from SEQ ID NOs:1-84 and 100-192 is SEQ ID NO: 52.
   Embodiment 141 The method or composition of any one of embodiments 1-88, wherein the sequence selected from SEQ ID NOs:1-84 and 100-192 is SEQ ID NO: 53.
   Embodiment 142 The method or composition of any one of embodiments 1-88, wherein the sequence selected from SEQ ID NOs:1-84 and 100-192 is SEQ ID NO: 54.
   Embodiment 143 The method or composition of any one of embodiments 1-88, wherein the sequence selected from SEQ ID NOs:1-84 and 100-192 is SEQ ID NO: 55.
   Embodiment 144 The method or composition of any one of embodiments 1-88, wherein the sequence selected from SEQ ID NOs:1-84 and 100-192 is SEQ ID NO: 56.
   Embodiment 145 The method or composition of any one of embodiments 1-88, wherein the sequence selected from SEQ ID NOs:1-84 and 100-192 is SEQ ID NO: 57.
   Embodiment 146 The method or composition of any one of embodiments 1-88, wherein the sequence selected from SEQ ID NOs:1-84 and 100-192 is SEQ ID NO: 58.
   Embodiment 147 The method or composition of any one of embodiments 1-88, wherein the sequence selected from SEQ ID NOs:1-84 and 100-192 is SEQ ID NO: 59.
   Embodiment 148 The method or composition of any one of embodiments 1-88, wherein the sequence selected from SEQ ID NOs:1-84 and 100-192 is SEQ ID NO: 60.
   Embodiment 149 The method or composition of any one of embodiments 1-88, wherein the sequence selected from SEQ ID NOs:1-84 and 100-192 is SEQ ID NO: 61.
   Embodiment 150 The method or composition of any one of embodiments 1-88, wherein the sequence selected from SEQ ID NOs:1-84 and 100-192 is SEQ ID NO: 62.
   Embodiment 151 The method or composition of any one of embodiments 1-88, wherein the sequence selected from SEQ ID NOs:1-84 and 100-192 is SEQ ID NO: 63.
   Embodiment 152 The method or composition of any one of embodiments 1-88, wherein the sequence selected from SEQ ID NOs:1-84 and 100-192 is SEQ ID NO: 64.
   Embodiment 153 The method or composition of any one of embodiments 1-88, wherein the sequence selected from SEQ ID NOs:1-84 and 100-192 is SEQ ID NO: 65.
   Embodiment 154 The method or composition of any one of embodiments 1-88, wherein the sequence selected from SEQ ID NOs:1-84 and 100-192 is SEQ ID NO: 66.
   Embodiment 155 The method or composition of any one of embodiments 1-88, wherein the sequence selected from SEQ ID NOs:1-84 and 100-192 is SEQ ID NO: 67.
   Embodiment 156 The method or composition of any one of embodiments 1-88, wherein the sequence selected from SEQ ID NOs:1-84 and 100-192 is SEQ ID NO: 68.
   Embodiment 157 The method or composition of any one of embodiments 1-88, wherein the sequence selected from SEQ ID NOs:1-84 and 100-192 is SEQ ID NO: 69.
   Embodiment 158 The method or composition of any one of embodiments 1-88, wherein the sequence selected from SEQ ID NOs:1-84 and 100-192 is SEQ ID NO: 70.
   Embodiment 159 The method or composition of any one of embodiments 1-88, wherein the sequence selected from SEQ ID NOs:1-84 and 100-192 is SEQ ID NO: 71.
   Embodiment 160 The method or composition of any one of embodiments 1-88, wherein the sequence selected from SEQ ID NOs:1-84 and 100-192 is SEQ ID NO: 72.
   Embodiment 161 The method or composition of any one of embodiments 1-88, wherein the sequence selected from SEQ ID NOs:1-84 and 100-192 is SEQ ID NO: 73.
   Embodiment 162 The method or composition of any one of embodiments 1-88, wherein the sequence selected from SEQ ID NOs:1-84 and 100-192 is SEQ ID NO: 74.
   Embodiment 163 The method or composition of any one of embodiments 1-88, wherein the sequence selected from SEQ ID NOs:1-84 and 100-192 is SEQ ID NO: 75.
   Embodiment 164 The method or composition of any one of embodiments 1-88, wherein the sequence selected from SEQ ID NOs:1-84 and 100-192 is SEQ ID NO: 76.
   Embodiment 165 The method or composition of any one of embodiments 1-88, wherein the sequence selected from SEQ ID NOs:1-84 and 100-192 is SEQ ID NO: 77.
   Embodiment 166 The method or composition of any one of embodiments 1-88, wherein the sequence selected from SEQ ID NOs:1-84 and 100-192 is SEQ ID NO: 78.
   Embodiment 167 The method or composition of any one of embodiments 1-88, wherein the sequence selected from SEQ ID NOs:1-84 and 100-192 is SEQ ID NO: 79.
   Embodiment 168 The method or composition of any one of embodiments 1-88, wherein the sequence selected from SEQ ID NOs:1-84 and 100-192 is SEQ ID NO: 80.
   Embodiment 169 The method or composition of any one of embodiments 1-88, wherein the sequence selected from SEQ ID NOs:1-84 and 100-192 is SEQ ID NO: 81.
   Embodiment 170 The method or composition of any one of embodiments 1-88, wherein the sequence selected from SEQ ID NOs:1-84 and 100-192 is SEQ ID NO: 82.
   Embodiment 171 The method or composition of any one of embodiments 1-88, wherein the sequence selected from SEQ ID NOs:1-84 and 100-192 is SEQ ID NO: 83.
   Embodiment 172 The method or composition of any one of embodiments 1-88, wherein the sequence selected from SEQ ID NOs:1-84 and 100-192 is SEQ ID NO: 84.
   Embodiment 173 The method or composition of any one of embodiments 1-88, wherein the sequence selected from SEQ ID NOs:1-84 and 100-192 is SEQ ID NO: 103.
   Embodiment 174 The method or composition of any one of embodiments 1-88, wherein the sequence selected from SEQ ID NOs:1-84 and 100-192 is SEQ ID NO: 109.
   Embodiment 175 The method or composition of any one of embodiments 1-88, wherein the sequence selected from SEQ ID NOs:1-84 and 100-192 is SEQ ID NO: 123.
   Embodiment 176 The method or composition of any one of embodiments 1-88, wherein the sequence selected from SEQ ID NOs:1-84 and 100-192 is SEQ ID NO: 133.
   Embodiment 177 The method or composition of any one of embodiments 1-88, wherein the sequence selected from SEQ ID NOs:1-84 and 100-192 is SEQ ID NO: 149.
   Embodiment 178 The method or composition of any one of embodiments 1-88, wherein the sequence selected from SEQ ID NOs:1-84 and 100-192 is SEQ ID NO: 156.
   Embodiment 179 The method or composition of any one of embodiments 1-88, wherein the sequence selected from SEQ ID NOs:1-84 and 100-192 is SEQ ID NO: 166.
   Embodiment 180 The method or composition of any one of embodiments 1-88, wherein the guide sequence comprises any one of SEQ ID NOs: 2, 9, 13, 16, 22, 24, 25, 27, 30, 31, 32, 33, 35, 36, 40, 44, 45, 53, 55, 57, 60, 61-63, 65, 67, 69, 70, 71, 73, 76, 78, 79, 80, 82-84, 103, 109, 123, 133, 149, 156, and 166.
   Embodiment 181 The method or composition of any one of embodiments 1-88, wherein the guide sequence comprises any one of SEQ ID NOs: 100-102, 104-108, 110-122, 124-132, 134-148, 150-155, 157-165, and 167-192.
   Embodiment 182 The method or composition of any one of embodiments 1-88, wherein the guide sequence comprises any one of SEQ ID NOs: 1, 5, 7, 8, 14, 23, 25, 27, 32, 45, 48, 62, 66, 68, 70, 73, 75, 76, 77, 78, 80, 103, 109, 123, 133, 149, 153, 156, and 184.
   Embodiment 183 The method or composition of any one of embodiments 1-88, wherein the guide sequence comprises any one of SEQ ID NOs: 1, 5, 7, 8, 14, 23, 25, 27, 32, 45, 48, 103, and 123.
   Embodiment 184 The method or composition of any one of embodiments 1-88, wherein the guide RNA is an sgRNA comprising any one of SEQ ID NOs: 86-90.
   Embodiment 185 The method or composition of any one of embodiments 1-88, wherein the guide RNA is an sgRNA comprising SEQ ID NO: 89.
   Embodiment 186 The method or composition of any one of embodiments 1-88, wherein the guide RNA is an sgRNA comprising SEQ ID NO: 1001 or 2001.
   Embodiment 187 The method or composition of any one of embodiments 1-88, wherein the guide RNA is an sgRNA comprising SEQ ID NO: 1005 or 2005.
   Embodiment 188 The method or composition of any one of embodiments 1-88, wherein the guide RNA is an sgRNA comprising SEQ ID NO: 1007 or 2007.
   Embodiment 189 The method or composition of any one of embodiments 1-88, wherein the guide RNA is an sgRNA comprising SEQ ID NO: 1008 or 2008.
   Embodiment 190 The method or composition of any one of embodiments 1-88, wherein the guide RNA is an sgRNA comprising SEQ ID NO: 1014 or 2014.
   Embodiment 191 The method or composition of any one of embodiments 1-88, wherein the guide RNA is an sgRNA comprising SEQ ID NO: 1023 or 2023.
   Embodiment 192 The method or composition of any one of embodiments 1-88, wherein the guide RNA is an sgRNA comprising SEQ ID NO: 1027 or 2027.
   Embodiment 193 The method or composition of any one of embodiments 1-88, wherein the guide RNA is an sgRNA comprising SEQ ID NO: 1032 or 2032.
   Embodiment 194 The method or composition of any one of embodiments 1-88, wherein the guide RNA is an sgRNA comprising SEQ ID NO: 1045 or 2045.
   Embodiment 195 The method or composition of any one of embodiments 1-88, wherein the guide RNA is an sgRNA comprising SEQ ID NO: 1048 or 2048.
   Embodiment 196 The method or composition of any one of embodiments 1-88, wherein the guide RNA is an sgRNA comprising SEQ ID NO: 1063 or 2063.
   Embodiment 197 The method or composition of any one of embodiments 1-88, wherein the guide RNA is an sgRNA comprising SEQ ID NO: 1067 or 2067.
   Embodiment 198 The method or composition of any one of embodiments 1-88, wherein the guide RNA is an sgRNA comprising SEQ ID NO: 1069 or 2069.
   Embodiment 199 The method or composition of any one of embodiments 1-88, wherein the guide RNA is an sgRNA comprising SEQ ID NO: 1071 or 2071.
   Embodiment 200 The method or composition of any one of embodiments 1-88, wherein the guide RNA is an sgRNA comprising SEQ ID NO: 1074 or 2074.
   Embodiment 201 The method or composition of any one of embodiments 1-88, wherein the guide RNA is an sgRNA comprising SEQ ID NO: 1076 or 2076.
   Embodiment 202 The method or composition of any one of embodiments 1-88, wherein the guide RNA is an sgRNA comprising SEQ ID NO: 1077 or 2077.
   Embodiment 203 The method or composition of any one of embodiments 1-88, wherein the guide RNA is an sgRNA comprising SEQ ID NO: 1078 or 2078.
   Embodiment 204 The method or composition of any one of embodiments 1-88, wherein the guide RNA is an sgRNA comprising SEQ ID NO: 1079 or 2079.
   Embodiment 205 The method or composition of any one of embodiments 1-88, wherein the guide RNA is an sgRNA comprising SEQ ID NO: 1081 or 2081.
   Embodiment 206 The method or composition of any one of embodiments 1-205, wherein the composition is administered as a single dose.
   Embodiment 207 The method or composition of any one of embodiments 1-206, wherein the composition is administered one time.
   Embodiment 208 The method or composition of any one of embodiments 206 or 207, wherein the single dose or one time administration:
a. induces a DSB; and/or
b. reduces expression of LDHA gene; and/or
c. treats or prevents hyperoxaluria; and/or
d. treats or prevents ESRD caused by hyperoxaluria; and/or
e. treats or prevents calcium oxalate production and deposition; and/or
f. treats or prevents primary hyperoxaluria (including PH1, PH2, and PH3); and/or
g. treats or prevents oxalosis; and/or
h. treats and prevents hematuria; and/or
i. treats or prevents enteric hyperoxaluria; and/or
j. treats or prevents hyperoxaluria related to eating high-oxalate foods; and/or
k. delays or ameliorates the need for kidney or liver transplant; and/or
l. increases serum glycolate concentration; and/or
m. reduces oxylate in urine.
   Embodiment 209 The method or composition of embodiment 208, wherein the single dose or one time administration achieves any one or more of a)-m) for 3, 4, 5, 6, 7, 8, 9, 10, 11, 12, 13, 14, or 15 weeks.
   Embodiment 210 The method or composition of embodiment 208, wherein the single dose or one time administration achieves a durable effect.
   Embodiment 211 The method or composition of any one of embodiments 1-208, further comprising achieving a durable effect.
   Embodiment 212 The method or composition of embodiment 210 or 211, wherein the durable effect persists at least 1 month, at least 3 months, at least 6 months, at least one year, or at least 5 years.
   Embodiment 213 The method or composition of any one of embodiments 1-212, wherein administration of the composition results in a therapeutically relevant reduction of oxalate in urine.
   Embodiment 214 The method or composition of any one of embodiments 1-213, wherein administration of the composition results in urinary oxalate levels within a therapeutic range.
   Embodiment 215 The method or composition of any one of embodiments 1-214, wherein administration of the composition results in oxalate levels within 100, 120, or 150% of normal range.
   Embodiment 216 Use of a composition or formulation of any of embodiments 9-215 for the preparation of a medicament for treating a human subject having hyperoxaluria.

Also disclosed is the use of a composition or formulation of any of the foregoing embodiments for the preparation of a medicament for treating a human subject having hyperoxaluria. Also disclosed are any of the foregoing compositions or formulations for use in treating hyperoxaluria or for use in modifying (e.g., forming an indel in, or forming a frameshift or nonsense mutation in) a LDHA gene.

### BRIEF DESCRIPTION OF THE DRAWINGS

FIG. 1 shows off-target analysis of certain sgRNAs targeting LDHA.

FIG. 2 shows dose response curves of editing % of certain sgRNAs targeting LDHA in PHH.

FIG. 3 shows dose response curves of editing % of certain sgRNAs targeting LDHA in PCH.

FIG. 4 shows Western Blot analysis of LDHA-targeted modified sgRNAs (listed in Table 2) in PHH.

FIG. 5 shows urine oxalate levels after treatment with LNPs comprising a modified sgRNAs in vivo in AGT-deficient mice.

FIG. 6 shows urine oxalate levels after treatment with LNPs comprising a modified sgRNA in vivo in AGT-deficient mice in a 15-week study.

FIG. 7 shows Western Blot analysis after treatment with LNPs comprising a modified sgRNA in vivo in AGT-deficient mice in a 15-week study.

FIG. 8 shows immunohistochemical staining of LDHA protein in vivo in livers of AGT-deficient mice.

FIG. 9 shows the correlation between the editing and protein levels depicted in Table 19.

FIG. 10 labels the 10 conserved region YA sites in an exemplary sgRNA sequence from 1 to 10 (SEQ ID NO: 2082). The numbers 25, 45, 50, 56, 64, 67, and 83 indicate the position of the pyrimidine of YA sites 1, 5, 6, 7, 8, 9, and 10 in an sgRNA with a guide region indicated as (N)x, e.g., wherein x is optionally 20.

FIG. 11 shows an exemplary sgRNA (SEQ ID NO: 401; not all modifications are shown) in a possible secondary structure with labels designating individual nucleotides of the conserved region of the sgRNA, including the lower stem, bulge, upper stem, nexus (the nucleotides of which can be referred to as N1 through N18, respectively, in the 5′ to 3′ direction), hairpin 1, and hairpin 2 regions. A nucleotide between hairpin 1 and hairpin 2 is labeled n. A guide region may be present on an sgRNA and is indicated in this figure as “(N)x” preceding the conserved region of the sgRNA.

FIGS. 12A-12C show dose response curves of percent editing of certain sgRNAs targeting LDHA in primary cynomologous hepatocytes.

FIGS. 13A-13B show dose response curves of relative reduction in LDHA expression after lipofection treatment comprising certain sgRNAs in primary human and cynomolgus hepatocytes.

FIGS. 14A-14C show dose-dependent urine oxalate levels, percent editing, and correlation between the urine oxalate levels and percent editing, respectively, after treatment with LNPs comprising a certain sgRNA of AGT-deficient mice.

FIGS. 15A-15B show LDHA activity in liver and muscle samples after treatment with LNPs comprising a certain sgRNA of AGT-deficient mice in the 15-week durability study as described in Example 4.

FIGS. 16A-16B show pyruvate levels in liver and plasma samples, after treatment with LNPs comprising a certain sgRNA of AGT-deficient mice in the 15-week durability study as described in Example 4.

FIG. 17 shows the average plasma lactate clearance function in mice that had undergone either 5/6 nephrectomy or sham surgeries after treatment with LNPs comprising a certain sgRNA.

### DETAILED DESCRIPTION

Reference will now be made in detail to certain embodiments of the invention, examples of which are illustrated in the accompanying drawings. While the invention is described in conjunction with the illustrated embodiments, it will be understood that they are not intended to limit the invention to those embodiments. On the contrary, the invention is intended to cover all alternatives, modifications, and equivalents, which may be included within the invention as defined by the appended claims and included embodiments.

Before describing the present teachings in detail, it is to be understood that the disclosure is not limited to specific compositions or process steps, as such may vary. It should be noted that, as used in this specification and the appended claims, the singular form “a”, “an” and “the” include plural references unless the context clearly dictates otherwise. Thus, for example, reference to “a conjugate” includes a plurality of conjugates and reference to “a cell” includes a plurality of cells and the like.

Numeric ranges are inclusive of the numbers defining the range. Measured and measurable values are understood to be approximate, taking into account significant digits and the error associated with the measurement. Also, the use of “comprise”, “comprises”, “comprising”, “contain”, “contains”, “containing”, “include”, “includes”, and “including” are not intended to be limiting. It is to be understood that both the foregoing general description and detailed description are exemplary and explanatory only and are not restrictive of the teachings.

Unless specifically noted in the specification, embodiments in the specification that recite “comprising” various components are also contemplated as “consisting of” or “consisting essentially of” the recited components; embodiments in the specification that recite “consisting of” various components are also contemplated as “comprising” or “consisting essentially of” the recited components; and embodiments in the specification that recite “consisting essentially of” various components are also contemplated as “consisting of” or “comprising” the recited components (this interchangeability does not apply to the use of these terms in the claims). The term “or” is used in an inclusive sense, i.e., equivalent to “and/or,” unless the context clearly indicates otherwise.

The section headings used herein are for organizational purposes only and are not to be construed as limiting the desired subject matter in any way. In the event that any material incorporated by reference contradicts any term defined in this specification or any other express content of this specification, this specification controls. While the present teachings are described in conjunction with various embodiments, it is not intended that the present teachings be limited to such embodiments. On the contrary, the present teachings encompass various alternatives, modifications, and equivalents, as will be appreciated by those of skill in the art.

### I. Definitions

Unless stated otherwise, the following terms and phrases as used herein are intended to have the following meanings:

“Polynucleotide” and “nucleic acid” are used herein to refer to a multimeric compound comprising nucleosides or nucleoside analogs which have nitrogenous heterocyclic bases or base analogs linked together along a backbone, including conventional RNA, DNA, mixed RNA-DNA, and polymers that are analogs thereof. A nucleic acid “backbone” can be made up of a variety of linkages, including one or more of sugar-phosphodiester linkages, peptide-nucleic acid bonds (“peptide nucleic acids” or PNA; PCT No. WO 95/32305), phosphorothioate linkages, methylphosphonate linkages, or combinations thereof. Sugar moieties of a nucleic acid can be ribose, deoxyribose, or similar compounds with substitutions, e.g., 2′ methoxy or 2′ halide substitutions. Nitrogenous bases can be conventional bases (A, G, C, T, U), analogs thereof (e.g., modified uridines such as 5-methoxyuridine, pseudouridine, or N1-methylpseudouridine, or others); inosine; derivatives of purines or pyrimidines (e.g., N⁴-methyl deoxyguanosine, deaza- or aza-purines, deaza- or aza-pyrimidines, pyrimidine bases with substituent groups at the 5 or 6 position (e.g., 5-methylcytosine), purine bases with a substituent at the 2, 6, or 8 positions, 2-amino-6-methylaminopurine, O⁶-methylguanine, 4-thio-pyrimidines, 4-amino-pyrimidines, 4-dimethylhydrazine-pyrimidines, and O⁴-alkyl-pyrimidines; U.S. Pat. No. 5,378,825 and PCT No. WO 93/13121). For general discussion see The Biochemistry of the Nucleic Acids 5-36, Adams et al., ed., 11^{th} ed., 1992). Nucleic acids can include one or more “abasic” residues where the backbone includes no nitrogenous base for position(s) of the polymer (U.S. Pat. No. 5,585,481). A nucleic acid can comprise only conventional RNA or DNA sugars, bases and linkages, or can include both conventional components and substitutions (e.g., conventional bases with 2′ methoxy linkages, or polymers containing both conventional bases and one or more base analogs). Nucleic acid includes “locked nucleic acid” (LNA), an analogue containing one or more LNA nucleotide monomers with a bicyclic furanose unit locked in an RNA mimicking sugar conformation, which enhance hybridization affinity toward complementary RNA and DNA sequences (Vester and Wengel, 2004, Biochemistry 43(42):13233-41). RNA and DNA have different sugar moieties and can differ by the presence of uracil or analogs thereof in RNA and thymine or analogs thereof in DNA.

“Guide RNA”, “gRNA”, and “guide” are used herein interchangeably to refer to either a crRNA (also known as CRISPR RNA), or the combination of a crRNA and a trRNA (also known as tracrRNA). The crRNA and trRNA may be associated as a single RNA molecule (single guide RNA, sgRNA) or in two separate RNA molecules (dual guide RNA, dgRNA). “Guide RNA” or “gRNA” refers to each type. The trRNA may be a naturally-occurring sequence, or a trRNA sequence with modifications or variations compared to naturally-occurring sequences.

As used herein, a “guide sequence” refers to a sequence within a guide RNA that is complementary to a target sequence and functions to direct a guide RNA to a target sequence for binding or modification (e.g., cleavage) by an RNA-guided DNA binding agent. A “guide sequence” may also be referred to as a “targeting sequence,” or a “spacer sequence.” A guide sequence can be 20 base pairs in length, e.g., in the case of Streptococcus pyogenes (i.e., Spy Cas9) and related Cas9 homologs/orthologs. Shorter or longer sequences can also be used as guides, e.g., 15-, 16-, 17-, 18-, 19-, 21-, 22-, 23-, 24-, or 25-nucleotides in length. For example, in some embodiments, the guide sequence comprises at least 17, 18, 19, or 20 contiguous nucleotides of a sequence selected from SEQ ID NOs:1-84. In some embodiments, the target sequence is in a gene or on a chromosome, for example, and is complementary to the guide sequence. In some embodiments, the degree of complementarity or identity between a guide sequence and its corresponding target sequence may be about 75%, 80%, 85%, 90%, 95%, 96%, 97%, 98%, 99%, or 100%. For example, in some embodiments, the guide sequence comprises a sequence with about 75%, 80%, 85%, 90%, 95%, 96%, 97%, 98%, 99%, or 100% identity to at least 17, 18, 19, or 20 contiguous nucleotides of a sequence selected from SEQ ID NOs:1-84. In some embodiments, the guide sequence and the target region may be 100% complementary or identical. In other embodiments, the guide sequence and the target region may contain at least one mismatch. For example, the guide sequence and the target sequence may contain 1, 2, 3, or 4 mismatches, where the total length of the target sequence is at least 17, 18, 19, 20 or more base pairs. In some embodiments, the guide sequence and the target region may contain 1-4 mismatches where the guide sequence comprises at least 17, 18, 19, 20 or more nucleotides. In some embodiments, the guide sequence and the target region may contain 1, 2, 3, or 4 mismatches where the guide sequence comprises 20 nucleotides.

Target sequences for RNA-guided DNA binding agents include both the positive and negative strands of genomic DNA (i.e., the sequence given and the sequence's reverse compliment), as a nucleic acid substrate for an RNA-guided DNA binding agent is a double stranded nucleic acid. Accordingly, where a guide sequence is said to be “complementary to a target sequence”, it is to be understood that the guide sequence may direct a guide RNA to bind to the reverse complement of a target sequence. Thus, in some embodiments, where the guide sequence binds the reverse complement of a target sequence, the guide sequence is identical to certain nucleotides of the target sequence (e.g., the target sequence not including the PAM) except for the substitution of U for T in the guide sequence.

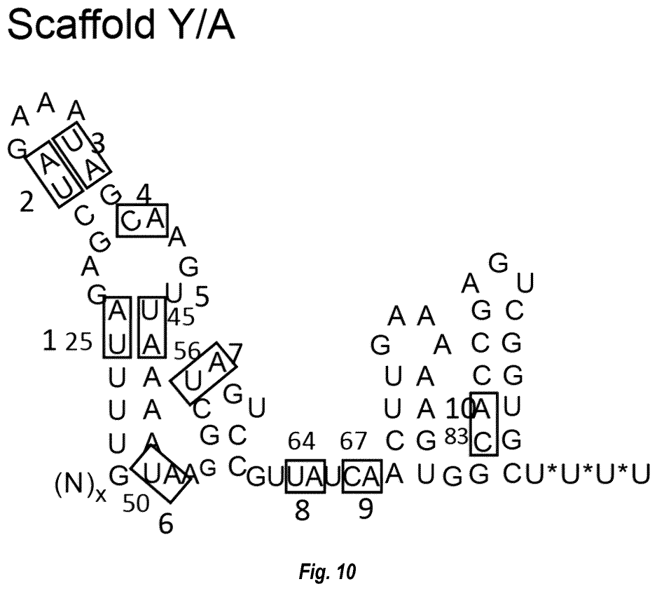

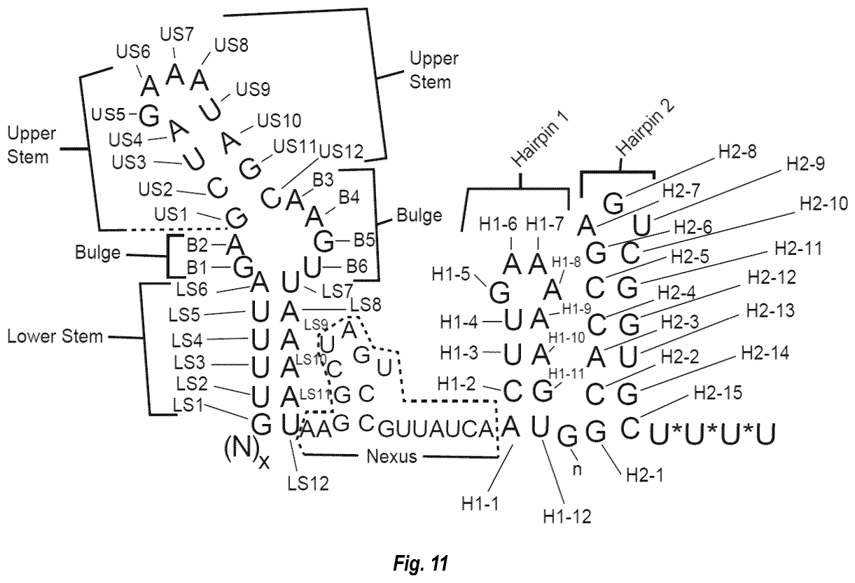

As used herein, a “YA site” refers to a 5′-pyrimidine-adenine-3′ dinucleotide. A “conserved region YA site” is present in the conserved region of an sgRNA. A “guide region YA site” is present in the guide region of an sgRNA. An unmodified YA site in an sgRNA may be susceptible to cleavage by RNase-A like endonucleases, e.g., RNase A. In some embodiments, an sgRNA comprises about 10 YA sites in its conserved region. In some embodiments, an sgRNA comprises 1, 2, 3, 4, 5, 6, 7, 8, 9, or 10 YA sites in its conserved region. Exemplary conserved region YA sites are indicated in FIG. 10. Exemplary guide region YA sites are not shown in FIG. 10, as the guide region may be any sequence, including any number of YA sites. In some embodiments, an sgRNA comprises 1, 2, 3, 4, 5, 6, 7, 8, 9, or 10 of the YA sites indicated in FIG. 10. In some embodiments, an sgRNA comprises 1, 2, 3, 4, 5, 6, 7, 8, 9, or 10 YA sites at the following positions or a subset thereof: LS5-LS6; US3-US4; US9-US10; US12-B3; LS7-LS8; LS12-N1; N6-N7; N14-N15; N17-N18; and H2-2 to H2-3. In some embodiments, a YA site comprises a modification, meaning that at least one nucleotide of the YA site is modified. In some embodiments, the pyrimidine (also called the pyrimidine position) of the YA site comprises a modification (which includes a modification altering the internucleoside linkage immediately 3′ of the sugar of the pyrimidine). In some embodiments, the adenine (also called the adenine position) of the YA site comprises a modification (which includes a modification altering the internucleoside linkage immediately 3′ of the sugar of the adenine). In some embodiments, the pyrimidine position and the adenine position of the YA site comprise modifications.

As used herein, an “RNA-guided DNA binding agent” means a polypeptide or complex of polypeptides having RNA and DNA binding activity, or a DNA-binding subunit of such a complex, wherein the DNA binding activity is sequence-specific and depends on the sequence of the RNA. Exemplary RNA-guided DNA binding agents include Cas cleavases/nickases and inactivated forms thereof (“dCas DNA binding agents”). “Cas nuclease”, also called “Cas protein” as used herein, encompasses Cas cleavases, Cas nickases, and dCas DNA binding agents. Cas cleavases/nickases and dCas DNA binding agents include a Csm or Cmr complex of a type III CRISPR system, the Cas10, Csm1, or Cmr2 subunit thereof, a Cascade complex of a type I CRISPR system, the Cas3 subunit thereof, and Class 2 Cas nucleases. As used herein, a “Class 2 Cas nuclease” is a single-chain polypeptide with RNA-guided DNA binding activity, such as a Cas9 nuclease or a Cpf1 nuclease. Class 2 Cas nucleases include Class 2 Cas cleavases and Class 2 Cas nickases (e.g., H840A, D10A, or N863A variants), which further have RNA-guided DNA cleavases or nickase activity, and Class 2 dCas DNA binding agents, in which cleavase/nickase activity is inactivated. Class 2 Cas nucleases include, for example, Cas9, Cpf1, C2c1, C2c2, C2c3, HF Cas9 (e.g., N497A, R661A, Q695A, Q926A variants), HypaCas9 (e.g., N692A, M694A, Q695A, H698A variants), eSPCas9(1.0) (e.g., K810A, K1003A, R1060A variants), and eSPCas9(1.1) (e.g., K848A, K1003A, R1060A variants) proteins and modifications thereof. Cpf1 protein, Zetsche et al., Cell, 163: 1-13 (2015), is homologous to Cas9, and contains a RuvC-like nuclease domain. Cpf1 sequences of Zetsche are incorporated by reference in their entirety. See, e.g., Zetsche, Tables S1 and S3. “Cas9” encompasses Spy Cas9, the variants of Cas9 listed herein, and equivalents thereof. See, e.g., Makarova et al., Nat Rev Microbiol, 13(11): 722-36 (2015); Shmakov et al., Molecular Cell, 60:385-397 (2015).

As used herein, “ribonucleoprotein” (RNP) or “RNP complex” refers to a guide RNA together with an RNA-guided DNA binding agent, such as a Cas nuclease, e.g., a Cas cleavase, Cas nickase, or dCas DNA binding agent (e.g., Cas9). In some embodiments, the guide RNA guides the RNA-guided DNA binding agent such as Cas9 to a target sequence, and the guide RNA hybridizes with and the agent binds to the target sequence; in cases where the agent is a cleavase or nickase, binding can be followed by cleaving or nicking.

As used herein, a first sequence is considered to “comprise a sequence with at least X % identity to” a second sequence if an alignment of the first sequence to the second sequence shows that X % or more of the positions of the second sequence in its entirety are matched by the first sequence. For example, the sequence AAGA comprises a sequence with 100% identity to the sequence AAG because an alignment would give 100% identity in that there are matches to all three positions of the second sequence. The differences between RNA and DNA (generally the exchange of uridine for thymidine or vice versa) and the presence of nucleoside analogs such as modified uridines do not contribute to differences in identity or complementarity among polynucleotides as long as the relevant nucleotides (such as thymidine, uridine, or modified uridine) have the same complement (e.g., adenosine for all of thymidine, uridine, or modified uridine; another example is cytosine and 5-methylcytosine, both of which have guanosine or modified guanosine as a complement). Thus, for example, the sequence 5′-AXG where X is any modified uridine, such as pseudouridine, N1-methyl pseudouridine, or 5-methoxyuridine, is considered 100% identical to AUG in that both are perfectly complementary to the same sequence (5′-CAU). Exemplary alignment algorithms are the Smith-Waterman and Needleman-Wunsch algorithms, which are well-known in the art. One skilled in the art will understand what choice of algorithm and parameter settings are appropriate for a given pair of sequences to be aligned; for sequences of generally similar length and expected identity >50% for amino acids or >75% for nucleotides, the Needleman-Wunsch algorithm with default settings of the Needleman-Wunsch algorithm interface provided by the EBI at the www.ebi.ac.uk web server is generally appropriate.

“mRNA” is used herein to refer to a polynucleotide that is RNA or modified RNA and comprises an open reading frame that can be translated into a polypeptide (i.e., can serve as a substrate for translation by a ribosome and amino-acylated tRNAs). mRNA can comprise a phosphate-sugar backbone including ribose residues or analogs thereof, e.g., 2′-methoxy ribose residues. In some embodiments, the sugars of an mRNA phosphate-sugar backbone consist essentially of ribose residues, 2′-methoxy ribose residues, or a combination thereof.

Guide sequences useful in the guide RNA compositions and methods described herein are shown in Table 1 and throughout the application.

As used herein, “indels” refer to insertion/deletion mutations consisting of a number of nucleotides that are either inserted or deleted at the site of double-stranded breaks (DSBs) in a target nucleic acid.

As used herein, “knockdown” refers to a decrease in expression of a particular gene product (e.g., protein, mRNA, or both). Knockdown of a protein can be measured by detecting total cellular amount of the protein from a tissue or cell population of interest. Methods for measuring knockdown of mRNA are known and include sequencing of mRNA isolated from a tissue or cell population of interest. In some embodiments, “knockdown” may refer to some loss of expression of a particular gene product, for example a decrease in the amount of mRNA transcribed or a decrease in the amount of protein expressed by a population of cells (including in vivo populations such as those found in tissues).

As used herein, “knockout” refers to a loss of expression of a particular protein in a cell. Knockout can be measured either by detecting total cellular amount of a protein in a cell, a tissue or a population of cells. In some embodiments, the methods of the disclosure “knockout” LDHA in one or more cells (e.g., in a population of cells including in vivo populations such as those found in tissues). In some embodiments, a knockout is not the formation of mutant LDHA protein, for example, created by indels, but rather the complete loss of expression of LDH protein in a cell. As used herein, “LDH” refers to lactate dehydrogenase, which is the gene product of a LDHA gene. The human wild-type LDHA sequence is available at NCBI Gene ID: 3939; Ensembl ENSG00000134333.

“Hyperoxaluria” is a condition characterized by excess oxalate in the urine. Exemplary types of hyperoxaluria include primary hyperoxaluria (including types 1 (PH1), 2 (PH2), and 3 (PH3)), oxalosis, enteric hyperoxaluria, and hyperoxaluria related to eating high-oxalate foods. Hyperoxaluria may be idiopathic. High oxalate levels lead to calcium oxalate stone formation and renal parenchyma damage, which results in progressive deterioration of renal function and, eventually, end-stage renal disease. Thus, hyperoxaluria may result in excessive oxalate production and deposition of calcium oxalate crystals in the kidneys and urinary tract. Renal damage from oxalate is caused by a combination of tubular toxicity, calcium oxalate deposition in the kidneys, and urinary obstruction by calcium oxalate stones. Compromised kidney function exacerbates the disease as the excess oxalate can no longer be effectively excreted, resulting in subsequent accumulation and crystallization of oxalate in bones, eyes, skin, and heart, and other organs leading to severe illness and death. Kidney failure and end stage renal disease may occur. There are no approved pharmaceutical therapies for hyperoxaluria.

“Primary Hyperoxaluria Type 1 (PH1)” is an autosomal recessive disorder due to mutation of the AGXT gene, which encodes the liver peroxisomal alanine-glyoxylate aminotransferase (AGT) enzyme. AGT metabolizes glyoxylate to glycine. The lack of AGT activity, or its mistargeting to mitochondria, allows the oxidation of glyoxylate to oxalate, which can only be excreted in the urine.

Disrupting lactate dehydrogenase (LDH), a hepatic, peroxisomal enzyme that converts glyoxylate to oxylate before excretion by the kidney, is one possible mechanism for blocking oxalate synthesis in diseased livers, to potentially prevent the pathology that develops in hyperoxaluria. LDH, encoded by the lactate dehydrogenase gene (LDHA) gene, catalyzes the conversion of glyoxylate to oxalate. Suppression of LDH activity should inhibit oxalate production resulting in decreased urinary oxalate levels while causing an accumulation of glyoxylate that may be converted to glycolate by glyoxylate reductase/hydroxypyruvate reductase (GRHPR). Unlike oxalate, glycolate is soluble and readily excreted in the urine. Currently there are no known negative side effects of elevated glycolate levels. Thus, in some embodiments, methods for inhibiting LDH activity are provided, wherein once inhibited, oxalate production is inhibited and glycolate production is increased.

Oxalate, an oxidation product of glyoxylate, can only be excreted in the urine. High levels of oxalate in the urine (“hyperoxaluria”) is a symptom of hyperoxaluria. Thus, increased oxalate in the urine is a symptom of hyperoxaluria. Oxalate can combine with calcium to form calcium oxalate, which is the main component of kidney and bladder stones. Deposits of calcium oxalate in the kidneys and other tissues can lead to blood in the urine (hematuria), urinary tract infections, kidney damage, end stage renal disease and others. Over time, oxalate levels in the blood may rise and calcium oxalate may be deposited in other organs throughout the body (oxalosis or systemic oxalosis).

As used herein, a “target sequence” refers to a sequence of nucleic acid in a target gene that has complementarity to the guide sequence of the gRNA. The interaction of the target sequence and the guide sequence directs an RNA-guided DNA binding agent to bind, and potentially nick or cleave (depending on the activity of the agent), within the target sequence.

As used herein, “treatment” refers to any administration or application of a therapeutic for disease or disorder in a subject, and includes inhibiting the disease, arresting its development, relieving one or more symptoms of the disease, curing the disease, or preventing reoccurrence of one or more symptoms of the disease. For example, treatment of hyperoxaluria may comprise alleviating symptoms of hyperoxaluria.

The term “therapeutically relevant reduction of oxalate,” or “oxalate levels within a therapeutic range,” as used herein, means a greater than 30% reduction of urinary oxalate excretion as compared to baseline. See, Leumann and Hoppe (1999) Nephrol Dial Transplant 14:2556-2558 at 2557, second column. For example, achieving oxalate levels within a therapeutic range means reducing urinary oxalate greater than 30% from baseline. In some embodiments, a “normal oxalate level” or a “normal oxalate range” is between about 80 to about 122 μg oxalate/mg creatinine. See, Li et al. (2016) Biochim Biophys Acta 1862(2):233-239. In some embodiments, a therapeutically relevant reduction of oxalate achieves levels of less than or within 200%, 150%, 125%, 120%, 115%, 110%, 105%, or 100% of normal.

The term “about” or “approximately” means an acceptable error for a particular value as determined by one of ordinary skill in the art, which depends in part on how the value is measured or determined.

### II. Compositions

A. Compositions Comprising Guide RNA (gRNAs)

Provided herein are compositions useful for inducing a double-stranded break (DSB) within the LDHA gene, e.g., using a guide RNA with an RNA-guided DNA binding agent (e.g., a CRISPR/Cas system). The compositions may be administered to subjects having or suspected of having hyperoxaluria. The compositions may be administered to subjects having increased urinary oxalate output or decreased serum glycolate output. Guide sequences targeting the LDHA gene are shown in Table 1 at SEQ ID NOs:1-84.

Each of the guide sequences shown in Table 1 at SEQ ID NOs:1-84 and 100-192 may further comprise additional nucleotides to form a crRNA, e.g., with the following exemplary nucleotide sequence following the guide sequence at its 3′ end: GUUJUUAGAGCUAUGCUGUUUUG (SEQ ID NO: 200) in 5′ to 3′ orientation. In the case of a sgRNA, the above guide sequences may further comprise additional nucleotides to form a sgRNA, e.g., with the following exemplary nucleotide sequence following the 3′ end of the guide sequence: GUUUUAGAGCUAGAAAUAGCAAGUUAAAAUAAGGCUAGUCCGUUAUCAACUU GAAAAAGUGGCACCGAGUCGGUGCUUUU (SEQ ID NO: 201) or GUUUUAGAGCUAGAAAUAGCAAGUUAAAAUAAGGCUAGUCCGUUAUCAACUU GAAAAAGUGGCACCGAGUCGGUGC (SEQ ID NO: 203, which is SEQ ID NO: 201 without the four terminal U's) in 5′ to 3′ orientation. In some embodiments, the four terminal U's of SEQ ID NO: 201 are not present. In some embodiments, only 1, 2, or 3 of the four terminal U's of SEQ ID NO: 201 are present.

In some embodiments, LDHA short-single guide RNAs (LDHA short-sgRNAs) are provided comprising a guide sequence as described herein and a “conserved portion of an sgRNA” comprising a hairpin region, wherein the hairpin region lacks at least 5-10 nucleotides or 6-10 nucleotides. In certain embodiments, a hairpin region of the LDHA short-single guide RNAs lacks 5-10 nucleotides with reference to the conserved portion of an sgRNA, e.g. nucleotides H1-1 to H2-15 in Table 2B. In certain embodiments, a hairpin 1 region of the LDHA short-single guide RNAs lacks 5-10 nucleotides with reference to the conserved portion of an sgRNA, e.g. nucleotides H1-1 to H1-12 in Table 2B.

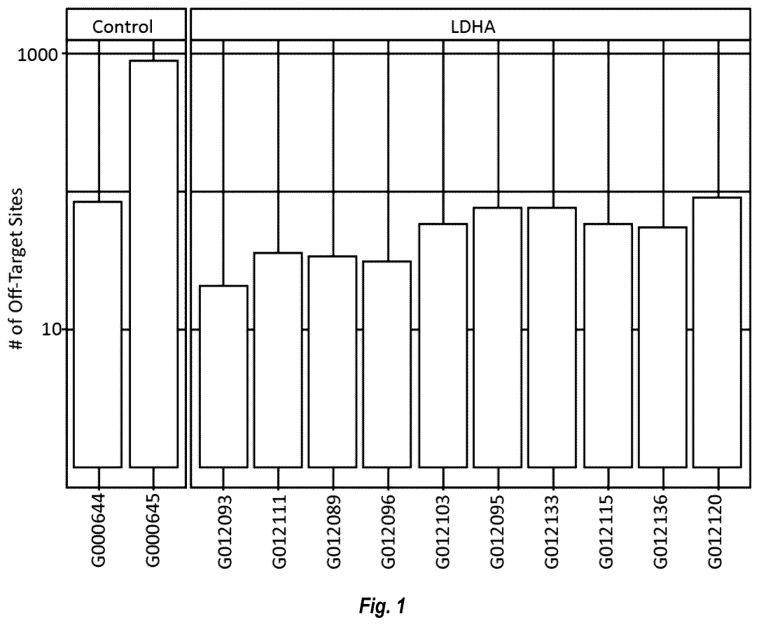

An exemplary “conserved portion of an sgRNA” is shown in Table 2A, which shows a “conserved region” of a S. pyogenes Cas9 (“spyCas9” (also referred to as “spCas9”)) sgRNA. The first row shows the numbering of the nucleotides, the second row shows the sequence (SEQ ID NO: 700); and the third row shows “domains.” Briner A E et al., Molecular Cell 56:333-339 (2014) describes functional domains of sgRNAs, referred to herein as “domains”, including the “spacer” domain responsible for targeting, the “lower stem”, the “bulge”, “upper stem” (which may include a tetraloop), the “nexus”, and the “hairpin 1” and “hairpin 2” domains. See, Briner et al. at page 334, FIG. 1A.

Table 2B provides a schematic of the domains of an sgRNA as used herein. In Table 2B, the “n” between regions represents a variable number of nucleotides, for example, from 0 to 1, 2, 3, 4, 5, 6, 7, 8, 9, 10, 11, 12, 13, 14, 15, 16, 17, 18, 19, 20, or more. In some embodiments, n equals 0. In some embodiments, n equals 1.

In some embodiments, the LDHA sgRNA is from S. pyogenes Cas9 (“spyCas9”) or a spyCas9 equivalent. In some embodiments, the sgRNA is not from S. pyogenes (“non-spyCas9”). In some embodiments, the 5-10 nucleotides or 6-10 nucleotides are consecutive.

In some embodiments, an LDHA short-sgRNA lacks at least nucleotides 54-58 (AAAAA) of the conserved portion of a S. pyogenes Cas9 (“spyCas9”) sgRNA, as shown in Table 2A. In some embodiments, an LDHA short-sgRNA is a non-spyCas9 sgRNA that lacks at least nucleotides corresponding to nucleotides 54-58 (AAAAA) of the conserved portion of a spyCas9 as determined, for example, by pairwise or structural alignment. In some embodiments, the non-spyCas9 sgRNA is Staphylococcus aureus Cas9 (“saCas9”) sgRNA.

In some embodiments, an LDHA short-sgRNA lacks at least nucleotides 54-61 (AAAAAGUG) of the conserved portion of a spyCas9 sgRNA. In some embodiments, an LDHA short-sgRNA lacks at least nucleotides 53-60 (GAAAAAGU) of the conserved portion of a spyCas9 sgRNA. In some embodiments, an LDHA short-sgRNA lacks 4, 5, 6, 7, or 8 nucleotides of nucleotides 53-60 (GAAAAAGU) or nucleotides 54-61 (AAAAAGUG) of the conserved portion of a spyCas9 sgRNA, or the corresponding nucleotides of the conserved portion of a non-spyCas9 sgRNA as determined, for example, by pairwise or structural alignment.

In some embodiments, the sgRNA comprises any one of the guide sequences of SEQ ID NOs: 1-146 and additional nucleotides to form a crRNA, e.g., with the following exemplary nucleotide sequence following the guide sequence at its 3′ end: GUUUUAGAGCUAGAAAUAGCAAGUUAAAAUAAGGCUAGUCCGUUAUCAACUU GGCACCGAGUCGGUGC (SEQ ID NO: 202) in 5′ to 3′ orientation. SEQ ID NO: 202 lacks 8 nucleotides with reference to a wild-type guide RNA conserved sequence:

(SEQ ID NO: 203)
GUUUUAGAGCUAGAAAUAGCAAGUUAAAAUAAGGCUAGUCCGUUAUCAA
CUUGAAAAAGUGGCACCGAGUCGGUGC.

TABLE 1
LDHA targeted guide sequences and chromosomal coordinates for human
and cynomolgus monkey

| | | Exemplary Genomic Coordinates | |
|---|---|---|---|
| | | (“Hs” indicates human; “Cyno” | |
| | | indicates cynomolgus monkey; no | SEQ |
| Guide ID | Guide Sequence | designation is human) | ID NO: |
| G012089 | ACAUAGACCUACCUUAAUCA | chr11: 18405564-18405584 | 1 |
| G012090 | AAAUAACUUAUGCUUACCAC | Hs: chr11: 18403010-18403030 | 2 |
| | | Cyno: chr14: 49278339-49278359 | |
| G012091 | AUGCAGUCAAAAGCCUCACC | chr11: 18401009-18401029 | 3 |
| G012092 | UCAGGGUCUUUACGGAAUAA | chr11: 18407112-18407132 | 4 |
| G012093 | CCUAUCAUACAGUGCUUAUG | chr11: 18405436-18405456 | 5 |
| G012094 | CCGAUUCCGUUACCUAAUGG | chr11: 18402924-18402944 | 6 |
| G012095 | UAGACCUACCUUAAUCAUGG | chr11: 18405561-18405581 | 7 |
| G012096 | UACAGAGAGUCCAAUAGCCC | chr11: 18405486-18405506 | 8 |
| G012097 | CUUUUAGUGCCUGUAUGGAG | Hs: chr11: 18403686-18403706 | 9 |
| | | Cyno: chr14: 49277655-49277675 | |
| G012098 | CCCGAUUCCGUUACCUAAUG | chr11: 18402923-18402943 | 10 |
| G012099 | GGCUGGGGCACGUCAGCAAG | chr11: 18400876-18400896 | 11 |
| G012100 | CCCCAUUAGGUAACGGAAUC | chr11: 18402926-18402946 | 12 |
| G012101 | AAGCUGGUCAUUAUCACGGC | Hs: chr11: 18400859-18400879 | 13 |
| | | Cyno: chr14: 49280125-49280145 | |
| G012103 | UACACUUUGGGGGAUCCAAA | chr11: 18407244-18407264 | 14 |
| G012104 | AUUUGAUGUCUUUUAGGACU | chr11: 18399414-18399434 | 15 |
| G012105 | CUCCAAGCUGGUCAUUAUCA | Hs: chr11: 18400855-18400875 | 16 |
| | | Cyno: chr14: 49280129-49280149 | |
| G012106 | GUCCAAUAUGGCAACUCUAA | chr11: 18396835-18396855 | 17 |
| G012107 | GGCUACACAUCCUGGGCUAU | chr11: 18405473-18405493 | 18 |
| G009440 | UACCUUCAUUAAGAUACUGA | chr11: 18396951-18396971 | 19 |
| G012108 | AGCCCGAUUCCGUUACCUAA | chr11: 18402921-18402941 | 20 |
| G012109 | GCCUUUCCCCCAUUAGGUAA | chr11: 18402933-18402953 | 21 |
| G012110 | UACGCUGGACCAAAUUAAGA | Hs: chr11: 18400909-18400929 | 22 |
| | | Cyno: chr14: 49280075-49280095 | |
| G012111 | UAUUUCUUUUAGUGCCUGUA | chr11: 18403681-18403701 | 23 |
| G012112 | AGCUGGUCAUUAUCACGGCU | Hs: chr11: 18400860-18400880 | 24 |
| | | Cyno: chr14: 49280124-49280144 | |
| G012113 | GCUGGUCAUUAUCACGGCUG | Hs: chr11: 18400861-18400881 | 25 |
| | | Cyno: chr14: 49280123-49280143 | |
| G012114 | GCUGGGGCACGUCAGCAAGA | chr11: 18400877-18400897 | 26 |
| G012115 | CUUUAUCAGUCCCUAAAUCU | Hs: chr11: 18403748-18403768 | 27 |
| | | Cyno: chr14: 49277593-49277613 | |
| G012116 | GCCCGAUUCCGUUACCUAAU | chr11: 18402922-18402942 | 28 |
| G012117 | UUUCAUCUUCAGGGUCUUUA | chr11: 18407104-18407124 | 29 |
| G012118 | ACAACUGUAAUCUUAUUCUG | Hs: chr11: 18396899-18396919 | 30 |
| | | Cyno: chr14: 49282661-49282681 | |
| G012119 | CAUUAAGAUACUGAUGGCAC | Hs: chr11: 18396945-18396965 | 31 |
| | | Cyno: chr17: 59812521-59812541 | |
| G012120 | UUUAGGGACUGAUAAAGAUA | Hs: chr11: 18403751-18403771 | 32 |
| | | Cyno: chr14: 49277590-49277610 | |
| G012121 | CUGAUAAAGAUAAGGAACAG | Hs: chr11: 18403759-18403779 | 33 |
| | | Cyno: chr14: 49277582-49277602 | |
| G012122 | UUACCUAAUGGGGGAAAGGC | chr11: 18402933-18402953 | 34 |
| G012123 | UGGAGUGGAAUGAAUGUUGC | Hs: chr11: 18403701-18403721 | 35 |
| | | Cyno: chr14: 49277640-49277660 | |
| G012124 | UCUUUAUCAGUCCCUAAAUC | Hs: chr11: 18403749-18403769 | 36 |
| | | Cyno: chr14: 49277592-49277612 | |
| G012125 | UCCGUUACCUAAUGGGGGAA | chr11: 18402929-18402949 | 37 |
| G012126 | UAUCUGCACUCUUCUUCAAA | chr11: 18407226-18407246 | 38 |
| G012127 | UACCUAAUGGGGGAAAGGCU | chr11: 18402934-18402954 | 39 |
| G012128 | AGCCGUGAUAAUGACCAGCU | Hs: chr11: 18400860-18400880 | 40 |
| | | Cyno: chr14: 49280124-49280144 | |
| G012129 | CCCCCAUUAGGUAACGGAAU | chr11: 18402927-18402947 | 41 |
| G012130 | UUUAAAAUUGCAGCUCCUUU | chr11: 18407262-18407282 | 42 |
| G012131 | GCUGAUUUAUAAUCUUCUAA | chr11: 18396862-18396882 | 43 |
| G012132 | ACAUUCAUUCCACUCCAUAC | Hs: chr11: 18403698-18403718 | 44 |
| | | Cyno: chr14: 49277643-49277663 | |
| G012133 | CCUUAAUCAUGGUGGAAACU | Hs: chr11: 18405553-18405573 | 45 |
| | | Cyno: chr12: 38488548-38488568 | |
| G012134 | ACCUUAAUCAUGGUGGAAAC | chr11: 18405554-18405574 | 46 |
| G012135 | CCUUUGCCAGAGACAAUCUU | chr11: 18399529-18399549 | 47 |
| G012136 | GAAGGUGACUCUGACUUCUG | chr11: 18407193-18407213 | 48 |
| G012137 | UAUUGGAAGCGGUUGCAAUC | chr11: 18402894-18402914 | 49 |
| G012138 | AAGUCAGAGUCACCUUCACA | chr11: 18407190-18407210 | 50 |
| G012139 | GACUCUGACUUCUGAGGAAG | chr11: 18407199-18407219 | 51 |
| G012140 | UGCAACCGCUUCCAAUAACA | chr11: 18402891-18402911 | 52 |
| G012141 | UAUUUUCUCCUUUUUCAUAG | Hs: chr11: 18402819-18402839 | 53 |
| | | Cyno: chr14: 49278530-49278550 | |
| G012142 | UUUUUUUCAUUUCAUCUUCA | chr11: 18407095-18407115 | 54 |
| G012143 | ACCAAAGUAGUCACUGUUCA | Cyno: chr14: 49274629-49274649 | 55 |
| G012145 | ACGCAGUUAAAAGGCUCACC | chr14: 49279975-49279995 | 56 |
| G012146 | UUGCUUAUUGUUUCAAAUCC | Cyno: chr14: 49279996-49280016 | 57 |
| | | Hs: chr11: 18400988-18401008 | |
| G012147 | UUCCCCCUAUAGAUUCCUUC | chr14: 49282754-49282774 | 58 |
| G012148 | UCGAGCUUUGUGGCAGUUAG | chr14: 49283162-49283182 | 59 |
| G012149 | UUGGGGUUAAUAAACCGCGA | Cyno: chr14: 49283034-49283054 | 60 |
| | | Hs: chr11: 18396528-18396548 | |
| G012150 | UGAAGGCCCAUACCUUAGCG | Cyno: chr14: 49282959-49282979 | 61 |
| | | Hs: chr11: 18396603-18396623 | |
| G012151 | CGGUUUAUUAACCCCAAGUG | Cyno: chr14: 49283037-49283057 | 62 |
| | | Hs: chr11: 18396525-18396545 | |
| G012152 | CCCAUACCUUAGCGUGGAAA | Cyno: chr14: 49282965-49282985 | 63 |
| | | Hs: chr11: 18396597-18396617 | |
| G012153 | GGCUUUUCUGCACGUACCUC | chr14: 49283141-49283161 | 64 |
| G012154 | GAAAAGGAAUAUCGACGUUU | Cyno: chr14: 49282981-49283001 | 65 |
| | | Hs: chr11: 18396581-18396601 | |
| G012155 | ACCGCGAUGGGUGAGCCCUC | chr14: 49283021-49283041 | 66 |
| G012156 | GCGGUUUAUUAACCCCAAGU | Cyno: chr14: 49283036-49283056 | 67 |
| | | Hs: chr11: 18396526-18396546 | |
| G012157 | ACCGCACGCUUCAGUGCCUU | chr14: 49283186-49283206 | 68 |
| G012158 | GGAAAAGGAAUAUCGACGUU | Cyno: chr14: 49282980-49283000 | 69 |
| | | Hs: chr11: 18396582-18396602 | |
| G012159 | GUGUAAGUAUAGCCUCCUGA | Cyno: chr14: 49283003-49283023 | 70 |
| | | Hs: chr11: 18396559-18396579 | |
| G012160 | GAUAUUCCUUUUCCACGCUA | Cyno: chr14: 49282974-49282994 | 71 |
| | | Hs: chr11: 18396588-18396608 | |
| G012161 | GCGAUGGGUGAGCCCUCAGG | chr14: 49283018-49283038 | 72 |
| G012162 | GGAAAGGCCAGCCCCACUUG | Cyno: chr14: 49283051-49283071 | 73 |
| | | Hs: chr11: 18396511-18396531 | |
| G012163 | CACCGCACGCUUCAGUGCCU | chr14: 49283187-49283207 | 74 |
| G012164 | UGCCACAAAGCUCGAGCCCA | chr14: 49283167-49283187 | 75 |
| G012165 | GGUGUAAGUAUAGCCUCCUG | Cyno: chr14: 49283002-49283022 | 76 |
| | | Hs: chr11: 18396560-18396580 | |
| G012166 | UCCUGAGGGCUCACCCAUCG | chr14: 49283017-49283037 | 77 |
| G012167 | AGGAAAGGCCAGCCCCACUU | Cyno: chr14: 49283052-49283072 | 78 |
| | | Hs: chr11: 18396510-18396530 | |
| G012168 | UUAUUAACCCCAAGUGGGGC | Cyno: chr14: 49283041-49283061 | 79 |
| | | Hs: chr11: 18396521-18396541 | |
| G012169 | GAGGAAAGGCCAGCCCCACU | Cyno: chr14: 49283053-49283073 | 80 |
| | | Hs: chr11: 18396509-18396529 | |
| G012170 | GCUCAAAGUGAUCUUGUCUG | chr14: 49283072-49283092 | 81 |
| G012171 | CCUGGCUGUGUCCUUGCUGU | Cyno: chr14: 49283105-49283125 | 82 |
| | | Hs: chr11: 18396457-18396477 | |
| G012172 | CGCGGUUUAUUAACCCCAAG | Cyno: chr14: 49283035-49283055 | 83 |
| | | Hs: chr11: 18396527-18396547 | |
| G012173 | UGGGGUUAAUAAACCGCGAU | Cyno: chr14: 49283033-49283053 | 84 |
| | | Hs: chr11: 18396529-18396549 | |
| G015538 | UUUCCCAAAAACCGUGUUAU | Cyno: chr14: 49278472-49278492 | 100 |
| G015539 | GAAAGAGGUUCACAAGCAGG | Cyno: chr14: 49277560-49277580 | 101 |
| G015540 | GUGGAAAGAGGUUCACAAGC | Cyno: chr14: 49277563-49277583 | 102 |
| G015541 | GAGAUGAUGGAUCUCCAACA | Cyno: chr12: 38487918-38487938 | 103 |
| | | Hs: chr11: 18399484-18399504 | |
| G015542 | UAAGGAAAAGGCUGCCAUGU | Cyno: chr17: 59812615-59812635 | 104 |
| G015543 | UGUAACUGCAAACUCCAAGC | Cyno: chr14: 49280141-49280161 | 105 |
| G015544 | CUUCCAAUAACACGGUUUUU | Cyno: chr14: 49278466-49278486 | 106 |
| G015545 | AAAAACCGUGUUAUUGGAAG | Cyno: chr14: 49278466-49278486 | 107 |
| G015546 | GUUCACCCAUUAAGCUGUCA | Cyno: chr14: 49278391-49278411 | 108 |
| G015547 | UUCACCCAUUAAGCUGUCAU | Cyno: chr14: 49278390-49278410 | 109 |
| | | Hs: chr11: 18402959-18402979 | |
| G015548 | ACCCAUUAAGCUGUCAUGGG | Cyno: chr14: 49278387-49278407 | 110 |
| G015549 | UGGAAUCUCCAUGUUCCCCA | Cyno: chr14: 49278359-49278379 | 111 |
| G015550 | AGAGUAUAAUGAAGAAUCUU | Cyno: chr12: 38488514-38488534 | 112 |
| G015551 | GCUGAUUCAUAAUCUUCUAA | Cyno: chr14: 49282698-49282718 | 113 |
| G015552 | CAAAUUGAAGGGAGAGAUGA | Cyno: chr12: 38487905-38487925 | 114 |
| G015553 | UCUUUGGUGUUCUAAGGAAA | Cyno: chr12: 38487947-38487967 | 115 |
| G015554 | CAAUAAGCAACUUGCAGUUC | Cyno: chr14: 49280006-49280026 | 116 |
| G015555 | ACAAUAAGCAACUUGCAGUU | Cyno: chr14: 49280005-49280025 | 117 |
| G015556 | GCUUAUUGUUUCAAAUCCAG | Cyno: chr12: 38488136-38488156 | 118 |
| G015557 | ACUUCCAAUAACACGGUUUU | Cyno: chr14: 49278465-49278485 | 119 |
| G015558 | CCCAUUAAGCUGUCAUGGGU | Cyno: chr14: 49278386-49278406 | 120 |
| G015559 | UCCACUCCAUACAGGCACAC | Cyno: chr12: 38488327-38488347 | 121 |
| G015560 | AAGACUCUGCACCCAGAUUU | Cyno: chr14: 49277607-49277627 | 122 |
| G015561 | AGACUCUGCACCCAGAUUUA | Cyno: chr14: 49277606-49277626 | 123 |
| | | Hs: chr11: 18403735-18403755 | |
| G015562 | CCAGUUUCCACCAUGAUUAA | Cyno: chr12: 38488546-38488566 | 124 |
| G015563 | ACCAUGAUUAAGGGUCUCUA | Cyno: chr12: 38488555-38488575 | 125 |
| G015564 | AUAGAGACCCUUAAUCAUGG | Cyno: chr12: 38488556-38488576 | 126 |
| G015565 | UCCAUAGAGACCCUUAAUCA | Cyno: chr12: 38488559-38488579 | 127 |
| G015566 | UAAGGGUCUCUAUGGAAUAA | Cyno: chr12: 38488563-38488583 | 128 |
| G015567 | AGAUAAGGAACAGUGGAAAG | Cyno: chr14: 49277575-49277595 | 129 |
| G015568 | CAGAAUAAGAUUACAGUUGU | Cyno: chr14: 49282661-49282681 | 130 |
| G015569 | AGAAUAAGAUUACAGUUGUU | Cyno: chr14: 49282660-49282680 | 131 |
| G015570 | AACAACUGUAAUCUUAUUCU | Cyno: chr14: 49282660-49282680 | 132 |
| G015571 | GAAUAAGAUUACAGUUGUUG | Cyno: chr14: 49282659-49282679 | 133 |
| | | Hs: chr11: 18396901-18396921 | |
| G015572 | CAACAACUGUAAUCUUAUUC | Cyno: chr14: 49282659-49282679 | 134 |
| G015573 | AAGAUUACAGUUGUUGGGGU | Cyno: chr14: 49282655-49282675 | 135 |
| G015574 | GUUGUUGGGGUUGGUGCUGU | Cyno: chr14: 49282646-49282666 | 136 |
| G015575 | UGCCAUCAGUAUCUUAAUGA | Cyno: chr17: 59812522-59812542 | 137 |
| G015576 | GUCCUUCAUUAAGAUACUGA | Cyno: chr17: 59812527-59812547 | 138 |
| G015577 | CAGUAUCUUAAUGAAGGACU | Cyno: chr17: 59812528-59812548 | 139 |
| G015578 | UGUCAUCGAAGACAAAUUGA | Cyno: chr12: 38487893-38487913 | 140 |
| G015579 | GUCAUCGAAGACAAAUUGAA | Cyno: chr12: 38487894-38487914 | 141 |
| G015580 | AGACAAUCUUUGGUGUUCUA | Cyno: chr12: 38487953-38487973 | 142 |
| G015581 | AGAACACCAAAGAUUGUCUC | Cyno: chr12: 38487954-38487974 | 143 |
| G015582 | GGCUGGGGCACGUCAACAAG | Cyno: chr14: 49280108-49280128 | 144 |
| G015583 | GCUGGGGCACGUCAACAAGA | Cyno: chr14: 49280107-49280127 | 145 |
| G015584 | GGGAGAAAGCCGUCUUAAUU | Cyno: chr14: 49280087-49280107 | 146 |
| G015585 | UAAAGAUGUUCACGUUACGC | Cyno: chr14: 49280060-49280080 | 147 |
| G015586 | GGGCUGUAUUUUACAACAUU | Cyno: chr14: 49280026-49280046 | 148 |
| G015587 | UACGUGGCUUGGAAGAUAAG | Cyno: chr14: 49278496-49278516 | 149 |
| | | Hs: chr11: 18402853-18402873 | |
| G015588 | ACUUAUCUUCCAAGCCACGU | Cyno: chr14: 49278495-49278515 | 150 |
| G015589 | UGCAACCACUUCCAAUAACA | Cyno: chr14: 49278458-49278478 | 151 |
| G015590 | AGCCAGAUUCCGUUACCUGA | Cyno: chr14: 49278428-49278448 | 152 |
| G015591 | GCCAGAUUCCGUUACCUGAU | Cyno: chr14: 49278427-49278447 | 153 |
| G015592 | CCAGAUUCCGUUACCUGAUG | Cyno: chr14: 49278426-49278446 | 154 |
| G015593 | CCCCAUCAGGUAACGGAAUC | Cyno: chr14: 49278423-49278443 | 155 |
| G015594 | CCCACCCAUGACAGCUUAAU | Cyno: chr14: 49278383-49278403 | 156 |
| | | Hs: chr11: 18402966-18402986 | |
| G015595 | ACCCACCCAUGACAGCUUAA | Cyno: chr14: 49278382-49278402 | 157 |
| G015596 | AGCUGUCAUGGGUGGGUCCU | Cyno: chr14: 49278379-49278399 | 158 |
| G015597 | GCUGUCAUGGGUGGGUCCUU | Cyno: chr14: 49278378-49278398 | 159 |
| G015598 | CUGUCAUGGGUGGGUCCUUG | Cyno: chr14: 49278377-49278397 | 160 |
| G015599 | GGGUGGGUCCUUGGGGAACA | Cyno: chr14: 49278370-49278390 | 161 |
| G015600 | GAGAUUCCAGUGUGCCUGUA | Cyno: chr12: 38488318-38488338 | 162 |
| G015601 | UCCAGUGUGCCUGUAUGGAG | Cyno: chr12: 38488323-38488343 | 163 |
| G015602 | AUCUGGGUGCAGAGUCUUCA | Cyno: chr14: 49277609-49277629 | 164 |
| G015603 | AAUCUGGGUGCAGAGUCUUC | Cyno: chr14: 49277608-49277628 | 165 |
| G015604 | UAUGAGGUGAUCAAACUCAA | Cyno: chr12: 38488447-38488467 | 166 |
| | | Hs: chr11: 18405452-18405472 | |
| G015605 | UGGACUCUCUGUAGCAGAUU | Cyno: chr12: 38488488-38488508 | 167 |
| G015606 | CCCAGUUUCCACCAUGAUUA | Cyno: chr12: 38488545-38488565 | 168 |
| G015607 | UGGGGUUGGUGCUGUUGGCA | Cyno: chr12: 38487815-38487835 | 169 |
| G015608 | GAACACCAAAGAUUGUCUCU | Cyno: chr17: 59812635-59812655 | 170 |
| G015609 | CAGAUUCCGUUACCUGAUGG | Cyno: chr14: 49278425-49278445 | 171 |
| G015610 | UUACCUGAUGGGGGAAAGAC | Cyno: chr14: 49278416-49278436 | 172 |
| G015611 | GUCUUUCCCCCAUCAGGUAA | Cyno: chr14: 49278416-49278436 | 173 |
| G015612 | UACCUGAUGGGGGAAAGACU | Cyno: chr14: 49278415-49278435 | 174 |
| G015613 | CUCCCAGUCUUUCCCCCAUC | Cyno: chr14: 49278410-49278430 | 175 |
| G015614 | AACUCAAAGGCUACACAUCC | Cyno: chr17: 59813145-59813165 | 176 |
| G015615 | ACUCAAAGGCUACACAUCCU | Cyno: chr17: 59813146-59813166 | 177 |
| G015616 | GGCUACACAUCCUGGGCCAU | Cyno: chr17: 59813153-59813173 | 178 |
| G015617 | UACAGAGAGUCCAAUGGCCC | Cyno: chr17: 59813166-59813186 | 179 |
| G015618 | AUCUGCUACAGAGAGUCCAA | Cyno: chr17: 59813172-59813192 | 180 |
| G015619 | CCCUUAAUCAUGGUGGAAAC | Cyno: chr12: 38488549-38488569 | 181 |
| G015620 | CCUUGCAUUUUGGGACAGAA | Cyno: chr17: 59813291-59813311 | 182 |
| G015621 | CCAUUCUGUCCCAAAAUGCA | Cyno: chr17: 59813294-59813314 | 183 |
| G015622 | AGUGGAUAUCUUGACCUACG | Cyno: chr14: 49278512-49278532 | 184 |
| G015623 | AUAUCUUGACCUACGUGGCU | Cyno: chr14: 49278507-49278527 | 185 |
| G015624 | UAUUGGAAGUGGUUGCAAUC | Cyno: chr14: 49278455-49278475 | 186 |
| G015625 | UCUUUCCCAGAGACAAUCUU | Cyno: chr17: 59812643-59812663 | 187 |
| G015626 | GGUGGUUGAGAGUGCUUAUG | Cyno: chr17: 59813116-59813136 | 188 |
| G015627 | CCUCAGUGUUCCUUGCAUUU | Cyno: chr17: 59813281-59813301 | 189 |
| G015628 | CUCAGUGUUCCUUGCAUUUU | Cyno: chr17: 59813282-59813302 | 190 |
| G015629 | CCAAAAUGCAAGGAACACUG | Cyno: chr17: 59813284-59813304 | 191 |
| G015630 | ACGUAGGUCAAGAUAUCCAC | Cyno: chr12: 38488155-38488175 | 192 |

TABLE 2
LDHA targeted gRNA and sgRNA nomenclature and sequence

| | Guide | | | | |
|---|---|---|---|---|---|
| Guide ID | ID | | SEQ ID | | SEQ ID |
| (sgRNA) | (crRNA) | sgRNA Sequence-unmodified | NO | sgRNA Sequence-modified | NO |
| G012089 | CR0011780 | ACAUAGACCUACCUUAAUCAGUUUUAGAGCUAGAAA | 1001 | mA*mC*mA*UAGACCUACCUUAAUCAGUUUUAGAmGmCmUmAmGmAmAm | 2001 |
| | | UAGCAAGUUAAAAUAAGGCUAGUCCGUUAUCAACUU | | AmUmAmGmCAAGUUAAAAUAAGGCUAGUCCGUUAUCAmAmCmUmUmGmA | |
| | | GAAAAAGUGGCACCGAGUCGGUGCUUUU | | mAmAmAmAmGmUmGmGmCmAmCmCmGmAmGmUmCmGmGmUmGmCmU*mU | |
| | | | | *mU*mU | |
| G012093 | CR0011784 | CCUAUCAUACAGUGCUUAUGGUUUUAGAGCUAGAAA | 1005 | mC*mC*mU*AUCAUACAGUGCUUAUGGUUUUAGAmGmCmUmAmGmAmAm | 2005 |
| | | UAGCAAGUUAAAAUAAGGCUAGUCCGUUAUCAACUU | | AmUmAmGmCAAGUUAAAAUAAGGCUAGUCCGUUAUCAmAmCmUmUmGmA | |
| | | GAAAAAGUGGCACCGAGUCGGUGCUUUU | | mAmAmAmAmGmUmGmGmCmAmCmCmGmAmGmUmCmGmGmUmGmCmU*mU | |
| | | | | *mU*mU | |
| G012095 | CR0011786 | UAGACCUACCUUAAUCAUGGGUUUUAGAGCUAGAAA | 1007 | mU*mA*mG*ACCUACCUUAAUCAUGGGUUUUAGAmGmCmUmAmGmAmAm | 2007 |
| | | UAGCAAGUUAAAAUAAGGCUAGUCCGUUAUCAACUU | | AmUmAmGmCAAGUUAAAAUAAGGCUAGUCCGUUAUCAmAmCmUmUmGmA | |
| | | GAAAAAGUGGCACCGAGUCGGUGCUUUU | | mAmAmAmAmGmUmGmGmCmAmCmCmGmAmGmUmCmGmGmUmGmCmU*mU | |
| | | | | *mU*mU | |
| G012096 | CR0011787 | UACAGAGAGUCCAAUAGCCCGUUUUAGAGCUAGAAA | 1008 | mU*mA*mC*AGAGAGUCCAAUAGCCCGUUUUAGAmGmCmUmAmGmAmAm | 2008 |
| | | UAGCAAGUUAAAAUAAGGCUAGUCCGUUAUCAACUU | | AmUmAmGmCAAGUUAAAAUAAGGCUAGUCCGUUAUCAmAmCmUmUmGmA | |
| | | GAAAAAGUGGCACCGAGUCGGUGCUUUU | | mAmAmAmAmGmUmGmGmCmAmCmCmGmAmGmUmCmGmGmUmGmCmU*mU | |
| | | | | *mU*mU | |
| G012103 | CR0011793 | UACACUUUGGGGGAUCCAAAUUUUAGAGCUAGAAAU | 1014 | mU*mA*mC*ACUUUGGGGGAUCCAAAGUUUUAGAmGmCmUmAmGmAmAm | 2014 |
| | | AGCAAGUUAAAAUAAGGCUAGUCCGUUAUCAACUUG | | AmUmAmGmCAAGUUAAAAUAAGGCUAGUCCGUUAUCAmAmCmUmUmGmA | |
| | | AAAAAGUGGCACCGAGUCGGUGCUUUU | | mAmAmAmAmGmUmGmGmCmAmCmCmGmAmGmUmCmGmGmUmGmCmU*mU | |
| | | | | *mU*mU | |
| G012111 | CR0011801 | UAUUUCUUUUAGUGCCUGUAGUUUUAGAGCUAGAAA | 1023 | mU*mA*mU*UUCUUUUAGUGCCUGUAGUUUUAGAmGmCmUmAmGmAmAm | 2023 |
| | | UAGCAAGUUAAAAUAAGGCUAGUCCGUUAUCAACUU | | AmUmAmGmCAAGUUAAAAUAAGGCUAGUCCGUUAUCAmAmCmUmUmGmA | |
| | | GAAAAAGUGGCACCGAGUCGGUGCUUUU | | mAmAmAmAmGmUmGmGmCmAmCmCmGmAmGmUmCmGmGmUmGmCmU*mU | |
| | | | | *mU*mU | |
| G012115 | CR0011805 | CUUUAUCAGUCCCUAAAUCUUUUUAGAGCUAGAAAU | 1027 | mC*mU*mU*UUUAUCAGUCCCUAAAUCUGUUUUAGAmGmCmUmAmGmAm | 2027 |
| | | AGCAAGUUAAAAUAAGGCUAGUCCGUUAUCAACUUG | | AmAmUmAmGmCAAGUUAAAAUAAGGCUAGUCCGUUAUCAmAmCmUmUmG | |
| | | AAAAAGUGGCACCGAGUCGGUGCUUUU | | mAmAmAmAmAmGmUmGmGmCmAmCmCmGmAmGmUmCmGmGmUmGmCmU* | |
| | | | | mU*mU*mU | |
| G012120 | CR0011810 | UUUAGGGACUGAUAAAGAUAUUUUAGAGCUAGAAAU | 1032 | mU*mU*mU*AGGGACUGAUAAAGAUAGUUUUAGAmGmCmUmAmGmAmAm | 2032 |
| | | AGCAAGUUAAAAUAAGGCUAGUCCGUUAUCAACUUG | | AmUmAmGmCAAGUUAAAAUAAGGCUAGUCCGUUAUCAmAmCmUmUmGmA | |
| | | AAAAAGUGGCACCGAGUCGGUGCUUUU | | mAmAmAmAmGmUmGmGmCmAmCmCmGmAmGmUmCmGmGmUmGmCmU*mU | |
| | | | | *mU*mU | |
| G012133 | CR0011823 | CCUUAAUCAUGGUGGAAACUUUUUAGAGCUAGAAAU | 1045 | mC*mC*mU*UAAUCAUGGUGGAAACUGUUUUAGAmGmCmUmAmGmAmAm | 2045 |
| | | AGCAAGUUAAAAUAAGGCUAGUCCGUUAUCAACUUG | | AmUmAmGmCAAGUUAAAAUAAGGCUAGUCCGUUAUCAmAmCmUmUmGmA | |
| | | AAAAAGUGGCACCGAGUCGGUGCUUUU | | mAmAmAmAmGmUmGmGmCmAmCmCmGmAmGmUmCmGmGmUmGmCmU*mU | |
| | | | | *mU*mU | |
| G012136 | CR0011826 | GAAGGUGACUCUGACUUCUGGUUUUAGAGCUAGAAA | 1048 | mG*mA*mA*GGUGACUCUGACUUCUGUUUUAGAmGmCmUmAmGmAmAmA | 2048 |
| | | UAGCAAGUUAAAAUAAGGCUAGUCCGUUAUCAACUU | | mUmAmGmCAAGUUAAAAUAAGGCUAGUCCGUUAUCAmAmCmUmUmGmAm | |
| | | GAAAAAGUGGCACCGAGUCGGUGCUUUU | | AmAmAmAmGmUmGmGmCmAmCmCmGmAmGmUmCmGmGmUmGmCmU*mU* | |
| | | | | mU*mU | |
| G012151 | CR0011840 | CGGUUUAUUAACCCCAAGUGGUUUUAGAGCUAGAAA | 1063 | mC*mG*mG*UUUAUUAACCCCAAGUG | 2063 |
| | | UAGCAAGUUAAAAUAAGGCUAGUCCGUUAUCAACUU | | GUUUUAGAmGmCmUmAmGmAmAmAmUmAmGmCAAGUUAAAAUAAGGCUA | |
| | | GAAAAAGUGGCACCGAGUCGGUGCUUUU | | GUCCGUUAUCAmAmCmUmUmGmAmAmAmAmAmGmUmGmGmCmAmCmCmG | |
| | | | | mAmGmUmCmGmGmUmGmCmU*mU*mU*mU | |
| G012155 | CR0011844 | ACCGCGAUGGGUGAGCCCUCGUUUUAGAGCUAGAAA | 1067 | mA*mC*mC*GCGAUGGGUGAGCCCUC | 2067 |
| | | UAGCAAGUUAAAAUAAGGCUAGUCCGUUAUCAACUU | | GUUUUAGAmGmCmUmAmGmAmAmAmUmAmGmCAAGUUAAAAUAAGGCUA | |
| | | GAAAAAGUGGCACCGAGUCGGUGCUUUU | | GUCCGUUAUCAmAmCmUmUmGmAmAmAmAmAmGmUmGmGmCmAmCmCmG | |
| | | | | mAmGmUmCmGmGmUmGmCmU*mU*mU*mU | |
| G012157 | CR0011846 | ACCGCACGCUUCAGUGCCUUGUUUUAGAGCUAGAAA | 1069 | mA*mC*mC*GCACGCUUCAGUGCCUU | 2069 |
| | | UAGCAAGUUAAAAUAAGGCUAGUCCGUUAUCAACUU | | GUUUUAGAmGmCmUmAmGmAmAmAmUmAmGmCAAGUUAAAAUAAGGCUA | |
| | | GAAAAAGUGGCACCGAGUCGGUGCUUUU | | GUCCGUUAUCAmAmCmUmUmGmAmAmAmAmAmGmUmGmGmCmAmCmCmG | |
| | | | | mAmGmUmCmGmGmUmGmCmU*mU*mU*mU | |
| G012159 | CR0011848 | GUGUAAGUAUAGCCUCCUGAGUUUUAGAGCUAGAAA | 1071 | mG*mU*mG*UAAGUAUAGCCUCCUGA | 2071 |
| | | UAGCAAGUUAAAAUAAGGCUAGUCCGUUAUCAACUU | | GUUUUAGAmGmCmUmAmGmAmAmAmUmAmGmCAAGUUAAAAUAAGGCUA | |
| | | GAAAAAGUGGCACCGAGUCGGUGCUUUU | | GUCCGUUAUCAmAmCmUmUmGmAmAmAmAmAmGmUmGmGmCmAmCmCmG | |
| | | | | mAmGmUmCmGmGmUmGmCmU*mU*mU*mU | |
| G012162 | CR0011851 | GGAAAGGCCAGCCCCACUUGGUUUUAGAGCUAGAAA | 1074 | mG*mG*mA*AAGGCCAGCCCCACUUG | 2074 |
| | | UAGCAAGUUAAAAUAAGGCUAGUCCGUUAUCAACUU | | GUUUUAGAmGmCmUmAmGmAmAmAmUmAmGmCAAGUUAAAAUAAGGCUA | |
| | | GAAAAAGUGGCACCGAGUCGGUGCUUUU | | GUCCGUUAUCAmAmCmUmUmGmAmAmAmAmAmGmUmGmGmCmAmCmCmG | |
| | | | | mAmGmUmCmGmGmUmGmCmU*mU*mU*mU | |
| G012164 | CR0011853 | UGCCACAAAGCUCGAGCCCAGUUUUAGAGCUAGAAA | 1076 | mU*mG*mC*CACAAAGCUCGAGCCCA | 2076 |
| | | UAGCAAGUUAAAAUAAGGCUAGUCCGUUAUCAACUU | | GUUUUAGAmGmCmUmAmGmAmAmAmUmAmGmCAAGUUAAAAUAAGGCUA | |
| | | GAAAAAGUGGCACCGAGUCGGUGCUUUU | | GUCCGUUAUCAmAmCmUmUmGmAmAmAmAmAmGmUmGmGmCmAmCmCmG | |
| | | | | mAmGmUmCmGmGmUmGmCmU*mU*mU*mU | |
| G012165 | CR0011854 | GGUGUAAGUAUAGCCUCCUGGUUUUAGAGCUAGAAA | 1077 | mG*mG*mU*GUAAGUAUAGCCUCCUG | 2077 |
| | | UAGCAAGUUAAAAUAAGGCUAGUCCGUUAUCAACUU | | GUUUUAGAmGmCmUmAmGmAmAmAmUmAmGmCAAGUUAAAAUAAGGCUA | |
| | | GAAAAAGUGGCACCGAGUCGGUGCUUUU | | GUCCGUUAUCAmAmCmUmUmGmAmAmAmAmAmGmUmGmGmCmAmCmCmG | |
| | | | | mAmGmUmCmGmGmUmGmCmU*mU*mU*mU | |
| G012166 | CR0011855 | UCCUGAGGGCUCACCCAUCGUUUUAGAGCUAGAAAU | 1078 | mU*mC*mC*UGAGGGCUCACCCAUCG | 2078 |
| | | AGCAAGUUAAAAUAAGGCUAGUCCGUUAUCAACUUG | | GUUUUAGAmGmCmUmAmGmAmAmAmUmAmGmCAAGUUAAAAUAAGGCUA | |
| | | AAAAAGUGGCACCGAGUCGGUGCUUUU | | GUCCGUUAUCAmAmCmUmUmGmAmAmAmAmAmGmUmGmGmCmAmCmCmG | |
| | | | | mAmGmUmCmGmGmUmGmCmU*mU*mU*mU | |
| G012167 | CR0011856 | AGGAAAGGCCAGCCCCACUUGUUUUAGAGCUAGAAA | 1079 | mA*mG*mG*AAAGGCCAGCCCCACUU | 2079 |
| | | UAGCAAGUUAAAAUAAGGCUAGUCCGUUAUCAACUU | | GUUUUAGAmGmCmUmAmGmAmAmAmUmAmGmCAAGUUAAAAUAAGGCUA | |
| | | GAAAAAGUGGCACCGAGUCGGUGCUUUU | | GUCCGUUAUCAmAmCmUmUmGmAmAmAmAmAmGmUmGmGmCmAmCmCmG | |
| | | | | mAmGmUmCmGmGmUmGmCmU*mU*mU*mU | |
| G012169 | CR0011858 | GAGGAAAGGCCAGCCCCACUGUUUUAGAGCUAGAAA | 1081 | mG*mA*mG*GAAAGGCCAGCCCCACU | 2081 |
| | | UAGCAAGUUAAAAUAAGGCUAGUCCGUUAUCAACUU | | GUUUUAGAmGmCmUmAmGmAmAmAmUmAmGmCAAGUUAAAAUAAGGCUA | |
| | | GAAAAAGUGGCACCGAGUCGGUGCUUUU | | GUCCGUUAUCAmAmCmUmUmGmAmAmAmAmAmGmUmGmGmCmAmCmCmG | |
| | | | | mAmGmUmCmGmGmUmGmCmU*mU*mU*mU | |

TABLE 2A
(Conserved Portion of a spyCas9 sgRNA; SEQ ID NO: 400)

| 1 | 2 | 3 | 4 | 5 | 6 | 7 | 8 | 9 | 10 | 11 | 12 | 13 | 14 | 15 |
|---|---|---|---|---|---|---|---|---|---|---|---|---|---|---|
| G | U | U | U | U | A | G | A | G | C | U | A | G | A | A |
| LS1-LS6 | | | | | | B1-B2 | | US1-US12 | | | | | | |
| 16 | 17 | 18 | 19 | 20 | 21 | 22 | 23 | 24 | 25 | 26 | 27 | 28 | 29 | 30 |
| A | U | A | G | C | A | A | G | U | U | A | A | A | A | U |
| US1-US12 | | | | | B2-B6 | | | | LS7-LS12 | | | | | |
| 31 | 32 | 33 | 34 | 35 | 36 | 37 | 38 | 39 | 40 | 41 | 42 | 43 | 44 | 45 |
| A | A | G | G | C | U | A | G | U | C | C | G | U | U | A |

Nexus

| 46 | 47 | 48 | 49 | 50 | 51 | 52 | 53 | 54 | 55 | 56 | 57 | 58 | 59 | 60 |
|---|---|---|---|---|---|---|---|---|---|---|---|---|---|---|
| U | C | A | A | C | U | U | G | A | A | A | A | A | G | U |

| | |
|---|---|
| Nexus | H1-1 through H1-12 |

| 61 | 62 | 63 | 64 | 65 | 66 | 67 | 68 |
|---|---|---|---|---|---|---|---|
| G | G | C | A | C | C | G | A |
| N | H2-1 through H2-15 | | | | | | |
| 69 | 70 | 71 | 72 | 73 | 74 | 75 | 76 |
| G | U | C | G | G | U | G | C |

H2-1 through H2-15

TABLE 2B

| | LS1-6 | | B1 -2 | | US1-12 | | B3-6 | |
|---|---|---|---|---|---|---|---|---|
| 5′ terminus (n) | lower stem | n | bulge | n | upper stem | n | bulge | n |

| LS7-12 | | N1-18 | | H1-1 thru H1-12 | | H2-1 thru H2-15 | |
|---|---|---|---|---|---|---|---|
| lower stem | n | nexus | n | hairpin 1 | n | hairpin 2 | 3′ terminus |

In some embodiments, the invention provides a composition comprising one or more guide RNA (gRNA) comprising guide sequences that direct an RNA-guided DNA binding agent, which can be a nuclease (e.g., a Cas nuclease such as Cas9), to a target DNA sequence in LDHA. The gRNA may comprise a crRNA comprising a guide sequence shown in Table 1. The gRNA may comprise a crRNA comprising 17, 18, 19, or 20 contiguous nucleotides of a guide sequence shown in Table 1. In some embodiments, the gRNA comprises a crRNA comprising a sequence with about 75%, 80%, 85%, 90%, 95%, 96%, 97%, 98%, 99%, or 100% identity to at least 17, 18, 19, or 20 contiguous nucleotides of a guide sequence shown in Table 1. In some embodiments, the gRNA comprises a crRNA comprising a sequence with about 75%, 80%, 85%, 90%, 95%, 96%, 97%, 98%, 99%, or 100% identity to a guide sequence shown in Table 1. The gRNA may further comprise a trRNA. In each composition and method embodiment described herein, the crRNA and trRNA may be associated as a single RNA (sgRNA) or may be on separate RNAs (dgRNA). In the context of sgRNAs, the crRNA and trRNA components may be covalently linked, e.g., via a phosphodiester bond or other covalent bond.

In each of the composition, use, and method embodiments described herein, the guide RNA may comprise two RNA molecules as a “dual guide RNA” or “dgRNA”. The dgRNA comprises a first RNA molecule comprising a crRNA comprising, e.g., a guide sequence shown in Table 1, and a second RNA molecule comprising a trRNA. The first and second RNA molecules may not be covalently linked but may form an RNA duplex via the base pairing between portions of the crRNA and the trRNA.

In each of the composition, use, and method embodiments described herein, the guide RNA may comprise a single RNA molecule as a “single guide RNA” or “sgRNA”. The sgRNA may comprise a crRNA (or a portion thereof) comprising a guide sequence shown in Table 1 covalently linked to a trRNA. The sgRNA may comprise 17, 18, 19, or 20 contiguous nucleotides of a guide sequence shown in Table 1. In some embodiments, the crRNA and the trRNA are covalently linked via a linker. In some embodiments, the sgRNA forms a stem-loop structure via the base pairing between portions of the crRNA and the trRNA. In some embodiments, the crRNA and the trRNA are covalently linked via one or more bonds that are not a phosphodiester bond.

In some embodiments, the trRNA may comprise all or a portion of a trRNA sequence derived from a naturally-occurring CRISPR/Cas system. In some embodiments, the trRNA comprises a truncated or modified wild type trRNA. The length of the trRNA depends on the CRISPR/Cas system used. In some embodiments, the trRNA comprises or consists of 5, 6, 7, 8, 9, 10, 11, 12, 13, 14, 15, 16, 17, 18, 19, 20, 25, 30, 40, 50, 60, 70, 80, 90, 100, or more than 100 nucleotides. In some embodiments, the trRNA may comprise certain secondary structures, such as, for example, one or more hairpin or stem-loop structures, or one or more bulge structures.

In some embodiments, the invention provides a composition comprising one or more guide RNAs comprising a guide sequence of any one of SEQ ID NOs:1-84.

In some embodiments, the invention provides a composition comprising one or more sgRNAs comprising any one of SEQ ID NOs: 1001, 1005, 1007, 1008, 1014, 1023, 1027, 1032, 1045, 1048, 1063, 1067, 1069, 1071, 1074, 1076, 1077, 1078, 1079, and 1081, or modified versions thereof as shown, e.g., in SEQ ID NOs: 2001, 2005, 2007, 2008, 2014, 2023, 2027, 2032, 2045, 2048, 2063, 2067, 2069, 2071, 2074, 2076, 2077, 2078, 2079, and 2081.

In one aspect, the invention provides a composition comprising a gRNA that comprises a guide sequence that is at least 99%, 98%, 97%, 96%, 95%, 94%, 93%, 92%, 91%, or 90% identical to any of the nucleic acids of SEQ ID NOs:1-84.

In other embodiments, the composition comprises at least one, e.g., at least two gRNA's comprising guide sequences selected from any two or more of the guide sequences of SEQ ID NOs:1-84. In some embodiments, the composition comprises at least two gRNA's that each comprise a guide sequence at least 99%, 98%, 97%, 96%, 95%, 94%, 93%, 92%, 91%, or 90% identical to any of the nucleic acids of SEQ ID NOs:1-84.

The guide RNA compositions of the present invention are designed to recognize (e.g., hybridize to) a target sequence in the LDHA gene. For example, the LDHA target sequence may be recognized and cleaved by a provided Cas cleavase comprising a guide RNA. In some embodiments, an RNA-guided DNA binding agent, such as a Cas cleavase, may be directed by a guide RNA to a target sequence of the LDHA gene, where the guide sequence of the guide RNA hybridizes with the target sequence and the RNA-guided DNA binding agent, such as a Cas cleavase, cleaves the target sequence.

In some embodiments, the selection of the one or more guide RNAs is determined based on target sequences within the LDHA gene.

Without being bound by any particular theory, mutations (e.g., frameshift mutations resulting from indels occurring as a result of a nuclease-mediated DSB) in certain regions of the gene may be less tolerable than mutations in other regions of the gene, thus the location of a DSB is an important factor in the amount or type of protein knockdown that may result. In some embodiments, a gRNA complementary or having complementarity to a target sequence within LDHA is used to direct the RNA-guided DNA binding agent to a particular location in the LDHA gene. In some embodiments, gRNAs are designed to have guide sequences that are complementary or have complementarity to target sequences in exon 1, exon 2, exon 3, exon 4, exon 5, exon 6, exon 7 or exon 8 of LDHA.

In some embodiments, the guide sequence is at least 99%, 98%, 97%, 96%, 95%, 94%, 93%, 92%, 91%, or 90% identical to a target sequence present in the human LDHA gene. In some embodiments, the target sequence may be complementary to the guide sequence of the guide RNA. In some embodiments, the degree of complementarity or identity between a guide sequence of a guide RNA and its corresponding target sequence may be at least 80%, 85%, 90%, 95%, 96%, 97%, 98%, 99%, or 100%. In some embodiments, the target sequence and the guide sequence of the gRNA may be 100% complementary or identical. In other embodiments, the target sequence and the guide sequence of the gRNA may contain at least one mismatch. For example, the target sequence and the guide sequence of the gRNA may contain 1, 2, 3, or 4 mismatches, where the total length of the guide sequence is 20. In some embodiments, the target sequence and the guide sequence of the gRNA may contain 1-4 mismatches where the guide sequence is 20 nucleotides.

In some embodiments, a composition or formulation disclosed herein comprises an mRNA comprising an open reading frame (ORF) encoding an RNA-guided DNA binding agent, such as a Cas nuclease as described herein. In some embodiments, an mRNA comprising an ORF encoding an RNA-guided DNA binding agent, such as a Cas nuclease, is provided, used, or administered.

B. Modified gRNAs and mRNAs

In some embodiments, the gRNA is chemically modified. A gRNA comprising one or more modified nucleosides or nucleotides is called a “modified” gRNA or “chemically modified” gRNA, to describe the presence of one or more non-naturally and/or naturally occurring components or configurations that are used instead of or in addition to the canonical A, G, C, and U residues. In some embodiments, a modified gRNA is synthesized with a non-canonical nucleoside or nucleotide, is here called “modified.” Modified nucleosides and nucleotides can include one or more of: (i) alteration, e.g., replacement, of one or both of the non-linking phosphate oxygens and/or of one or more of the linking phosphate oxygens in the phosphodiester backbone linkage (an exemplary backbone modification); (ii) alteration, e.g., replacement, of a constituent of the ribose sugar, e.g., of the 2′ hydroxyl on the ribose sugar (an exemplary sugar modification); (iii) wholesale replacement of the phosphate moiety with “dephospho” linkers (an exemplary backbone modification); (iv) modification or replacement of a naturally occurring nucleobase, including with a non-canonical nucleobase (an exemplary base modification); (v) replacement or modification of the ribose-phosphate backbone (an exemplary backbone modification); (vi) modification of the 3′ end or 5′ end of the oligonucleotide, e.g., removal, modification or replacement of a terminal phosphate group or conjugation of a moiety, cap or linker (such 3′ or 5′ cap modifications may comprise a sugar and/or backbone modification); and (vii) modification or replacement of the sugar (an exemplary sugar modification).

Chemical modifications such as those listed above can be combined to provide modified gRNAs and/or mRNAs comprising nucleosides and nucleotides (collectively “residues”) that can have two, three, four, or more modifications. For example, a modified residue can have a modified sugar and a modified nucleobase. In some embodiments, every base of a gRNA is modified, e.g., all bases have a modified phosphate group, such as a phosphorothioate group. In certain embodiments, all, or substantially all, of the phosphate groups of an gRNA molecule are replaced with phosphorothioate groups. In some embodiments, modified gRNAs comprise at least one modified residue at or near the 5′ end of the RNA. In some embodiments, modified gRNAs comprise at least one modified residue at or near the 3′ end of the RNA.

In some embodiments, the gRNA comprises one, two, three or more modified residues. In some embodiments, at least 5% (e.g., at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or 100%) of the positions in a modified gRNA are modified nucleosides or nucleotides.

Unmodified nucleic acids can be prone to degradation by, e.g., intracellular nucleases or those found in serum. For example, nucleases can hydrolyze nucleic acid phosphodiester bonds. Accordingly, in one aspect the gRNAs described herein can contain one or more modified nucleosides or nucleotides, e.g., to introduce stability toward intracellular or serum-based nucleases. In some embodiments, the modified gRNA molecules described herein can exhibit a reduced innate immune response when introduced into a population of cells, both in vivo and ex vivo. The term “innate immune response” includes a cellular response to exogenous nucleic acids, including single stranded nucleic acids, which involves the induction of cytokine expression and release, particularly the interferons, and cell death.

In some embodiments of a backbone modification, the phosphate group of a modified residue can be modified by replacing one or more of the oxygens with a different substituent. Further, the modified residue, e.g., modified residue present in a modified nucleic acid, can include the wholesale replacement of an unmodified phosphate moiety with a modified phosphate group as described herein. In some embodiments, the backbone modification of the phosphate backbone can include alterations that result in either an uncharged linker or a charged linker with unsymmetrical charge distribution.

Examples of modified phosphate groups include, phosphorothioate, phosphoroselenates, borano phosphates, borano phosphate esters, hydrogen phosphonates, phosphoroamidates, alkyl or aryl phosphonates and phosphotriesters. The phosphorous atom in an unmodified phosphate group is achiral. However, replacement of one of the non-bridging oxygens with one of the above atoms or groups of atoms can render the phosphorous atom chiral. The stereogenic phosphorous atom can possess either the “R” configuration (herein Rp) or the “S” configuration (herein Sp). The backbone can also be modified by replacement of a bridging oxygen, (i.e., the oxygen that links the phosphate to the nucleoside), with nitrogen (bridged phosphoroamidates), sulfur (bridged phosphorothioates) and carbon (bridged methylenephosphonates). The replacement can occur at either linking oxygen or at both of the linking oxygens.

The phosphate group can be replaced by non-phosphorus containing connectors in certain backbone modifications. In some embodiments, the charged phosphate group can be replaced by a neutral moiety. Examples of moieties which can replace the phosphate group can include, without limitation, e.g., methyl phosphonate, hydroxylamino, siloxane, carbonate, carboxymethyl, carbamate, amide, thioether, ethylene oxide linker, sulfonate, sulfonamide, thioformacetal, formacetal, oxime, methyleneimino, methylenemethylimino, methylenehydrazo, methylenedimethylhydrazo and methyleneoxymethylimino.

Scaffolds that can mimic nucleic acids can also be constructed wherein the phosphate linker and ribose sugar are replaced by nuclease resistant nucleoside or nucleotide surrogates. Such modifications may comprise backbone and sugar modifications. In some embodiments, the nucleobases can be tethered by a surrogate backbone. Examples can include, without limitation, the morpholino, cyclobutyl, pyrrolidine and peptide nucleic acid (PNA) nucleoside surrogates.

The modified nucleosides and modified nucleotides can include one or more modifications to the sugar group, i.e. at sugar modification. For example, the 2′ hydroxyl group (OH) can be modified, e.g. replaced with a number of different “oxy” or “deoxy” substituents. In some embodiments, modifications to the 2′ hydroxyl group can enhance the stability of the nucleic acid since the hydroxyl can no longer be deprotonated to form a 2′-alkoxide ion.

Examples of 2′ hydroxyl group modifications can include alkoxy or aryloxy (OR, wherein “R” can be, e.g., alkyl, cycloalkyl, aryl, aralkyl, heteroaryl or a sugar); polyethyleneglycols (PEG), O(CH₂CH₂O)ₙCH₂CH₂OR wherein R can be, e.g., H or optionally substituted alkyl, and n can be an integer from 0 to 20 (e.g., from 0 to 4, from 0 to 8, from 0 to 10, from 0 to 16, from 1 to 4, from 1 to 8, from 1 to 10, from 1 to 16, from 1 to 20, from 2 to 4, from 2 to 8, from 2 to 10, from 2 to 16, from 2 to 20, from 4 to 8, from 4 to 10, from 4 to 16, and from 4 to 20). In some embodiments, the 2′ hydroxyl group modification can be 2′-O-Me. In some embodiments, the 2′ hydroxyl group modification can be a 2′-fluoro modification, which replaces the 2′ hydroxyl group with a fluoride. In some embodiments, the 2′ hydroxyl group modification can include “locked” nucleic acids (LNA) in which the 2′ hydroxyl can be connected, e.g., by a C₁₋₆ alkylene or C₁₋₆ heteroalkylene bridge, to the 4′ carbon of the same ribose sugar, where exemplary bridges can include methylene, propylene, ether, or amino bridges; O-amino (wherein amino can be, e.g., NH₂; alkylamino, dialkylamino, heterocyclyl, arylamino, diarylamino, heteroarylamino, or diheteroarylamino, ethylenediamine, or polyamino) and aminoalkoxy, O(CH₂)n-amino, (wherein amino can be, e.g., NH₂; alkylamino, dialkylamino, heterocyclyl, arylamino, diarylamino, heteroarylamino, or diheteroarylamino, ethylenediamine, or polyamino). In some embodiments, the 2′ hydroxyl group modification can include “unlocked” nucleic acids (UNA) in which the ribose ring lacks the C2′-C3′ bond. In some embodiments, the 2′ hydroxyl group modification can include the methoxyethyl group (MOE), (OCH₂CH₂OCH₃, e.g., a PEG derivative).

“Deoxy” 2′ modifications can include hydrogen (i.e. deoxyribose sugars, e.g., at the overhang portions of partially dsRNA); halo (e.g., bromo, chloro, fluoro, or iodo); amino (wherein amino can be, e.g., NH₂; alkylamino, dialkylamino, heterocyclyl, arylamino, diarylamino, heteroarylamino, diheteroarylamino, or amino acid); NH(CH₂CH₂NH)˜CH2CH₂— amino (wherein amino can be, e.g., as described herein), —NHC(O)R (wherein R can be, e.g., alkyl, cycloalkyl, aryl, aralkyl, heteroaryl or sugar), cyano; mercapto; alkyl-thio-alkyl; thioalkoxy; and alkyl, cycloalkyl, aryl, alkenyl and alkynyl, which may be optionally substituted with e.g., an amino as described herein.

The sugar modification can comprise a sugar group which may also contain one or more carbons that possess the opposite stereochemical configuration than that of the corresponding carbon in ribose. Thus, a modified nucleic acid can include nucleotides containing e.g., arabinose, as the sugar. The modified nucleic acids can also include abasic sugars. These abasic sugars can also be further modified at one or more of the constituent sugar atoms. The modified nucleic acids can also include one or more sugars that are in the L form, e.g. L-nucleosides.

The modified nucleosides and modified nucleotides described herein, which can be incorporated into a modified nucleic acid, can include a modified base, also called a nucleobase. Examples of nucleobases include, but are not limited to, adenine (A), guanine (G), cytosine (C), and uracil (U). These nucleobases can be modified or wholly replaced to provide modified residues that can be incorporated into modified nucleic acids. The nucleobase of the nucleotide can be independently selected from a purine, a pyrimidine, a purine analog, or pyrimidine analog. In some embodiments, the nucleobase can include, for example, naturally-occurring and synthetic derivatives of a base.

In embodiments employing a dual guide RNA, each of the crRNA and the tracr RNA can contain modifications. Such modifications may be at one or both ends of the crRNA and/or tracr RNA. In embodiments comprising an sgRNA, one or more residues at one or both ends of the sgRNA may be chemically modified, and/or internal nucleosides may be modified, and/or the entire sgRNA may be chemically modified. Certain embodiments comprise a 5′ end modification. Certain embodiments comprise a 3′ end modification.

In some embodiments, the guide RNAs disclosed herein comprise one of the modification patterns disclosed in WO2018/107028 A1, filed Dec. 8, 2017, titled “Chemically Modified Guide RNAs,” the contents of which are hereby incorporated by reference in their entirety. In some embodiments, the guide RNAs disclosed herein comprise one of the structures/modification patterns disclosed in US20170114334, the contents of which are hereby incorporated by reference in their entirety. In some embodiments, the guide RNAs disclosed herein comprise one of the structures/modification patterns disclosed in WO2017/136794, the contents of which are hereby incorporated by reference in their entirety.

### C. YA Modifications

A modification at a YA site (also referred to herein as “YA modification”) can be a modification of the internucleoside linkage, a modification of the base (pyrimidine or adenine), e.g. by chemical modification, substitution, or otherwise, and/or a modification of the sugar (e.g. at the 2′ position, such as 2′-O-alkyl, 2′-F, 2′-moe, 2′-F arabinose, 2′-H (deoxyribose), and the like). In some embodiments, a “YA modification” is any modification that alters the structure of the dinucleotide motif to reduce RNA endonuclease activity, e.g., by interfering with recognition or cleavage of a YA site by an RNase and/or by stabilizing an RNA structure (e.g., secondary structure) that decreases accessibility of a cleavage site to an RNase. See Peacock et al., J Org Chem. 76: 7295-7300 (2011); Behlke, Oligonucleotides 18:305-320 (2008); Ku et al., Adv. Drug Delivery Reviews 104: 16-28 (2016); Ghidini et al., Chem. Commun., 2013, 49, 9036. Peacock et al., Belhke, Ku, and Ghidini provide exemplary modifications suitable as YA modifications. Modifications known to those of skill in the art to reduce endonucleolytic degradation are encompassed. Exemplary 2′ ribose modifications that affect the 2′ hydroxyl group involved in RNase cleavage are 2′-H and 2′-O-alkyl, including 2′-O-Me. Modifications such as bicyclic ribose analogs, UNA, and modified internucleoside linkages of the residues at the YA site can be YA modifications. Exemplary base modifications that can stabilize RNA structures are pseudouridine and 5-methylcytosine. In some embodiments, at least one nucleotide of the YA site is modified. In some embodiments, the pyrimidine (also called “pyrimidine position”) of the YA site comprises a modification (which includes a modification altering the internucleoside linkage immediately 3′ of the sugar of the pyrimidine, a modification of the pyrimidine base, and a modification of the ribose, e.g. at its 2′ position). In some embodiments, the adenine (also called “adenine position”) of the YA site comprises a modification (which includes a modification altering the internucleoside linkage immediately 3′ of the sugar of the pyrimidine, a modification of the pyrimidine base, and a modification of the ribose, e.g. at its 2′ position). In some embodiments, the pyrimidine and the adenine of the YA site comprise modifications. In some embodiments, the YA modification reduces RNA endonuclease activity.

In some embodiments, an sgRNA comprises modifications at 1, 2, 3, 4, 5, 6, 7, 8, 9, 10, 11, 12, 13, 14, 15, 16, or more YA sites. In some embodiments, the pyrimidine of the YA site comprises a modification (which includes a modification altering the internucleoside linkage immediately 3′ of the sugar of the pyrimidine). In some embodiments, the adenine of the YA site comprises a modification (which includes a modification altering the internucleoside linkage immediately 3′ of the sugar of the adenine). In some embodiments, the pyrimidine and the adenine of the YA site comprise modifications, such as sugar, base, or internucleoside linkage modifications. The YA modifications can be any of the types of modifications set forth herein. In some embodiments, the YA modifications comprise one or more of phosphorothioate, 2′-OMe, or 2′-fluoro. In some embodiments, the YA modifications comprise pyrimidine modifications comprising one or more of phosphorothioate, 2′-OMe, or 2′-fluoro. In some embodiments, the YA modification comprises a bicyclic ribose analog (e.g., an LNA, BNA, or ENA) within an RNA duplex region that contains one or more YA sites. In some embodiments, the YA modification comprises a bicyclic ribose analog (e.g., an LNA, BNA, or ENA) within an RNA duplex region that contains a YA site, wherein the YA modification is distal to the YA site.

In some embodiments, the sgRNA comprises a guide region YA site modification. In some embodiments, the guide region comprises 1, 2, 3, 4, 5, or more YA sites (“guide region YA sites”) that may comprise YA modifications. In some embodiments, one or more YA sites located at 5-end, 6-end, 7-end, 8-end, 9-end, or 10-end from the 5′ end of the 5′ terminus (where “5-end”, etc., refers to position 5 to the 3′ end of the guide region, i.e., the most 3′ nucleotide in the guide region) comprise YA modifications. In some embodiments, two or more YA sites located at 5-end, 6-end, 7-end, 8-end, 9-end, or 10-end from the 5′ end of the 5′ terminus comprise YA modifications. In some embodiments, three or more YA sites located at 5-end, 6-end, 7-end, 8-end, 9-end, or 10-end from the 5′ end of the 5′ terminus comprise YA modifications. In some embodiments, four or more YA sites located at 5-end, 6-end, 7-end, 8-end, 9-end, or 10-end from the 5′ end of the 5′ terminus comprise YA modifications. In some embodiments, five or more YA sites located at 5-end, 6-end, 7-end, 8-end, 9-end, or 10-end from the 5′ end of the 5′ terminus comprise YA modifications. A modified guide region YA site comprises a YA modification.

In some embodiments, a modified guide region YA site is within 17, 16, 15, 14, 13, 12, 11, 10, or 9 nucleotides of the 3′ terminal nucleotide of the guide region. For example, if a modified guide region YA site is within 10 nucleotides of the 3′ terminal nucleotide of the guide region and the guide region is 20 nucleotides long, then the modified nucleotide of the modified guide region YA site is located at any of positions 11-20. In some embodiments, a YA modification is located within a YA site 20, 19, 18, 17, 16, 15, 14, 13, 12, 11, 10, 9, 8, 7, 6, 5, 4, 3, 2, or 1 nucleotides from the 3′ terminal nucleotide of the guide region. In some embodiments, a YA modification is located 20, 19, 18, 17, 16, 15, 14, 13, 12, 11, 10, 9, 8, 7, 6, 5, 4, 3, 2, or 1 nucleotides from the 3′ terminal nucleotide of the guide region.

In some embodiments, a modified guide region YA site is at or after nucleotide 4, 5, 6, 7, 8, 9, 10, or 11 from the 5′ end of the 5′ terminus.

In some embodiments, a modified guide region YA site is other than a 5′ end modification. For example, an sgRNA can comprise a 5′ end modification as described herein and further comprise a modified guide region YA site. Alternatively, an sgRNA can comprise an unmodified 5′ end and a modified guide region YA site. Alternatively, an sgRNA can comprise a modified 5′ end and an unmodified guide region YA site.

In some embodiments, a modified guide region YA site comprises a modification that at least one nucleotide located 5′ of the guide region YA site does not comprise. For example, if nucleotides 1-3 comprise phosphorothioates, nucleotide 4 comprises only a 2′-OMe modification, and nucleotide 5 is the pyrimidine of a YA site and comprises a phosphorothioate, then the modified guide region YA site comprises a modification (phosphorothioate) that at least one nucleotide located 5′ of the guide region YA site (nucleotide 4) does not comprise. In another example, if nucleotides 1-3 comprise phosphorothioates, and nucleotide 4 is the pyrimidine of a YA site and comprises a 2′-OMe, then the modified guide region YA site comprises a modification (2′-OMe) that at least one nucleotide located 5′ of the guide region YA site (any of nucleotides 1-3) does not comprise. This condition is also always satisfied if an unmodified nucleotide is located 5′ of the modified guide region YA site.

In some embodiments, the modified guide region YA sites comprise modifications as described for YA sites above.

Additional embodiments of guide region YA site modifications are set forth in the summary above. Any embodiments set forth elsewhere in this disclosure may be combined to the extent feasible with any of the foregoing embodiments.

In some embodiments, the sgRNA comprises a conserved region YA site modification. Conserved region YA sites 1-10 are illustrated in FIG. 10. In some embodiments, 1, 2, 3, 4, 5, 6, 7, 8, 9, or 10 conserved region YA sites comprise modifications.

In some embodiments, conserved region YA sites 1, 8, or 1 and 8 comprise YA modifications. In some embodiments, conserved region YA sites 1, 2, 3, 4, and 10 comprise YA modifications. In some embodiments, YA sites 2, 3, 4, 8, and 10 comprise YA modifications. In some embodiments, conserved region YA sites 1, 2, 3, and 10 comprise YA modifications. In some embodiments, YA sites 2, 3, 8, and 10 comprise YA modifications. In some embodiments, YA sites 1, 2, 3, 4, 8, and 10 comprise YA modifications. In some embodiments, 1, 2, 3, 4, 5, 6, 7, or 8 additional conserved region YA sites comprise YA modifications.

In some embodiments, 1, 2, 3, or 4 of conserved region YA sites 2, 3, 4, and 10 comprise YA modifications. In some embodiments, 1, 2, 3, 4, 5, 6, 7, or 8 additional conserved region YA sites comprise YA modifications.

In some embodiments, the modified conserved region YA sites comprise modifications as described for YA sites above.

Additional embodiments of conserved region YA site modifications are set forth in the summary above. Any embodiments set forth elsewhere in this disclosure may be combined to the extent feasible with any of the foregoing embodiments.

In some embodiments, the sgRNA comprises any of the modification patterns shown above in Table 2, or below in Table 3, where N, if present, is any natural or non-natural nucleotide, and wherein the totality of the N's comprise an LDHA guide sequence as described herein in Table 1. Table 3 does not depict the guide sequence portion of the sgRNA. The modifications remain as shown in Table 3 despite the substitution of N's for the nucleotides of a guide. That is, although the nucleotides of the guide replace the “N's”, the nucleotides are modified as shown in Table 3. When the guide sequence is appended to the 5′ end, the 5′ end (or 5′ terminus) of the guide sequence may be modified. In some embodiments, the modifications comprise 2′-O-Me and/or PS-bonds. In some embodiments, the 2′-O-Me and/or PS-bonds are at the first 1 to 7, 1 to 6, 1 to 5, 1 to 4, or 1 to 3 nucleotides of the guide sequence at its 5′ end.

TABLE 3
LDHA sgRNA modification patterns. The guide sequence is not shown
and will append the shown sequence at its 5′ end.

| SEQ ID | | |
|---|---|---|
| NO | Name | Sequence |
| 400 | G000262-mod | GUUUUAGAGCUAGAAAUAGCAAGUUAAAAUAAGGCUAGUCCGUUAUCAA |
| | only | CUUGAAAAAGUmGmGmCmAmCmCmGmAmGmUmCmGmGmUmGmCmU*mU |
| | | *mU*mU |
| 401 | G000263-mod | GUUUUAGAGCUAGAAAUAGCAAGUUAAAAUAAGGCUAGUCCGUUAUCAm |
| | only | AmCmUmUmGmAmAmAmAmAmGmUmGmGmCmAmCmCmGmAmGmUmCmG |
| | | mGmUmGmCmU*mU*mU*mU |
| 402 | G000264-mod | GUUUUAGAGCUAmGmAmAmAUAGCAAGUUAAAAUAAGGCUAGUCCGUU |
| | only | AUCAACUUGAAAAAGUGGCACCGAGUCGGUGCmU*mU*mU*U |
| 403 | G000265-mod | GUUUUAGAmGmCmUmAGAAAmUmAmGmCAAGUUAAAAUAAGGCUAGUC |
| | only | CGUUAUCAACUUGAAAAAGUGGCACCGAGUCGGUGCmU*mU*mU*U |
| 404 | G000266-mod | GUUUUAGAmGmCmUmAmGmAmAmAmUmAmGmCAAGUUAAAAUAAGGCU |
| | only | AGUCCGUUAUCAACUUGAAAAAGUGGCACCGAGUCGGUGCmU*mU*mU*U |
| 405 | G000267-mod | GUUUUAGAmGmCmUmAmGmAmAmAmUmAmGmCAAGUUAAAAUAAGGCU |
| | only | AGUCCGUUAUCAmAmCmUmUmGmAmAmAmAmAmGmUmGmGmCmAmCmC |
| | | mGmAmGmUmCmGmGmUmGmCmU*mU*mU*mU |
| 406 | G000331- | mGUUUUmAGAmGmCmUmAmGmAmAmAmUmAmGmCAAGUUAAAAUAAG |
| | mod only | GCUAGUCCGUUAUCAmAmCmUmUmGmAmAmAmAmAmGmUmGmGmCmA |
| | | mCmCmGmAmGmUmCmGmGmUmGmCmU*mU*mU*mU |
| 407 | G000332- | fGfUfUfUfUfAGAmGmCmUmAmGmAmAmAmUmAmGmCAAGUUAAAAUAA |
| | mod only | GGCUAGUCCGUUAUCAmAmCmUmUmGmAmAmAmAmAmGmUmGmGmCm |
| | | AmCmCmGmAmGmUmCmGmGmUmGmCmU*mU*mU*mU |
| 408 | G000333- | mGfUfUfUfUmAGAmGmCmUmAmGmAmAmAmUmAmGmCAAGUUAAAAUA |
| | mod only | AGGCUAGUCCGUUAUCAmAmCmUmUmGmAmAmAmAmAmGmUmGmGmC |
| | | mAmCmCmGmAmGmUmCmGmGmUmGmCmU*mU*mU*mU |
| 409 | G000334- | GUUUUAGAmGmCmUmAmGmAmAmAmUmAmGmCAAGUmUmAAAmAmUA |
| | mod only | AGGCUAGUCCGUUAUCAmAmCmUmUmGmAmAmAmAmAmGmUmGmGmC |
| | | mAmCmCmGmAmGmUmCmGmGmUmGmCmU*mU*mU*mU |
| 410 | G000335- | GUUUUAGAmGmCmUmAmGmAmAmAmUmAmGmCAAGUmUmAfAfAmAmU |
| | mod only | AAGGCUAGUCCGUUAUCAmAmCmUmUmGmAmAmAmAmAmGmUmGmGm |
| | | CmAmCmCmGmAmGmUmCmGmGmUmGmCmU*mU*mU*mU |
| 411 | G000336- | GUUUUAGAmGmCmUmAmGmAmAmAmUmAmGmCAAGUfUmAfAmAfAmU |
| | mod only | AAGGCUAGUCCGUUAUCAmAmCmUmUmGmAmAmAmAmAmGmUmGmGm |
| | | CmAmCmCmGmAmGmUmCmGmGmUmGmCmU*mU*mU*mU |
| 412 | G000337- | mGUUUUmAGAmGmCmUmAmGmAmAmAmUmAmGmCAAGUmUmAAAmA |
| | mod only | mUAAGGCUAGUCCGUUAUCAmAmCmUmUmGmAmAmAmAmAmGmUmGm |
| | | GmCmAmCmCmGmAmGmUmCmGmGmUmGmCmU*mU*mU*mU |
| 413 | G000338- | mGUUUUmAGAmGmCmUmAmGmAmAmAmUmAmGmCAAGUmUmAfAfAmA |
| | mod only | mUAAGGCUAGUCCGUUAUCAmAmCmUmUmGmAmAmAmAmAmGmUmGm |
| | | GmCmAmCmCmGmAmGmUmCmGmGmUmGmCmU*mU*mU*mU |
| 414 | G000339- | mGUUUUmAGAmGmCmUmAmGmAmAmAmUmAmGmCAAGUfUmAfAmAfA |
| | mod only | mUAAGGCUAGUCCGUUAUCAmAmCmUmUmGmAmAmAmAmAmGmUmGm |
| | | GmCmAmCmCmGmAmGmUmCmGmGmUmGmCmU*mU*mU*mU |
| 415 | G000340- | fGfUfUfUfUfAGAmGmCmUmAmGmAmAmAmUmAmGmCAAGUmUmAAAmA |
| | mod only | mUAAGGCUAGUCCGUUAUCAmAmCmUmUmGmAmAmAmAmAmGmUmGm |
| | | GmCmAmCmCmGmAmGmUmCmGmGmUmGmCmU*mU*mU*mU |
| 416 | G000341- | fGfUfUfUfUfAGAmGmCmUmAmGmAmAmAmUmAmGmCAAGUmUmAfAfAm |
| | mod only | AmUAAGGCUAGUCCGUUAUCAmAmCmUmUmGmAmAmAmAmAmGmUmG |
| | | mGmCmAmCmCmGmAmGmUmCmGmGmUmGmCmU*mU*mU*mU |
| 417 | G000342- | fGfUfUfUfUfAGAmGmCmUmAmGmAmAmAmUmAmGmCAAGUfUmAfAmAfA |
| | mod only | mUAAGGCUAGUCCGUUAUCAmAmCmUmUmGmAmAmAmAmAmGmUmGm |
| | | GmCmAmCmCmGmAmGmUmCmGmGmUmGmCmU*mU*mU*mU |
| 418 | G000343- | GUUUUAmGmAmGmCmUmAmGmAmAmAmUmAmGmCAAGUUAAAAUAAG |
| | mod only | GCUAGUCCGUUAUCAmAmCmUmUmGmAmAmAmAmAmGmUmGmGmCmA |
| | | mCmCmGmAmGmUmCmGmGmUmGmCmU*mU*mU*mU |
| 419 | G000344- | GUUUUAGAmGmCmUmAmGmAmAmAmUmAmGmCmAmAmGmUUAAAAUA |
| | mod only | AGGCUAGUCCGUUAUCAmAmCmUmUmGmAmAmAmAmAmGmUmGmGmC |
| | | mAmCmCmGmAmGmUmCmGmGmUmGmCmU*mU*mU*mU |
| 420 | G000345- | GUUUUAGAmGmCmUmAmGmAmAmAmUmAmGmCAAGUUAAAAUAAGGC |
| | mod only | UAGUCCGUUfAfUfCfAmAmCmUmUmGmAmAmAmAmAmGmUmGmGmCmA |
| | | mCmCmGmAmGmUmCmGmGmUmGmCmU*mU*mU*mU |
| 421 | G000346- | GUUUUAGAmGmCmUmAmGmAmAmAmUmAmGmCAAGUUAAAAUAAGGC |
| | mod only | UAGUCCGUUAmUmCmAmAmCmUmUmGmAmAmAmAmAmGmUmGmGmCm |
| | | AmCmCmGmAmGmUmCmGmGmUmGmCmU*mU*mU*mU |
| 422 | G000347- | fGfUfUfUfUfAmGmAmGmCmUmAmGmAmAmAmUmAmGmCmAmAmGmUmU |
| | mod only | mAfAfAmAmUAAGGCUAGUCCGUUAmUmCmAmAmCmUmUmGmAmAmAm |
| | | AmAmGmUmGmGmCmAmCmCmGmAmGmUmCmGmGmUmGmCmU*mU*mU |
| | | *mU |
| 423 | G000348- | GUUUUAGAmGmCmUmAmGmAmAmAmUmAmGmCAAGUUAAAAUAAGGC |
| | mod only | UAGUCCGUUAUCAmAmCmUmUmGmAmAmAmAmAmGmUmGmGmCmAmC |
| | | mCmGmAmGmUmCmGmGmUmGmCmUmUmUmU |
| 424 | G000349- | GUUUUAGAmGmCmUmAmGmAmAmAmUmAmGmCAAGUUAAAAUAAGGC |
| | mod only | UAGUCCGUUAUCAmAmCmUmUmGmAmAmAmAmAmGmUmGmGmCmAmC |
| | | mCmGmAmGmUmCmGmGmUmGmCmUmU*mU*mU |
| 425 | G000350- | GUUUUAGAmGmCmUmAmGmAmAmAmUmAmGmCAAGUUAAAAUAAGGC |
| | mod only | UAGUCCGUUAUCAmAmCmUmUmGmAmAmAmAmAmGmUmGmGmCmAmC |
| | | mCmGmAmGfUfCfGfGfUfGfCfU*fU*fU*mU |
| 426 | G000351- | GUUUUAGAmGmCmUmAmGmAmAmAmUmAmGmCAAGUUAAAAUAAGGC |
| | mod only | UAGUCCGUUAUCAfAmCfUmUfGmAfAmAfAmAfGmUfGmGfCmAfCmCfGmA |
| | | fGmUfCmGfGmUfGmCfU*mU*fU*mU |
| 427 | G000352- | mGUUUUmAGAmGmCmUmAmGmAmAmAmUmAmGmCAAGUUAAAAUAAG |
| | mod only | GCUAGUCCGUUAUCAmAmCmUmUmGmAmAmAmAmAmGmUmGmGmCmA |
| | | mCmCmGmAmGmUmCmGmGmUmGmCmU*mU*mU*mU |
| 428 | G000353- | fGfUfUfUfUfAGAmGmCmUmAmGmAmAmAmUmAmGmCAAGUUAAAAUAA |
| | mod only | GGCUAGUCCGUUAUCAmAmCmUmUmGmAmAmAmAmAmGmUmGmGmCm |
| | | AmCmCmGmAmGmUmCmGmGmUmGmCmU*mU*mU*mU |
| 429 | G000354- | mGfUfUfUfUmAGAmGmCmUmAmGmAmAmAmUmAmGmCAAGUUAAAAUA |
| | mod only | AGGCUAGUCCGUUAUCAmAmCmUmUmGmAmAmAmAmAmGmUmGmGmC |
| | | mAmCmCmGmAmGmUmCmGmGmUmGmCmU*mU*mU*mU |
| 430 | G000355- | GUUUUAGAmGmCmUmAmGmAmAmAmUmAmGmCAAGUmUmAAAmAmUA |
| | mod only | AGGCUAGUCCGUUAUCAmAmCmUmUmGmAmAmAmAmAmGmUmGmGmC |
| | | mAmCmCmGmAmGmUmCmGmGmUmGmCmU*mU*mU*mU |
| 431 | G000356- | GUUUUAGAmGmCmUmAmGmAmAmAmUmAmGmCAAGUmUmAfAfAmAmU |
| | mod only | AAGGCUAGUCCGUUAUCAmAmCmUmUmGmAmAmAmAmAmGmUmGmGm |
| | | CmAmCmCmGmAmGmUmCmGmGmUmGmCmU*mU*mU*mU |
| 432 | G000357- | GUUUUAGAmGmCmUmAmGmAmAmAmUmAmGmCAAGUfUmAfAmAfAmU |
| | mod only | AAGGCUAGUCCGUUAUCAmAmCmUmUmGmAmAmAmAmAmGmUmGmGm |
| | | CmAmCmCmGmAmGmUmCmGmGmUmGmCmU*mU*mU*mU |
| 433 | G000358- | mGUUUUmAGAmGmCmUmAmGmAmAmAmUmAmGmCAAGUmUmAAAmA |
| | mod only | mUAAGGCUAGUCCGUUAUCAmAmCmUmUmGmAmAmAmAmAmGmUmGm |
| | | GmCmAmCmCmGmAmGmUmCmGmGmUmGmCmU*mU*mU*mU |
| 434 | G000359- | mGUUUUmAGAmGmCmUmAmGmAmAmAmUmAmGmCAAGUmUmAfAfAmA |
| | mod only | mUAAGGCUAGUCCGUUAUCAmAmCmUmUmGmAmAmAmAmAmGmUmGm |
| | | GmCmAmCmCmGmAmGmUmCmGmGmUmGmCmU*mU*mU*mU |
| 435 | G000360- | mGUUUUmAGAmGmCmUmAmGmAmAmAmUmAmGmCAAGUfUmAfAmAfA |
| | mod only | mUAAGGCUAGUCCGUUAUCAmAmCmUmUmGmAmAmAmAmAmGmUmGm |
| | | GmCmAmCmCmGmAmGmUmCmGmGmUmGmCmU*mU*mU*mU |
| 436 | G000361- | fGfUfUfUfUfAGAmGmCmUmAmGmAmAmAmUmAmGmCAAGUmUmAAAmA |
| | mod only | mUAAGGCUAGUCCGUUAUCAmAmCmUmUmGmAmAmAmAmAmGmUmGm |
| | | GmCmAmCmCmGmAmGmUmCmGmGmUmGmCmU*mU*mU*mU |
| 437 | G000362- | fGfUfUfUfUfAGAmGmCmUmAmGmAmAmAmUmAmGmCAAGUmUmAfAfAm |
| | mod only | AmUAAGGCUAGUCCGUUAUCAmAmCmUmUmGmAmAmAmAmAmGmUmG |
| | | mGmCmAmCmCmGmAmGmUmCmGmGmUmGmCmU*mU*mU*mU |
| 438 | G000363- | fGfUfUfUfUfAGAmGmCmUmAmGmAmAmAmUmAmGmCAAGUfUmAfAmAfA |
| | mod only | mUAAGGCUAGUCCGUUAUCAmAmCmUmUmGmAmAmAmAmAmGmUmGm |
| | | GmCmAmCmCmGmAmGmUmCmGmGmUmGmCmU*mU*mU*mU |
| 439 | G000364- | GUUUUAmGmAmGmCmUmAmGmAmAmAmUmAmGmCAAGUUAAAAUAAG |
| | mod only | GCUAGUCCGUUAUCAmAmCmUmUmGmAmAmAmAmAmGmUmGmGmCmA |
| | | mCmCmGmAmGmUmCmGmGmUmGmCmU*mU*mU*mU |
| 440 | G000365- | GUUUUAGAmGmCmUmAmGmAmAmAmUmAmGmCmAmAmGmUUAAAAUA |
| | mod only | AGGCUAGUCCGUUAUCAmAmCmUmUmGmAmAmAmAmAmGmUmGmGmC |
| | | mAmCmCmGmAmGmUmCmGmGmUmGmCmU*mU*mU*mU |
| 441 | G000366- | GUUUUAGAmGmCmUmAmGmAmAmAmUmAmGmCAAGUUAAAAUAAGGC |
| | mod only | UAGUCCGUUfAfUfCfAmAmCmUmUmGmAmAmAmAmAmGmUmGmGmCmA |
| | | mCmCmGmAmGmUmCmGmGmUmGmCmU*mU*mU*mU |
| 442 | G000367- | GUUUUAGAmGmCmUmAmGmAmAmAmUmAmGmCAAGUUAAAAUAAGGC |
| | mod only | UAGUCCGUUAmUmCmAmAmCmUmUmGmAmAmAmAmAmGmUmGmGmCm |
| | | AmCmCmGmAmGmUmCmGmGmUmGmCmU*mU*mU*mU |
| 443 | G000368- | fGfUfUfUfUfAmGmAmGmCmUmAmGmAmAmAmUmAmGmCmAmAmGmUmU |
| | mod only | mAfAfAmAmUAAGGCUAGUCCGUUAmUmCmAmAmCmUmUmGmAmAmAm |
| | | AmAmGmUmGmGmCmAmCmCmGmAmGmUmCmGmGmUmGmCmU*mU*mU |
| | | *mU |
| 444 | G000369- | GUUUUAGAmGmCmUmAmGmAmAmAmUmAmGmCAAGUUAAAAUAAGGC |
| | mod only | UAGUCCGUUAUCAmAmCmUmUmGmAmAmAmAmAmGmUmGmGmCmAmC |
| | | mCmGmAmGmUmCmGmGmUmGmCmUmUmUmU |
| 445 | G000370- | GUUUUAGAmGmCmUmAmGmAmAmAmUmAmGmCAAGUUAAAAUAAGGC |
| | mod only | UAGUCCGUUAUCAmAmCmUmUmGmAmAmAmAmAmGmUmGmGmCmAmC |
| | | mCmGmAmGmUmCmGmGmUmGmCmUmU*mU*mU |
| 446 | G000371- | GUUUUAGAmGmCmUmAmGmAmAmAmUmAmGmCAAGUUAAAAUAAGGC |
| | mod only | UAGUCCGUUAUCAmAmCmUmUmGmAmAmAmAmAmGmUmGmGmCmAmC |
| | | mCmGmAmGfUfCfGfGfUfGfCfU*fU*fU*mU |
| 447 | G000372- | GUUUUAGAmGmCmUmAmGmAmAmAmUmAmGmCAAGUUAAAAUAAGGC |
| | mod only | UAGUCCGUUAUCAfAmCfUmUfGmAfAmAfAmAfGmUfGmGfCmAfCmCfGmA |
| | | fGmUfCmGfGmUfGmCfU*mU*fU*mU |
| 448 | Exemplary- | mN*mN*mN*mNNN*N*fN*fN*fN*fNNfNfNNNfNfNNN |
| | guide region | |
| | mod only | |
| 449 | Exemplary- | mN*mN*mN*mNNN*N*fN*fN*fN*fNNfNfNNN*fNfNNN |
| | guide region | |
| | mod only | |
| 450 | Exemplary- | GUUUUAGAmGmCmUmAmGmAmAmAmUmAmGmCAAGUUAAAAUAAGGCU |
| | mod only | AGUCCGUUAUCAACUUGGCACCGAGUCGG*mU*mG*mC |

In some embodiments, the modified sgRNA comprises the following sequence: mN*mN*mN*NNNNNNNNNNNNNNNNNGUUUUAGAmGmCmUmAmGmAmAmAmU mAmGmCAAGUUAAAAUAAGGCUAGUCCGUUAUCAmAmCmUmUmGmAmAmAm AmAmGmUmGmGmCmAmCmCmGmAmGmUmCmGmGmUmGmCmU*mU*mU*mU (SEQ ID NO: 300), where “N” may be any natural or non-natural nucleotide, and wherein the totality of N's comprise an LDHA guide sequence as described in Table 1. For example, encompassed herein is SEQ ID NO: 300, where the N's are replaced with any of the guide sequences disclosed herein in Table 1 (SEQ ID NOs: 1-84).

Any of the modifications described below may be present in the gRNAs and mRNAs described herein.

The terms “mA,” “mC,” “mU,” or “mG” may be used to denote a nucleotide that has been modified with 2′-O-Me.

Modification of 2′-O-methyl can be depicted as follows:

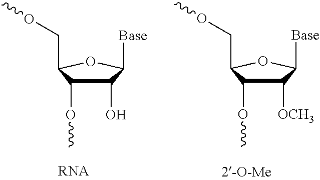

Another chemical modification that has been shown to influence nucleotide sugar rings is halogen substitution. For example, 2′-fluoro (2′-F) substitution on nucleotide sugar rings can increase oligonucleotide binding affinity and nuclease stability.

In this application, the terms “fA,” “fC,” “fU,” or “fG” may be used to denote a nucleotide that has been substituted with 2′-F.

Substitution of 2′-F can be depicted as follows:

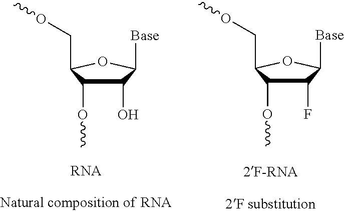

Phosphorothioate (PS) linkage or bond refers to a bond where a sulfur is substituted for one nonbridging phosphate oxygen in a phosphodiester linkage, for example in the bonds between nucleotides bases. When phosphorothioates are used to generate oligonucleotides, the modified oligonucleotides may also be referred to as S-oligos.

A “*” may be used to depict a PS modification. In this application, the terms A*, C*, U*, or G* may be used to denote a nucleotide that is linked to the next (e.g., 3′) nucleotide with a PS bond.

In this application, the terms “mA*,” “mC*,” “mU*,” or “mG*” may be used to denote a nucleotide that has been substituted with 2′-O-Me and that is linked to the next (e.g., 3′) nucleotide with a PS bond.

The diagram below shows the substitution of S- into a nonbridging phosphate oxygen, generating a PS bond in lieu of a phosphodiester bond:

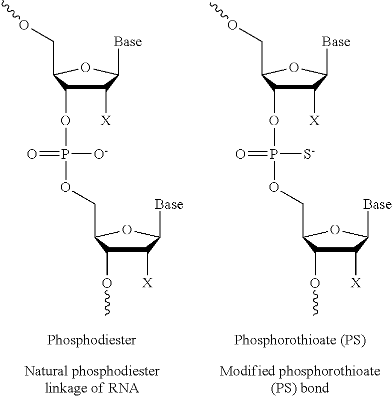

Abasic nucleotides refer to those which lack nitrogenous bases. The figure below depicts an oligonucleotide with an abasic (also known as apurinic) site that lacks a base:

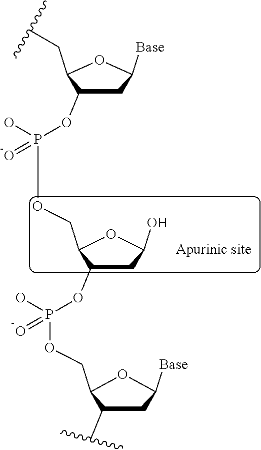

Inverted bases refer to those with linkages that are inverted from the normal 5′ to 3′ linkage (i.e either a 5′ to 5′ linkage or a 3′ to 3′ linkage). For example:

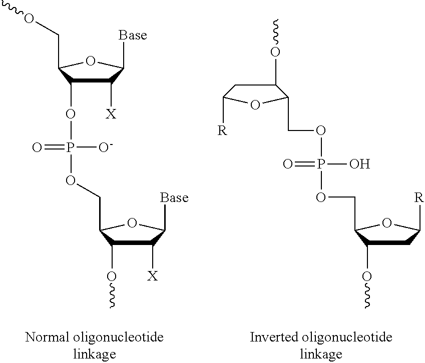

An abasic nucleotide can be attached with an inverted linkage. For example, an abasic nucleotide may be attached to the terminal 5′ nucleotide via a 5′ to 5′ linkage, or an abasic nucleotide may be attached to the terminal 3′ nucleotide via a 3′ to 3′ linkage. An inverted abasic nucleotide at either the terminal 5′ or 3′ nucleotide may also be called an inverted abasic end cap.

In some embodiments, one or more of the first three, four, or five nucleotides at the 5′ terminus, and one or more of the last three, four, or five nucleotides at the 3′ terminus are modified. In some embodiments, the modification is a 2′-O-Me, 2′-F, inverted abasic nucleotide, PS bond, or other nucleotide modification well known in the art to increase stability and/or performance.

In some embodiments, the first four nucleotides at the 5′ terminus, and the last four nucleotides at the 3′ terminus are linked with phosphorothioate (PS) bonds.

In some embodiments, the first three nucleotides at the 5′ terminus, and the last three nucleotides at the 3′ terminus comprise a 2′-O-methyl (2′-O-Me) modified nucleotide. In some embodiments, the first three nucleotides at the 5′ terminus, and the last three nucleotides at the 3′ terminus comprise a 2′-fluoro (2′-F) modified nucleotide. In some embodiments, the first three nucleotides at the 5′ terminus, and the last three nucleotides at the 3′ terminus comprise an inverted abasic nucleotide.

In some embodiments, the guide RNA comprises a modified sgRNA. In some embodiments, the sgRNA comprises the modification pattern shown in SEQ ID No: 201, 202, or 203, where N is any natural or non-natural nucleotide, and where the totality of the N's comprise a guide sequence that directs a nuclease to a target sequence in LDHA, e.g., as shown in Table 1.

In some embodiments, the guide RNA comprises a sgRNA shown in any one of SEQ ID NOs: 1001, 1005, 1007, 1008, 1014, 1023, 1027, 1032, 1045, 1048, 1063, 1067, 1069, 1071, 1074, 1076, 1077, 1078, 1079, and 1081, or modified versions thereof as shown, e.g., in SEQ ID NOs: 2001, 2005, 2007, 2008, 2014, 2023, 2027, 2032, 2045, 2048, 2063, 2067, 2069, 2071, 2074, 2076, 2077, 2078, 2079, and 2081. In some embodiments, the guide RNA comprises a sgRNA comprising any one of the guide sequences of SEQ ID No: 1-84 and 100-192 and the nucleotides of SEQ ID No: 201, 202, or 203, wherein the nucleotides of SEQ ID No: 201, 202, or 203 are on the 3′ end of the guide sequence, and wherein the sgRNA may be modified as shown in Table 3 or SEQ ID NO: 300.

As noted above, in some embodiments, a composition or formulation disclosed herein comprises an mRNA comprising an open reading frame (ORF) encoding an RNA-guided DNA binding agent, such as a Cas nuclease as described herein. In some embodiments, an mRNA comprising an ORF encoding an RNA-guided DNA binding agent, such as a Cas nuclease, is provided, used, or administered. In some embodiments, the ORF encoding an RNA-guided DNA nuclease is a “modified RNA-guided DNA binding agent ORF” or simply a “modified ORF,” which is used as shorthand to indicate that the ORF is modified.

In some embodiments, the modified ORF may comprise a modified uridine at least at one, a plurality of, or all uridine positions. In some embodiments, the modified uridine is a uridine modified at the 5 position, e.g., with a halogen, methyl, or ethyl. In some embodiments, the modified uridine is a pseudouridine modified at the 1 position, e.g., with a halogen, methyl, or ethyl. The modified uridine can be, for example, pseudouridine, N1-methyl-pseudouridine, 5-methoxyuridine, 5-iodouridine, or a combination thereof. In some embodiments, the modified uridine is 5-methoxyuridine. In some embodiments, the modified uridine is 5-iodouridine. In some embodiments, the modified uridine is pseudouridine. In some embodiments, the modified uridine is N1-methyl-pseudouridine. In some embodiments, the modified uridine is a combination of pseudouridine and N1-methyl-pseudouridine. In some embodiments, the modified uridine is a combination of pseudouridine and 5-methoxyuridine. In some embodiments, the modified uridine is a combination of N1-methyl pseudouridine and 5-methoxyuridine. In some embodiments, the modified uridine is a combination of 5-iodouridine and N1-methyl-pseudouridine. In some embodiments, the modified uridine is a combination of pseudouridine and 5-iodouridine. In some embodiments, the modified uridine is a combination of 5-iodouridine and 5-methoxyuridine.

In some embodiments, an mRNA disclosed herein comprises a 5′ cap, such as a Cap0, Cap1, or Cap2. A 5′ cap is generally a 7-methylguanine ribonucleotide (which may be further modified, as discussed below e.g. with respect to ARCA) linked through a 5′-triphosphate to the 5′ position of the first nucleotide of the 5′-to-3′ chain of the mRNA, i.e., the first cap-proximal nucleotide. In Cap0, the riboses of the first and second cap-proximal nucleotides of the mRNA both comprise a 2′-hydroxyl. In Cap1, the riboses of the first and second transcribed nucleotides of the mRNA comprise a 2′-methoxy and a 2′-hydroxyl, respectively. In Cap2, the riboses of the first and second cap-proximal nucleotides of the mRNA both comprise a 2′-methoxy. See, e.g., Katibah et al. (2014) Proc Natl Acad Sci USA 111(33):12025-30; Abbas et al. (2017) Proc Natl Acad Sci USA 114(11):E2106-E2115. Most endogenous higher eukaryotic mRNAs, including mammalian mRNAs such as human mRNAs, comprise Cap1 or Cap2. Cap0 and other cap structures differing from Cap1 and Cap2 may be immunogenic in mammals, such as humans, due to recognition as “non-self” by components of the innate immune system such as IFIT-1 and IFIT-5, which can result in elevated cytokine levels including type I interferon. Components of the innate immune system such as IFIT-1 and IFIT-5 may also compete with eIF4E for binding of an mRNA with a cap other than Cap1 or Cap2, potentially inhibiting translation of the mRNA.

A cap can be included co-transcriptionally. For example, ARCA (anti-reverse cap analog; Thermo Fisher Scientific Cat. No. AM8045) is a cap analog comprising a 7-methylguanine 3′-methoxy-5′-triphosphate linked to the 5′ position of a guanine ribonucleotide which can be incorporated in vitro into a transcript at initiation. ARCA results in a Cap0 cap in which the 2′ position of the first cap-proximal nucleotide is hydroxyl. See, e.g., Stepinski et al., (2001) “Synthesis and properties of mRNAs containing the novel ‘anti-reverse’ cap analogs 7-methyl(3′-O-methyl)GpppG and 7-methyl(3′deoxy)GpppG,” RNA 7: 1486-1495. The ARCA structure is shown below.

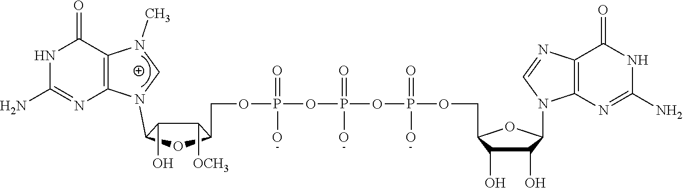

CleanCap™ AG (m7G(5′)ppp(5′)(2′OMeA)pG; TriLink Biotechnologies Cat. No. N-7113) or CleanCap™ GG (m7G(5′)ppp(5′)(2′OMeG)pG; TriLink Biotechnologies Cat. No. N-7133) can be used to provide a Cap1 structure co-transcriptionally. 3′-O-methylated versions of CleanCap™ AG and CleanCap™ GG are also available from TriLink Biotechnologies as Cat. Nos. N-7413 and N-7433, respectively. The CleanCap™ AG structure is shown below.

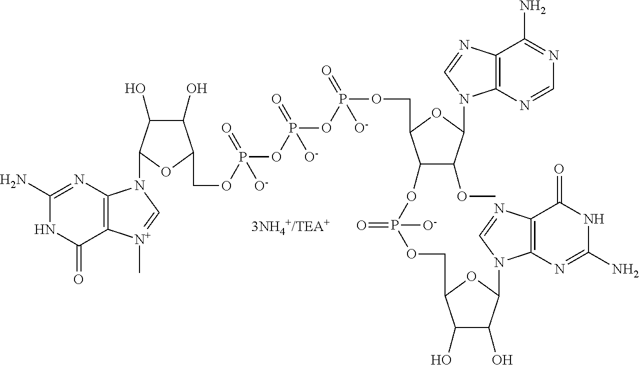 [SMILES: COC1C(OP(=O)([O-])OCC2OC(n3cnc4c(=O)[nH]c(N)nc43)C(O)C2O)C(COP(=O)([O-])OP(=O)([O-])OP(=O)([O-])OCC2OC(n3c[n+](C)c4c(=O)[nH]c(N)nc43)C(O)C2O)OC1n1cnc2c(N)ncnc21]

Alternatively, a cap can be added to an RNA post-transcriptionally. For example, Vaccinia capping enzyme is commercially available (New England Biolabs Cat. No. M2080S) and has RNA triphosphatase and guanylyltransferase activities, provided by its D1 subunit, and guanine methyltransferase, provided by its D12 subunit. As such, it can add a 7-methylguanine to an RNA, so as to give Cap0, in the presence of S-adenosyl methionine and GTP. See, e.g., Guo, P. and Moss, B. (1990) Proc. Natl. Acad. Sci. USA 87, 4023-4027; Mao, X. and Shuman, S. (1994) J. Biol. Chem. 269, 24472-24479.

In some embodiments, the mRNA further comprises a poly-adenylated (poly-A) tail. In some embodiments, the poly-A tail comprises at least 20, 30, 40, 50, 60, 70, 80, 90, or 100 adenines, optionally up to 300 adenines. In some embodiments, the poly-A tail comprises 95, 96, 97, 98, 99, or 100 adenine nucleotides.

### D. Ribonucleoprotein Complex

In some embodiments, a composition is encompassed comprising one or more gRNAs comprising one or more guide sequences from Table 1 or one or more sgRNAs from Table 2 and an RNA-guided DNA binding agent, e.g., a nuclease, such as a Cas nuclease, such as Cas9. In some embodiments, the RNA-guided DNA-binding agent has cleavase activity, which can also be referred to as double-strand endonuclease activity. In some embodiments, the RNA-guided DNA-binding agent comprises a Cas nuclease. Examples of Cas9 nucleases include those of the type II CRISPR systems of S. pyogenes, S. aureus, and other prokaryotes (see, e.g., the list in the next paragraph), and modified (e.g., engineered or mutant) versions thereof. See, e.g., US2016/0312198 A1; US 2016/0312199 A1. Other examples of Cas nucleases include a Csm or Cmr complex of a type III CRISPR system or the Cas10, Csm1, or Cmr2 subunit thereof, and a Cascade complex of a type I CRISPR system, or the Cas3 subunit thereof. In some embodiments, the Cas nuclease may be from a Type-IIA, Type-IIB, or Type-IIC system. For discussion of various CRISPR systems and Cas nucleases see, e.g., Makarova et al., NAT. REV. MICROBIOL. 9:467-477 (2011); Makarova et al., NAT. REV. MICROBIOL, 13: 722-36 (2015); Shmakov et al., MOLECULAR CELL, 60:385-397 (2015).

Non-limiting exemplary species that the Cas nuclease can be derived from include Streptococcus pyogenes, Streptococcus thermophilus, Streptococcus sp., Staphylococcus aureus, Listeria innocua, Lactobacillus gasseri, Francisella novicida, Wolinella succinogenes, Sutterella wadsworthensis, Gammaproteobacterium, Neisseria meningitidis, Campylobacter jejuni, Pasteurella multocida, Fibrobacter succinogene, Rhodospirillum rubrum, Nocardiopsis dassonvillei, Streptomyces pristinaespiralis, Streptomyces viridochromogenes, Streptomyces viridochromogenes, Streptosporangium roseum, Streptosporangium roseum, Alicyclobacillus acidocaldarius, Bacillus pseudomycoides, Bacillus selenitireducens, Exiguobacterium sibiricum, Lactobacillus delbrueckii, Lactobacillus salivarius, Lactobacillus buchneri, Treponema denticola, Microscilla marina, Burkholderiales bacterium, Polaromonas naphthalenivorans, Polaromonas sp., Crocosphaera watsonii, Cyanothece sp., Microcystis aeruginosa, Synechococcus sp., Acetohalobium arabaticum, Ammonifex degensii, Caldicelulosiruptor becscii, Candidatus Desulforudis, Clostridium botulinum, Clostridium difficile, Finegoldia magna, Natranaerobius thermophilus, Pelotomaculum thermopropionicum, Acidithiobacillus caldus, Acidithiobacillus ferrooxidans, Allochromatium vinosum, Marinobacter sp., Nitrosococcus halophilus, Nitrosococcus watsoni, Pseudoalteromonas haloplanktis, Ktedonobacter racemifer, Methanohalobium evestigatum, Anabaena variabilis, Nodularia spumigena, Nostoc sp., Arthrospira maxima, Arthrospira platensis, Arthrospira sp., Lyngbya sp., Microcoleus chthonoplastes, Oscillatoria sp., Petrotoga mobilis, Thermosipho africanus, Streptococcus pasteurianus, Neisseria cinerea, Campylobacter lari, Parvibaculum lavamentivorans, Corynebacterium diphtheria, Acidaminococcus sp., Lachnospiraceae bacterium ND2006, and Acaryochloris marina.

In some embodiments, the Cas nuclease is the Cas9 nuclease from Streptococcus pyogenes. In some embodiments, the Cas nuclease is the Cas9 nuclease from Streptococcus thermophilus. In some embodiments, the Cas nuclease is the Cas9 nuclease from Neisseria meningitidis. In some embodiments, the Cas nuclease is the Cas9 nuclease is from Staphylococcus aureus. In some embodiments, the Cas nuclease is the Cpf1 nuclease from Francisella novicida. In some embodiments, the Cas nuclease is the Cpf1 nuclease from Acidaminococcus sp. In some embodiments, the Cas nuclease is the Cpf1 nuclease from Lachnospiraceae bacterium ND2006. In further embodiments, the Cas nuclease is the Cpf1 nuclease from Francisella tularensis, Lachnospiraceae bacterium, Butyrivibrio proteoclasticus, Peregrinibacteria bacterium, Parcubacteria bacterium, Smithella, Acidaminococcus, Candidatus Methanoplasma termitum, Eubacterium eligens, Moraxella bovoculi, Leptospira inadai, Porphyromonas crevioricanis, Prevotella disiens, or Porphyromonas macacae. In certain embodiments, the Cas nuclease is a Cpf1 nuclease from an Acidaminococcus or Lachnospiraceae.

In some embodiments, the gRNA together with an RNA-guided DNA binding agent is called a ribonucleoprotein complex (RNP). In some embodiments, the RNA-guided DNA binding agent is a Cas nuclease. In some embodiments, the gRNA together with a Cas nuclease is called a Cas RNP. In some embodiments, the RNP comprises Type-I, Type-II, or Type-III components. In some embodiments, the Cas nuclease is the Cas9 protein from the Type-II CRISPR/Cas system. In some embodiment, the gRNA together with Cas9 is called a Cas9 RNP.

Wild type Cas9 has two nuclease domains: RuvC and HNH. The RuvC domain cleaves the non-target DNA strand, and the HNH domain cleaves the target strand of DNA. In some embodiments, the Cas9 protein comprises more than one RuvC domain and/or more than one HNH domain. In some embodiments, the Cas9 protein is a wild type Cas9. In each of the composition, use, and method embodiments, the Cas induces a double strand break in target DNA.

In some embodiments, chimeric Cas nucleases are used, where one domain or region of the protein is replaced by a portion of a different protein. In some embodiments, a Cas nuclease domain may be replaced with a domain from a different nuclease such as Fok1. In some embodiments, a Cas nuclease may be a modified nuclease.

In other embodiments, the Cas nuclease may be from a Type-I CRISPR/Cas system. In some embodiments, the Cas nuclease may be a component of the Cascade complex of a Type-I CRISPR/Cas system. In some embodiments, the Cas nuclease may be a Cas3 protein. In some embodiments, the Cas nuclease may be from a Type-III CRISPR/Cas system. In some embodiments, the Cas nuclease may have an RNA cleavage activity.

In some embodiments, the RNA-guided DNA-binding agent has single-strand nickase activity, i.e., can cut one DNA strand to produce a single-strand break, also known as a “nick.” In some embodiments, the RNA-guided DNA-binding agent comprises a Cas nickase. A nickase is an enzyme that creates a nick in dsDNA, i.e., cuts one strand but not the other of the DNA double helix. In some embodiments, a Cas nickase is a version of a Cas nuclease (e.g., a Cas nuclease discussed above) in which an endonucleolytic active site is inactivated, e.g., by one or more alterations (e.g., point mutations) in a catalytic domain. See, e.g., U.S. Pat. No. 8,889,356 for discussion of Cas nickases and exemplary catalytic domain alterations. In some embodiments, a Cas nickase such as a Cas9 nickase has an inactivated RuvC or HNH domain.

In some embodiments, the RNA-guided DNA-binding agent is modified to contain only one functional nuclease domain. For example, the agent protein may be modified such that one of the nuclease domains is mutated or fully or partially deleted to reduce its nucleic acid cleavage activity. In some embodiments, a nickase is used having a RuvC domain with reduced activity. In some embodiments, a nickase is used having an inactive RuvC domain. In some embodiments, a nickase is used having an HNH domain with reduced activity. In some embodiments, a nickase is used having an inactive HNH domain.

In some embodiments, a conserved amino acid within a Cas protein nuclease domain is substituted to reduce or alter nuclease activity. In some embodiments, a Cas nuclease may comprise an amino acid substitution in the RuvC or RuvC-like nuclease domain. Exemplary amino acid substitutions in the RuvC or RuvC-like nuclease domain include D10A (based on the S. pyogenes Cas9 protein). See, e.g., Zetsche et al. (2015) Cell October 22:163(3): 759-771. In some embodiments, the Cas nuclease may comprise an amino acid substitution in the HNH or HNH-like nuclease domain. Exemplary amino acid substitutions in the HNH or HNH-like nuclease domain include E762A, H840A, N863A, H983A, and D986A (based on the S. pyogenes Cas9 protein). See, e.g., Zetsche et al. (2015). Further exemplary amino acid substitutions include D917A, E1006A, and D1255A (based on the Francisella novicida U112 Cpf1 (FnCpf1) sequence (UniProtKB—AOQ7Q2 (CPF1_FRATN)).

In some embodiments, an mRNA encoding a nickase is provided in combination with a pair of guide RNAs that are complementary to the sense and antisense strands of the target sequence, respectively. In this embodiment, the guide RNAs direct the nickase to a target sequence and introduce a DSB by generating a nick on opposite strands of the target sequence (i.e., double nicking). In some embodiments, use of double nicking may improve specificity and reduce off-target effects. In some embodiments, a nickase is used together with two separate guide RNAs targeting opposite strands of DNA to produce a double nick in the target DNA. In some embodiments, a nickase is used together with two separate guide RNAs that are selected to be in close proximity to produce a double nick in the target DNA.

In some embodiments, the RNA-guided DNA-binding agent lacks cleavase and nickase activity. In some embodiments, the RNA-guided DNA-binding agent comprises a dCas DNA-binding polypeptide. A dCas polypeptide has DNA-binding activity while essentially lacking catalytic (cleavase/nickase) activity. In some embodiments, the dCas polypeptide is a dCas9 polypeptide. In some embodiments, the RNA-guided DNA-binding agent lacking cleavase and nickase activity or the dCas DNA-binding polypeptide is a version of a Cas nuclease (e.g., a Cas nuclease discussed above) in which its endonucleolytic active sites are inactivated, e.g., by one or more alterations (e.g., point mutations) in its catalytic domains. See, e.g., US 2014/0186958 A1; US 2015/0166980 A1.

In some embodiments, the RNA-guided DNA-binding agent comprises one or more heterologous functional domains (e.g., is or comprises a fusion polypeptide).

In some embodiments, the heterologous functional domain may facilitate transport of the RNA-guided DNA-binding agent into the nucleus of a cell. For example, the heterologous functional domain may be a nuclear localization signal (NLS). In some embodiments, the RNA-guided DNA-binding agent may be fused with 1-10 NLS(s). In some embodiments, the RNA-guided DNA-binding agent may be fused with 1-5 NLS(s). In some embodiments, the RNA-guided DNA-binding agent may be fused with one NLS. Where one NLS is used, the NLS may be linked at the N-terminus or the C-terminus of the RNA-guided DNA-binding agent sequence. It may also be inserted within the RNA-guided DNA binding agent sequence. In other embodiments, the RNA-guided DNA-binding agent may be fused with more than one NLS. In some embodiments, the RNA-guided DNA-binding agent may be fused with 2, 3, 4, or 5 NLSs. In some embodiments, the RNA-guided DNA-binding agent may be fused with two NLSs. In certain circumstances, the two NLSs may be the same (e.g., two SV40 NLSs) or different. In some embodiments, the RNA-guided DNA-binding agent is fused to two SV40 NLS sequences linked at the carboxy terminus. In some embodiments, the RNA-guided DNA-binding agent may be fused with two NLSs, one linked at the N-terminus and one at the C-terminus. In some embodiments, the RNA-guided DNA-binding agent may be fused with 3 NLSs. In some embodiments, the RNA-guided DNA-binding agent may be fused with no NLS. In some embodiments, the NLS may be a monopartite sequence, such as, e.g., the SV40 NLS, PKKKRKV (SEQ ID NO: 600) or PKKKRRV (SEQ ID NO: 601). In some embodiments, the NLS may be a bipartite sequence, such as the NLS of nucleoplasmin, KRPAATKKAGQAKKKK (SEQ ID NO: 602). In a specific embodiment, a single PKKKRKV (SEQ ID NO: 600) NLS may be linked at the C-terminus of the RNA-guided DNA-binding agent. One or more linkers are optionally included at the fusion site.

In some embodiments, the heterologous functional domain may be capable of modifying the intracellular half-life of the RNA-guided DNA binding agent. In some embodiments, the half-life of the RNA-guided DNA binding agent may be increased. In some embodiments, the half-life of the RNA-guided DNA-binding agent may be reduced. In some embodiments, the heterologous functional domain may be capable of increasing the stability of the RNA-guided DNA-binding agent. In some embodiments, the heterologous functional domain may be capable of reducing the stability of the RNA-guided DNA-binding agent. In some embodiments, the heterologous functional domain may act as a signal peptide for protein degradation. In some embodiments, the protein degradation may be mediated by proteolytic enzymes, such as, for example, proteasomes, lysosomal proteases, or calpain proteases. In some embodiments, the heterologous functional domain may comprise a PEST sequence. In some embodiments, the RNA-guided DNA-binding agent may be modified by addition of ubiquitin or a polyubiquitin chain. In some embodiments, the ubiquitin may be a ubiquitin-like protein (UBL). Non-limiting examples of ubiquitin-like proteins include small ubiquitin-like modifier (SUMO), ubiquitin cross-reactive protein (UCRP, also known as interferon-stimulated gene-15 (ISG15)), ubiquitin-related modifier-1 (URM1), neuronal-precursor-cell-expressed developmentally downregulated protein-8 (NEDD8, also called RubI in S. cerevisiae), human leukocyte antigen F-associated (FAT10), autophagy-8 (ATG8) and -12 (ATG12), Fau ubiquitin-like protein (FUB1), membrane-anchored UBL (MUB), ubiquitin fold-modifier-1 (UFM1), and ubiquitin-like protein-5 (UBL5).

In some embodiments, the heterologous functional domain may be a marker domain. Non-limiting examples of marker domains include fluorescent proteins, purification tags, epitope tags, and reporter gene sequences. In some embodiments, the marker domain may be a fluorescent protein. Non-limiting examples of suitable fluorescent proteins include green fluorescent proteins (e.g., GFP, GFP-2, tagGFP, turboGFP, sfGFP, EGFP, Emerald, Azami Green, Monomeric Azami Green, CopGFP, AceGFP, ZsGreen1), yellow fluorescent proteins (e.g., YFP, EYFP, Citrine, Venus, YPet, PhiYFP, ZsYellowl), blue fluorescent proteins (e.g., EBFP, EBFP2, Azurite, mKalamal, GFPuv, Sapphire, T-sapphire,), cyan fluorescent proteins (e.g., ECFP, Cerulean, CyPet, AmCyanl, Midoriishi-Cyan), red fluorescent proteins (e.g., mKate, mKate2, mPlum, DsRed monomer, mCherry, mRFP1, DsRed-Express, DsRed2, DsRed-Monomer, HcRed-Tandem, HcRedl, AsRed2, eqFP611, mRasberry, mStrawberry, Jred), and orange fluorescent proteins (mOrange, mKO, Kusabira-Orange, Monomeric Kusabira-Orange, mTangerine, tdTomato) or any other suitable fluorescent protein. In other embodiments, the marker domain may be a purification tag and/or an epitope tag. Non-limiting exemplary tags include glutathione-S-transferase (GST), chitin binding protein (CBP), maltose binding protein (MBP), thioredoxin (TRX), poly(NANP), tandem affinity purification (TAP) tag, myc, AcV5, AUl, AU5, E, ECS, E2, FLAG, HA, nus, Softag 1, Softag 3, Strep, SBP, Glu-Glu, HSV, KT3, S, S1, T7, V5, VSV-G, 6×His, 8×His, biotin carboxyl carrier protein (BCCP), poly-His, and calmodulin. Non-limiting exemplary reporter genes include glutathione-S-transferase (GST), horseradish peroxidase (HRP), chloramphenicol acetyltransferase (CAT), beta-galactosidase, beta-glucuronidase, luciferase, or fluorescent proteins.

In additional embodiments, the heterologous functional domain may target the RNA-guided DNA-binding agent to a specific organelle, cell type, tissue, or organ. In some embodiments, the heterologous functional domain may target the RNA-guided DNA-binding agent to mitochondria.

In further embodiments, the heterologous functional domain may be an effector domain. When the RNA-guided DNA-binding agent is directed to its target sequence, e.g., when a Cas nuclease is directed to a target sequence by a gRNA, the effector domain may modify or affect the target sequence. In some embodiments, the effector domain may be chosen from a nucleic acid binding domain, a nuclease domain (e.g., a non-Cas nuclease domain), an epigenetic modification domain, a transcriptional activation domain, or a transcriptional repressor domain. In some embodiments, the heterologous functional domain is a nuclease, such as a Fok1 nuclease. See, e.g., U.S. Pat. No. 9,023,649. In some embodiments, the heterologous functional domain is a transcriptional activator or repressor. See, e.g., Qi et al., “Repurposing CRISPR as an RNA-guided platform for sequence-specific control of gene expression,” Cell 152:1173-83 (2013); Perez-Pinera et al., “RNA-guided gene activation by CRISPR-Cas9-based transcription factors,” Nat. Methods 10:973-6 (2013); Mali et al., “CAS9 transcriptional activators for target specificity screening and paired nickases for cooperative genome engineering,” Nat. Biotechnol. 31:833-8 (2013); Gilbert et al., “CRISPR-mediated modular RNA-guided regulation of transcription in eukaryotes,” Cell 154:442-51 (2013). As such, the RNA-guided DNA-binding agent essentially becomes a transcription factor that can be directed to bind a desired target sequence using a guide RNA.

E. Determination of Efficacy of gRNAs

In some embodiments, the efficacy of a gRNA is determined when delivered or expressed together with other components forming an RNP. In some embodiments, the gRNA is expressed together with an RNA-guided DNA binding agent, such as a Cas protein, e.g. Cas9. In some embodiments, the gRNA is delivered to or expressed in a cell line that already stably expresses an RNA-guided DNA nuclease, such as a Cas nuclease or nickase, e.g. Cas9 nuclease or nickase. In some embodiments the gRNA is delivered to a cell as part of an RNP. In some embodiments, the gRNA is delivered to a cell along with a mRNA encoding an RNA-guided DNA nuclease, such as a Cas nuclease or nickase, e.g. Cas9 nuclease or nickase.

As described herein, use of an RNA-guided DNA nuclease and a guide RNA disclosed herein can lead to double-stranded breaks in the DNA which can produce errors in the form of insertion/deletion (indel) mutations upon repair by cellular machinery. Many mutations due to indels alter the reading frame or introduce premature stop codons and, therefore, produce a non-functional protein.

In some embodiments, the efficacy of particular gRNAs is determined based on in vitro models. In some embodiments, the in vitro model is HEK293 cells stably expressing Cas9 (HEK293_Cas9). In some embodiments, the in vitro model is HUH7 human hepatocarcinoma cells. In some embodiments, the in vitro model is HepG2 cells. In some embodiments, the in vitro model is primary human hepatocytes. In some embodiments, the in vitro model is primary cynomolgus hepatocytes. With respect to using primary human hepatocytes, commercially available primary human hepatocytes can be used to provide greater consistency between experiments. In some embodiments, the number of off-target sites at which a deletion or insertion occurs in an in vitro model (e.g., in primary human hepatocytes) is determined, e.g., by analyzing genomic DNA from primary human hepatocytes transfected in vitro with Cas9 mRNA and the guide RNA. In some embodiments, such a determination comprises analyzing genomic DNA from primary human hepatocytes transfected in vitro with Cas9 mRNA, the guide RNA, and a donor oligonucleotide. Exemplary procedures for such determinations are provided in the working examples below.

In some embodiments, the efficacy of particular gRNAs is determined across multiple in vitro cell models for a gRNA selection process. In some embodiments, a cell line comparison of data with selected gRNAs is performed. In some embodiments, cross screening in multiple cell models is performed.

In some embodiments, the efficacy of particular gRNAs is determined based on in vivo models. In some embodiments, the in vivo model is a rodent model. In some embodiments, the rodent model is a mouse which expresses a LDHA gene. In some embodiments, the rodent model is a mouse which expresses a human LDHA gene. In some embodiments, the in vivo model is a non-human primate, for example cynomolgus monkey.

In some embodiments, the efficacy of a guide RNA is measured by percent editing of LDHA. In some embodiments, the percent editing of LDHA is compared to the percent editing necessary to achieve knockdown of LDHA protein, e.g., from whole cell lysates in the case of an in vitro model or in tissue in the case of an in vivo model.

In some embodiments, the efficacy of a guide RNA is measured by the number and/or frequency of indels at off-target sequences within the genome of the target cell type. In some embodiments, efficacious guide RNAs are provided which produce indels at off target sites at very low frequencies (e.g., <5%) in a cell population and/or relative to the frequency of indel creation at the target site. Thus, the disclosure provides for guide RNAs which do not exhibit off-target indel formation in the target cell type (e.g., a hepatocyte), or which produce a frequency of off-target indel formation of <5% in a cell population and/or relative to the frequency of indel creation at the target site. In some embodiments, the disclosure provides guide RNAs which do not exhibit any off target indel formation in the target cell type (e.g., hepatocyte). In some embodiments, guide RNAs are provided which produce indels at less than 5 off-target sites, e.g., as evaluated by one or more methods described herein. In some embodiments, guide RNAs are provided which produce indels at less than or equal to 4, 3, 2, or 1 off-target site(s) e.g., as evaluated by one or more methods described herein. In some embodiments, the off-target site(s) does not occur in a protein coding region in the target cell (e.g., hepatocyte) genome.

In some embodiments, detecting gene editing events, such as the formation of insertion/deletion (“indel”) mutations and homology directed repair (HDR) events in target DNA utilize linear amplification with a tagged primer and isolating the tagged amplification products (herein after referred to as “LAM-PCR,” or “Linear Amplification (LA)” method).

In some embodiments, the efficacy of a guide RNA is measured by mearing levels of glycolate and/or levels of oxalate in a sample such as a body fluid, e.g., serum, plasma, blood, or urine. In some embodiments, the efficacy of a guide RNA is measured by mearing levels of glycolate in the serum or plasma and/or levels of oxalate in the urine. An increase in the levels of glycolate in the serum or plasma and/or a decrease in the level of oxalate in the urine is indicative of an effective guide RNA. In some embodiments, urinary oxalate is reduced below 0.7 mmol/24 hrs/1.73 m². In some embodiments, levels of glycolate and oxalate are measured using an enzyme-linked immunosorbent assay (ELISA) assay with cell culture media or serum or plasma. In some embodiments, levels of glycolate and oxalate are measured in the same in vitro or in vivo systems or models used to measure editing. In some embodiments, levels of glycolate and oxalate are measured in cells, e.g., primary human hepatocytes. In some embodiments, levels of glycolate and oxalate are measured in H1UH7 cells. In some embodiments, levels of glycolate and oxalate are measured in HepG2 cells.

### III. Therapeutic Methods

The gRNAs and associated methods and compositions disclosed herein are useful in inducing a double-stranded break (DSB) within the LDHA gene and reducing the expression of the LDHA gene. The gRNAs and associated methods and compositions disclosed herein are useful in treating and preventing hyperoxaluria and preventing symptoms of hyperoxaluria. In some embodiments, the gRNAs disclosed herein are useful in treating and preventing calcium oxalate production, calcium oxalate deposition in organs, primary hyperoxaluria (including PH1, PH2, and PH3), oxalosis, including systemic oxalosis, and hematuria. In some embodiments, the gRNAs disclosed herein are useful in delaying or ameliorating the need for kidney or liver transplant. In some embodiments, the gRNAs disclosed herein are useful in preventing end stage renal disease (ESRD). Administration of the gRNAs disclosed herein will increase serum or plasma glycolate and decrease oxalate production or accumulation so that less oxalate is excreted in the urine. Therefore, in one aspect, effectiveness of treatment/prevention can be assessed by measuring serum or plasma glycolate, wherein an increase in glycolate levels indicates effectiveness. In some embodiments, effectiveness of treatment/prevention can be assessed by measuring oxalate in a sample, such as urinary oxalate, wherein a decrease in urinary oxalate indicates effectiveness.

Normal daily oxalate excretion in the urine of healthy subjects is less than about 45 mg, while concentrations exceeding about 45 mg per 24 hours are considered to be clinical hyperoxaluria (See e.g., Bhasin et al., World J Nephrol 2015 May 6; 4(2): 235-244; and Cochat P., Rumsby G. (2013). N Engl J Med 369:649-658). Accordingly, in some embodiments, administration of the gRNAs and compositions disclosed herein are useful for reducing levels of oxalate such that a subject no longer exhibits levels of urinary oxalate associated with clinical hyperoxaluria. In some embodiments, administration of the gRNAs and compositions disclosed herein reduces a subject's urinary oxalate to less than about 45 or 40 mg in a 24-hour period. In some embodiments, administration of the gRNAs and compositions disclosed herein reduces a subject's urinary oxalate to less than about 35, less than about 30, less than about 25, less than about 20, less than about 15, or less than about 10 mg in a 24-hour period.

In some embodiments, any one or more of the gRNAs, compositions, or pharmaceutical formulations described herein is for use in preparing a medicament for treating or preventing a disease or disorder in a subject. In some embodiments, treatment and/or prevention is accomplished with a single dose, e.g., one-time treatment, of medicament/composition. In some embodiments, the disease or disorder is hyperoxaluria.

In some embodiments, the invention comprises a method of treating or preventing a disease or disorder in subject comprising administering any one or more of the gRNAs, compositions, or pharmaceutical formulations described herein. In some embodiments, the disease or disorder is hyperoxaluria. In some embodiments, the gRNAs, compositions, or pharmaceutical formulations described herein are administered as a single dose, e.g., at one time. In some embodiments, the single dose achieves durable treatment and/or prevention. In some embodiments, the method achieves durable treatment and/or prevention. Durable treatment and/or prevention, as used herein, includes treatment and/or prevention that extends at least i) 3, 4, 5, 6, 7, 8, 9, 10, 11, 12, 13, 14, or 15 weeks; ii) 1, 2, 3, 4, 5, 6, 7, 8, 9, 10, 11, 12, 18, 24, 30, or 36 months; or iii) 1, 2, 3, 4, 5, 6, 7, 8, 9, or 10 years. In some embodiments, a single dose of the gRNAs, compositions, or pharmaceutical formulations described herein is sufficient to treat and/or prevent any of the indications described herein for the duration of the subject's life.

In some embodiments, the invention comprises a method or use of modifying (e.g., creating a double strand break) a target DNA comprising, administering or delivering any one or more of the gRNAs, compositions, or pharmaceutical formulations described herein. In some embodiments, the target DNA is the LDHA gene. In some embodiments, the target DNA is in an exon of the LDHA gene. In some embodiments, the target DNA is in exon 1, 2, 3, 4, 5, 6, 7, or 8 of the LDHA gene.

In some embodiments, the invention comprises a method or use for modulation of a target gene comprising, administering or delivering any one or more of the gRNAs, compositions, or pharmaceutical formulations described herein. In some embodiments, the modulation is editing of the LDHA target gene. In some embodiments, the modulation is a change in expression of the protein encoded by the LDHA target gene.

In some embodiments, the method or use results in gene editing. In some embodiments, the method or use results in a double-stranded break within the target LDHA gene. In some embodiments, the method or use results in formation of indel mutations during non-homologous end joining of the DSB. In some embodiments, the method or use results in an insertion or deletion of nucleotides in a target LDHA gene. In some embodiments, the insertion or deletion of nucleotides in a target LDHA gene leads to a frameshift mutation or premature stop codon that results in a non-functional protein. In some embodiments, the insertion or deletion of nucleotides in a target LDHA gene leads to a knockdown or elimination of target gene expression. In some embodiments, the method or use comprises homology directed repair of a DSB.

In some embodiments, the method or use results in LDHA gene modulation. In some embodiments, the LDHA gene modulation is a decrease in gene expression. In some embodiments, the method or use results in decreased expression of the protein encoded by the target gene.

In some embodiments, a method of inducing a double-stranded break (DSB) within the LDHA gene is provided comprising administering a composition comprising a guide RNA comprising any one or more guide sequences of SEQ ID NOs:1-84, or any one or more of the sgRNAs of SEQ ID NOs: 1001, 1005, 1007, 1008, 1014, 1023, 1027, 1032, 1045, 1048, 1063, 1067, 1069, 1071, 1074, 1076, 1077, 1078, 1079, and 1081, or modified versions thereof as shown, e.g., in SEQ ID NOs: 2001, 2005, 2007, 2008, 2014, 2023, 2027, 2032, 2045, 2048, 2063, 2067, 2069, 2071, 2074, 2076, 2077, 2078, 2079, and 2081. In some embodiments, gRNAs comprising any one or more of the guide sequences of SEQ ID NOs:1-84 and 100-192 are administered to induce a DSB in the LDHA gene. The guide RNAs may be administered together with an RNA-guided DNA nuclease such as a Cas nuclease (e.g., Cas9) or an mRNA or vector encoding an RNA-guided DNA nuclease such as a Cas nuclease (e.g., Cas9).

In some embodiments, a method of modifying the LDHA gene is provided comprising administering a composition comprising a guide RNA comprising any one or more of the guide sequences of SEQ ID NOs:1-84, or any one or more of the sgRNAs of SEQ ID NOs: 1001, 1005, 1007, 1008, 1014, 1023, 1027, 1032, 1045, 1048, 1063, 1067, 1069, 1071, 1074, 1076, 1077, 1078, 1079, and 1081, or modified versions thereof as shown, e.g., in SEQ ID NOs: 2001, 2005, 2007, 2008, 2014, 2023, 2027, 2032, 2045, 2048, 2063, 2067, 2069, 2071, 2074, 2076, 2077, 2078, 2079, and 2081. In some embodiments, gRNAs comprising any one or more of the guide sequences of SEQ ID NOs:1-84, or any one or more of the sgRNAs of SEQ ID NOs: 1001, 1005, 1007, 1008, 1014, 1023, 1027, 1032, 1045, 1048, 1063, 1067, 1069, 1071, 1074, 1076, 1077, 1078, 1079, and 1081, or modified versions thereof as shown, e.g., in SEQ ID NOs: 2001, 2005, 2007, 2008, 2014, 2023, 2027, 2032, 2045, 2048, 2063, 2067, 2069, 2071, 2074, 2076, 2077, 2078, 2079, and 2081, are administered to modify the LDHA gene. The guide RNAs may be administered together with an RNA-guided DNA nuclease such as a Cas nuclease (e.g., Cas9) or an mRNA or vector encoding an RNA-guided DNA nuclease such as a Cas nuclease (e.g., Cas9).

In some embodiments, a method of treating or preventing hyperoxaluria is provided comprising administering a composition comprising a guide RNA comprising any one or more of the guide sequences of SEQ ID NOs:1-84, or any one or more of the sgRNAs of SEQ ID NOs: 1001, 1005, 1007, 1008, 1014, 1023, 1027, 1032, 1045, 1048, 1063, 1067, 1069, 1071, 1074, 1076, 1077, 1078, 1079, and 1081, or modified versions thereof as shown, e.g., in SEQ ID NOs: 2001, 2005, 2007, 2008, 2014, 2023, 2027, 2032, 2045, 2048, 2063, 2067, 2069, 2071, 2074, 2076, 2077, 2078, 2079, and 2081. In some embodiments, gRNAs comprising any one or more of the guide sequences of SEQ ID NOs:1-84, or any one or more of the sgRNAs of SEQ ID NOs: 1001, 1005, 1007, 1008, 1014, 1023, 1027, 1032, 1045, 1048, 1063, 1067, 1069, 1071, 1074, 1076, 1077, 1078, 1079, and 1081, or modified versions thereof as shown, e.g., in SEQ ID NOs: 2001, 2005, 2007, 2008, 2014, 2023, 2027, 2032, 2045, 2048, 2063, 2067, 2069, 2071, 2074, 2076, 2077, 2078, 2079, and 2081 are administered to treat or prevent hyperoxaluria. The guide RNAs may be administered together with an RNA-guided DNA nuclease such as a Cas nuclease (e.g., Cas9) or an mRNA or vector encoding an RNA-guided DNA nuclease such as a Cas nuclease (e.g., Cas9). In some embodiments, the hyperoxaluria is primary hyperoxaluria. In some embodiments, the primary hyperoxaluria is type 1 (PH1), type 2 (PH2), or type 3 (PH3). In some embodiments, the hyperoxaluria is idiopathic.

In some embodiments, a method of decreasing or eliminating calcium oxalate production and/or deposition is provided comprising administering a guide RNA comprising any one or more of the guide sequences of SEQ ID NOs:1-84, or any one or more of the sgRNAs of SEQ ID NOs: 1001, 1005, 1007, 1008, 1014, 1023, 1027, 1032, 1045, 1048, 1063, 1067, 1069, 1071, 1074, 1076, 1077, 1078, 1079, and 1081, or modified versions thereof as shown, e.g., in SEQ ID NOs: 2001, 2005, 2007, 2008, 2014, 2023, 2027, 2032, 2045, 2048, 2063, 2067, 2069, 2071, 2074, 2076, 2077, 2078, 2079, and 2081. The guide RNAs may be administered together with an RNA-guided DNA nuclease such as a Cas nuclease (e.g., Cas9) or an mRNA or vector encoding an RNA-guided DNA nuclease such as a Cas nuclease (e.g., Cas9).

In some embodiments, a method of treating or preventing primary hyperoxaluria, including PH1, PH2, or PH3, is provided comprising administering a guide RNA comprising any one or more of the guide sequences of SEQ ID NOs:1-84, or any one or more of the sgRNAs of SEQ ID NOs: 1001, 1005, 1007, 1008, 1014, 1023, 1027, 1032, 1045, 1048, 1063, 1067, 1069, 1071, 1074, 1076, 1077, 1078, 1079, and 1081, or modified versions thereof as shown, e.g., in SEQ ID NOs: 2001, 2005, 2007, 2008, 2014, 2023, 2027, 2032, 2045, 2048, 2063, 2067, 2069, 2071, 2074, 2076, 2077, 2078, 2079, and 2081. The guide RNAs may be administered together with an RNA-guided DNA nuclease such as a Cas nuclease (e.g., Cas9) or an mRNA or vector encoding an RNA-guided DNA nuclease such as a Cas nuclease (e.g., Cas9).

In some embodiments, a method of treating or preventing oxalosis, including systemic oxalosis is provided comprising administering a guide RNA comprising any one or more of the guide sequences of SEQ ID NOs:1-84, or any one or more of the sgRNAs of SEQ ID NOs: 1001, 1005, 1007, 1008, 1014, 1023, 1027, 1032, 1045, 1048, 1063, 1067, 1069, 1071, 1074, 1076, 1077, 1078, 1079, and 1081, or modified versions thereof as shown, e.g., in SEQ ID NOs: 2001, 2005, 2007, 2008, 2014, 2023, 2027, 2032, 2045, 2048, 2063, 2067, 2069, 2071, 2074, 2076, 2077, 2078, 2079, and 2081. The guide RNAs may be administered together with an RNA-guided DNA nuclease such as a Cas nuclease (e.g., Cas9) or an mRNA or vector encoding an RNA-guided DNA nuclease such as a Cas nuclease (e.g., Cas9).

In some embodiments, a method of treating or preventing hematuria is provided comprising administering a guide RNA comprising any one or more of the guide sequences of SEQ ID NOs:1-84, or any one or more of the sgRNAs of SEQ ID NOs: 1001, 1005, 1007, 1008, 1014, 1023, 1027, 1032, 1045, 1048, 1063, 1067, 1069, 1071, 1074, 1076, 1077, 1078, 1079, and 1081, or modified versions thereof as shown, e.g., in SEQ ID NOs: 2001, 2005, 2007, 2008, 2014, 2023, 2027, 2032, 2045, 2048, 2063, 2067, 2069, 2071, 2074, 2076, 2077, 2078, 2079, and 2081. The guide RNAs may be administered together with an RNA-guided DNA nuclease such as a Cas nuclease (e.g., Cas9) or an mRNA or vector encoding an RNA-guided DNA nuclease such as a Cas nuclease (e.g., Cas9).

In some embodiments, gRNAs comprising any one or more of the guide sequences of SEQ ID NOs:1-84 and 100-192 or any one or more of the sgRNAs of SEQ ID NOs: 1001, 1005, 1007, 1008, 1014, 1023, 1027, 1032, 1045, 1048, 1063, 1067, 1069, 1071, 1074, 1076, 1077, 1078, 1079, and 1081, or modified versions thereof as shown, e.g., in SEQ ID NOs: 2001, 2005, 2007, 2008, 2014, 2023, 2027, 2032, 2045, 2048, 2063, 2067, 2069, 2071, 2074, 2076, 2077, 2078, 2079, and 2081 are administered to reduce oxalate levels in the urine. The gRNAs may be administered together with an RNA-guided DNA nuclease such as a Cas nuclease (e.g., Cas9) or an mRNA or vector encoding an RNA-guided DNA nuclease such as a Cas nuclease (e.g., Cas9).

In some embodiments, gRNAs comprising any one or more of the guide sequences of SEQ ID NOs:1-84 and 100-192 or any one or more of the sgRNAs of SEQ ID NOs: 1001, 1005, 1007, 1008, 1014, 1023, 1027, 1032, 1045, 1048, 1063, 1067, 1069, 1071, 1074, 1076, 1077, 1078, 1079, and 1081, or modified versions thereof as shown, e.g., in SEQ ID NOs: 2001, 2005, 2007, 2008, 2014, 2023, 2027, 2032, 2045, 2048, 2063, 2067, 2069, 2071, 2074, 2076, 2077, 2078, 2079, and 2081 are administered to increase serum glycolate in the serum or plasma. The gRNAs may be administered together with an RNA-guided DNA nuclease such as a Cas nuclease (e.g., Cas9) or an mRNA or vector encoding an RNA-guided DNA nuclease such as a Cas nuclease (e.g., Cas9).

In some embodiments, the gRNAs comprising the guide sequences of Table 1 together with an RNA-guided DNA nuclease such as a Cas nuclease induce DSBs, and non-homologous ending joining (NHEJ) during repair leads to a mutation in the LDHA gene. In some embodiments, NHEJ leads to a deletion or insertion of a nucleotide(s), which induces a frame shift or nonsense mutation in the LDHA gene.

In some embodiments, administering the guide RNAs of the invention (e.g., in a composition provided herein) increases levels (e.g., serum or plasma levels) of glycolate in the subject, and therefore prevents oxalate accumulation.

In some embodiments, increasing serum glycolate results in a decrease of urinary oxalate. In some embodiments, reduction of urinary oxalate reduces or eliminate calcium oxalate formation and deposition in organs.

In some embodiments, the subject is mammalian. In some embodiments, the subject is human. In some embodiments, the subject is cow, pig, monkey, sheep, dog, cat, fish, or poultry.

In some embodiments, the use of a guide RNAs comprising any one or more of the guide sequences in Table 1 or one or more sgRNAs from Table 2 (e.g., in a composition provided herein) is provided for the preparation of a medicament for treating a human subject having hyperoxaluria.

In some embodiments, the guide RNAs, compositions, and formulations are administered intravenously. In some embodiments, the guide RNAs, compositions, and formulations are administered into the hepatic circulation.

In some embodiments, a single administration of a composition comprising a guide RNA provided herein is sufficient to knock down expression of the mutant protein. In other embodiments, more than one administration of a composition comprising a guide RNA provided herein may be beneficial to maximize therapeutic effects.

In some embodiments, treatment slows or halts hyperoxaluria disease progression.

In some embodiments, treatment slows or halts progression of end stage renal disease (ESRD). In some embodiments, treatment slows or halts the need for kidney and/or liver transplant. In some embodiments, treatment results in improvement, stabilization, or slowing of change in symptoms of hyperoxaluria.

### A. Combination Therapy

In some embodiments, the invention comprises combination therapies comprising any one of the gRNAs comprising any one or more of the guide sequences disclosed in Table 1 (e.g., in a composition provided herein) together with an additional therapy suitable for alleviating hyperoxaluria and its symptoms, as described above.

In some embodiments, the additional therapy for hyperoxaluria is vitamin B6, hydration, renal dialysis, or liver or kidney transplant. In some embodiments, the additional therapy is another agent that disrupts the LDHA gene, such as, for example, an siRNA directed to the LDHA gene. In some embodiments the siRNA directed to the LDHA gene is DCR-PHXC. In some embodiments, such as when the hyperoxaluria is caused by PH1, the additional therapy is an agent that disrupts the HAO gene, such as, for example, an siRNA directed to the HA01 gene. In some embodiments, the HAO1 siRNA is lumasiran (ALN-GO1; Alnylam).

In some embodiments, the combination therapy comprises any one of the gRNAs comprising any one or more of the guide sequences disclosed in Table 1 together with a siRNA that targets HA01 or LDHA. In some embodiments, the siRNA is any siRNA capable of further reducing or eliminating the expression of LDHA. In some embodiments, the siRNA is administered after any one of the gRNAs comprising any one or more of the guide sequences disclosed in Table 1 (e.g., in a composition provided herein). In some embodiments, the siRNA is administered on a regular basis following treatment with any of the gRNA compositions provided herein.

In some embodiments, the combination therapy comprises any one of the gRNAs comprising any one or more of the guide sequences disclosed in Table 1 (e.g., in a composition provided herein) together with antisense nucleotide that targets LDHA. In some embodiments, the antisense nucleotide is any antisense nucleotide capable of further reducing or eliminating the expression of LDHA. In some embodiments, the antisense nucleotide is administered after any one of the gRNAs comprising any one or more of the guide sequences disclosed in Table 1 (e.g., in a composition provided herein). In some embodiments, the antisense nucleotide is administered on a regular basis following treatment with any of the gRNA compositions provided herein.

B. Delivery of gRNA Compositions

Lipid nanoparticles (LNPs) are a well-known means for delivery of nucleotide and protein cargo, and may be used for delivery of the guide RNAs, compositions, or pharmaceutical formulations disclosed herein. In some embodiments, the LNPs deliver nucleic acid, protein, or nucleic acid together with protein.

In some embodiments, the invention comprises a method for delivering any one of the gRNAs disclosed herein to a subject, wherein the gRNA is associated with an LNP. In some embodiments, the gRNA/LNP is also associated with a Cas9 or an mRNA encoding Cas9.

In some embodiments, the invention comprises a composition comprising any one of the gRNAs disclosed and an LNP. In some embodiments, the composition further comprises a Cas9 or an mRNA encoding Cas9.

In some embodiments, the LNPs comprise cationic lipids. In some embodiments, the LNPs comprise (9Z,12Z)-3-((4,4-bis(octyloxy)butanoyl)oxy)-2-((((3-(diethylamino)propoxy)carbonyl)oxy)methyl)propyl octadeca-9,12-dienoate, also called 3-((4,4-bis(octyloxy)butanoyl)oxy)-2-((((3-(diethylamino)propoxy)carbonyl)oxy)methyl)propyl (9Z,12Z)-octadeca-9,12-dienoate) or another ionizable lipid. See, e.g., lipids of WO/2017/173054 and references described therein. In some embodiments, the LNPs comprise molar ratios of a cationic lipid amine to RNA phosphate (N.P) of about 4.5, 5.0, 5.5, 6.0, or 6.5. In some embodiments, the term cationic and ionizable in the context of LNP lipids is interchangeable, e.g., wherein ionizable lipids are cationic depending on the pH.

In some embodiments, LNPs associated with the gRNAs disclosed herein are for use in preparing a medicament for treating a disease or disorder.

Electroporation is a well-known means for delivery of cargo, and any electroporation methodology may be used for delivery of any one of the gRNAs disclosed herein. In some embodiments, electroporation may be used to deliver any one of the gRNAs disclosed herein and Cas9 or an mRNA encoding Cas9.

In some embodiments, the invention comprises a method for delivering any one of the gRNAs disclosed herein to an ex vivo cell, wherein the gRNA is associated with an LNP or not associated with an LNP. In some embodiments, the gRNA/LNP or gRNA is also associated with a Cas9 or an mRNA encoding Cas9.

In some embodiments, the guide RNA compositions described herein, alone or encoded on one or more vectors, are formulated in or administered via a lipid nanoparticle; see e.g., WO/2017/173054, filed Mar. 30, 2017 and published May 10, 2017 entitled “LIPID NANOPARTICLE FORMULATIONS FOR CRISPR/CAS COMPONENTS,” the contents of which are hereby incorporated by reference in their entirety.

In certain embodiments, the invention comprises DNA or RNA vectors encoding any of the guide RNAs comprising any one or more of the guide sequences described herein. In some embodiments, in addition to guide RNA sequences, the vectors further comprise nucleic acids that do not encode guide RNAs. Nucleic acids that do not encode guide RNA include, but are not limited to, promoters, enhancers, regulatory sequences, and nucleic acids encoding an RNA-guided DNA nuclease, which can be a nuclease such as Cas9. In some embodiments, the vector comprises one or more nucleotide sequence(s) encoding a crRNA, a trRNA, or a crRNA and trRNA. In some embodiments, the vector comprises one or more nucleotide sequence(s) encoding a sgRNA and an mRNA encoding an RNA-guided DNA nuclease, which can be a Cas nuclease, such as Cas9 or Cpf1. In some embodiments, the vector comprises one or more nucleotide sequence(s) encoding a crRNA, a trRNA, and an mRNA encoding an RNA-guided DNA nuclease, which can be a Cas protein, such as, Cas9. In one embodiment, the Cas9 is from Streptococcus pyogenes (i.e., Spy Cas9). In some embodiments, the nucleotide sequence encoding the crRNA, trRNA, or crRNA and trRNA (which may be a sgRNA) comprises or consists of a guide sequence flanked by all or a portion of a repeat sequence from a naturally-occurring CRISPR/Cas system. The nucleic acid comprising or consisting of the crRNA, trRNA, or crRNA and trRNA may further comprise a vector sequence wherein the vector sequence comprises or consists of nucleic acids that are not naturally found together with the crRNA, trRNA, or crRNA and trRNA.

This description and exemplary embodiments should not be taken as limiting. For the purposes of this specification and appended claims, unless otherwise indicated, all numbers expressing quantities, percentages, or proportions, and other numerical values used in the specification and claims, are to be understood as being modified in all instances by the term “about,” to the extent they are not already so modified. Accordingly, unless indicated to the contrary, the numerical parameters set forth in the following specification and attached claims are approximations that may vary depending upon the desired properties sought to be obtained. At the very least, and not as an attempt to limit the application of the doctrine of equivalents to the scope of the claims, each numerical parameter should at least be construed in light of the number of reported significant digits and by applying ordinary rounding techniques.

It is noted that, as used in this specification and the appended claims, the singular forms “a,” “an,” and “the,” and any singular use of any word, include plural referents unless expressly and unequivocally limited to one referent. As used herein, the term “include” and its grammatical variants are intended to be non-limiting, such that recitation of items in a list is not to the exclusion of other like items that can be substituted or added to the listed items.

### EXAMPLES

The following examples are provided to illustrate certain disclosed embodiments and are not to be construed as limiting the scope of this disclosure in any way.

### Example 1—Materials and Methods

In Vitro Transcription (“IVT”) of Nuclease mRNA

Capped and polyadenylated Streptococcus pyogenes (“Spy”) Cas9 mRNA containing N1-methyl pseudo-U was generated by in vitro transcription using a linearized plasmid DNA template and T7 RNA polymerase. Plasmid DNA containing a T7 promoter and a sequence for transcription (for producing mRNA comprising an mRNA described herein (see SEQ ID NOs: 501-515 in Table 24 below for exemplary ORFs) was linearized by incubating at 37° C. to complete digestion with XbaI with the following conditions: 200 ng/μL plasmid, 2 U/μL XbaI (NEB), and 1× reaction buffer. The XbaI was inactivated by heating the reaction at 65° C. for 20 min. The linearized plasmid was purified from enzyme and buffer salts using a silica maxi spin column (Epoch Life Sciences) and analyzed by agarose gel to confirm linearization. The IVT reaction to generate Cas9 modified mRNA was incubated at 37° C. for 4 hours in the following conditions: 50 ng/μL linearized plasmid; 2 mM each of GTP, ATP, CTP, and N1-methyl pseudo-UTP (Trilink); 10 mM ARCA (Trilink); 5 U/μL T7 RNA polymerase (NEB); 1 U/μL Murine RNase inhibitor (NEB); 0.004 U/μL Inorganic E. coli pyrophosphatase (NEB); and 1× reaction buffer. After the 4-hour incubation, TURBO DNase (ThermoFisher) was added to a final concentration of 0.01 U/μL, and the reaction was incubated for an additional 30 minutes to remove the DNA template. The Cas9 mRNA was purified from enzyme and nucleotides using a MegaClear Transcription Clean-up kit according to the manufacturer's protocol (ThermoFisher). Alternatively, the Cas9 mRNA was purified with a LiCl precipitation method, which in some cases was followed by further purification by tangential flow filtration. The transcript concentration was determined by measuring the light absorbance at 260 nm (Nanodrop), and the transcript was analyzed by capillary electrophoresis by Bioanlayzer (Agilent).

The sequence for transcription of Cas9 mRNA used in the Examples comprised a sequence selected from SEQ ID NO: 501-515 as shown in Table 24.

TABLE 24
Exemplary Cas9 mRNA Sequences

| SEQ | |
|---|---|
| ID NO | Sequence |
| 501 | GGGTCCCGCAGTCGGCGTCCAGCGGCTCTGCTTGTTCGTGTGTGTGTCGTTGCAGGCCTTATTCGGATCCGCCACCATGGAC |
| | AAGAAGTACAGCATCGGACTGGACATCGGAACAAACAGCGTCGGATGGGCAGTCATCACAGACGAATACAAGGTCCCGAG |
| | CAAGAAGTTCAAGGTCCTGGGAAACACAGACAGACACAGCATCAAGAAGAACCTGATCGGAGCACTGCTGTTCGACAGCG |
| | GAGAAACAGCAGAAGCAACAAGACTGAAGAGAACAGCAAGAAGAAGATACACAAGAAGAAAGAACAGAATCTGCTACCT |
| | GCAGGAAATCTTCAGCAACGAAATGGCAAAGGTCGACGACAGCTTCTTCCACAGACTGGAAGAAAGCTTCCTGGTCGAAG |
| | AAGACAAGAAGCACGAAAGACACCCGATCTTCGGAAACATCGTCGACGAAGTCGCATACCACGAAAAGTACCCGACAATC |
| | TACCACCTGAGAAAGAAGCTGGTCGACAGCACAGACAAGGCAGACCTGAGACTGATCTACCTGGCACTGGCACACATGAT |
| | CAAGTTCAGAGGACACTTCCTGATCGAAGGAGACCTGAACCCGGACAACAGCGACGTCGACAAGCTGTTCATCCAGCTGG |
| | TCCAGACATACAACCAGCTGTTCGAAGAAAACCCGATCAACGCAAGCGGAGTCGACGCAAAGGCAATCCTGAGCGCAAGA |
| | CTGAGCAAGAGCAGAAGACTGGAAAACCTGATCGCACAGCTGCCGGGAGAAAAGAAGAACGGACTGTTCGGAAACCTGA |
| | TCGCACTGAGCCTGGGACTGACACCGAACTTCAAGAGCAACTTCGACCTGGCAGAAGACGCAAAGCTGCAGCTGAGCAAG |
| | GACACATACGACGACGACCTGGACAACCTGCTGGCACAGATCGGAGACCAGTACGCAGACCTGTTCCTGGCAGCAAAGAA |
| | CCTGAGCGACGCAATCCTGCTGAGCGACATCCTGAGAGTCAACACAGAAATCACAAAGGCACCGCTGAGCGCAAGCATGA |
| | TCAAGAGATACGACGAACACCACCAGGACCTGACACTGCTGAAGGCACTGGTCAGACAGCAGCTGCCGGAAAAGTACAAG |
| | GAAATCTTCTTCGACCAGAGCAAGAACGGATACGCAGGATACATCGACGGAGGAGCAAGCCAGGAAGAATTCTACAAGTT |
| | CATCAAGCCGATCCTGGAAAAGATGGACGGAACAGAAGAACTGCTGGTCAAGCTGAACAGAGAAGACCTGCTGAGAAAG |
| | CAGAGAACATTCGACAACGGAAGCATCCCGCACCAGATCCACCTGGGAGAACTGCACGCAATCCTGAGAAGACAGGAAG |
| | ACTTCTACCCGTTCCTGAAGGACAACAGAGAAAAGATCGAAAAGATCCTGACATTCAGAATCCCGTACTACGTCGGACCGC |
| | TGGCAAGAGGAAACAGCAGATTCGCATGGATGACAAGAAAGAGCGAAGAAACAATCACACCGTGGAACTTCGAAGAAGT |
| | CGTCGACAAGGGAGCAAGCGCACAGAGCTTCATCGAAAGAATGACAAACTTCGACAAGAACCTGCCGAACGAAAAGGTCC |
| | TGCCGAAGCACAGCCTGCTGTACGAATACTTCACAGTCTACAACGAACTGACAAAGGTCAAGTACGTCACAGAAGGAATG |
| | AGAAAGCCGGCATTCCTGAGCGGAGAACAGAAGAAGGCAATCGTCGACCTGCTGTTCAAGACAAACAGAAAGGTCACAGT |
| | CAAGCAGCTGAAGGAAGACTACTTCAAGAAGATCGAATGCTTCGACAGCGTCGAAATCAGCGGAGTCGAAGACAGATTCA |
| | ACGCAAGCCTGGGAACATACCACGACCTGCTGAAGATCATCAAGGACAAGGACTTCCTGGACAACGAAGAAAACGAAGAC |
| | ATCCTGGAAGACATCGTCCTGACACTGACACTGTTCGAAGACAGAGAAATGATCGAAGAAAGACTGAAGACATACGCACA |
| | CCTGTTCGACGACAAGGTCATGAAGCAGCTGAAGAGAAGAAGATACACAGGATGGGGAAGACTGAGCAGAAAGCTGATC |
| | AACGGAATCAGAGACAAGCAGAGCGGAAAGACAATCCTGGACTTCCTGAAGAGCGACGGATTCGCAAACAGAAACTTCAT |
| | GCAGCTGATCCACGACGACAGCCTGACATTCAAGGAAGACATCCAGAAGGCACAGGTCAGCGGACAGGGAGACAGCCTG |
| | CACGAACACATCGCAAACCTGGCAGGAAGCCCGGCAATCAAGAAGGGAATCCTGCAGACAGTCAAGGTCGTCGACGAACT |
| | GGTCAAGGTCATGGGAAGACACAAGCCGGAAAACATCGTCATCGAAATGGCAAGAGAAAACCAGACAACACAGAAGGGA |
| | CAGAAGAACAGCAGAGAAAGAATGAAGAGAATCGAAGAAGGAATCAAGGAACTGGGAAGCCAGATCCTGAAGGAACAC |
| | CCGGTCGAAAACACACAGCTGCAGAACGAAAAGCTGTACCTGTACTACCTGCAGAACGGAAGAGACATGTACGTCGACCA |
| | GGAACTGGACATCAACAGACTGAGCGACTACGACGTCGACCACATCGTCCCGCAGAGCTTCCTGAAGGACGACAGCATCG |
| | ACAACAAGGTCCTGACAAGAAGCGACAAGAACAGAGGAAAGAGCGACAACGTCCCGAGCGAAGAAGTCGTCAAGAAGAT |
| | GAAGAACTACTGGAGACAGCTGCTGAACGCAAAGCTGATCACACAGAGAAAGTTCGACAACCTGACAAAGGCAGAGAGA |
| | GGAGGACTGAGCGAACTGGACAAGGCAGGATTCATCAAGAGACAGCTGGTCGAAACAAGACAGATCACAAAGCACGTCG |
| | CACAGATCCTGGACAGCAGAATGAACACAAAGTACGACGAAAACGACAAGCTGATCAGAGAAGTCAAGGTCATCACACTG |
| | AAGAGCAAGCTGGTCAGCGACTTCAGAAAGGACTTCCAGTTCTACAAGGTCAGAGAAATCAACAACTACCACCACGCACA |
| | CGACGCATACCTGAACGCAGTCGTCGGAACAGCACTGATCAAGAAGTACCCGAAGCTGGAAAGCGAATTCGTCTACGGAG |
| | ACTACAAGGTCTACGACGTCAGAAAGATGATCGCAAAGAGCGAACAGGAAATCGGAAAGGCAACAGCAAAGTACTTCTTC |
| | TACAGCAACATCATGAACTTCTTCAAGACAGAAATCACACTGGCAAACGGAGAAATCAGAAAGAGACCGCTGATCGAAAC |
| | AAACGGAGAAACAGGAGAAATCGTCTGGGACAAGGGAAGAGACTTCGCAACAGTCAGAAAGGTCCTGAGCATGCCGCAG |
| | GTCAACATCGTCAAGAAGACAGAAGTCCAGACAGGAGGATTCAGCAAGGAAAGCATCCTGCCGAAGAGAAACAGCGACA |
| | AGCTGATCGCAAGAAAGAAGGACTGGGACCCGAAGAAGTACGGAGGATTCGACAGCCCGACAGTCGCATACAGCGTCCTG |
| | GTCGTCGCAAAGGTCGAAAAGGGAAAGAGCAAGAAGCTGAAGAGCGTCAAGGAACTGCTGGGAATCACAATCATGGAAA |
| | GAAGCAGCTTCGAAAAGAACCCGATCGACTTCCTGGAAGCAAAGGGATACAAGGAAGTCAAGAAGGACCTGATCATCAAG |
| | CTGCCGAAGTACAGCCTGTTCGAACTGGAAAACGGAAGAAAGAGAATGCTGGCAAGCGCAGGAGAACTGCAGAAGGGAA |
| | ACGAACTGGCACTGCCGAGCAAGTACGTCAACTTCCTGTACCTGGCAAGCCACTACGAAAAGCTGAAGGGAAGCCCGGAA |
| | GACAACGAACAGAAGCAGCTGTTCGTCGAACAGCACAAGCACTACCTGGACGAAATCATCGAACAGATCAGCGAATTCAG |
| | CAAGAGAGTCATCCTGGCAGACGCAAACCTGGACAAGGTCCTGAGCGCATACAACAAGCACAGAGACAAGCCGATCAGA |
| | GAACAGGCAGAAAACATCATCCACCTGTTCACACTGACAAACCTGGGAGCACCGGCAGCATTCAAGTACTTCGACACAAC |
| | AATCGACAGAAAGAGATACACAAGCACAAAGGAAGTCCTGGACGCAACACTGATCCACCAGAGCATCACAGGACTGTACG |
| | AAACAAGAATCGACCTGAGCCAGCTGGGAGGAGACGGAGGAGGAAGCCCGAAGAAGAAGAGAAAGGTCTAGCTAGCCAT |
| | CACATTTAAAAGCATCTCAGCCTACCATGAGAATAAGAGAAAGAAAATGAAGATCAATAGCTTATTCATCTCTTTTTCTTTT |
| | TCGTTGGTGTAAAGCCAACACCCTGTCTAAAAAACATAAATTTCTTTAATCATTTTGCCTCTTTTCTCTGTGCTTCAATTAAT |
| | AAAAAATGGAAAGAACCTCGAG |
| 502 | AUGGACAAGAAGUACAGCAUCGGACUGGACAUCGGAACAAACAGCGUCGGAUGGGCAGUCAUCACAGACGAAUACAAG |
| | AGGUCCUGGGAAACACAGACAGACACAGCAUCAAGAAGAACCUGAUCGGAGCACUGCUGUUCGACAGCGGAGAAACAG |
| | GAGAACAGCAAGAAGAAGAUACACAAGAAGAAAGAACAGAAUCUGCUACCUGCAGGAAAUCUUCAGCAACGAAAUGGC |
| | UUCCACAGACUGGAAGAAAGCUUCCUGGUCGAAGAAGACAAGAAGCACGAAAGACACCCGAUCUUCGGAAACAUCGUC |
| | AAAAGUACCCGACAAUCUACCACCUGAGAAAGAAGCUGGUCGACAGCACAGACAAGGCAGACCUGAGACUGAUCUACCU |
| | AAGUUCAGAGGACACUUCCUGAUCGAAGGAGACCUGAACCCGGACAACAGCGACGUCGACAAGCUGUUCAUCCAGCUGG |
| | GUUCGAAGAAAACCCGAUCAACGCAAGCGGAGUCGACGCAAAGGCAAUCCUGAGCGCAAGACUGAGCAAGAGCAGAAG |
| | CAGCUGCCGGGAGAAAAGAAGAACGGACUGUUCGGAAACCUGAUCGCACUGAGCCUGGGACUGACACCGAACUUCAAG |
| | AAGACGCAAAGCUGCAGCUGAGCAAGGACACAUACGACGACGACCUGGACAACCUGCUGGCACAGAUCGGAGACCAGUA |
| | GCAAAGAACCUGAGCGACGCAAUCCUGCUGAGCGACAUCCUGAGAGUCAACACAGAAAUCACAAAGGCACCGCUGAGCG |
| | CGACGAACACCACCAGGACCUGACACUGCUGAAGGCACUGGUCAGACAGCAGCUGCCGGAAAAGUACAAGGAAAUCUU |
| | GGAUACGCAGGAUACAUCGACGGAGGAGCAAGCCAGGAAGAAUUCUACAAGUUCAUCAAGCCGAUCCUGGAAAAGAUG |
| | UGGUCAAGCUGAACAGAGAAGACCUGCUGAGAAAGCAGAGAACAUUCGACAACGGAAGCAUCCCGCACCAGAUCCACC |
| | CCUGAGAAGACAGGAAGACUUCUACCCGUUCCUGAAGGACAACAGAGAAAAGAUCGAAAAGAUCCUGACAUUCAGAAU |
| | CUGGCAAGAGGAAACAGCAGAUUCGCAUGGAUGACAAGAAAGAGCGAAGAAACAAUCACACCGUGGAACUUCGAAGAA |
| | GCGCACAGAGCUUCAUCGAAAGAAUGACAAACUUCGACAAGAACCUGCCGAACGAAAAGGUCCUGCCGAAGCACAGCCU |
| | GUCUACAACGAACUGACAAAGGUCAAGUACGUCACAGAAGGAAUGAGAAAGCCGGCAUUCCUGAGCGGAGAACAGAAG |
| | UGUUCAAGACAAACAGAAAGGUCACAGUCAAGCAGCUGAAGGAAGACUACUUCAAGAAGAUCGAAUGCUUCGACAGCG |
| | AGACAGAUUCAACGCAAGCCUGGGAACAUACCACGACCUGCUGAAGAUCAUCAAGGACAAGGACUUCCUGGACAACGA |
| | GAAGACAUCGUCCUGACACUGACACUGUUCGAAGACAGAGAAAUGAUCGAAGAAAGACUGAAGACAUACGCACACCUG |
| | AGCAGCUGAAGAGAAGAAGAUACACAGGAUGGGGAAGACUGAGCAGAAAGCUGAUCAACGGAAUCAGAGACAAGCAGA |
| | CUUCCUGAAGAGCGACGGAUUCGCAAACAGAAACUUCAUGCAGCUGAUCCACGACGACAGCCUGACAUUCAAGGAAGA |
| | AGCGGACAGGGAGACAGCCUGCACGAACACAUCGCAAACCUGGCAGGAAGCCCGGCAAUCAAGAAGGGAAUCCUGCAGA |
| | ACUGGUCAAGGUCAUGGGAAGACACAAGCCGGAAAACAUCGUCAUCGAAAUGGCAAGAGAAAACCAGACAACACAGAA |
| | GAAAGAAUGAAGAGAAUCGAAGAAGGAAUCAAGGAACUGGGAAGCCAGAUCCUGAAGGAACACCCGGUCGAAAACACA |
| | UGUACCUGUACUACCUGCAGAACGGAAGAGACAUGUACGUCGACCAGGAACUGGACAUCAACAGACUGAGCGACUACG |
| | GCAGAGCUUCCUGAAGGACGACAGCAUCGACAACAAGGUCCUGACAAGAAGCGACAAGAACAGAGGAAAGAGCGACAAC |
| | UCAAGAAGAUGAAGAACUACUGGAGACAGCUGCUGAACGCAAAGCUGAUCACACAGAGAAAGUUCGACAACCUGACAA |
| | GAGCGAACUGGACAAGGCAGGAUUCAUCAAGAGACAGCUGGUCGAAACAAGACAGAUCACAAAGCACGUCGCACAGAU |
| | ACAAAGUACGACGAAAACGACAAGCUGAUCAGAGAAGUCAAGGUCAUCACACUGAAGAGCAAGCUGGUCAGCGACUUC |
| | ACAAGGUCAGAGAAAUCAACAACUACCACCACGCACACGACGCAUACCUGAACGCAGUCGUCGGAACAGCACUGAUCAA |
| | AGCGAAUUCGUCUACGGAGACUACAAGGUCUACGACGUCAGAAAGAUGAUCGCAAAGAGCGAACAGGAAAUCGGAAAG |
| | UCUACAGCAACAUCAUGAACUUCUUCAAGACAGAAAUCACACUGGCAAACGGAGAAAUCAGAAAGAGACCGCUGAUCG |
| | AGAAAUCGUCUGGGACAAGGGAAGAGACUUCGCAACAGUCAGAAAGGUCCUGAGCAUGCCGCAGGUCAACAUCGUCAA |
| | GGAGGAUUCAGCAAGGAAAGCAUCCUGCCGAAGAGAAACAGCGACAAGCUGAUCGCAAGAAAGAAGGACUGGGACCCG |
| | ACAGCCCGACAGUCGCAUACAGCGUCCUGGUCGUCGCAAAGGUCGAAAAGGGAAAGAGCAAGAAGCUGAAGAGCGUCA |
| | AAUCAUGGAAAGAAGCAGCUUCGAAAAGAACCCGAUCGACUUCCUGGAAGCAAAGGGAUACAAGGAAGUCAAGAAGGA |
| | AAGUACAGCCUGUUCGAACUGGAAAACGGAAGAAAGAGAAUGCUGGCAAGCGCAGGAGAACUGCAGAAGGGAAACGAA |
| | ACGUCAACUUCCUGUACCUGGCAAGCCACUACGAAAAGCUGAAGGGAAGCCCGGAAGACAACGAACAGAAGCAGCUGUU |
| | UACCUGGACGAAAUCAUCGAACAGAUCAGCGAAUUCAGCAAGAGAGUCAUCCUGGCAGACGCAAACCUGGACAAGGUCC |
| | CAGAGACAAGCCGAUCAGAGAACAGGCAGAAAACAUCAUCCACCUGUUCACACUGACAAACCUGGGAGCACCGGCAGCA |
| | CAAUCGACAGAAAGAGAUACACAAGCACAAAGGAAGUCCUGGACGCAACACUGAUCCACCAGAGCAUCACAGGACUGU |
| | GAGCCAGCUGGGAGGAGACUAG |
| 503 | GACAAGAAGUACAGCAUCGGACUGGACAUCGGAACAAACAGCGUCGGAUGGGCAGUCAUCACAGACGAAUACAAGGUC |
| | CCGAGCAAGAAGUUCAAGGUCCUGGGAAACACAGACAGACACAGCAUCAAGAAGAACCUGAUCGGAGCACUGCUGUUC |
| | GACAGCGGAGAAACAGCAGAAGCAACAAGACUGAAGAGAACAGCAAGAAGAAGAUACACAAGAAGAAAGAACAGAAUC |
| | UGCUACCUGCAGGAAAUCUUCAGCAACGAAAUGGCAAAGGUCGACGACAGCUUCUUCCACAGACUGGAAGAAAGCUUC |
| | CUGGUCGAAGAAGACAAGAAGCACGAAAGACACCCGAUCUUCGGAAACAUCGUCGACGAAGUCGCAUACCACGAAAAG |
| | UACCCGACAAUCUACCACCUGAGAAAGAAGCUGGUCGACAGCACAGACAAGGCAGACCUGAGACUGAUCUACCUGGCA |
| | CUGGCACACAUGAUCAAGUUCAGAGGACACUUCCUGAUCGAAGGAGACCUGAACCCGGACAACAGCGACGUCGACAAG |
| | CUGUUCAUCCAGCUGGUCCAGACAUACAACCAGCUGUUCGAAGAAAACCCGAUCAACGCAAGCGGAGUCGACGCAAAG |
| | GCAAUCCUGAGCGCAAGACUGAGCAAGAGCAGAAGACUGGAAAACCUGAUCGCACAGCUGCCGGGAGAAAAGAAGAAC |
| | GGACUGUUCGGAAACCUGAUCGCACUGAGCCUGGGACUGACACCGAACUUCAAGAGCAACUUCGACCUGGCAGAAGAC |
| | GCAAAGCUGCAGCUGAGCAAGGACACAUACGACGACGACCUGGACAACCUGCUGGCACAGAUCGGAGACCAGUACGCA |
| | GACCUGUUCCUGGCAGCAAAGAACCUGAGCGACGCAAUCCUGCUGAGCGACAUCCUGAGAGUCAACACAGAAAUCACA |
| | AAGGCACCGCUGAGCGCAAGCAUGAUCAAGAGAUACGACGAACACCACCAGGACCUGACACUGCUGAAGGCACUGGUC |
| | AGACAGCAGCUGCCGGAAAAGUACAAGGAAAUCUUCUUCGACCAGAGCAAGAACGGAUACGCAGGAUACAUCGACGGA |
| | GGAGCAAGCCAGGAAGAAUUCUACAAGUUCAUCAAGCCGAUCCUGGAAAAGAUGGACGGAACAGAAGAACUGCUGGUC |
| | AAGCUGAACAGAGAAGACCUGCUGAGAAAGCAGAGAACAUUCGACAACGGAAGCAUCCCGCACCAGAUCCACCUGGGA |
| | GAACUGCACGCAAUCCUGAGAAGACAGGAAGACUUCUACCCGUUCCUGAAGGACAACAGAGAAAAGAUCGAAAAGAUC |
| | CUGACAUUCAGAAUCCCGUACUACGUCGGACCGCUGGCAAGAGGAAACAGCAGAUUCGCAUGGAUGACAAGAAAGAGC |
| | GAAGAAACAAUCACACCGUGGAACUUCGAAGAAGUCGUCGACAAGGGAGCAAGCGCACAGAGCUUCAUCGAAAGAAUG |
| | ACAAACUUCGACAAGAACCUGCCGAACGAAAAGGUCCUGCCGAAGCACAGCCUGCUGUACGAAUACUUCACAGUCUAC |
| | AACGAACUGACAAAGGUCAAGUACGUCACAGAAGGAAUGAGAAAGCCGGCAUUCCUGAGCGGAGAACAGAAGAAGGCA |
| | AUCGUCGACCUGCUGUUCAAGACAAACAGAAAGGUCACAGUCAAGCAGCUGAAGGAAGACUACUUCAAGAAGAUCGAA |
| | UGCUUCGACAGCGUCGAAAUCAGCGGAGUCGAAGACAGAUUCAACGCAAGCCUGGGAACAUACCACGACCUGCUGAAG |
| | AUCAUCAAGGACAAGGACUUCCUGGACAACGAAGAAAACGAAGACAUCCUGGAAGACAUCGUCCUGACACUGACACUG |
| | UUCGAAGACAGAGAAAUGAUCGAAGAAAGACUGAAGACAUACGCACACCUGUUCGACGACAAGGUCAUGAAGCAGCUG |
| | AAGAGAAGAAGAUACACAGGAUGGGGAAGACUGAGCAGAAAGCUGAUCAACGGAAUCAGAGACAAGCAGAGCGGAAA |
| | GACAAUCCUGGACUUCCUGAAGAGCGACGGAUUCGCAAACAGAAACUUCAUGCAGCUGAUCCACGACGACAGCCUGAC |
| | AUUCAAGGAAGACAUCCAGAAGGCACAGGUCAGCGGACAGGGAGACAGCCUGCACGAACACAUCGCAAACCUGGCAGG |
| | AAGCCCGGCAAUCAAGAAGGGAAUCCUGCAGACAGUCAAGGUCGUCGACGAACUGGUCAAGGUCAUGGGAAGACACAA |
| | GCCGGAAAACAUCGUCAUCGAAAUGGCAAGAGAAAACCAGACAACACAGAAGGGACAGAAGAACAGCAGAGAAAGAAU |
| | GAAGAGAAUCGAAGAAGGAAUCAAGGAACUGGGAAGCCAGAUCCUGAAGGAACACCCGGUCGAAAACACACAGCUGCA |
| | GAACGAAAAGCUGUACCUGUACUACCUGCAGAACGGAAGAGACAUGUACGUCGACCAGGAACUGGACAUCAACAGACU |
| | GAGCGACUACGACGUCGACCACAUCGUCCCGCAGAGCUUCCUGAAGGACGACAGCAUCGACAACAAGGUCCUGACAAG |
| | AAGCGACAAGAACAGAGGAAAGAGCGACAACGUCCCGAGCGAAGAAGUCGUCAAGAAGAUGAAGAACUACUGGAGACA |
| | GCUGCUGAACGCAAAGCUGAUCACACAGAGAAAGUUCGACAACCUGACAAAGGCAGAGAGAGGAGGACUGAGCGAACU |
| | GGACAAGGCAGGAUUCAUCAAGAGACAGCUGGUCGAAACAAGACAGAUCACAAAGCACGUCGCACAGAUCCUGGACAG |
| | CAGAAUGAACACAAAGUACGACGAAAACGACAAGCUGAUCAGAGAAGUCAAGGUCAUCACACUGAAGAGCAAGCUGGU |
| | CAGCGACUUCAGAAAGGACUUCCAGUUCUACAAGGUCAGAGAAAUCAACAACUACCACCACGCACACGACGCAUACCU |
| | GAACGCAGUCGUCGGAACAGCACUGAUCAAGAAGUACCCGAAGCUGGAAAGCGAAUUCGUCUACGGAGACUACAAGGU |
| | CUACGACGUCAGAAAGAUGAUCGCAAAGAGCGAACAGGAAAUCGGAAAGGCAACAGCAAAGUACUUCUUCUACAGCAA |
| | CAUCAUGAACUUCUUCAAGACAGAAAUCACACUGGCAAACGGAGAAAUCAGAAAGAGACCGCUGAUCGAAACAAACGG |
| | AGAAACAGGAGAAAUCGUCUGGGACAAGGGAAGAGACUUCGCAACAGUCAGAAAGGUCCUGAGCAUGCCGCAGGUCAA |
| | CAUCGUCAAGAAGACAGAAGUCCAGACAGGAGGAUUCAGCAAGGAAAGCAUCCUGCCGAAGAGAAACAGCGACAAGCU |
| | GAUCGCAAGAAAGAAGGACUGGGACCCGAAGAAGUACGGAGGAUUCGACAGCCCGACAGUCGCAUACAGCGUCCUGGU |
| | CGUCGCAAAGGUCGAAAAGGGAAAGAGCAAGAAGCUGAAGAGCGUCAAGGAACUGCUGGGAAUCACAAUCAUGGAAAG |
| | AAGCAGCUUCGAAAAGAACCCGAUCGACUUCCUGGAAGCAAAGGGAUACAAGGAAGUCAAGAAGGACCUGAUCAUCAA |
| | GCUGCCGAAGUACAGCCUGUUCGAACUGGAAAACGGAAGAAAGAGAAUGCUGGCAAGCGCAGGAGAACUGCAGAAGGG |
| | AAACGAACUGGCACUGCCGAGCAAGUACGUCAACUUCCUGUACCUGGCAAGCCACUACGAAAAGCUGAAGGGAAGCCC |
| | GGAAGACAACGAACAGAAGCAGCUGUUCGUCGAACAGCACAAGCACUACCUGGACGAAAUCAUCGAACAGAUCAGCGA |
| | AUUCAGCAAGAGAGUCAUCCUGGCAGACGCAAACCUGGACAAGGUCCUGAGCGCAUACAACAAGCACAGAGACAAGCC |
| | GAUCAGAGAACAGGCAGAAAACAUCAUCCACCUGUUCACACUGACAAACCUGGGAGCACCGGCAGCAUUCAAGUACUU |
| | CGACACAACAAUCGACAGAAAGAGAUACACAAGCACAAAGGAAGUCCUGGACGCAACACUGAUCCACCAGAGCAUCAC |
| | AGGACUGUACGAAACAAGAAUCGACCUGAGCCAGCUGGGAGGAGAC |
| 504 | AUGGACAAGAAGUACAGCAUCGGACUGGACAUCGGAACAAACAGCGUCGGAUGGGCAGUCAUCACAGACGAAUACAAG |
| | GUCCCGAGCAAGAAGUUCAAGGUCCUGGGAAACACAGACAGACACAGCAUCAAGAAGAACCUGAUCGGAGCACUGCUG |
| | UUCGACAGCGGAGAAACAGCAGAAGCAACAAGACUGAAGAGAACAGCAAGAAGAAGAUACACAAGAAGAAAGAACAGA |
| | AUCUGCUACCUGCAGGAAAUCUUCAGCAACGAAAUGGCAAAGGUCGACGACAGCUUCUUCCACAGACUGGAAGAAAGC |
| | UUCCUGGUCGAAGAAGACAAGAAGCACGAAAGACACCCGAUCUUCGGAAACAUCGUCGACGAAGUCGCAUACCACGAA |
| | AAGUACCCGACAAUCUACCACCUGAGAAAGAAGCUGGUCGACAGCACAGACAAGGCAGACCUGAGACUGAUCUACCUG |
| | GCACUGGCACACAUGAUCAAGUUCAGAGGACACUUCCUGAUCGAAGGAGACCUGAACCCGGACAACAGCGACGUCGAC |
| | AAGCUGUUCAUCCAGCUGGUCCAGACAUACAACCAGCUGUUCGAAGAAAACCCGAUCAACGCAAGCGGAGUCGACGCA |
| | AAGGCAAUCCUGAGCGCAAGACUGAGCAAGAGCAGAAGACUGGAAAACCUGAUCGCACAGCUGCCGGGAGAAAAGAAG |
| | AACGGACUGUUCGGAAACCUGAUCGCACUGAGCCUGGGACUGACACCGAACUUCAAGAGCAACUUCGACCUGGCAGAA |
| | GACGCAAAGCUGCAGCUGAGCAAGGACACAUACGACGACGACCUGGACAACCUGCUGGCACAGAUCGGAGACCAGUAC |
| | GCAGACCUGUUCCUGGCAGCAAAGAACCUGAGCGACGCAAUCCUGCUGAGCGACAUCCUGAGAGUCAACACAGAAAUC |
| | ACAAAGGCACCGCUGAGCGCAAGCAUGAUCAAGAGAUACGACGAACACCACCAGGACCUGACACUGCUGAAGGCACUG |
| | GUCAGACAGCAGCUGCCGGAAAAGUACAAGGAAAUCUUCUUCGACCAGAGCAAGAACGGAUACGCAGGAUACAUCGAC |
| | GGAGGAGCAAGCCAGGAAGAAUUCUACAAGUUCAUCAAGCCGAUCCUGGAAAAGAUGGACGGAACAGAAGAACUGCUG |
| | GUCAAGCUGAACAGAGAAGACCUGCUGAGAAAGCAGAGAACAUUCGACAACGGAAGCAUCCCGCACCAGAUCCACCUG |
| | GGAGAACUGCACGCAAUCCUGAGAAGACAGGAAGACUUCUACCCGUUCCUGAAGGACAACAGAGAAAAGAUCGAAAAG |
| | AUCCUGACAUUCAGAAUCCCGUACUACGUCGGACCGCUGGCAAGAGGAAACAGCAGAUUCGCAUGGAUGACAAGAAAG |
| | AGCGAAGAAACAAUCACACCGUGGAACUUCGAAGAAGUCGUCGACAAGGGAGCAAGCGCACAGAGCUUCAUCGAAAGA |
| | AUGACAAACUUCGACAAGAACCUGCCGAACGAAAAGGUCCUGCCGAAGCACAGCCUGCUGUACGAAUACUUCACAGUC |
| | UACAACGAACUGACAAAGGUCAAGUACGUCACAGAAGGAAUGAGAAAGCCGGCAUUCCUGAGCGGAGAACAGAAGAAG |
| | GCAAUCGUCGACCUGCUGUUCAAGACAAACAGAAAGGUCACAGUCAAGCAGCUGAAGGAAGACUACUUCAAGAAGAUC |
| | GAAUGCUUCGACAGCGUCGAAAUCAGCGGAGUCGAAGACAGAUUCAACGCAAGCCUGGGAACAUACCACGACCUGCUG |
| | AAGAUCAUCAAGGACAAGGACUUCCUGGACAACGAAGAAAACGAAGACAUCCUGGAAGACAUCGUCCUGACACUGACA |
| | CUGUUCGAAGACAGAGAAAUGAUCGAAGAAAGACUGAAGACAUACGCACACCUGUUCGACGACAAGGUCAUGAAGCAG |
| | CUGAAGAGAAGAAGAUACACAGGAUGGGGAAGACUGAGCAGAAAGCUGAUCAACGGAAUCAGAGACAAGCAGAGCGG |
| | AAAGACAAUCCUGGACUUCCUGAAGAGCGACGGAUUCGCAAACAGAAACUUCAUGCAGCUGAUCCACGACGACAGCCU |
| | GACAUUCAAGGAAGACAUCCAGAAGGCACAGGUCAGCGGACAGGGAGACAGCCUGCACGAACACAUCGCAAACCUGGC |
| | AGGAAGCCCGGCAAUCAAGAAGGGAAUCCUGCAGACAGUCAAGGUCGUCGACGAACUGGUCAAGGUCAUGGGAAGACA |
| | CAAGCCGGAAAACAUCGUCAUCGAAAUGGCAAGAGAAAACCAGACAACACAGAAGGGACAGAAGAACAGCAGAGAAAG |
| | AAUGAAGAGAAUCGAAGAAGGAAUCAAGGAACUGGGAAGCCAGAUCCUGAAGGAACACCCGGUCGAAAACACACAGCU |
| | GCAGAACGAAAAGCUGUACCUGUACUACCUGCAGAACGGAAGAGACAUGUACGUCGACCAGGAACUGGACAUCAACAG |
| | ACUGAGCGACUACGACGUCGACCACAUCGUCCCGCAGAGCUUCCUGAAGGACGACAGCAUCGACAACAAGGUCCUGAC |
| | AAGAAGCGACAAGAACAGAGGAAAGAGCGACAACGUCCCGAGCGAAGAAGUCGUCAAGAAGAUGAAGAACUACUGGAG |
| | ACAGCUGCUGAACGCAAAGCUGAUCACACAGAGAAAGUUCGACAACCUGACAAAGGCAGAGAGAGGAGGACUGAGCGA |
| | ACUGGACAAGGCAGGAUUCAUCAAGAGACAGCUGGUCGAAACAAGACAGAUCACAAAGCACGUCGCACAGAUCCUGGA |
| | CAGCAGAAUGAACACAAAGUACGACGAAAACGACAAGCUGAUCAGAGAAGUCAAGGUCAUCACACUGAAGAGCAAGCU |
| | GGUCAGCGACUUCAGAAAGGACUUCCAGUUCUACAAGGUCAGAGAAAUCAACAACUACCACCACGCACACGACGCAUA |
| | CCUGAACGCAGUCGUCGGAACAGCACUGAUCAAGAAGUACCCGAAGCUGGAAAGCGAAUUCGUCUACGGAGACUACAA |
| | GGUCUACGACGUCAGAAAGAUGAUCGCAAAGAGCGAACAGGAAAUCGGAAAGGCAACAGCAAAGUACUUCUUCUACAG |
| | CAACAUCAUGAACUUCUUCAAGACAGAAAUCACACUGGCAAACGGAGAAAUCAGAAAGAGACCGCUGAUCGAAACAAA |
| | CGGAGAAACAGGAGAAAUCGUCUGGGACAAGGGAAGAGACUUCGCAACAGUCAGAAAGGUCCUGAGCAUGCCGCAGGU |
| | CAACAUCGUCAAGAAGACAGAAGUCCAGACAGGAGGAUUCAGCAAGGAAAGCAUCCUGCCGAAGAGAAACAGCGACAA |
| | GCUGAUCGCAAGAAAGAAGGACUGGGACCCGAAGAAGUACGGAGGAUUCGACAGCCCGACAGUCGCAUACAGCGUCCU |
| | GGUCGUCGCAAAGGUCGAAAAGGGAAAGAGCAAGAAGCUGAAGAGCGUCAAGGAACUGCUGGGAAUCACAAUCAUGGA |
| | AAGAAGCAGCUUCGAAAAGAACCCGAUCGACUUCCUGGAAGCAAAGGGAUACAAGGAAGUCAAGAAGGACCUGAUCAU |
| | CAAGCUGCCGAAGUACAGCCUGUUCGAACUGGAAAACGGAAGAAAGAGAAUGCUGGCAAGCGCAGGAGAACUGCAGAA |
| | GGGAAACGAACUGGCACUGCCGAGCAAGUACGUCAACUUCCUGUACCUGGCAAGCCACUACGAAAAGCUGAAGGGAAG |
| | CCCGGAAGACAACGAACAGAAGCAGCUGUUCGUCGAACAGCACAAGCACUACCUGGACGAAAUCAUCGAACAGAUCAG |
| | CGAAUUCAGCAAGAGAGUCAUCCUGGCAGACGCAAACCUGGACAAGGUCCUGAGCGCAUACAACAAGCACAGAGACAA |
| | GCCGAUCAGAGAACAGGCAGAAAACAUCAUCCACCUGUUCACACUGACAAACCUGGGAGCACCGGCAGCAUUCAAGUA |
| | CUUCGACACAACAAUCGACAGAAAGAGAUACACAAGCACAAAGGAAGUCCUGGACGCAACACUGAUCCACCAGAGCAU |
| | CACAGGACUGUACGAAACAAGAAUCGACCUGAGCCAGCUGGGAGGAGACGGAAGCGGAAGCCCGAAGAAGAAGAGAAA |
| | GGUCGACGGAAGCCCGAAGAAGAAGAGAAAGGUCGACAGCGGAUAG |
| 505 | GACAAGAAGUACAGCAUCGGACUGGACAUCGGAACAAACAGCGUCGGAUGGGCAGUCAUCACAGACGAAUACAAGGUC |
| | CCGAGCAAGAAGUUCAAGGUCCUGGGAAACACAGACAGACACAGCAUCAAGAAGAACCUGAUCGGAGCACUGCUGUUC |
| | GACAGCGGAGAAACAGCAGAAGCAACAAGACUGAAGAGAACAGCAAGAAGAAGAUACACAAGAAGAAAGAACAGAAUC |
| | UGCUACCUGCAGGAAAUCUUCAGCAACGAAAUGGCAAAGGUCGACGACAGCUUCUUCCACAGACUGGAAGAAAGCUUC |
| | CUGGUCGAAGAAGACAAGAAGCACGAAAGACACCCGAUCUUCGGAAACAUCGUCGACGAAGUCGCAUACCACGAAAAG |
| | UACCCGACAAUCUACCACCUGAGAAAGAAGCUGGUCGACAGCACAGACAAGGCAGACCUGAGACUGAUCUACCUGGCA |
| | CUGGCACACAUGAUCAAGUUCAGAGGACACUUCCUGAUCGAAGGAGACCUGAACCCGGACAACAGCGACGUCGACAAG |
| | CUGUUCAUCCAGCUGGUCCAGACAUACAACCAGCUGUUCGAAGAAAACCCGAUCAACGCAAGCGGAGUCGACGCAAAG |
| | GCAAUCCUGAGCGCAAGACUGAGCAAGAGCAGAAGACUGGAAAACCUGAUCGCACAGCUGCCGGGAGAAAAGAAGAAC |
| | GGACUGUUCGGAAACCUGAUCGCACUGAGCCUGGGACUGACACCGAACUUCAAGAGCAACUUCGACCUGGCAGAAGAC |
| | GCAAAGCUGCAGCUGAGCAAGGACACAUACGACGACGACCUGGACAACCUGCUGGCACAGAUCGGAGACCAGUACGCA |
| | GACCUGUUCCUGGCAGCAAAGAACCUGAGCGACGCAAUCCUGCUGAGCGACAUCCUGAGAGUCAACACAGAAAUCACA |
| | AAGGCACCGCUGAGCGCAAGCAUGAUCAAGAGAUACGACGAACACCACCAGGACCUGACACUGCUGAAGGCACUGGUC |
| | AGACAGCAGCUGCCGGAAAAGUACAAGGAAAUCUUCUUCGACCAGAGCAAGAACGGAUACGCAGGAUACAUCGACGGA |
| | GGAGCAAGCCAGGAAGAAUUCUACAAGUUCAUCAAGCCGAUCCUGGAAAAGAUGGACGGAACAGAAGAACUGCUGGUC |
| | AAGCUGAACAGAGAAGACCUGCUGAGAAAGCAGAGAACAUUCGACAACGGAAGCAUCCCGCACCAGAUCCACCUGGGA |
| | GAACUGCACGCAAUCCUGAGAAGACAGGAAGACUUCUACCCGUUCCUGAAGGACAACAGAGAAAAGAUCGAAAAGAUC |
| | CUGACAUUCAGAAUCCCGUACUACGUCGGACCGCUGGCAAGAGGAAACAGCAGAUUCGCAUGGAUGACAAGAAAGAGC |
| | GAAGAAACAAUCACACCGUGGAACUUCGAAGAAGUCGUCGACAAGGGAGCAAGCGCACAGAGCUUCAUCGAAAGAAUG |
| | ACAAACUUCGACAAGAACCUGCCGAACGAAAAGGUCCUGCCGAAGCACAGCCUGCUGUACGAAUACUUCACAGUCUAC |
| | AACGAACUGACAAAGGUCAAGUACGUCACAGAAGGAAUGAGAAAGCCGGCAUUCCUGAGCGGAGAACAGAAGAAGGCA |
| | AUCGUCGACCUGCUGUUCAAGACAAACAGAAAGGUCACAGUCAAGCAGCUGAAGGAAGACUACUUCAAGAAGAUCGAA |
| | UGCUUCGACAGCGUCGAAAUCAGCGGAGUCGAAGACAGAUUCAACGCAAGCCUGGGAACAUACCACGACCUGCUGAAG |
| | AUCAUCAAGGACAAGGACUUCCUGGACAACGAAGAAAACGAAGACAUCCUGGAAGACAUCGUCCUGACACUGACACUG |
| | UUCGAAGACAGAGAAAUGAUCGAAGAAAGACUGAAGACAUACGCACACCUGUUCGACGACAAGGUCAUGAAGCAGCUG |
| | AAGAGAAGAAGAUACACAGGAUGGGGAAGACUGAGCAGAAAGCUGAUCAACGGAAUCAGAGACAAGCAGAGCGGAAA |
| | GACAAUCCUGGACUUCCUGAAGAGCGACGGAUUCGCAAACAGAAACUUCAUGCAGCUGAUCCACGACGACAGCCUGAC |
| | AUUCAAGGAAGACAUCCAGAAGGCACAGGUCAGCGGACAGGGAGACAGCCUGCACGAACACAUCGCAAACCUGGCAGG |
| | AAGCCCGGCAAUCAAGAAGGGAAUCCUGCAGACAGUCAAGGUCGUCGACGAACUGGUCAAGGUCAUGGGAAGACACAA |
| | GCCGGAAAACAUCGUCAUCGAAAUGGCAAGAGAAAACCAGACAACACAGAAGGGACAGAAGAACAGCAGAGAAAGAAU |
| | GAAGAGAAUCGAAGAAGGAAUCAAGGAACUGGGAAGCCAGAUCCUGAAGGAACACCCGGUCGAAAACACACAGCUGCA |
| | GAACGAAAAGCUGUACCUGUACUACCUGCAGAACGGAAGAGACAUGUACGUCGACCAGGAACUGGACAUCAACAGACU |
| | GAGCGACUACGACGUCGACCACAUCGUCCCGCAGAGCUUCCUGAAGGACGACAGCAUCGACAACAAGGUCCUGACAAG |
| | AAGCGACAAGAACAGAGGAAAGAGCGACAACGUCCCGAGCGAAGAAGUCGUCAAGAAGAUGAAGAACUACUGGAGACA |
| | GCUGCUGAACGCAAAGCUGAUCACACAGAGAAAGUUCGACAACCUGACAAAGGCAGAGAGAGGAGGACUGAGCGAACU |
| | GGACAAGGCAGGAUUCAUCAAGAGACAGCUGGUCGAAACAAGACAGAUCACAAAGCACGUCGCACAGAUCCUGGACAG |
| | CAGAAUGAACACAAAGUACGACGAAAACGACAAGCUGAUCAGAGAAGUCAAGGUCAUCACACUGAAGAGCAAGCUGGU |
| | CAGCGACUUCAGAAAGGACUUCCAGUUCUACAAGGUCAGAGAAAUCAACAACUACCACCACGCACACGACGCAUACCU |
| | GAACGCAGUCGUCGGAACAGCACUGAUCAAGAAGUACCCGAAGCUGGAAAGCGAAUUCGUCUACGGAGACUACAAGGU |
| | CUACGACGUCAGAAAGAUGAUCGCAAAGAGCGAACAGGAAAUCGGAAAGGCAACAGCAAAGUACUUCUUCUACAGCAA |
| | CAUCAUGAACUUCUUCAAGACAGAAAUCACACUGGCAAACGGAGAAAUCAGAAAGAGACCGCUGAUCGAAACAAACGG |
| | AGAAACAGGAGAAAUCGUCUGGGACAAGGGAAGAGACUUCGCAACAGUCAGAAAGGUCCUGAGCAUGCCGCAGGUCAA |
| | CAUCGUCAAGAAGACAGAAGUCCAGACAGGAGGAUUCAGCAAGGAAAGCAUCCUGCCGAAGAGAAACAGCGACAAGCU |
| | GAUCGCAAGAAAGAAGGACUGGGACCCGAAGAAGUACGGAGGAUUCGACAGCCCGACAGUCGCAUACAGCGUCCUGGU |
| | CGUCGCAAAGGUCGAAAAGGGAAAGAGCAAGAAGCUGAAGAGCGUCAAGGAACUGCUGGGAAUCACAAUCAUGGAAAG |
| | AAGCAGCUUCGAAAAGAACCCGAUCGACUUCCUGGAAGCAAAGGGAUACAAGGAAGUCAAGAAGGACCUGAUCAUCAA |
| | GCUGCCGAAGUACAGCCUGUUCGAACUGGAAAACGGAAGAAAGAGAAUGCUGGCAAGCGCAGGAGAACUGCAGAAGGG |
| | AAACGAACUGGCACUGCCGAGCAAGUACGUCAACUUCCUGUACCUGGCAAGCCACUACGAAAAGCUGAAGGGAAGCCC |
| | GGAAGACAACGAACAGAAGCAGCUGUUCGUCGAACAGCACAAGCACUACCUGGACGAAAUCAUCGAACAGAUCAGCGA |
| | AUUCAGCAAGAGAGUCAUCCUGGCAGACGCAAACCUGGACAAGGUCCUGAGCGCAUACAACAAGCACAGAGACAAGCC |
| | GAUCAGAGAACAGGCAGAAAACAUCAUCCACCUGUUCACACUGACAAACCUGGGAGCACCGGCAGCAUUCAAGUACUU |
| | CGACACAACAAUCGACAGAAAGAGAUACACAAGCACAAAGGAAGUCCUGGACGCAACACUGAUCCACCAGAGCAUCAC |
| | AGGACUGUACGAAACAAGAAUCGACCUGAGCCAGCUGGGAGGAGACGGAAGCGGAAGCCCGAAGAAGAAGAGAAAGGU |
| | CGACGGAAGCCCGAAGAAGAAGAGAAAGGUCGACAGCGGA |
| 506 | GGGTCCCGCAGTCGGCGTCCAGCGGCTCTGCTTGTTCGTGTGTGTGTCGTTGCAGGCCTTATTCGGATCCATGGACAAGAAG |
| | TACAGCATCGGACTGGACATCGGAACAAACAGCGTCGGATGGGCAGTCATCACAGACGAATACAAGGTCCCGAGCAAGAA |
| | GTTCAAGGTCCTGGGAAACACAGACAGACACAGCATCAAGAAGAACCTGATCGGAGCACTGCTGTTCGACAGCGGAGAAA |
| | CAGCAGAAGCAACAAGACTGAAGAGAACAGCAAGAAGAAGATACACAAGAAGAAAGAACAGAATCTGCTACCTGCAGGA |
| | AATCTTCAGCAACGAAATGGCAAAGGTCGACGACAGCTTCTTCCACAGACTGGAAGAAAGCTTCCTGGTCGAAGAAGACA |
| | AGAAGCACGAAAGACACCCGATCTTCGGAAACATCGTCGACGAAGTCGCATACCACGAAAAGTACCCGACAATCTACCAC |
| | CTGAGAAAGAAGCTGGTCGACAGCACAGACAAGGCAGACCTGAGACTGATCTACCTGGCACTGGCACACATGATCAAGTT |
| | CAGAGGACACTTCCTGATCGAAGGAGACCTGAACCCGGACAACAGCGACGTCGACAAGCTGTTCATCCAGCTGGTCCAGA |
| | CATACAACCAGCTGTTCGAAGAAAACCCGATCAACGCAAGCGGAGTCGACGCAAAGGCAATCCTGAGCGCAAGACTGAGC |
| | AAGAGCAGAAGACTGGAAAACCTGATCGCACAGCTGCCGGGAGAAAAGAAGAACGGACTGTTCGGAAACCTGATCGCAC |
| | TGAGCCTGGGACTGACACCGAACTTCAAGAGCAACTTCGACCTGGCAGAAGACGCAAAGCTGCAGCTGAGCAAGGACACA |
| | TACGACGACGACCTGGACAACCTGCTGGCACAGATCGGAGACCAGTACGCAGACCTGTTCCTGGCAGCAAAGAACCTGAG |
| | CGACGCAATCCTGCTGAGCGACATCCTGAGAGTCAACACAGAAATCACAAAGGCACCGCTGAGCGCAAGCATGATCAAGA |
| | GATACGACGAACACCACCAGGACCTGACACTGCTGAAGGCACTGGTCAGACAGCAGCTGCCGGAAAAGTACAAGGAAATC |
| | TTCTTCGACCAGAGCAAGAACGGATACGCAGGATACATCGACGGAGGAGCAAGCCAGGAAGAATTCTACAAGTTCATCAA |
| | GCCGATCCTGGAAAAGATGGACGGAACAGAAGAACTGCTGGTCAAGCTGAACAGAGAAGACCTGCTGAGAAAGCAGAGA |
| | ACATTCGACAACGGAAGCATCCCGCACCAGATCCACCTGGGAGAACTGCACGCAATCCTGAGAAGACAGGAAGACTTCTA |
| | CCCGTTCCTGAAGGACAACAGAGAAAAGATCGAAAAGATCCTGACATTCAGAATCCCGTACTACGTCGGACCGCTGGCAA |
| | GAGGAAACAGCAGATTCGCATGGATGACAAGAAAGAGCGAAGAAACAATCACACCGTGGAACTTCGAAGAAGTCGTCGA |
| | CAAGGGAGCAAGCGCACAGAGCTTCATCGAAAGAATGACAAACTTCGACAAGAACCTGCCGAACGAAAAGGTCCTGCCGA |
| | AGCACAGCCTGCTGTACGAATACTTCACAGTCTACAACGAACTGACAAAGGTCAAGTACGTCACAGAAGGAATGAGAAAG |
| | CCGGCATTCCTGAGCGGAGAACAGAAGAAGGCAATCGTCGACCTGCTGTTCAAGACAAACAGAAAGGTCACAGTCAAGCA |
| | GCTGAAGGAAGACTACTTCAAGAAGATCGAATGCTTCGACAGCGTCGAAATCAGCGGAGTCGAAGACAGATTCAACGCAA |
| | GCCTGGGAACATACCACGACCTGCTGAAGATCATCAAGGACAAGGACTTCCTGGACAACGAAGAAAACGAAGACATCCTG |
| | GAAGACATCGTCCTGACACTGACACTGTTCGAAGACAGAGAAATGATCGAAGAAAGACTGAAGACATACGCACACCTGTT |
| | CGACGACAAGGTCATGAAGCAGCTGAAGAGAAGAAGATACACAGGATGGGGAAGACTGAGCAGAAAGCTGATCAACGGA |
| | ATCAGAGACAAGCAGAGCGGAAAGACAATCCTGGACTTCCTGAAGAGCGACGGATTCGCAAACAGAAACTTCATGCAGCT |
| | GATCCACGACGACAGCCTGACATTCAAGGAAGACATCCAGAAGGCACAGGTCAGCGGACAGGGAGACAGCCTGCACGAA |
| | CACATCGCAAACCTGGCAGGAAGCCCGGCAATCAAGAAGGGAATCCTGCAGACAGTCAAGGTCGTCGACGAACTGGTCAA |
| | GGTCATGGGAAGACACAAGCCGGAAAACATCGTCATCGAAATGGCAAGAGAAAACCAGACAACACAGAAGGGACAGAAG |
| | AACAGCAGAGAAAGAATGAAGAGAATCGAAGAAGGAATCAAGGAACTGGGAAGCCAGATCCTGAAGGAACACCCGGTCG |
| | AAAACACACAGCTGCAGAACGAAAAGCTGTACCTGTACTACCTGCAGAACGGAAGAGACATGTACGTCGACCAGGAACTG |
| | GACATCAACAGACTGAGCGACTACGACGTCGACCACATCGTCCCGCAGAGCTTCCTGAAGGACGACAGCATCGACAACAA |
| | GGTCCTGACAAGAAGCGACAAGAACAGAGGAAAGAGCGACAACGTCCCGAGCGAAGAAGTCGTCAAGAAGATGAAGAAC |
| | TACTGGAGACAGCTGCTGAACGCAAAGCTGATCACACAGAGAAAGTTCGACAACCTGACAAAGGCAGAGAGAGGAGGAC |
| | TGAGCGAACTGGACAAGGCAGGATTCATCAAGAGACAGCTGGTCGAAACAAGACAGATCACAAAGCACGTCGCACAGATC |
| | CTGGACAGCAGAATGAACACAAAGTACGACGAAAACGACAAGCTGATCAGAGAAGTCAAGGTCATCACACTGAAGAGCA |
| | AGCTGGTCAGCGACTTCAGAAAGGACTTCCAGTTCTACAAGGTCAGAGAAATCAACAACTACCACCACGCACACGACGCA |
| | TACCTGAACGCAGTCGTCGGAACAGCACTGATCAAGAAGTACCCGAAGCTGGAAAGCGAATTCGTCTACGGAGACTACAA |
| | GGTCTACGACGTCAGAAAGATGATCGCAAAGAGCGAACAGGAAATCGGAAAGGCAACAGCAAAGTACTTCTTCTACAGCA |
| | ACATCATGAACTTCTTCAAGACAGAAATCACACTGGCAAACGGAGAAATCAGAAAGAGACCGCTGATCGAAACAAACGGA |
| | GAAACAGGAGAAATCGTCTGGGACAAGGGAAGAGACTTCGCAACAGTCAGAAAGGTCCTGAGCATGCCGCAGGTCAACAT |
| | CGTCAAGAAGACAGAAGTCCAGACAGGAGGATTCAGCAAGGAAAGCATCCTGCCGAAGAGAAACAGCGACAAGCTGATC |
| | GCAAGAAAGAAGGACTGGGACCCGAAGAAGTACGGAGGATTCGACAGCCCGACAGTCGCATACAGCGTCCTGGTCGTCGC |
| | AAAGGTCGAAAAGGGAAAGAGCAAGAAGCTGAAGAGCGTCAAGGAACTGCTGGGAATCACAATCATGGAAAGAAGCAGC |
| | TTCGAAAAGAACCCGATCGACTTCCTGGAAGCAAAGGGATACAAGGAAGTCAAGAAGGACCTGATCATCAAGCTGCCGAA |
| | GTACAGCCTGTTCGAACTGGAAAACGGAAGAAAGAGAATGCTGGCAAGCGCAGGAGAACTGCAGAAGGGAAACGAACTG |
| | GCACTGCCGAGCAAGTACGTCAACTTCCTGTACCTGGCAAGCCACTACGAAAAGCTGAAGGGAAGCCCGGAAGACAACGA |
| | ACAGAAGCAGCTGTTCGTCGAACAGCACAAGCACTACCTGGACGAAATCATCGAACAGATCAGCGAATTCAGCAAGAGAG |
| | TCATCCTGGCAGACGCAAACCTGGACAAGGTCCTGAGCGCATACAACAAGCACAGAGACAAGCCGATCAGAGAACAGGC |
| | AGAAAACATCATCCACCTGTTCACACTGACAAACCTGGGAGCACCGGCAGCATTCAAGTACTTCGACACAACAATCGACA |
| | GAAAGAGATACACAAGCACAAAGGAAGTCCTGGACGCAACACTGATCCACCAGAGCATCACAGGACTGTACGAAACAAG |
| | AATCGACCTGAGCCAGCTGGGAGGAGACGGAGGAGGAAGCCCGAAGAAGAAGAGAAAGGTCTAGCTAGCCATCACATTT |
| | AAAAGCATCTCAGCCTACCATGAGAATAAGAGAAAGAAAATGAAGATCAATAGCTTATTCATCTCTTTTTCTTTTTCGTTGG |
| | TGTAAAGCCAACACCCTGTCTAAAAAACATAAATTTCTTTAATCATTTTGCCTCTTTTCTCTGTGCTTCAATTAATAAAAAAT |
| | GGAAAGAACCTCGAG |
| 507 | ATGGACAAGAAGTACAGCATCGGACTGGACATCGGAACAAACAGCGTCGGATGGGCAGTCATCACAGACGAATACAAGGT |
| | CCCGAGCAAGAAGTTCAAGGTCCTGGGAAACACAGACAGACACAGCATCAAGAAGAACCTGATCGGAGCACTGCTGTTCG |
| | ACAGCGGAGAAACAGCAGAAGCAACAAGACTGAAGAGAACAGCAAGAAGAAGATACACAAGAAGAAAGAACAGAATCT |
| | GCTACCTGCAGGAAATCTTCAGCAACGAAATGGCAAAGGTCGACGACAGCTTCTTCCACcggCTGGAAGAAAGCTTCCTGGT |
| | CGAAGAAGACAAGAAGCACGAAAGACACCCGATCTTCGGAAACATCGTCGACGAAGTCGCATACCACGAAAAGTACCCG |
| | ACAATCTACCACCTGAGAAAGAAGCTGGTCGACAGCACAGACAAGGCAGACCTGAGACTGATCTACCTGGCACTGGCACA |
| | CATGATCAAGTTCAGAGGACACTTCCTGATCGAAGGAGACCTGAACCCGGACAACAGCGACGTCGACAAGCTGTTCATCC |
| | AGCTGGTCCAGACATACAACCAGCTGTTCGAAGAAAACCCGATCAACGCAAGCGGAGTCGACGCAAAGGCAATCCTGAGC |
| | GCAAGACTGAGCAAGAGCAGAAGACTGGAAAACCTGATCGCACAGCTGCCGGGAGAAAAGAAGAACGGACTGTTCGGAA |
| | ACCTGATCGCACTGAGCCTGGGACTGACACCGAACTTCAAGAGCAACTTCGACCTGGCAGAAGACGCAAAGCTGCAGCTG |
| | AGCAAGGACACATACGACGACGACCTGGACAACCTGCTGGCACAGATCGGAGACCAGTACGCAGACCTGTTCCTGGCAGC |
| | AAAGAACCTGAGCGACGCAATCCTGCTGAGCGACATCCTGAGAGTCAACACAGAAATCACAAAGGCACCGCTGAGCGCAA |
| | GCATGATCAAGAGATACGACGAACACCACCAGGACCTGACACTGCTGAAGGCACTGGTCAGACAGCAGCTGCCGGAAAAG |
| | TACAAGGAAATCTTCTTCGACCAGAGCAAGAACGGATACGCAGGATACATCGACGGAGGAGCAAGCCAGGAAGAATTCTA |
| | CAAGTTCATCAAGCCGATCCTGGAAAAGATGGACGGAACAGAAGAACTGCTGGTCAAGCTGAACAGAGAAGACCTGCTGA |
| | GAAAGCAGAGAACATTCGACAACGGAAGCATCCCGCACCAGATCCACCTGGGAGAACTGCACGCAATCCTGAGAAGACA |
| | GGAAGACTTCTACCCGTTCCTGAAGGACAACAGAGAAAAGATCGAAAAGATCCTGACATTCAGAATCCCGTACTACGTCG |
| | GACCGCTGGCAAGAGGAAACAGCAGATTCGCATGGATGACAAGAAAGAGCGAAGAAACAATCACACCGTGGAACTTCGA |
| | AGAAGTCGTCGACAAGGGAGCAAGCGCACAGAGCTTCATCGAAAGAATGACAAACTTCGACAAGAACCTGCCGAACGAA |
| | AAGGTCCTGCCGAAGCACAGCCTGCTGTACGAATACTTCACAGTCTACAACGAACTGACAAAGGTCAAGTACGTCACAGA |
| | AGGAATGAGAAAGCCGGCATTCCTGAGCGGAGAACAGAAGAAGGCAATCGTCGACCTGCTGTTCAAGACAAACAGAAAG |
| | GTCACAGTCAAGCAGCTGAAGGAAGACTACTTCAAGAAGATCGAATGCTTCGACAGCGTCGAAATCAGCGGAGTCGAAGA |
| | CAGATTCAACGCAAGCCTGGGAACATACCACGACCTGCTGAAGATCATCAAGGACAAGGACTTCCTGGACAACGAAGAAA |
| | ACGAAGACATCCTGGAAGACATCGTCCTGACACTGACACTGTTCGAAGACAGAGAAATGATCGAAGAAAGACTGAAGACA |
| | TACGCACACCTGTTCGACGACAAGGTCATGAAGCAGCTGAAGAGAAGAAGATACACAGGATGGGGAAGACTGAGCAGAA |
| | AGCTGATCAACGGAATCAGAGACAAGCAGAGCGGAAAGACAATCCTGGACTTCCTGAAGAGCGACGGATTCGCAAACAG |
| | AAACTTCATGCAGCTGATCCACGACGACAGCCTGACATTCAAGGAAGACATCCAGAAGGCACAGGTCAGCGGACAGGGAG |
| | ACAGCCTGCACGAACACATCGCAAACCTGGCAGGAAGCCCGGCAATCAAGAAGGGAATCCTGCAGACAGTCAAGGTCGTC |
| | GACGAACTGGTCAAGGTCATGGGAAGACACAAGCCGGAAAACATCGTCATCGAAATGGCAAGAGAAAACCAGACAACAC |
| | AGAAGGGACAGAAGAACAGCAGAGAAAGAATGAAGAGAATCGAAGAAGGAATCAAGGAACTGGGAAGCCAGATCCTGA |
| | AGGAACACCCGGTCGAAAACACACAGCTGCAGAACGAAAAGCTGTACCTGTACTACCTGCAaAACGGAAGAGACATGTAC |
| | GTCGACCAGGAACTGGACATCAACAGACTGAGCGACTACGACGTCGACCACATCGTCCCGCAGAGCTTCCTGAAGGACGA |
| | CAGCATCGACAACAAGGTCCTGACAAGAAGCGACAAGAACAGAGGAAAGAGCGACAACGTCCCGAGCGAAGAAGTCGTC |
| | AAGAAGATGAAGAACTACTGGAGACAGCTGCTGAACGCAAAGCTGATCACACAGAGAAAGTTCGACAACCTGACAAAGG |
| | CAGAGAGAGGAGGACTGAGCGAACTGGACAAGGCAGGATTCATCAAGAGACAGCTGGTCGAAACAAGACAGATCACAAA |
| | GCACGTCGCACAGATCCTGGACAGCAGAATGAACACAAAGTACGACGAAAACGACAAGCTGATCAGAGAAGTCAAGGTC |
| | ATCACACTGAAGAGCAAGCTGGTCAGCGACTTCAGAAAGGACTTCCAGTTCTACAAGGTCAGAGAAATCAACAACTACCA |
| | CCACGCACACGACGCATACCTGAACGCAGTCGTCGGAACAGCACTGATCAAGAAGTACCCGAAGCTGGAAAGCGAATTCG |
| | TCTACGGAGACTACAAGGTCTACGACGTCAGAAAGATGATCGCAAAGAGCGAACAGGAAATCGGAAAGGCAACAGCAAA |
| | GTACTTCTTCTACAGCAACATCATGAACTTCTTCAAGACAGAAATCACACTGGCAAACGGAGAAATCAGAAAGAGACCGCT |
| | GATCGAAACAAACGGAGAAACAGGAGAAATCGTCTGGGACAAGGGAAGAGACTTCGCAACAGTCAGAAAGGTCCTGAGC |
| | ATGCCGCAGGTCAACATCGTCAAGAAGACAGAAGTCCAGACAGGAGGATTCAGCAAGGAAAGCATCCTGCCGAAGAGAA |
| | ACAGCGACAAGCTGATCGCAAGAAAGAAGGACTGGGACCCGAAGAAGTACGGAGGATTCGACAGCCCGACAGTCGCATA |
| | CAGCGTCCTGGTCGTCGCAAAGGTCGAAAAGGGAAAGAGCAAGAAGCTGAAGAGCGTCAAGGAACTGCTGGGAATCACA |
| | ATCATGGAAAGAAGCAGCTTCGAAAAGAACCCGATCGACTTCCTGGAAGCAAAGGGATACAAGGAAGTCAAGAAGGACC |
| | TGATCATCAAGCTGCCGAAGTACAGCCTGTTCGAACTGGAAAACGGAAGAAAGAGAATGCTGGCAAGCGCAGGAGAACTG |
| | CAGAAGGGAAACGAACTGGCACTGCCGAGCAAGTACGTCAACTTCCTGTACCTGGCAAGCCACTACGAAAAGCTGAAGGG |
| | AAGCCCGGAAGACAACGAACAGAAGCAGCTGTTCGTCGAACAGCACAAGCACTACCTGGACGAAATCATCGAACAGATCA |
| | GCGAATTCAGCAAGAGAGTCATCCTGGCAGACGCAAACCTGGACAAGGTCCTGAGCGCATACAACAAGCACAGAGACAAG |
| | CCGATCAGAGAACAGGCAGAAAACATCATCCACCTGTTCACACTGACAAACCTGGGAGCACCGGCAGCATTCAAGTACTT |
| | CGACACAACAATCGACAGAAAGAGATACACAAGCACAAAGGAAGTCCTGGACGCAACACTGATCCACCAGAGCATCACA |
| | GGACTGTACGAAACAAGAATCGACCTGAGCCAGCTGGGAGGAGACGGAGGAGGAAGCCCGAAGAAGAAGAGAAAGGTCT |
| | AG |
| 508 | ATGGACAAGAAGTACAGCATCGGCCTGGACATCGGCACCAACAGCGTGGGCTGGGCCGTGATCACCGACGAGTACAAGGT |
| | GCCCAGCAAGAAGTTCAAGGTGCTGGGCAACACCGACAGACACAGCATCAAGAAGAACCTGATCGGCGCCCTGCTGTTCG |
| | ACAGCGGCGAGACCGCCGAGGCCACCAGACTGAAGAGAACCGCCAGAAGAAGATACACCAGAAGAAAGAACAGAATCTG |
| | CTACCTGCAGGAGATCTTCAGCAACGAGATGGCCAAGGTGGACGACAGCTTCTTCCACAGACTGGAGGAGAGCTTCCTGGT |
| | GGAGGAGGACAAGAAGCACGAGAGACACCCCATCTTCGGCAACATCGTGGACGAGGTGGCCTACCACGAGAAGTACCCC |
| | ACCATCTACCACCTGAGAAAGAAGCTGGTGGACAGCACCGACAAGGCCGACCTGAGACTGATCTACCTGGCCCTGGCCCA |
| | CATGATCAAGTTCAGAGGCCACTTCCTGATCGAGGGCGACCTGAACCCCGACAACAGCGACGTGGACAAGCTGTTCATCCA |
| | GCTGGTGCAGACCTACAACCAGCTGTTCGAGGAGAACCCCATCAACGCCAGCGGCGTGGACGCCAAGGCCATCCTGAGCG |
| | CCAGACTGAGCAAGAGCAGAAGACTGGAGAACCTGATCGCCCAGCTGCCCGGCGAGAAGAAGAACGGCCTGTTCGGCAA |
| | CCTGATCGCCCTGAGCCTGGGCCTGACCCCCAACTTCAAGAGCAACTTCGACCTGGCCGAGGACGCCAAGCTGCAGCTGAG |
| | CAAGGACACCTACGACGACGACCTGGACAACCTGCTGGCCCAGATCGGCGACCAGTACGCCGACCTGTTCCTGGCCGCCA |
| | AGAACCTGAGCGACGCCATCCTGCTGAGCGACATCCTGAGAGTGAACACCGAGATCACCAAGGCCCCCCTGAGCGCCAGC |
| | ATGATCAAGAGATACGACGAGCACCACCAGGACCTGACCCTGCTGAAGGCCCTGGTGAGACAGCAGCTGCCCGAGAAGTA |
| | CAAGGAGATCTTCTTCGACCAGAGCAAGAACGGCTACGCCGGCTACATCGACGGCGGCGCCAGCCAGGAGGAGTTCTACA |
| | AGTTCATCAAGCCCATCCTGGAGAAGATGGACGGCACCGAGGAGCTGCTGGTGAAGCTGAACAGAGAGGACCTGCTGAGA |
| | AAGCAGAGAACCTTCGACAACGGCAGCATCCCCCACCAGATCCACCTGGGCGAGCTGCACGCCATCCTGAGAAGACAGGA |
| | GGACTTCTACCCCTTCCTGAAGGACAACAGAGAGAAGATCGAGAAGATCCTGACCTTCAGAATCCCCTACTACGTGGGCCC |
| | CCTGGCCAGAGGCAACAGCAGATTCGCCTGGATGACCAGAAAGAGCGAGGAGACCATCACCCCCTGGAACTTCGAGGAGG |
| | TGGTGGACAAGGGCGCCAGCGCCCAGAGCTTCATCGAGAGAATGACCAACTTCGACAAGAACCTGCCCAACGAGAAGGTG |
| | CTGCCCAAGCACAGCCTGCTGTACGAGTACTTCACCGTGTACAACGAGCTGACCAAGGTGAAGTACGTGACCGAGGGCAT |
| | GAGAAAGCCCGCCTTCCTGAGCGGCGAGCAGAAGAAGGCCATCGTGGACCTGCTGTTCAAGACCAACAGAAAGGTGACCG |
| | TGAAGCAGCTGAAGGAGGACTACTTCAAGAAGATCGAGTGCTTCGACAGCGTGGAGATCAGCGGCGTGGAGGACAGATTC |
| | AACGCCAGCCTGGGCACCTACCACGACCTGCTGAAGATCATCAAGGACAAGGACTTCCTGGACAACGAGGAGAACGAGGA |
| | CATCCTGGAGGACATCGTGCTGACCCTGACCCTGTTCGAGGACAGAGAGATGATCGAGGAGAGACTGAAGACCTACGCCC |
| | ACCTGTTCGACGACAAGGTGATGAAGCAGCTGAAGAGAAGAAGATACACCGGCTGGGGCAGACTGAGCAGAAAGCTGAT |
| | CAACGGCATCAGAGACAAGCAGAGCGGCAAGACCATCCTGGACTTCCTGAAGAGCGACGGCTTCGCCAACAGAAACTTCA |
| | TGCAGCTGATCCACGACGACAGCCTGACCTTCAAGGAGGACATCCAGAAGGCCCAGGTGAGCGGCCAGGGCGACAGCCTG |
| | CACGAGCACATCGCCAACCTGGCCGGCAGCCCCGCCATCAAGAAGGGCATCCTGCAGACCGTGAAGGTGGTGGACGAGCT |
| | GGTGAAGGTGATGGGCAGACACAAGCCCGAGAACATCGTGATCGAGATGGCCAGAGAGAACCAGACCACCCAGAAGGGC |
| | CAGAAGAACAGCAGAGAGAGAATGAAGAGAATCGAGGAGGGCATCAAGGAGCTGGGCAGCCAGATCCTGAAGGAGCACC |
| | CCGTGGAGAACACCCAGCTGCAGAACGAGAAGCTGTACCTGTACTACCTGCAGAACGGCAGAGACATGTACGTGGACCAG |
| | GAGCTGGACATCAACAGACTGAGCGACTACGACGTGGACCACATCGTGCCCCAGAGCTTCCTGAAGGACGACAGCATCGA |
| | CAACAAGGTGCTGACCAGAAGCGACAAGAACAGAGGCAAGAGCGACAACGTGCCCAGCGAGGAGGTGGTGAAGAAGATG |
| | AAGAACTACTGGAGACAGCTGCTGAACGCCAAGCTGATCACCCAGAGAAAGTTCGACAACCTGACCAAGGCCGAGAGAGG |
| | CGGCCTGAGCGAGCTGGACAAGGCCGGCTTCATCAAGAGACAGCTGGTGGAGACCAGACAGATCACCAAGCACGTGGCCC |
| | AGATCCTGGACAGCAGAATGAACACCAAGTACGACGAGAACGACAAGCTGATCAGAGAGGTGAAGGTGATCACCCTGAA |
| | GAGCAAGCTGGTGAGCGACTTCAGAAAGGACTTCCAGTTCTACAAGGTGAGAGAGATCAACAACTACCACCACGCCCACG |
| | ACGCCTACCTGAACGCCGTGGTGGGCACCGCCCTGATCAAGAAGTACCCCAAGCTGGAGAGCGAGTTCGTGTACGGCGAC |
| | TACAAGGTGTACGACGTGAGAAAGATGATCGCCAAGAGCGAGCAGGAGATCGGCAAGGCCACCGCCAAGTACTTCTTCTA |
| | CAGCAACATCATGAACTTCTTCAAGACCGAGATCACCCTGGCCAACGGCGAGATCAGAAAGAGACCCCTGATCGAGACCA |
| | ACGGCGAGACCGGCGAGATCGTGTGGGACAAGGGCAGAGACTTCGCCACCGTGAGAAAGGTGCTGAGCATGCCCCAGGTG |
| | AACATCGTGAAGAAGACCGAGGTGCAGACCGGCGGCTTCAGCAAGGAGAGCATCCTGCCCAAGAGAAACAGCGACAAGC |
| | TGATCGCCAGAAAGAAGGACTGGGACCCCAAGAAGTACGGCGGCTTCGACAGCCCCACCGTGGCCTACAGCGTGCTGGTG |
| | GTGGCCAAGGTGGAGAAGGGCAAGAGCAAGAAGCTGAAGAGCGTGAAGGAGCTGCTGGGCATCACCATCATGGAGAGAA |
| | GCAGCTTCGAGAAGAACCCCATCGACTTCCTGGAGGCCAAGGGCTACAAGGAGGTGAAGAAGGACCTGATCATCAAGCTG |
| | CCCAAGTACAGCCTGTTCGAGCTGGAGAACGGCAGAAAGAGAATGCTGGCCAGCGCCGGCGAGCTGCAGAAGGGCAACG |
| | AGCTGGCCCTGCCCAGCAAGTACGTGAACTTCCTGTACCTGGCCAGCCACTACGAGAAGCTGAAGGGCAGCCCCGAGGAC |
| | AACGAGCAGAAGCAGCTGTTCGTGGAGCAGCACAAGCACTACCTGGACGAGATCATCGAGCAGATCAGCGAGTTCAGCAA |
| | GAGAGTGATCCTGGCCGACGCCAACCTGGACAAGGTGCTGAGCGCCTACAACAAGCACAGAGACAAGCCCATCAGAGAGC |
| | AGGCCGAGAACATCATCCACCTGTTCACCCTGACCAACCTGGGCGCCCCCGCCGCCTTCAAGTACTTCGACACCACCATCG |
| | ACAGAAAGAGATACACCAGCACCAAGGAGGTGCTGGACGCCACCCTGATCCACCAGAGCATCACCGGCCTGTACGAGACC |
| | AGAATCGACCTGAGCCAGCTGGGCGGCGACGGCGGCGGCAGCCCCAAGAAGAAGAGAAAGGTGTGA |
| 509 | GGGTCCCGCAGTCGGCGTCCAGCGGCTCTGCTTGTTCGTGTGTGTGTCGTTGCAGGCCTTATTCGGATCCGCCACCATGGAC |
| | AAGAAGTACAGCATCGGCCTGGACATCGGCACCAACAGCGTGGGCTGGGCCGTGATCACCGACGAGTACAAGGTGCCCAG |
| | CAAGAAGTTCAAGGTGCTGGGCAACACCGACAGACACAGCATCAAGAAGAACCTGATCGGCGCCCTGCTGTTCGACAGCG |
| | GCGAGACCGCCGAGGCCACCAGACTGAAGAGAACCGCCAGAAGAAGATACACCAGAAGAAAGAACAGAATCTGCTACCT |
| | GCAGGAGATCTTCAGCAACGAGATGGCCAAGGTGGACGACAGCTTCTTCCACAGACTGGAGGAGAGCTTCCTGGTGGAGG |
| | AGGACAAGAAGCACGAGAGACACCCCATCTTCGGCAACATCGTGGACGAGGTGGCCTACCACGAGAAGTACCCCACCATC |
| | TACCACCTGAGAAAGAAGCTGGTGGACAGCACCGACAAGGCCGACCTGAGACTGATCTACCTGGCCCTGGCCCACATGAT |
| | CAAGTTCAGAGGCCACTTCCTGATCGAGGGCGACCTGAACCCCGACAACAGCGACGTGGACAAGCTGTTCATCCAGCTGGT |
| | GCAGACCTACAACCAGCTGTTCGAGGAGAACCCCATCAACGCCAGCGGCGTGGACGCCAAGGCCATCCTGAGCGCCAGAC |
| | TGAGCAAGAGCAGAAGACTGGAGAACCTGATCGCCCAGCTGCCCGGCGAGAAGAAGAACGGCCTGTTCGGCAACCTGATC |
| | GCCCTGAGCCTGGGCCTGACCCCCAACTTCAAGAGCAACTTCGACCTGGCCGAGGACGCCAAGCTGCAGCTGAGCAAGGA |
| | CACCTACGACGACGACCTGGACAACCTGCTGGCCCAGATCGGCGACCAGTACGCCGACCTGTTCCTGGCCGCCAAGAACCT |
| | GAGCGACGCCATCCTGCTGAGCGACATCCTGAGAGTGAACACCGAGATCACCAAGGCCCCCCTGAGCGCCAGCATGATCA |
| | AGAGATACGACGAGCACCACCAGGACCTGACCCTGCTGAAGGCCCTGGTGAGACAGCAGCTGCCCGAGAAGTACAAGGA |
| | GATCTTCTTCGACCAGAGCAAGAACGGCTACGCCGGCTACATCGACGGCGGCGCCAGCCAGGAGGAGTTCTACAAGTTCAT |
| | CAAGCCCATCCTGGAGAAGATGGACGGCACCGAGGAGCTGCTGGTGAAGCTGAACAGAGAGGACCTGCTGAGAAAGCAG |
| | AGAACCTTCGACAACGGCAGCATCCCCCACCAGATCCACCTGGGCGAGCTGCACGCCATCCTGAGAAGACAGGAGGACTT |
| | CTACCCCTTCCTGAAGGACAACAGAGAGAAGATCGAGAAGATCCTGACCTTCAGAATCCCCTACTACGTGGGCCCCCTGGC |
| | CAGAGGCAACAGCAGATTCGCCTGGATGACCAGAAAGAGCGAGGAGACCATCACCCCCTGGAACTTCGAGGAGGTGGTGG |
| | ACAAGGGCGCCAGCGCCCAGAGCTTCATCGAGAGAATGACCAACTTCGACAAGAACCTGCCCAACGAGAAGGTGCTGCCC |
| | AAGCACAGCCTGCTGTACGAGTACTTCACCGTGTACAACGAGCTGACCAAGGTGAAGTACGTGACCGAGGGCATGAGAAA |
| | GCCCGCCTTCCTGAGCGGCGAGCAGAAGAAGGCCATCGTGGACCTGCTGTTCAAGACCAACAGAAAGGTGACCGTGAAGC |
| | AGCTGAAGGAGGACTACTTCAAGAAGATCGAGTGCTTCGACAGCGTGGAGATCAGCGGCGTGGAGGACAGATTCAACGCC |
| | AGCCTGGGCACCTACCACGACCTGCTGAAGATCATCAAGGACAAGGACTTCCTGGACAACGAGGAGAACGAGGACATCCT |
| | GGAGGACATCGTGCTGACCCTGACCCTGTTCGAGGACAGAGAGATGATCGAGGAGAGACTGAAGACCTACGCCCACCTGT |
| | TCGACGACAAGGTGATGAAGCAGCTGAAGAGAAGAAGATACACCGGCTGGGGCAGACTGAGCAGAAAGCTGATCAACGG |
| | CATCAGAGACAAGCAGAGCGGCAAGACCATCCTGGACTTCCTGAAGAGCGACGGCTTCGCCAACAGAAACTTCATGCAGC |
| | TGATCCACGACGACAGCCTGACCTTCAAGGAGGACATCCAGAAGGCCCAGGTGAGCGGCCAGGGCGACAGCCTGCACGAG |
| | CACATCGCCAACCTGGCCGGCAGCCCCGCCATCAAGAAGGGCATCCTGCAGACCGTGAAGGTGGTGGACGAGCTGGTGAA |
| | GGTGATGGGCAGACACAAGCCCGAGAACATCGTGATCGAGATGGCCAGAGAGAACCAGACCACCCAGAAGGGCCAGAAG |
| | AACAGCAGAGAGAGAATGAAGAGAATCGAGGAGGGCATCAAGGAGCTGGGCAGCCAGATCCTGAAGGAGCACCCCGTGG |
| | AGAACACCCAGCTGCAGAACGAGAAGCTGTACCTGTACTACCTGCAGAACGGCAGAGACATGTACGTGGACCAGGAGCTG |
| | GACATCAACAGACTGAGCGACTACGACGTGGACCACATCGTGCCCCAGAGCTTCCTGAAGGACGACAGCATCGACAACAA |
| | GGTGCTGACCAGAAGCGACAAGAACAGAGGCAAGAGCGACAACGTGCCCAGCGAGGAGGTGGTGAAGAAGATGAAGAAC |
| | TACTGGAGACAGCTGCTGAACGCCAAGCTGATCACCCAGAGAAAGTTCGACAACCTGACCAAGGCCGAGAGAGGCGGCCT |
| | GAGCGAGCTGGACAAGGCCGGCTTCATCAAGAGACAGCTGGTGGAGACCAGACAGATCACCAAGCACGTGGCCCAGATCC |
| | TGGACAGCAGAATGAACACCAAGTACGACGAGAACGACAAGCTGATCAGAGAGGTGAAGGTGATCACCCTGAAGAGCAA |
| | GCTGGTGAGCGACTTCAGAAAGGACTTCCAGTTCTACAAGGTGAGAGAGATCAACAACTACCACCACGCCCACGACGCCT |
| | ACCTGAACGCCGTGGTGGGCACCGCCCTGATCAAGAAGTACCCCAAGCTGGAGAGCGAGTTCGTGTACGGCGACTACAAG |
| | GTGTACGACGTGAGAAAGATGATCGCCAAGAGCGAGCAGGAGATCGGCAAGGCCACCGCCAAGTACTTCTTCTACAGCAA |
| | CATCATGAACTTCTTCAAGACCGAGATCACCCTGGCCAACGGCGAGATCAGAAAGAGACCCCTGATCGAGACCAACGGCG |
| | AGACCGGCGAGATCGTGTGGGACAAGGGCAGAGACTTCGCCACCGTGAGAAAGGTGCTGAGCATGCCCCAGGTGAACATC |
| | GTGAAGAAGACCGAGGTGCAGACCGGCGGCTTCAGCAAGGAGAGCATCCTGCCCAAGAGAAACAGCGACAAGCTGATCG |
| | CCAGAAAGAAGGACTGGGACCCCAAGAAGTACGGCGGCTTCGACAGCCCCACCGTGGCCTACAGCGTGCTGGTGGTGGCC |
| | AAGGTGGAGAAGGGCAAGAGCAAGAAGCTGAAGAGCGTGAAGGAGCTGCTGGGCATCACCATCATGGAGAGAAGCAGCT |
| | TCGAGAAGAACCCCATCGACTTCCTGGAGGCCAAGGGCTACAAGGAGGTGAAGAAGGACCTGATCATCAAGCTGCCCAAG |
| | TACAGCCTGTTCGAGCTGGAGAACGGCAGAAAGAGAATGCTGGCCAGCGCCGGCGAGCTGCAGAAGGGCAACGAGCTGG |
| | CCCTGCCCAGCAAGTACGTGAACTTCCTGTACCTGGCCAGCCACTACGAGAAGCTGAAGGGCAGCCCCGAGGACAACGAG |
| | CAGAAGCAGCTGTTCGTGGAGCAGCACAAGCACTACCTGGACGAGATCATCGAGCAGATCAGCGAGTTCAGCAAGAGAGT |
| | GATCCTGGCCGACGCCAACCTGGACAAGGTGCTGAGCGCCTACAACAAGCACAGAGACAAGCCCATCAGAGAGCAGGCCG |
| | AGAACATCATCCACCTGTTCACCCTGACCAACCTGGGCGCCCCCGCCGCCTTCAAGTACTTCGACACCACCATCGACAGAA |
| | AGAGATACACCAGCACCAAGGAGGTGCTGGACGCCACCCTGATCCACCAGAGCATCACCGGCCTGTACGAGACCAGAATC |
| | GACCTGAGCCAGCTGGGCGGCGACGGCGGCGGCAGCCCCAAGAAGAAGAGAAAGGTGTGACTAGCCATCACATTTAAAA |
| | GCATCTCAGCCTACCATGAGAATAAGAGAAAGAAAATGAAGATCAATAGCTTATTCATCTCTTTTTCTTTTTCGTTGGTGTA |
| | AAGCCAACACCCTGTCTAAAAAACATAAATTTCTTTAATCATTTTGCCTCTTTTCTCTGTGCTTCAATTAATAAAAAATGGA |
| | AAGAACCTCGAG |
| 510 | ATGGACAAGAAGTACTCTATCGGTTTGGACATCGGTACCAACTCTGTCGGTTGGGCCGTCATCACCGACGAATACAAGGTC |
| | CCATCTAAGAAGTTCAAGGTCTTGGGTAACACCGACAGACACTCTATCAAGAAGAACTTGATCGGTGCCTTGTTGTTCGAC |
| | TCTGGTGAAACCGCCGAAGCCACCAGATTGAAGAGAACCGCCAGAAGAAGATACACCAGAAGAAAGAACAGAATCTGCT |
| | ACTTGCAAGAAATCTTCTCTAACGAAATGGCCAAGGTCGACGACTCTTTCTTCCACAGATTGGAAGAATCTTTCTTGGTCGA |
| | AGAAGACAAGAAGCACGAAAGACACCCAATCTTCGGTAACATCGTCGACGAAGTCGCCTACCACGAAAAGTACCCAACCA |
| | TCTACCACTTGAGAAAGAAGTTGGTCGACTCTACCGACAAGGCCGACTTGAGATTGATCTACTTGGCCTTGGCCCACATGA |
| | TCAAGTTCAGAGGTCACTTCTTGATCGAAGGTGACTTGAACCCAGACAACTCTGACGTCGACAAGTTGTTCATCCAATTGGT |
| | CCAAACCTACAACCAATTGTTCGAAGAAAACCCAATCAACGCCTCTGGTGTCGACGCCAAGGCCATCTTGTCTGCCAGATT |
| | GTCTAAGAGCAGAAGATTGGAAAACTTGATCGCCCAATTGCCAGGTGAAAAGAAGAACGGTTTGTTCGGTAACTTGATCGC |
| | CTTGTCTTTGGGTTTGACCCCAAACTTCAAGTCTAACTTCGACTTGGCCGAAGACGCCAAGTTGCAATTGTCTAAGGACACC |
| | TACGACGACGACTTGGACAACTTGTTGGCCCAAATCGGTGACCAATACGCCGACTTGTTCTTGGCCGCCAAGAACTTGTCT |
| | GACGCCATCTTGTTGTCTGACATCTTGAGAGTCAACACCGAAATCACCAAGGCCCCATTGTCTGCCTCTATGATCAAGAGAT |
| | ACGACGAACACCACCAAGACTTGACCTTGTTGAAGGCCTTGGTCAGACAACAATTGCCAGAAAAGTACAAGGAAATCTTCT |
| | TCGACCAATCTAAGAACGGTTACGCCGGTTACATCGACGGTGGTGCCTCTCAAGAAGAATTCTACAAGTTCATCAAGCCAA |
| | TCTTGGAAAAGATGGACGGTACCGAAGAATTGTTGGTCAAGTTGAACAGAGAAGACTTGTTGAGAAAGCAAAGAACCTTC |
| | GACAACGGTTCTATCCCACACCAAATCCACTTGGGTGAATTGCACGCCATCTTGAGAAGACAAGAAGACTTCTACCCATTC |
| | TTGAAGGACAACAGAGAAAAGATCGAAAAGATCTTGACCTTCAGAATCCCATACTACGTCGGTCCATTGGCCAGAGGTAA |
| | CAGCAGATTCGCCTGGATGACCAGAAAGTCTGAAGAAACCATCACCCCATGGAACTTCGAAGAAGTCGTCGACAAGGGTG |
| | CCTCTGCCCAATCTTTCATCGAAAGAATGACCAACTTCGACAAGAACTTGCCAAACGAAAAGGTCTTGCCAAAGCACTCTT |
| | TGTTGTACGAATACTTCACCGTCTACAACGAATTGACCAAGGTCAAGTACGTCACCGAAGGTATGAGAAAGCCAGCCTTCT |
| | TGTCTGGTGAACAAAAGAAGGCCATCGTCGACTTGTTGTTCAAGACCAACAGAAAGGTCACCGTCAAGCAATTGAAGGAA |
| | GACTACTTCAAGAAGATCGAATGCTTCGACTCTGTCGAAATCTCTGGTGTCGAAGACAGATTCAACGCCTCTTTGGGTACCT |
| | ACCACGACTTGTTGAAGATCATCAAGGACAAGGACTTCTTGGACAACGAAGAAAACGAAGACATCTTGGAAGACATCGTC |
| | TTGACCTTGACCTTGTTCGAAGACAGAGAAATGATCGAAGAAAGATTGAAGACCTACGCCCACTTGTTCGACGACAAGGTC |
| | ATGAAGCAATTGAAGAGAAGAAGATACACCGGTTGGGGTAGATTGAGCAGAAAGTTGATCAACGGTATCAGAGACAAGC |
| | AATCTGGTAAGACCATCTTGGACTTCTTGAAGTCTGACGGTTTCGCCAACAGAAACTTCATGCAATTGATCCACGACGACTC |
| | TTTGACCTTCAAGGAAGACATCCAAAAGGCCCAAGTCTCTGGTCAAGGTGACTCTTTGCACGAACACATCGCCAACTTGGC |
| | CGGTTCTCCAGCCATCAAGAAGGGTATCTTGCAAACCGTCAAGGTCGTCGACGAATTGGTCAAGGTCATGGGTAGACACAA |
| | GCCAGAAAACATCGTCATCGAAATGGCCAGAGAAAACCAAACCACCCAAAAGGGTCAAAAGAACAGCAGAGAAAGAATG |
| | AAGAGAATCGAAGAAGGTATCAAGGAATTGGGTTCTCAAATCTTGAAGGAACACCCAGTCGAAAACACCCAATTGCAAAA |
| | CGAAAAGTTGTACTTGTACTACTTGCAAAACGGTAGAGACATGTACGTCGACCAAGAATTGGACATCAACAGATTGTCTGA |
| | CTACGACGTCGACCACATCGTCCCACAATCTTTCTTGAAGGACGACTCTATCGACAACAAGGTCTTGACCAGATCTGACAA |
| | GAACAGAGGTAAGTCTGACAACGTCCCATCTGAAGAAGTCGTCAAGAAGATGAAGAACTACTGGAGACAATTGTTGAACG |
| | CCAAGTTGATCACCCAAAGAAAGTTCGACAACTTGACCAAGGCCGAAAGAGGTGGTTTGTCTGAATTGGACAAGGCCGGT |
| | TTCATCAAGAGACAATTGGTCGAAACCAGACAAATCACCAAGCACGTCGCCCAAATCTTGGACAGCAGAATGAACACCAA |
| | GTACGACGAAAACGACAAGTTGATCAGAGAAGTCAAGGTCATCACCTTGAAGTCTAAGTTGGTCTCTGACTTCAGAAAGG |
| | ACTTCCAATTCTACAAGGTCAGAGAAATCAACAACTACCACCACGCCCACGACGCCTACTTGAACGCCGTCGTCGGTACCG |
| | CCTTGATCAAGAAGTACCCAAAGTTGGAATCTGAATTCGTCTACGGTGACTACAAGGTCTACGACGTCAGAAAGATGATCG |
| | CCAAGTCTGAACAAGAAATCGGTAAGGCCACCGCCAAGTACTTCTTCTACTCTAACATCATGAACTTCTTCAAGACCGAAA |
| | TCACCTTGGCCAACGGTGAAATCAGAAAGAGACCATTGATCGAAACCAACGGTGAAACCGGTGAAATCGTCTGGGACAAG |
| | GGTAGAGACTTCGCCACCGTCAGAAAGGTCTTGTCTATGCCACAAGTCAACATCGTCAAGAAGACCGAAGTCCAAACCGGT |
| | GGTTTCTCTAAGGAATCTATCTTGCCAAAGAGAAACTCTGACAAGTTGATCGCCAGAAAGAAGGACTGGGACCCAAAGAA |
| | GTACGGTGGTTTCGACTCTCCAACCGTCGCCTACTCTGTCTTGGTCGTCGCCAAGGTCGAAAAGGGTAAGTCTAAGAAGTT |
| | GAAGTCTGTCAAGGAATTGTTGGGTATCACCATCATGGAAAGATCTTCTTTCGAAAAGAACCCAATCGACTTCTTGGAAGC |
| | CAAGGGTTACAAGGAAGTCAAGAAGGACTTGATCATCAAGTTGCCAAAGTACTCTTTGTTCGAATTGGAAAACGGTAGAA |
| | AGAGAATGTTGGCCTCTGCCGGTGAATTGCAAAAGGGTAACGAATTGGCCTTGCCATCTAAGTACGTCAACTTCTTGTACTT |
| | GGCCTCTCACTACGAAAAGTTGAAGGGTTCTCCAGAAGACAACGAACAAAAGCAATTGTTCGTCGAACAACACAAGCACT |
| | ACTTGGACGAAATCATCGAACAAATCTCTGAATTCTCTAAGAGAGTCATCTTGGCCGACGCCAACTTGGACAAGGTCTTGT |
| | CTGCCTACAACAAGCACAGAGACAAGCCAATCAGAGAACAAGCCGAAAACATCATCCACTTGTTCACCTTGACCAACTTG |
| | GGTGCCCCAGCCGCCTTCAAGTACTTCGACACCACCATCGACAGAAAGAGATACACCTCTACCAAGGAAGTCTTGGACGCC |
| | ACCTTGATCCACCAATCTATCACCGGTTTGTACGAAACCAGAATCGACTTGTCTCAATTGGGTGGTGACGGTGGTGGTTCTC |
| | CAAAGAAGAAGAGAAAGGTCTAA |
| 511 | ATGGACAAGAAGTACTCCATCGGCCTGGACATCGGCACCAACTCCGTGGGCTGGGCCGTGATCACCGACGAGTACAAGGT |
| | GCCCTCCAAGAAGTTCAAGGTGCTGGGCAACACCGACCGGCACTCCATCAAGAAGAACCTGATCGGCGCCCTGCTGTTCGA |
| | CTCCGGCGAGACCGCCGAGGCCACCCGGCTGAAGCGGACCGCCCGGCGGCGGTACACCCGGCGGAAGAACCGGATCTGCT |
| | ACCTGCAGGAGATCTTCTCCAACGAGATGGCCAAGGTGGACGACTCCTTCTTCCACCGGCTGGAGGAGTCCTTCCTGGTGG |
| | AGGAGGACAAGAAGCACGAGCGGCACCCCATCTTCGGCAACATCGTGGACGAGGTGGCCTACCACGAGAAGTACCCCACC |
| | ATCTACCACCTGCGGAAGAAGCTGGTGGACTCCACCGACAAGGCCGACCTGCGGCTGATCTACCTGGCCCTGGCCCACATG |
| | ATCAAGTTCCGGGGCCACTTCCTGATCGAGGGCGACCTGAACCCCGACAACTCCGACGTGGACAAGCTGTTCATCCAGCTG |
| | GTGCAGACCTACAACCAGCTGTTCGAGGAGAACCCCATCAACGCCTCCGGCGTGGACGCCAAGGCCATCCTGTCCGCCCGG |
| | CTGTCCAAGTCCCGGCGGCTGGAGAACCTGATCGCCCAGCTGCCCGGCGAGAAGAAGAACGGCCTGTTCGGCAACCTGAT |
| | CGCCCTGTCCCTGGGCCTGACCCCCAACTTCAAGTCCAACTTCGACCTGGCCGAGGACGCCAAGCTGCAGCTGTCCAAGGA |
| | CACCTACGACGACGACCTGGACAACCTGCTGGCCCAGATCGGCGACCAGTACGCCGACCTGTTCCTGGCCGCCAAGAACCT |
| | GTCCGACGCCATCCTGCTGTCCGACATCCTGCGGGTGAACACCGAGATCACCAAGGCCCCCCTGTCCGCCTCCATGATCAA |
| | GCGGTACGACGAGCACCACCAGGACCTGACCCTGCTGAAGGCCCTGGTGCGGCAGCAGCTGCCCGAGAAGTACAAGGAGA |
| | TCTTCTTCGACCAGTCCAAGAACGGCTACGCCGGCTACATCGACGGCGGCGCCTCCCAGGAGGAGTTCTACAAGTTCATCA |
| | AGCCCATCCTGGAGAAGATGGACGGCACCGAGGAGCTGCTGGTGAAGCTGAACCGGGAGGACCTGCTGCGGAAGCAGCG |
| | GACCTTCGACAACGGCTCCATCCCCCACCAGATCCACCTGGGCGAGCTGCACGCCATCCTGCGGCGGCAGGAGGACTTCTA |
| | CCCCTTCCTGAAGGACAACCGGGAGAAGATCGAGAAGATCCTGACCTTCCGGATCCCCTACTACGTGGGCCCCCTGGCCCG |
| | GGGCAACTCCCGGTTCGCCTGGATGACCCGGAAGTCCGAGGAGACCATCACCCCCTGGAACTTCGAGGAGGTGGTGGACA |
| | AGGGCGCCTCCGCCCAGTCCTTCATCGAGCGGATGACCAACTTCGACAAGAACCTGCCCAACGAGAAGGTGCTGCCCAAG |
| | CACTCCCTGCTGTACGAGTACTTCACCGTGTACAACGAGCTGACCAAGGTGAAGTACGTGACCGAGGGCATGCGGAAGCCC |
| | GCCTTCCTGTCCGGCGAGCAGAAGAAGGCCATCGTGGACCTGCTGTTCAAGACCAACCGGAAGGTGACCGTGAAGCAGCT |
| | GAAGGAGGACTACTTCAAGAAGATCGAGTGCTTCGACTCCGTGGAGATCTCCGGCGTGGAGGACCGGTTCAACGCCTCCCT |
| | GGGCACCTACCACGACCTGCTGAAGATCATCAAGGACAAGGACTTCCTGGACAACGAGGAGAACGAGGACATCCTGGAGG |
| | ACATCGTGCTGACCCTGACCCTGTTCGAGGACCGGGAGATGATCGAGGAGCGGCTGAAGACCTACGCCCACCTGTTCGACG |
| | ACAAGGTGATGAAGCAGCTGAAGCGGCGGCGGTACACCGGCTGGGGCCGGCTGTCCCGGAAGCTGATCAACGGCATCCGG |
| | GACAAGCAGTCCGGCAAGACCATCCTGGACTTCCTGAAGTCCGACGGCTTCGCCAACCGGAACTTCATGCAGCTGATCCAC |
| | GACGACTCCCTGACCTTCAAGGAGGACATCCAGAAGGCCCAGGTGTCCGGCCAGGGCGACTCCCTGCACGAGCACATCGC |
| | CAACCTGGCCGGCTCCCCCGCCATCAAGAAGGGCATCCTGCAGACCGTGAAGGTGGTGGACGAGCTGGTGAAGGTGATGG |
| | GCCGGCACAAGCCCGAGAACATCGTGATCGAGATGGCCCGGGAGAACCAGACCACCCAGAAGGGCCAGAAGAACTCCCG |
| | GGAGCGGATGAAGCGGATCGAGGAGGGCATCAAGGAGCTGGGCTCCCAGATCCTGAAGGAGCACCCCGTGGAGAACACC |
| | CAGCTGCAGAACGAGAAGCTGTACCTGTACTACCTGCAGAACGGCCGGGACATGTACGTGGACCAGGAGCTGGACATCAA |
| | CCGGCTGTCCGACTACGACGTGGACCACATCGTGCCCCAGTCCTTCCTGAAGGACGACTCCATCGACAACAAGGTGCTGAC |
| | CCGGTCCGACAAGAACCGGGGCAAGTCCGACAACGTGCCCTCCGAGGAGGTGGTGAAGAAGATGAAGAACTACTGGCGGC |
| | AGCTGCTGAACGCCAAGCTGATCACCCAGCGGAAGTTCGACAACCTGACCAAGGCCGAGCGGGGCGGCCTGTCCGAGCTG |
| | GACAAGGCCGGCTTCATCAAGCGGCAGCTGGTGGAGACCCGGCAGATCACCAAGCACGTGGCCCAGATCCTGGACTCCCG |
| | GATGAACACCAAGTACGACGAGAACGACAAGCTGATCCGGGAGGTGAAGGTGATCACCCTGAAGTCCAAGCTGGTGTCCG |
| | ACTTCCGGAAGGACTTCCAGTTCTACAAGGTGCGGGAGATCAACAACTACCACCACGCCCACGACGCCTACCTGAACGCCG |
| | TGGTGGGCACCGCCCTGATCAAGAAGTACCCCAAGCTGGAGTCCGAGTTCGTGTACGGCGACTACAAGGTGTACGACGTGC |
| | GGAAGATGATCGCCAAGTCCGAGCAGGAGATCGGCAAGGCCACCGCCAAGTACTTCTTCTACTCCAACATCATGAACTTCT |
| | TCAAGACCGAGATCACCCTGGCCAACGGCGAGATCCGGAAGCGGCCCCTGATCGAGACCAACGGCGAGACCGGCGAGATC |
| | GTGTGGGACAAGGGCCGGGACTTCGCCACCGTGCGGAAGGTGCTGTCCATGCCCCAGGTGAACATCGTGAAGAAGACCGA |
| | GGTGCAGACCGGCGGCTTCTCCAAGGAGTCCATCCTGCCCAAGCGGAACTCCGACAAGCTGATCGCCCGGAAGAAGGACT |
| | GGGACCCCAAGAAGTACGGCGGCTTCGACTCCCCCACCGTGGCCTACTCCGTGCTGGTGGTGGCCAAGGTGGAGAAGGGC |
| | AAGTCCAAGAAGCTGAAGTCCGTGAAGGAGCTGCTGGGCATCACCATCATGGAGCGGTCCTCCTTCGAGAAGAACCCCAT |
| | CGACTTCCTGGAGGCCAAGGGCTACAAGGAGGTGAAGAAGGACCTGATCATCAAGCTGCCCAAGTACTCCCTGTTCGAGCT |
| | GGAGAACGGCCGGAAGCGGATGCTGGCCTCCGCCGGCGAGCTGCAGAAGGGCAACGAGCTGGCCCTGCCCTCCAAGTACG |
| | TGAACTTCCTGTACCTGGCCTCCCACTACGAGAAGCTGAAGGGCTCCCCCGAGGACAACGAGCAGAAGCAGCTGTTCGTGG |
| | AGCAGCACAAGCACTACCTGGACGAGATCATCGAGCAGATCTCCGAGTTCTCCAAGCGGGTGATCCTGGCCGACGCCAAC |
| | CTGGACAAGGTGCTGTCCGCCTACAACAAGCACCGGGACAAGCCCATCCGGGAGCAGGCCGAGAACATCATCCACCTGTT |
| | CACCCTGACCAACCTGGGCGCCCCCGCCGCCTTCAAGTACTTCGACACCACCATCGACCGGAAGCGGTACACCTCCACCAA |
| | GGAGGTGCTGGACGCCACCCTGATCCACCAGTCCATCACCGGCCTGTACGAGACCCGGATCGACCTGTCCCAGCTGGGCGG |
| | CGACGGCGGCGGCTCCCCCAAGAAGAAGCGGAAGGTGTGA |
| 512 | ATGGACAAGAAGTACAGCATCGGCCTGGACATCGGCACCAACAGCGTGGGCTGGGCCGTGATCACCGACGAGTACAAGGT |
| | GCCCAGCAAGAAGTTCAAGGTGCTGGGCAACACCGACCGGCACAGCATCAAGAAGAACCTGATCGGCGCCCTGCTGTTCG |
| | ACAGCGGCGAGACCGCCGAGGCCACCCGGCTGAAGCGGACCGCCCGGCGGCGGTACACCCGGCGGAAGAACCGGATCTG |
| | CTACCTGCAGGAGATCTTCAGCAACGAGATGGCCAAGGTGGACGACAGCTTCTTCCACCGGCTGGAGGAGAGCTTCCTGGT |
| | GGAGGAGGACAAGAAGCACGAGCGGCACCCCATCTTCGGCAACATCGTGGACGAGGTGGCCTACCACGAGAAGTACCCCA |
| | CCATCTACCACCTGCGGAAGAAGCTGGTGGACAGCACCGACAAGGCCGACCTGCGGCTGATCTACCTGGCCCTGGCCCAC |
| | ATGATCAAGTTCCGGGGCCACTTCCTGATCGAGGGCGACCTGAACCCCGACAACAGCGACGTGGACAAGCTGTTCATCCAG |
| | CTGGTGCAGACCTACAACCAGCTGTTCGAGGAGAACCCCATCAACGCCAGCGGCGTGGACGCCAAGGCCATCCTGAGCGC |
| | CCGGCTGAGCAAGAGCCGGCGGCTGGAGAACCTGATCGCCCAGCTGCCCGGCGAGAAGAAGAACGGCCTGTTCGGCAACC |
| | TGATCGCCCTGAGCCTGGGCCTGACCCCCAACTTCAAGAGCAACTTCGACCTGGCCGAGGACGCCAAGCTGCAGCTGAGCA |
| | AGGACACCTACGACGACGACCTGGACAACCTGCTGGCCCAGATCGGCGACCAGTACGCCGACCTGTTCCTGGCCGCCAAG |
| | AACCTGAGCGACGCCATCCTGCTGAGCGACATCCTGCGGGTGAACACCGAGATCACCAAGGCCCCCCTGAGCGCCAGCAT |
| | GATCAAGCGGTACGACGAGCACCACCAGGACCTGACCCTGCTGAAGGCCCTGGTGCGGCAGCAGCTGCCCGAGAAGTACA |
| | AGGAGATCTTCTTCGACCAGAGCAAGAACGGCTACGCCGGCTACATCGACGGCGGCGCCAGCCAGGAGGAGTTCTACAAG |
| | TTCATCAAGCCCATCCTGGAGAAGATGGACGGCACCGAGGAGCTGCTGGTGAAGCTGAACCGGGAGGACCTGCTGCGGAA |
| | GCAGCGGACCTTCGACAACGGCAGCATCCCCCACCAGATCCACCTGGGCGAGCTGCACGCCATCCTGCGGCGGCAGGAGG |
| | ACTTCTACCCCTTCCTGAAGGACAACCGGGAGAAGATCGAGAAGATCCTGACCTTCCGGATCCCCTACTACGTGGGCCCCC |
| | TGGCCCGGGGCAACAGCCGGTTCGCCTGGATGACCCGGAAGAGCGAGGAGACCATCACCCCCTGGAACTTCGAGGAGGTG |
| | GTGGACAAGGGCGCCAGCGCCCAGAGCTTCATCGAGCGGATGACCAACTTCGACAAGAACCTGCCCAACGAGAAGGTGCT |
| | GCCCAAGCACAGCCTGCTGTACGAGTACTTCACCGTGTACAACGAGCTGACCAAGGTGAAGTACGTGACCGAGGGCATGC |
| | GGAAGCCCGCCTTCCTGAGCGGCGAGCAGAAGAAGGCCATCGTGGACCTGCTGTTCAAGACCAACCGGAAGGTGACCGTG |
| | AAGCAGCTGAAGGAGGACTACTTCAAGAAGATCGAGTGCTTCGACAGCGTGGAGATCAGCGGCGTGGAGGACCGGTTCAA |
| | CGCCAGCCTGGGCACCTACCACGACCTGCTGAAGATCATCAAGGACAAGGACTTCCTGGACAACGAGGAGAACGAGGACA |
| | TCCTGGAGGACATCGTGCTGACCCTGACCCTGTTCGAGGACCGGGAGATGATCGAGGAGCGGCTGAAGACCTACGCCCAC |
| | CTGTTCGACGACAAGGTGATGAAGCAGCTGAAGCGGCGGCGGTACACCGGCTGGGGCCGGCTGAGCCGGAAGCTGATCAA |
| | CGGCATCCGGGACAAGCAGAGCGGCAAGACCATCCTGGACTTCCTGAAGAGCGACGGCTTCGCCAACCGGAACTTCATGC |
| | AGCTGATCCACGACGACAGCCTGACCTTCAAGGAGGACATCCAGAAGGCCCAGGTGAGCGGCCAGGGCGACAGCCTGCAC |
| | GAGCACATCGCCAACCTGGCCGGCAGCCCCGCCATCAAGAAGGGCATCCTGCAGACCGTGAAGGTGGTGGACGAGCTGGT |
| | GAAGGTGATGGGCCGGCACAAGCCCGAGAACATCGTGATCGAGATGGCCCGGGAGAACCAGACCACCCAGAAGGGCCAG |
| | AAGAACAGCCGGGAGCGGATGAAGCGGATCGAGGAGGGCATCAAGGAGCTGGGCAGCCAGATCCTGAAGGAGCACCCCG |
| | TGGAGAACACCCAGCTGCAGAACGAGAAGCTGTACCTGTACTACCTGCAGAACGGCCGGGACATGTACGTGGACCAGGAG |
| | CTGGACATCAACCGGCTGAGCGACTACGACGTGGACCACATCGTGCCCCAGAGCTTCCTGAAGGACGACAGCATCGACAA |
| | CAAGGTGCTGACCCGGAGCGACAAGAACCGGGGCAAGAGCGACAACGTGCCCAGCGAGGAGGTGGTGAAGAAGATGAAG |
| | AACTACTGGCGGCAGCTGCTGAACGCCAAGCTGATCACCCAGCGGAAGTTCGACAACCTGACCAAGGCCGAGCGGGGCGG |
| | CCTGAGCGAGCTGGACAAGGCCGGCTTCATCAAGCGGCAGCTGGTGGAGACCCGGCAGATCACCAAGCACGTGGCCCAGA |
| | TCCTGGACAGCCGGATGAACACCAAGTACGACGAGAACGACAAGCTGATCCGGGAGGTGAAGGTGATCACCCTGAAGAGC |
| | AAGCTGGTGAGCGACTTCCGGAAGGACTTCCAGTTCTACAAGGTGCGGGAGATCAACAACTACCACCACGCCCACGACGC |
| | CTACCTGAACGCCGTGGTGGGCACCGCCCTGATCAAGAAGTACCCCAAGCTGGAGAGCGAGTTCGTGTACGGCGACTACA |
| | AGGTGTACGACGTGCGGAAGATGATCGCCAAGAGCGAGCAGGAGATCGGCAAGGCCACCGCCAAGTACTTCTTCTACAGC |
| | AACATCATGAACTTCTTCAAGACCGAGATCACCCTGGCCAACGGCGAGATCCGGAAGCGGCCCCTGATCGAGACCAACGG |
| | CGAGACCGGCGAGATCGTGTGGGACAAGGGCCGGGACTTCGCCACCGTGCGGAAGGTGCTGAGCATGCCCCAGGTGAACA |
| | TCGTGAAGAAGACCGAGGTGCAGACCGGCGGCTTCAGCAAGGAGAGCATCCTGCCCAAGCGGAACAGCGACAAGCTGATC |
| | GCCCGGAAGAAGGACTGGGACCCCAAGAAGTACGGCGGCTTCGACAGCCCCACCGTGGCCTACAGCGTGCTGGTGGTGGC |
| | CAAGGTGGAGAAGGGCAAGAGCAAGAAGCTGAAGAGCGTGAAGGAGCTGCTGGGCATCACCATCATGGAGCGGAGCAGC |
| | TTCGAGAAGAACCCCATCGACTTCCTGGAGGCCAAGGGCTACAAGGAGGTGAAGAAGGACCTGATCATCAAGCTGCCCAA |
| | GTACAGCCTGTTCGAGCTGGAGAACGGCCGGAAGCGGATGCTGGCCAGCGCCGGCGAGCTGCAGAAGGGCAACGAGCTGG |
| | CCCTGCCCAGCAAGTACGTGAACTTCCTGTACCTGGCCAGCCACTACGAGAAGCTGAAGGGCAGCCCCGAGGACAACGAG |
| | CAGAAGCAGCTGTTCGTGGAGCAGCACAAGCACTACCTGGACGAGATCATCGAGCAGATCAGCGAGTTCAGCAAGCGGGT |
| | GATCCTGGCCGACGCCAACCTGGACAAGGTGCTGAGCGCCTACAACAAGCACCGGGACAAGCCCATCCGGGAGCAGGCCG |
| | AGAACATCATCCACCTGTTCACCCTGACCAACCTGGGCGCCCCCGCCGCCTTCAAGTACTTCGACACCACCATCGACCGGA |
| | AGCGGTACACCAGCACCAAGGAGGTGCTGGACGCCACCCTGATCCACCAGAGCATCACCGGCCTGTACGAGACCCGGATC |
| | GACCTGAGCCAGCTGGGCGGCGACGGCGGCGGCAGCCCCAAGAAGAAGCGGAAGGTGTGA |
| 513 | ATGGACAAGAAGTACTCCATCGGCCTGGACATCGGCACCAACTCCGTGGGCTGGGCCGTGATCACCGACGAGTACAAGGT |
| | GCCCTCCAAGAAGTTCAAGGTGCTGGGCAACACCGACCGGCACTCCATCAAGAAGAACCTGATCGGCGCCCTGCTGTTCGA |
| | CTCCGGCGAGACCGCCGAGGCCACCCGGCTGAAGCGGACCGCCCGGCGGCGGTACACCCGGCGGAAGAACCGGATCTGCT |
| | ACCTGCAGGAGATCTTCTCCAACGAGATGGCCAAGGTGGACGACTCCTTCTTCCACCGGCTGGAGGAGTCCTTCCTGGTGG |
| | AGGAGGACAAGAAGCACGAGCGGCACCCCATCTTCGGCAACATCGTGGACGAGGTGGCCTACCACGAGAAGTACCCCACC |
| | ATCTACCACCTGCGGAAGAAGCTGGTGGACTCCACCGACAAGGCCGACCTGCGGCTGATCTACCTGGCCCTGGCCCACATG |
| | ATCAAGTTCCGGGGCCACTTCCTGATCGAGGGCGACCTGAACCCCGACAACTCCGACGTGGACAAGCTGTTCATCCAGCTG |
| | GTGCAGACCTACAACCAGCTGTTCGAGGAGAACCCCATCAACGCCTCCGGCGTGGACGCCAAGGCCATCCTGTCCGCCCGG |
| | CTGTCCAAGTCCCGGCGGCTGGAGAACCTGATCGCCCAGCTGCCCGGCGAGAAGAAGAACGGCCTGTTCGGCAACCTGAT |
| | CGCCCTGTCCCTGGGCCTGACCCCCAACTTCAAGTCCAACTTCGACCTGGCCGAGGACGCCAAGCTGCAGCTGTCCAAGGA |
| | CACCTACGACGACGACCTGGACAACCTGCTGGCCCAGATCGGCGACCAGTACGCCGACCTGTTCCTGGCCGCCAAGAACCT |
| | GTCCGACGCCATCCTGCTGTCCGACATCCTGCGGGTGAACACCGAGATCACCAAGGCCCCCCTGTCCGCCTCCATGATCAA |
| | GCGGTACGACGAGCACCACCAGGACCTGACCCTGCTGAAGGCCCTGGTGCGGCAGCAGCTGCCCGAGAAGTACAAGGAGA |
| | TCTTCTTCGACCAGTCCAAGAACGGCTACGCCGGCTACATCGACGGCGGCGCCTCCCAGGAGGAGTTCTACAAGTTCATCA |
| | AGCCCATCCTGGAGAAGATGGACGGCACCGAGGAGCTGCTGGTGAAGCTGAACCGGGAGGACCTGCTGCGGAAGCAGCG |
| | GACCTTCGACAACGGCTCCATCCCCCACCAGATCCACCTGGGCGAGCTGCACGCCATCCTGCGGCGGCAGGAGGACTTCTA |
| | CCCCTTCCTGAAGGACAACCGGGAGAAGATCGAGAAGATCCTGACCTTCCGGATCCCCTACTACGTGGGCCCCCTGGCCCG |
| | GGGCAACTCCCGGTTCGCCTGGATGACCCGGAAGTCCGAGGAGACCATCACCCCCTGGAACTTCGAGGAGGTGGTGGACA |
| | AGGGCGCCTCCGCCCAGTCCTTCATCGAGCGGATGACCAACTTCGACAAGAACCTGCCCAACGAGAAGGTGCTGCCCAAG |
| | CACTCCCTGCTGTACGAGTACTTCACCGTGTACAACGAGCTGACCAAGGTGAAGTACGTGACCGAGGGCATGCGGAAGCCC |
| | GCCTTCCTGTCCGGCGAGCAGAAGAAGGCCATCGTGGACCTGCTGTTCAAGACCAACCGGAAGGTGACCGTGAAGCAGCT |
| | GAAGGAGGACTACTTCAAGAAGATCGAGTGCTTCGACTCCGTGGAGATCTCCGGCGTGGAGGACCGGTTCAACGCCTCCCT |
| | GGGCACCTACCACGACCTGCTGAAGATCATCAAGGACAAGGACTTCCTGGACAACGAGGAGAACGAGGACATCCTGGAGG |
| | ACATCGTGCTGACCCTGACCCTGTTCGAGGACCGGGAGATGATCGAGGAGCGGCTGAAGACCTACGCCCACCTGTTCGACG |
| | ACAAGGTGATGAAGCAGCTGAAGCGGCGGCGGTACACCGGCTGGGGCCGGCTGTCCCGGAAGCTGATCAACGGCATCCGG |
| | GACAAGCAGTCCGGCAAGACCATCCTGGACTTCCTGAAGTCCGACGGCTTCGCCAACCGGAACTTCATGCAGCTGATCCAC |
| | GACGACTCCCTGACCTTCAAGGAGGACATCCAGAAGGCCCAGGTGTCCGGCCAGGGCGACTCCCTGCACGAGCACATCGC |
| | CAACCTGGCCGGCTCCCCCGCCATCAAGAAGGGCATCCTGCAGACCGTGAAGGTGGTGGACGAGCTGGTGAAGGTGATGG |
| | GCCGGCACAAGCCCGAGAACATCGTGATCGAGATGGCCCGGGAGAACCAGACCACCCAGAAGGGCCAGAAGAACTCCCG |
| | GGAGCGGATGAAGCGGATCGAGGAGGGCATCAAGGAGCTGGGCTCCCAGATCCTGAAGGAGCACCCCGTGGAGAACACC |
| | CAGCTGCAGAACGAGAAGCTGTACCTGTACTACCTGCAGAACGGCCGGGACATGTACGTGGACCAGGAGCTGGACATCAA |
| | CCGGCTGTCCGACTACGACGTGGACCACATCGTGCCCCAGTCCTTCCTGAAGGACGACTCCATCGACAACAAGGTGCTGAC |
| | CCGGTCCGACAAGAACCGGGGCAAGTCCGACAACGTGCCCTCCGAGGAGGTGGTGAAGAAGATGAAGAACTACTGGCGGC |
| | AGCTGCTGAACGCCAAGCTGATCACCCAGCGGAAGTTCGACAACCTGACCAAGGCCGAGCGGGGCGGCCTGTCCGAGCTG |
| | GACAAGGCCGGCTTCATCAAGCGGCAGCTGGTGGAGACCCGGCAGATCACCAAGCACGTGGCCCAGATCCTGGACTCCCG |
| | GATGAACACCAAGTACGACGAGAACGACAAGCTGATCCGGGAGGTGAAGGTGATCACCCTGAAGTCCAAGCTGGTGTCCG |
| | ACTTCCGGAAGGACTTCCAGTTCTACAAGGTGCGGGAGATCAACAACTACCACCACGCCCACGACGCCTACCTGAACGCCG |
| | TGGTGGGCACCGCCCTGATCAAGAAGTACCCCAAGCTGGAGTCCGAGTTCGTGTACGGCGACTACAAGGTGTACGACGTGC |
| | GGAAGATGATCGCCAAGTCCGAGCAGGAGATCGGCAAGGCCACCGCCAAGTACTTCTTCTACTCCAACATCATGAACTTCT |
| | TCAAGACCGAGATCACCCTGGCCAACGGCGAGATCCGGAAGCGGCCCCTGATCGAGACCAACGGCGAGACCGGCGAGATC |
| | GTGTGGGACAAGGGCCGGGACTTCGCCACCGTGCGGAAGGTGCTGTCCATGCCCCAGGTGAACATCGTGAAGAAGACCGA |
| | GGTGCAGACCGGCGGCTTCTCCAAGGAGTCCATCCTGCCCAAGCGGAACTCCGACAAGCTGATCGCCCGGAAGAAGGACT |
| | GGGACCCCAAGAAGTACGGCGGCTTCGACTCCCCCACCGTGGCCTACTCCGTGCTGGTGGTGGCCAAGGTGGAGAAGGGC |
| | AAGTCCAAGAAGCTGAAGTCCGTGAAGGAGCTGCTGGGCATCACCATCATGGAGCGGTCCTCCTTCGAGAAGAACCCCAT |
| | CGACTTCCTGGAGGCCAAGGGCTACAAGGAGGTGAAGAAGGACCTGATCATCAAGCTGCCCAAGTACTCCCTGTTCGAGCT |
| | GGAGAACGGCCGGAAGCGGATGCTGGCCTCCGCCGGCGAGCTGCAGAAGGGCAACGAGCTGGCCCTGCCCTCCAAGTACG |
| | TGAACTTCCTGTACCTGGCCTCCCACTACGAGAAGCTGAAGGGCTCCCCCGAGGACAACGAGCAGAAGCAGCTGTTCGTGG |
| | AGCAGCACAAGCACTACCTGGACGAGATCATCGAGCAGATCTCCGAGTTCTCCAAGCGGGTGATCCTGGCCGACGCCAAC |
| | CTGGACAAGGTGCTGTCCGCCTACAACAAGCACCGGGACAAGCCCATCCGGGAGCAGGCCGAGAACATCATCCACCTGTT |
| | CACCCTGACCAACCTGGGCGCCCCCGCCGCCTTCAAGTACTTCGACACCACCATCGACCGGAAGCGGTACACCTCCACCAA |
| | GGAGGTGCTGGACGCCACCCTGATCCACCAGTCCATCACCGGCCTGTACGAGACCCGGATCGACCTGTCCCAGCTGGGCGG |
| | CGACGGCTCCGGCTCCCCCAAGAAGAAGCGGAAGGTGGACGGCTCCCCCAAGAAGAAGCGGAAGGTGGACTCCGGCTGA |
| 514 | ATGGACAAGAAGTACAGCATCGGCCTGGACATCGGCACCAACAGCGTGGGCTGGGCCGTGATCACCGACGAGTACAAGGT |
| | GCCCAGCAAGAAGTTCAAGGTGCTGGGCAACACCGACCGGCACAGCATCAAGAAGAACCTGATCGGCGCCCTGCTGTTCG |
| | ACAGCGGCGAGACCGCCGAGGCCACCCGGCTGAAGCGGACCGCCCGGCGGCGGTACACCCGGCGGAAGAACCGGATCTG |
| | CTACCTGCAGGAGATCTTCAGCAACGAGATGGCCAAGGTGGACGACAGCTTCTTCCACCGGCTGGAGGAGAGCTTCCTGGT |
| | GGAGGAGGACAAGAAGCACGAGCGGCACCCCATCTTCGGCAACATCGTGGACGAGGTGGCCTACCACGAGAAGTACCCCA |
| | CCATCTACCACCTGCGGAAGAAGCTGGTGGACAGCACCGACAAGGCCGACCTGCGGCTGATCTACCTGGCCCTGGCCCAC |
| | ATGATCAAGTTCCGGGGCCACTTCCTGATCGAGGGCGACCTGAACCCCGACAACAGCGACGTGGACAAGCTGTTCATCCAG |
| | CTGGTGCAGACCTACAACCAGCTGTTCGAGGAGAACCCCATCAACGCCAGCGGCGTGGACGCCAAGGCCATCCTGAGCGC |
| | CCGGCTGAGCAAGAGCCGGCGGCTGGAGAACCTGATCGCCCAGCTGCCCGGCGAGAAGAAGAACGGCCTGTTCGGCAACC |
| | TGATCGCCCTGAGCCTGGGCCTGACCCCCAACTTCAAGAGCAACTTCGACCTGGCCGAGGACGCCAAGCTGCAGCTGAGCA |
| | AGGACACCTACGACGACGACCTGGACAACCTGCTGGCCCAGATCGGCGACCAGTACGCCGACCTGTTCCTGGCCGCCAAG |
| | AACCTGAGCGACGCCATCCTGCTGAGCGACATCCTGCGGGTGAACACCGAGATCACCAAGGCCCCCCTGAGCGCCAGCAT |
| | GATCAAGCGGTACGACGAGCACCACCAGGACCTGACCCTGCTGAAGGCCCTGGTGCGGCAGCAGCTGCCCGAGAAGTACA |
| | AGGAGATCTTCTTCGACCAGAGCAAGAACGGCTACGCCGGCTACATCGACGGCGGCGCCAGCCAGGAGGAGTTCTACAAG |
| | TTCATCAAGCCCATCCTGGAGAAGATGGACGGCACCGAGGAGCTGCTGGTGAAGCTGAACCGGGAGGACCTGCTGCGGAA |
| | GCAGCGGACCTTCGACAACGGCAGCATCCCCCACCAGATCCACCTGGGCGAGCTGCACGCCATCCTGCGGCGGCAGGAGG |
| | ACTTCTACCCCTTCCTGAAGGACAACCGGGAGAAGATCGAGAAGATCCTGACCTTCCGGATCCCCTACTACGTGGGCCCCC |
| | TGGCCCGGGGCAACAGCCGGTTCGCCTGGATGACCCGGAAGAGCGAGGAGACCATCACCCCCTGGAACTTCGAGGAGGTG |
| | GTGGACAAGGGCGCCAGCGCCCAGAGCTTCATCGAGCGGATGACCAACTTCGACAAGAACCTGCCCAACGAGAAGGTGCT |
| | GCCCAAGCACAGCCTGCTGTACGAGTACTTCACCGTGTACAACGAGCTGACCAAGGTGAAGTACGTGACCGAGGGCATGC |
| | GGAAGCCCGCCTTCCTGAGCGGCGAGCAGAAGAAGGCCATCGTGGACCTGCTGTTCAAGACCAACCGGAAGGTGACCGTG |
| | AAGCAGCTGAAGGAGGACTACTTCAAGAAGATCGAGTGCTTCGACAGCGTGGAGATCAGCGGCGTGGAGGACCGGTTCAA |
| | CGCCAGCCTGGGCACCTACCACGACCTGCTGAAGATCATCAAGGACAAGGACTTCCTGGACAACGAGGAGAACGAGGACA |
| | TCCTGGAGGACATCGTGCTGACCCTGACCCTGTTCGAGGACCGGGAGATGATCGAGGAGCGGCTGAAGACCTACGCCCAC |
| | CTGTTCGACGACAAGGTGATGAAGCAGCTGAAGCGGCGGCGGTACACCGGCTGGGGCCGGCTGAGCCGGAAGCTGATCAA |
| | CGGCATCCGGGACAAGCAGAGCGGCAAGACCATCCTGGACTTCCTGAAGAGCGACGGCTTCGCCAACCGGAACTTCATGC |
| | AGCTGATCCACGACGACAGCCTGACCTTCAAGGAGGACATCCAGAAGGCCCAGGTGAGCGGCCAGGGCGACAGCCTGCAC |
| | GAGCACATCGCCAACCTGGCCGGCAGCCCCGCCATCAAGAAGGGCATCCTGCAGACCGTGAAGGTGGTGGACGAGCTGGT |
| | GAAGGTGATGGGCCGGCACAAGCCCGAGAACATCGTGATCGAGATGGCCCGGGAGAACCAGACCACCCAGAAGGGCCAG |
| | AAGAACAGCCGGGAGCGGATGAAGCGGATCGAGGAGGGCATCAAGGAGCTGGGCAGCCAGATCCTGAAGGAGCACCCCG |
| | TGGAGAACACCCAGCTGCAGAACGAGAAGCTGTACCTGTACTACCTGCAGAACGGCCGGGACATGTACGTGGACCAGGAG |
| | CTGGACATCAACCGGCTGAGCGACTACGACGTGGACCACATCGTGCCCCAGAGCTTCCTGAAGGACGACAGCATCGACAA |
| | CAAGGTGCTGACCCGGAGCGACAAGAACCGGGGCAAGAGCGACAACGTGCCCAGCGAGGAGGTGGTGAAGAAGATGAAG |
| | AACTACTGGCGGCAGCTGCTGAACGCCAAGCTGATCACCCAGCGGAAGTTCGACAACCTGACCAAGGCCGAGCGGGGCGG |
| | CCTGAGCGAGCTGGACAAGGCCGGCTTCATCAAGCGGCAGCTGGTGGAGACCCGGCAGATCACCAAGCACGTGGCCCAGA |
| | TCCTGGACAGCCGGATGAACACCAAGTACGACGAGAACGACAAGCTGATCCGGGAGGTGAAGGTGATCACCCTGAAGAGC |
| | AAGCTGGTGAGCGACTTCCGGAAGGACTTCCAGTTCTACAAGGTGCGGGAGATCAACAACTACCACCACGCCCACGACGC |
| | CTACCTGAACGCCGTGGTGGGCACCGCCCTGATCAAGAAGTACCCCAAGCTGGAGAGCGAGTTCGTGTACGGCGACTACA |
| | AGGTGTACGACGTGCGGAAGATGATCGCCAAGAGCGAGCAGGAGATCGGCAAGGCCACCGCCAAGTACTTCTTCTACAGC |
| | AACATCATGAACTTCTTCAAGACCGAGATCACCCTGGCCAACGGCGAGATCCGGAAGCGGCCCCTGATCGAGACCAACGG |
| | CGAGACCGGCGAGATCGTGTGGGACAAGGGCCGGGACTTCGCCACCGTGCGGAAGGTGCTGAGCATGCCCCAGGTGAACA |
| | TCGTGAAGAAGACCGAGGTGCAGACCGGCGGCTTCAGCAAGGAGAGCATCCTGCCCAAGCGGAACAGCGACAAGCTGATC |
| | GCCCGGAAGAAGGACTGGGACCCCAAGAAGTACGGCGGCTTCGACAGCCCCACCGTGGCCTACAGCGTGCTGGTGGTGGC |
| | CAAGGTGGAGAAGGGCAAGAGCAAGAAGCTGAAGAGCGTGAAGGAGCTGCTGGGCATCACCATCATGGAGCGGAGCAGC |
| | TTCGAGAAGAACCCCATCGACTTCCTGGAGGCCAAGGGCTACAAGGAGGTGAAGAAGGACCTGATCATCAAGCTGCCCAA |
| | GTACAGCCTGTTCGAGCTGGAGAACGGCCGGAAGCGGATGCTGGCCAGCGCCGGCGAGCTGCAGAAGGGCAACGAGCTGG |
| | CCCTGCCCAGCAAGTACGTGAACTTCCTGTACCTGGCCAGCCACTACGAGAAGCTGAAGGGCAGCCCCGAGGACAACGAG |
| | CAGAAGCAGCTGTTCGTGGAGCAGCACAAGCACTACCTGGACGAGATCATCGAGCAGATCAGCGAGTTCAGCAAGCGGGT |
| | GATCCTGGCCGACGCCAACCTGGACAAGGTGCTGAGCGCCTACAACAAGCACCGGGACAAGCCCATCCGGGAGCAGGCCG |
| | AGAACATCATCCACCTGTTCACCCTGACCAACCTGGGCGCCCCCGCCGCCTTCAAGTACTTCGACACCACCATCGACCGGA |
| | AGCGGTACACCAGCACCAAGGAGGTGCTGGACGCCACCCTGATCCACCAGAGCATCACCGGCCTGTACGAGACCCGGATC |
| | GACCTGAGCCAGCTGGGCGGCGACGGCAGCGGCAGCCCCAAGAAGAAGCGGAAGGTGGACGGCAGCCCCAAGAAGAAGC |
| | GGAAGGTGGACAGCGGCTGA |
| 515 | ATGGACAAGAAGTACAGCATCGGCCTGGACATCGGCACCAACAGCGTGGGCTGGGCCGTGATCACCGACGAGTACAAGGT |
| | GCCCAGCAAGAAGTTCAAGGTGCTGGGCAACACCGACCGGCACAGCATCAAGAAGAACCTGATCGGCGCCCTGCTGTTCG |
| | ACAGCGGCGAGACCGCCGAGGCCACCCGGCTGAAGCGGACCGCCCGGCGGCGGTACACCCGGCGGAAGAACCGGATCTG |
| | CTACCTGCAGGAGATCTTCAGCAACGAGATGGCCAAGGTGGACGACAGCTTCTTCCACCGGCTGGAGGAGAGCTTCCTGGT |
| | GGAGGAGGACAAGAAGCACGAGCGGCACCCCATCTTCGGCAACATCGTGGACGAGGTGGCCTACCACGAGAAGTACCCCA |
| | CCATCTACCACCTGCGGAAGAAGCTGGTGGACAGCACCGACAAGGCCGACCTGCGGCTGATCTACCTGGCCCTGGCCCAC |
| | ATGATCAAGTTCCGGGGCCACTTCCTGATCGAGGGCGACCTGAACCCCGACAACAGCGACGTGGACAAGCTGTTCATCCAG |
| | CTGGTGCAGACCTACAACCAGCTGTTCGAGGAGAACCCCATCAACGCCAGCGGCGTGGACGCCAAGGCCATCCTGAGCGC |
| | CCGGCTGAGCAAGAGCCGGCGGCTGGAGAACCTGATCGCCCAGCTGCCCGGCGAGAAGAAGAACGGCCTGTTCGGCAACC |
| | TGATCGCCCTGAGCCTGGGCCTGACCCCCAACTTCAAGAGCAACTTCGACCTGGCCGAGGACGCCAAGCTGCAGCTGAGCA |
| | AGGACACCTACGACGACGACCTGGACAACCTGCTGGCCCAGATCGGCGACCAGTACGCCGACCTGTTCCTGGCCGCCAAG |
| | AACCTGAGCGACGCCATCCTGCTGAGCGACATCCTGCGGGTGAACACCGAGATCACCAAGGCCCCCCTGAGCGCCAGCAT |
| | GATCAAGCGGTACGACGAGCACCACCAGGACCTGACCCTGCTGAAGGCCCTGGTGCGGCAGCAGCTGCCCGAGAAGTACA |
| | AGGAGATCTTCTTCGACCAGAGCAAGAACGGCTACGCCGGCTACATCGACGGCGGCGCCAGCCAGGAGGAGTTCTACAAG |
| | TTCATCAAGCCCATCCTGGAGAAGATGGACGGCACCGAGGAGCTGCTGGTGAAGCTGAACCGGGAGGACCTGCTGCGGAA |
| | GCAGCGGACCTTCGACAACGGCAGCATCCCCCACCAGATCCACCTGGGCGAGCTGCACGCCATCCTGCGGCGGCAGGAGG |
| | ACTTCTACCCCTTCCTGAAGGACAACCGGGAGAAGATCGAGAAGATCCTGACCTTCCGGATCCCCTACTACGTGGGCCCCC |
| | TGGCCCGGGGCAACAGCCGGTTCGCCTGGATGACCCGGAAGAGCGAGGAGACCATCACCCCCTGGAACTTCGAGGAGGTG |
| | GTGGACAAGGGCGCCAGCGCCCAGAGCTTCATCGAGCGGATGACCAACTTCGACAAGAACCTGCCCAACGAGAAGGTGCT |
| | GCCCAAGCACAGCCTGCTGTACGAGTACTTCACCGTGTACAACGAGCTGACCAAGGTGAAGTACGTGACCGAGGGCATGC |
| | GGAAGCCCGCCTTCCTGAGCGGCGAGCAGAAGAAGGCCATCGTGGACCTGCTGTTCAAGACCAACCGGAAGGTGACCGTG |
| | AAGCAGCTGAAGGAGGACTACTTCAAGAAGATCGAGTGCTTCGACAGCGTGGAGATCAGCGGCGTGGAGGACCGGTTCAA |
| | CGCCAGCCTGGGCACCTACCACGACCTGCTGAAGATCATCAAGGACAAGGACTTCCTGGACAACGAGGAGAACGAGGACA |
| | TCCTGGAGGACATCGTGCTGACCCTGACCCTGTTCGAGGACCGGGAGATGATCGAGGAGCGGCTGAAGACCTACGCCCAC |
| | CTGTTCGACGACAAGGTGATGAAGCAGCTGAAGCGGCGGCGGTACACCGGCTGGGGCCGGCTGAGCCGGAAGCTGATCAA |
| | CGGCATCCGGGACAAGCAGAGCGGCAAGACCATCCTGGACTTCCTGAAGAGCGACGGCTTCGCCAACCGGAACTTCATGC |
| | AGCTGATCCACGACGACAGCCTGACCTTCAAGGAGGACATCCAGAAGGCCCAGGTGAGCGGCCAGGGCGACAGCCTGCAC |
| | GAGCACATCGCCAACCTGGCCGGCAGCCCCGCCATCAAGAAGGGCATCCTGCAGACCGTGAAGGTGGTGGACGAGCTGGT |
| | GAAGGTGATGGGCCGGCACAAGCCCGAGAACATCGTGATCGAGATGGCCCGGGAGAACCAGACCACCCAGAAGGGCCAG |
| | AAGAACAGCCGGGAGCGGATGAAGCGGATCGAGGAGGGCATCAAGGAGCTGGGCAGCCAGATCCTGAAGGAGCACCCCG |
| | TGGAGAACACCCAGCTGCAGAACGAGAAGCTGTACCTGTACTACCTGCAGAACGGCCGGGACATGTACGTGGACCAGGAG |
| | CTGGACATCAACCGGCTGAGCGACTACGACGTGGACCACATCGTGCCCCAGAGCTTCCTGAAGGACGACAGCATCGACAA |
| | CAAGGTGCTGACCCGGAGCGACAAGAACCGGGGCAAGAGCGACAACGTGCCCAGCGAGGAGGTGGTGAAGAAGATGAAG |
| | AACTACTGGCGGCAGCTGCTGAACGCCAAGCTGATCACCCAGCGGAAGTTCGACAACCTGACCAAGGCCGAGCGGGGCGG |
| | CCTGAGCGAGCTGGACAAGGCCGGCTTCATCAAGCGGCAGCTGGTGGAGACCCGGCAGATCACCAAGCACGTGGCCCAGA |
| | TCCTGGACAGCCGGATGAACACCAAGTACGACGAGAACGACAAGCTGATCCGGGAGGTGAAGGTGATCACCCTGAAGAGC |
| | AAGCTGGTGAGCGACTTCCGGAAGGACTTCCAGTTCTACAAGGTGCGGGAGATCAACAACTACCACCACGCCCACGACGC |
| | CTACCTGAACGCCGTGGTGGGCACCGCCCTGATCAAGAAGTACCCCAAGCTGGAGAGCGAGTTCGTGTACGGCGACTACA |
| | AGGTGTACGACGTGCGGAAGATGATCGCCAAGAGCGAGCAGGAGATCGGCAAGGCCACCGCCAAGTACTTCTTCTACAGC |
| | AACATCATGAACTTCTTCAAGACCGAGATCACCCTGGCCAACGGCGAGATCCGGAAGCGGCCCCTGATCGAGACCAACGG |
| | CGAGACCGGCGAGATCGTGTGGGACAAGGGCCGGGACTTCGCCACCGTGCGGAAGGTGCTGAGCATGCCCCAGGTGAACA |
| | TCGTGAAGAAGACCGAGGTGCAGACCGGCGGCTTCAGCAAGGAGAGCATCCTGCCCAAGCGGAACAGCGACAAGCTGATC |
| | GCCCGGAAGAAGGACTGGGACCCCAAGAAGTACGGCGGCTTCGACAGCCCCACCGTGGCCTACAGCGTGCTGGTGGTGGC |
| | CAAGGTGGAGAAGGGCAAGAGCAAGAAGCTGAAGAGCGTGAAGGAGCTGCTGGGCATCACCATCATGGAGCGGAGCAGC |
| | TTCGAGAAGAACCCCATCGACTTCCTGGAGGCCAAGGGCTACAAGGAGGTGAAGAAGGACCTGATCATCAAGCTGCCCAA |
| | GTACAGCCTGTTCGAGCTGGAGAACGGCCGGAAGCGGATGCTGGCCAGCGCCGGCGAGCTGCAGAAGGGCAACGAGCTGG |
| | CCCTGCCCAGCAAGTACGTGAACTTCCTGTACCTGGCCAGCCACTACGAGAAGCTGAAGGGCAGCCCCGAGGACAACGAG |
| | CAGAAGCAGCTGTTCGTGGAGCAGCACAAGCACTACCTGGACGAGATCATCGAGCAGATCAGCGAGTTCAGCAAGCGGGT |
| | GATCCTGGCCGACGCCAACCTGGACAAGGTGCTGAGCGCCTACAACAAGCACCGGGACAAGCCCATCCGGGAGCAGGCCG |
| | AGAACATCATCCACCTGTTCACCCTGACCAACCTGGGCGCCCCCGCCGCCTTCAAGTACTTCGACACCACCATCGACCGGA |
| | AGCGGTACACCAGCACCAAGGAGGTGCTGGACGCCACCCTGATCCACCAGAGCATCACCGGCCTGTACGAGACCCGGATC |
| | GACCTGAGCCAGCTGGGCGGCGACTGA |

### Lipid Nanoparticle (LNP) Formulation

In general, the lipid nanoparticle components were dissolved in 100% ethanol at various molar ratios. The RNA cargos (e.g., Cas9 mRNA and sgRNA) were dissolved in 25 mM citrate, 100 mM NaCl, pH 5.0, resulting in a concentration of RNA cargo of approximately 0.45 mg/mL. The LNPs used in Examples 2-4 contained ionizable lipid ((9Z,12Z)-3-((4,4-bis(octyloxy)butanoyl)oxy)-2-((((3-(diethylamino)propoxy)carbonyl)oxy)methyl)propyl octadeca-9,12-dienoate, also called 3-((4,4-bis(octyloxy)butanoyl)oxy)-2-((((3-(diethylamino)propoxy)carbonyl)oxy)methyl)propyl (9Z,12Z)-octadeca-9,12-dienoate), cholesterol, DSPC, and PEG2k-DMG in a 50:38:9:3 molar ratio, respectively. The LNPs were formulated with a lipid amine to RNA phosphate (N:P) molar ratio of about 6, and a ratio of gRNA to mRNA of 1:1 by weight.

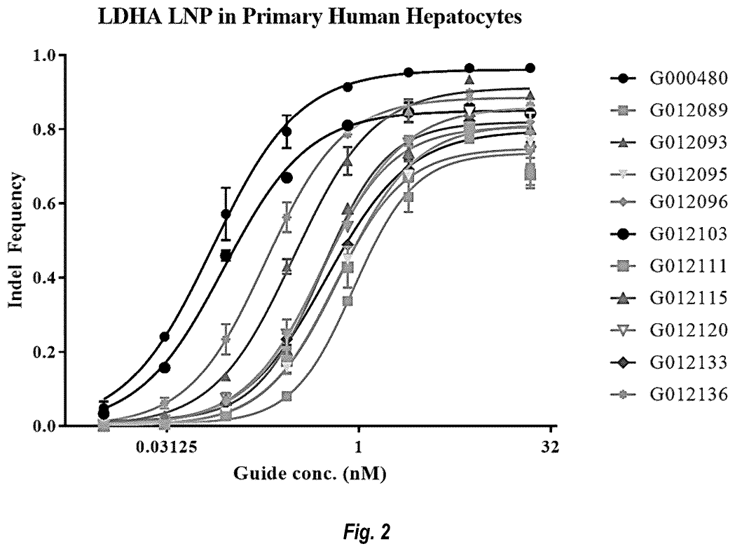

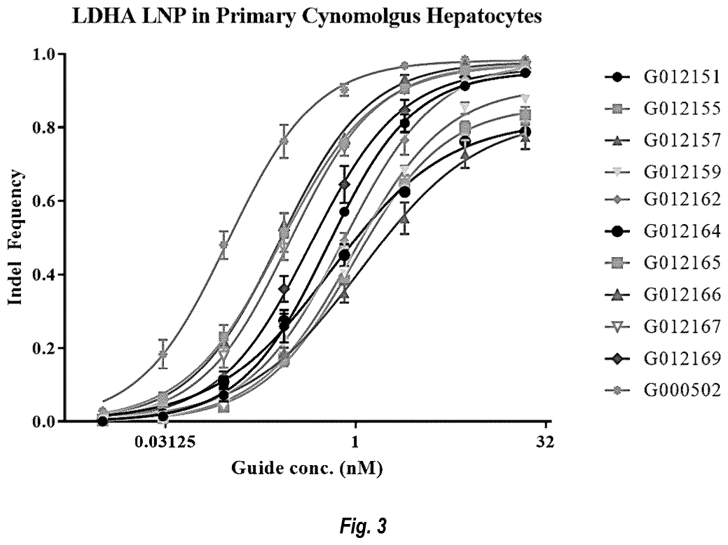

The LNPs were prepared using a cross-flow technique utilizing impinging jet mixing of the lipid in ethanol with two volumes of RNA solutions and one volume of water. The lipid in ethanol was mixed through a mixing cross with the two volumes of RNA solution. A fourth stream of water was mixed with the outlet stream of the cross through an inline tee (See WO2016010840 FIG. 2.). The LNPs were held for 1 hour at room temperature, and further diluted with water (approximately 1:1 v/v). Diluted LNPs were concentrated using tangential flow filtration on a flat sheet cartridge (Sartorius, 100kD MWCO) and then buffer exchanged using PD-10 desalting columns (GE) into 50 mM Tris, 45 mM NaCl, 5% (w/v) sucrose, pH 7.5 (TSS). The resulting mixture was then filtered using a 0.2 m sterile filter. The final LNP was stored at 4° C. or −80° C. until further use.

Human LDHA Guide Design and Human LDHA with Cynomolgus Homology Guide Design

Initial guide selection was performed in silico using a human reference genome (e.g., hg38) and user defined genomic regions of interest (e.g., LDHA protein coding exons), for identifying PAMs in the regions of interest. For each identified PAM, analyses were performed and statistics reported. gRNA molecules were further selected and rank-ordered based on a number of criteria known in the art (e.g., GC content, predicted on-target activity, and potential off-target activity).

A total of 84 guide RNAs were designed toward human LDHA (ENSG00000134333) targeting the protein exonic coding regions. Guides and corresponding genomic coordinates are provided above (Table 1). Forty of the guide RNAs have 100% homology with cynomolgus LDHA.

Additional guides were designed against a de novo Cynomolgus Macaque LDHA transcript. Raw data were obtained from published transcriptome sequencing of liver sample from a female Mauritian-origin Cynomolgus Macaque (NCBI SRA ID: SRR1758956; Peng et al. (2015), Nucleic Acids Research, Volume 43, Issue D1, Pages D737-D742). De novo transcriptome assembly was carried out using Trinity (v2.8.4; Grabherr et al. (2011), Nature Biotechnology, 29: 644-652) and SPAdes (v3.13.0; Bankevich et al. (2012), Journal of Computational Biology, 19:5). Both methods were able to assemble the LDHA transcripts, which were identified by comparing their sequences to LDHA protein (UniProt ID: Q9BE24) with BLAST (Altschul et al. (1990), Journal of Molecular Biology, 215:3, 403-410). Cas9 (mRNA/protein) and guide RNA delivery in vitro

Primary human liver hepatocytes (PHH) (Gibco, Lot #Hu8298 or Hu8296) and primary cynomolgus liver hepatocytes (PCH) (Gibco, Lot #Cy367 or In Vitro ADMET Laboratories, Inc. Lot #10281011) were thawed and resuspended in hepatocyte thawing medium with supplements (Gibco, Cat. CM7500) followed by centrifugation. The supernatant was discarded and the pelleted cells resuspended in hepatocyte plating medium plus supplement pack (Invitrogen, Cat. A1217601 and CM3000). Cells were counted and plated on Bio-coat collagen I coated 96-well plates (ThermoFisher, Cat. 877272) at a density of 33,000 cells/well for PHH and 50,000 cells/well for PCH. Plated cells were allowed to settle and adhere for 5 hours in a tissue culture incubator at 37° C. and 5% CO₂ atmosphere. After incubation cells were checked for monolayer formation and were washed once with hepatocyte culture medium (Takara, Cat. Y20020 and/or Invitrogen, Cat. A1217601 and CM4000).

For studies utilizing dgRNAs, individual crRNA and trRNA was pre-annealed by mixing equivalent amounts of reagent and incubating at 95° C. for 2 min and cooling to room temperature. The dual guide (dgRNA) consisting of pre-annealed crRNA and trRNA, was incubated with Spy Cas9 protein to form a ribonucleoprotein (RNP) complex. Cells were transfected with Lipofectamine RNAiMAX (ThermoFisher, Cat. 13778150) according to the manufacturer's protocol. Cells were transfected with an RNP containing Spy Cas9 (10 nM), individual guide (10 nM), tracer RNA (10 nM), Lipofectamine RNAiMAX (1.0 μL/well) and OptiMem.

For studies utilizing sgRNAs, guides were incubated with Spy Cas9 protein to form a ribonucleoprotein (RNP) complex. In studies utilizing RNP transfection, cells were transfected with Lipofectamine RNAiMAX (ThermoFisher, Cat. 13778150) according to the manufacturer's protocol. Cells were transfected with an RNP containing Spy Cas9 (10 nM), sgRNA (10 nM), Lipofectamine RNAiMAX (1.0 μL/well) and OptiMem. In studies utilizing electroporation, cells were electroporated with RNP containing Spy Cas9 (2 uM) and sgRNA (4 uM), utilizing the Lonza 4D-Nucleofector Core Unit (Cat. AAF-1002X), the 96-well Shuttle Device (Cat. AAM 10015), and the P3 Primary Cell Kit (Cat. V4XP-3960).

Primary human and cyno hepatocytes were also treated with LNPs as further described below. Cells were incubated at 37° C., 5% CO₂ for 48 hours prior to treatment with LNPs. LNPs were incubated in media containing 3% cynomolgus serum at 37° C. for 10 minutes and administered to cells in amounts as further provided herein.

Lipofection of Cas9 mRNA and gRNAs used pre-mixed lipid formulations in which the lipid components were reconstituted in 100% ethanol at a molar ratio of 50% Lipid A, 9% DSPC, 38% cholesterol, and 3% PEG2k-DMG. The lipid mixture was then mixed with RNA cargos (e.g., Cas9 mRNA and gRNA) at a lipid amine to RNA phosphate (N:P) molar ratio of about 6.0. Lipofections were performed with 6% cyno serum and a ratio of gRNA to mRNA of 1:1 by weight.

### Genomic DNA Isolation

PHH and PCH transfected cells were harvested post-transfection at 72 or 96 hours. The gDNA was extracted from each well of a 96-well plate using 50 μL/well BuccalAmp DNA Extraction solution (Epicentre, Cat. QE09050) according to manufacturer's protocol. All DNA samples were subjected to PCR and subsequent NGS analysis, as described herein.

### Next-Generation Sequencing (“NGS”) and Analysis for On-Target Cleavage Efficiency

To quantitatively determine the efficiency of editing at the target location in the genome, deep sequencing was utilized to identify the presence of insertions and deletions introduced by gene editing. PCR primers were designed around the target site within the gene of interest (e.g. LDHA), and the genomic area of interest was amplified. Primer sequence design was done as is standard in the field.

Additional PCR was performed according to the manufacturer's protocols (Illumina) to add chemistry for sequencing. The amplicons were sequenced on an Illumina MiSeq instrument. The reads were aligned to the reference genome (e.g., hg38) after eliminating those having low quality scores. The resulting files containing the reads were mapped to the reference genome (BAM files), where reads that overlapped the target region of interest were selected and the number of wild type reads versus the number of reads which contain an insertion or deletion (“indel”) was calculated.

The editing percentage (e.g., the “editing efficiency” or “percent editing”) is defined as the total number of sequence reads with insertions or deletions (“indels”) over the total number of sequence reads, including wild type.

### Lactate Dehydrogenase a (LDHA) Protein Analysis by Western Blot

Primary human hepatocytes were treated with LNP formulated with select guides from Table 1 as further described in Example 3. LNPs were incubated in media (Takara, Cat. Y20020) containing 3% cynomolgus serum at 37° C. for 10 minutes. Post-incubation the LNPs were added to the human hepatocytes. Twenty-one days post-transfection, the media was removed and the cells were lysed with 50 μL/well RIPA buffer (Boston Bio Products, Cat. BP-115) plus freshly added protease inhibitor mixture consisting of complete protease inhibitor cocktail (Sigma, Cat. 11697498001), 1 mM DTT, and 250 U/ml Benzonase (EMD Millipore, Cat. 71206-3). Cells were kept on ice for 30 minutes at which time NaCl (1 M final concentration) was added. Cell lysates were thoroughly mixed and retained on ice for 30 minutes. The whole cell extracts (“WCE”) were transferred to a PCR plate and centrifuged to pellet debris. A Bradford assay (Bio-Rad, Cat. 500-0001) was used to assess protein content of the lysates. The Bradford assay procedure was completed according to the manufacturer's protocol. Extracts were stored at −20° C. prior to use.

AGT-deficient mice were treated with LNP formulated with select guides as further described in Example 4. Livers were harvested from the mice post-treatment and 60 mg portions were used for protein extraction. The samples were placed in bead tubes (MP Biomedical, Cat. 6925-500) and lysed with 600 μL/sample of RIPA buffer (Boston Bio Products, Cat. BP-115) plus freshly added protease inhibitor mixture consisting of complete protease inhibitor cocktail (Sigma, Cat. 116974500) and homogenized at 5.0 m/sec. The samples were then centrifuged at 14,000 RPM for 10 min. at 4° C. and the liquid was transferred to a new tube. A final centrifugation was performed at 14,000 RPM for 10 min. and the samples were quantified using a Bradford assay as described above.

A western blot was performed to assess LDHA protein levels. Lysates were mixed with Laemmli buffer and denatured at 95° C. for 10 minutes. The blot was run using the NuPage system on 10% Bis-Tris gels (Thermo Fisher Scientific, Cat. NPO302BOX) according to the manufacturer's protocol followed by wet transfer onto 0.45 μm nitrocellulose membrane (Bio-Rad, Cat. 1620115). After the transfer membrane was rinsed thoroughly with water and stained with Ponceau S solution (Boston Bio Products, Cat. ST-180) to confirm complete and even transfer. The blot was blocked using 5% Dry Milk in TBS for 30 minutes on a lab rocker at room temperature. The blot was rinsed with TBST and probed with rabbit α-LDHA polyclonal antibody (Sigma, Cat. SAB2108638 for cell lysate or Genetex, Cat. GTX101416 for mouse liver lysate) at 1:1000 in TBST. For blots with in vitro cell lysate, beta-actin was used as a loading control (Novus, Cat. NB600-501) at 1:1000 in TBST and incubated simultaneously with the LDHA primary antibody. For blots with in vivo mouse liver extracts, GAPDH was used as a loading control (Abcam, ab8245) at 1:1000 in TBST and incubated simultaneously with the LDHA primary antibody. The blot was sealed in a bag and kept overnight at 4° C. on a lab rocker. After incubation, the blot was rinsed 3 times for 5 minutes each in TBST and probed with secondary antibodies to Mouse and Rabbit (Thermo Fisher Scientific, Cat. PI35518 and PISA535571) at 1:12,500 each in TBST for 30 minutes at room temperature. After incubation, the blot was rinsed 3 times for 5 minutes each in TBST and 2 times with PBS. The blot was visualized and analyzed using a Licor Odyssey system.

### Lactate Dehydrogenase a (LDHA) Protein Analysis by Immunohistochemical Staining

For visual LDHA protein analysis of mouse livers, standard immunohistochemical staining was conducted on a Lecia Bond Rxm. For antigen retrieval (HIER), slides were heated in a pH 9 EDTA-based buffer for 25 minutes at 94° C., followed by a 30 minute antibody incubation at 1:500 (Abcam Cat. Ab52488). Antibody binding was detected using an HRP-conjugated secondary polymer, followed by chromogenic visualization with diaminobenzidine.

Measurement of LDH Activity from Mouse Muscle and Liver

A biochemical method (e.g., Wood K D et al., Biochim Biophys Acta Mol Basis Dis. 2019 Sep. 1; 1865(9):2203-2209; PMC6613992) was used for lactate dehydrogenase activity. For measurement of lactate dehydrogenase activity, tissue was homogenized in iced cold lysis buffer (25 mM HEPES, pH 7.3, 0.1% Triton-X-100) with probe sonication to give a 10% wt/vol lysate. LDH activity was measured by the increased in absorbance at 340 nm with the reduction of NAD to NADH in the presence of lactate. Lactate to pyruvate activity of LDG was measured with 20 mM lactate, 100 mM Tris-HCL, pH 9.0, 2 mM NAD+, 0.01% liver lysate. A Cooomassie Plus protein assay kit (Pierce, Rockford, IL), with bovine serum albumin (BSA) as the standard, was used to determine protein concentration in tissue lysates.

Measurement of Oxalate, Creatinine, Pyruvate, and Lactate from Mouse Samples

For oxalate determination, part of the urine collection was acidified to pH between 1 and 2 with HCl prior to storage at −80° C. to prevent any possible oxalate crystallization that could occur with cold storage and/or oxalogensis associated with alkalinization. The remaining nonacidified urine was frozen at −80° C. for the measurement of creatinine. Plasma preparations were filtered through Nano-sep centrifugal filters (VWR International, Batavia, IL) with a 10,000 nominal molecular weight limit to remove macromolecules prior to ion chromatography coupled with mass spectrometry or ICMS (Thermo Fisher Scientific Inc., Waltham, MA). Centrifugal filters were washed with 10 mM HCl prior to sample filtration to remove any contaminating trace organic acids trapped in the filter device. Liver tissue was extracted with 10% (wt/vol) trichloroacetic acid (TCA) for organic acid analysis. These organic acids were measured by ICMS following removal of TCA by vigorous vortexing with an equal volume of 1,1,2-trichlorotrifluoroethane (Freon)-trioctylamine (3:1, vol/vol; Aldrich, Milwaukee, WI), centrifuging at 4° C. to promote phase separation, and collecting the upper aqueous layer for analysis. Urinary creatinine was measured on a chemical analyzer, and urinary oxalate by ICMS, as previously described.

Selected-ion monitoring (SIM) at the following mass/charge ratios and cone voltages were used to quantify lactate (SIM 89.0, 35 V) and 13C3-lactate (SIM 92.0, 35 V). Pyruviate was measured by IC/MS with an AS11-HC 4 m, 2×150 mm, anion exchange column at a controlled temperature of 30° C. and a Dionex™ ERS™ 500 anion electrolytically regenerated suppressor. A gradient of KOH from 0.5 to 80 mM over 60 min at a flow rate of 0.38 ml/min was used to separate sample anions. The mass spectrometer (MSQ-PLUS) was operated in ESI negative mode, needle voltage 1.5 V, 500° C. source temperature, and column eluent was mixed with 50% acetonitrile at 0.38 ml/min using a zero dead volume mixing tee prior to entry into the MSQ. Selected-ion monitoring (SIM) at the following mass/charge ratios and cone voltages were used to pyruvate (SIM 87.0, 30 V).

### Example 2—Screening and Guide Qualification

### Cross Screening of LDHA Guides in Primary Hepatocytes

Guides targeting human LDHA and those with homology in cynomolgus monkey were transfected into primary human (via RNP transfection) and cynomolgus hepatocytes (via RNP electroporation) as described in Example 1. Percent editing was determined for sgRNAs comprising each guide sequence across each cell type. The screening data for the guide sequences in Table 1 in both cell lines are listed below (Tables 4-5).

Table 4 shows the average and standard deviation of duplicate samples for 00 Edit, 0% Insertion (Ins), and 0% Deletion (Del) for the LDHA transfected as RNP into primary human hepatocytes. N=2.

TABLE 4
LDHA editing data for sgRNAs delivered to primary
human hepatocytes via RNP transfection

| | Avg | Std Dev | Avg | Std Dev | Avg | Std Dev |
|---|---|---|---|---|---|---|
| GUIDE ID | % Edit | % Edit | % Ins | % Ins | % Del | % Del |
| G009440 | 9.50 | 4.10 | 2.45 | 0.92 | 7.05 | 3.18 |
| G012089 | 38.15 | 3.18 | 12.10 | 0.14 | 26.00 | 3.25 |
| G012090 | 11.85 | 3.89 | 1.45 | 0.49 | 10.60 | 3.39 |
| G012092 | 22.75 | 2.76 | 4.00 | 0.14 | 19.65 | 2.62 |
| G012093 | 34.60 | 0.28 | 8.95 | 0.21 | 25.60 | 0.42 |
| G012094 | 20.50 | 0.42 | 14.30 | 0.85 | 6.35 | 1.34 |
| G012095 | 28.45 | 2.33 | 3.50 | 0.71 | 25.00 | 2.97 |
| G012096 | 32.30 | 0.42 | 0.70 | 0.00 | 31.75 | 0.49 |
| G012097 | 24.65 | 1.34 | 3.95 | 1.06 | 20.75 | 2.33 |
| G012098 | 6.25 | 1.77 | 2.10 | 0.57 | 4.30 | 1.13 |
| G012099 | 12.20 | 1.84 | 5.10 | 0.85 | 7.10 | 0.99 |
| G012100 | 9.40 | 1.13 | 6.95 | 0.78 | 2.45 | 0.35 |
| G012101 | 3.60 | 0.85 | 1.45 | 0.35 | 2.15 | 0.49 |
| G012103 | 34.90 | 3.11 | 2.30 | 0.00 | 32.70 | 3.25 |
| G012104 | 5.85 | 2.33 | 0.25 | 0.21 | 5.60 | 2.12 |
| G012105 | 23.45 | 0.78 | 8.45 | 0.49 | 15.15 | 1.34 |
| G012106 | 5.80 | 1.56 | 1.60 | 0.14 | 4.20 | 1.41 |
| G012107 | 2.85 | 0.21 | 0.75 | 0.21 | 2.20 | 0.28 |
| G012108 | 14.50 | 0.57 | 0.80 | 0.14 | 13.75 | 0.64 |
| G012109 | 12.40 | 0.71 | 0.65 | 0.07 | 11.80 | 0.71 |
| G012110 | 12.00 | 1.98 | 3.85 | 0.49 | 8.35 | 1.48 |
| G012111 | 27.20 | 0.28 | 16.40 | 0.14 | 10.85 | 0.07 |
| G012112 | 3.85 | 1.34 | 0.95 | 0.35 | 2.95 | 1.06 |
| G012113 | 9.45 | 2.62 | 2.05 | 1.06 | 7.40 | 1.56 |
| G012114 | 7.05 | 0.78 | 1.95 | 0.07 | 5.10 | 0.85 |
| G012115 | 31.10 | 7.64 | 12.40 | 3.25 | 18.90 | 4.24 |
| G012116 | 12.55 | 1.34 | 4.85 | 0.07 | 7.80 | 1.41 |
| G012117 | 10.40 | 1.41 | 3.40 | 0.00 | 7.40 | 1.56 |
| G012118 | 21.95 | 3.32 | 2.35 | 0.35 | 19.60 | 2.97 |
| G012119 | 15.50 | 3.68 | 0.50 | 0.14 | 14.95 | 3.46 |
| G012120 | 22.05 | 4.88 | 1.70 | 0.71 | 20.45 | 4.31 |
| G012121 | 10.90 | 0.28 | 3.45 | 0.21 | 7.65 | 0.64 |
| G012122 | 2.60 | 0.28 | 0.40 | 0.00 | 2.20 | 0.28 |
| G012123 | 6.80 | 0.85 | 1.90 | 0.14 | 4.90 | 0.71 |
| G012124 | 10.90 | 2.40 | 1.30 | 0.14 | 9.70 | 2.26 |
| G012125 | 6.10 | 0.42 | 0.85 | 0.21 | 5.35 | 0.64 |
| G012126 | 1.85 | 0.21 | 0.50 | 0.00 | 1.35 | 0.21 |
| G012127 | 10.05 | 1.20 | 0.85 | 0.21 | 9.30 | 1.41 |
| G012128 | 6.20 | 0.14 | 1.05 | 0.21 | 5.20 | 0.28 |
| G012129 | 6.40 | 0.71 | 0.45 | 0.07 | 6.00 | 0.57 |
| G012130 | 1.00 | 0.14 | 0.55 | 0.07 | 0.55 | 0.07 |
| G012131 | 3.15 | 0.21 | 0.70 | 0.28 | 2.55 | 0.35 |
| G012132 | 17.90 | 1.84 | 11.50 | 2.12 | 6.45 | 0.21 |
| G012133 | 23.45 | 0.64 | 6.70 | 0.14 | 16.75 | 0.49 |
| G012134 | 4.45 | 0.07 | 1.70 | 0.00 | 2.85 | 0.07 |
| G012135 | 16.80 | 0.71 | 4.30 | 0.42 | 12.60 | 0.42 |
| G012136 | 38.65 | 0.92 | 0.90 | 0.00 | 37.80 | 0.99 |
| G012137 | 1.10 | 0.28 | 0.30 | 0.14 | 0.80 | 0.14 |
| G012138 | 17.35 | 3.75 | 4.70 | 0.99 | 12.85 | 2.76 |
| G012139 | 6.30 | 0.57 | 0.45 | 0.35 | 5.85 | 0.21 |
| G012140 | 14.65 | 2.33 | 4.30 | 1.84 | 10.45 | 0.49 |
| G012141 | 0.95 | 0.07 | 0.35 | 0.07 | 0.65 | 0.07 |
| G012142 | 32.35 | 0.92 | 30.85 | 1.06 | 19.55 | 0.64 |
| G012143 | 3.35 | 0.07 | 1.75 | 0.07 | 1.60 | 0.00 |
| G012149 | 17.65 | 0.35 | 1.50 | 0.57 | 16.20 | 0.14 |
| G012150 | 12.65 | 0.64 | 9.50 | 0.85 | 3.20 | 0.14 |
| G012151 | 12.90 | 0.14 | 6.70 | 0.14 | 6.25 | 0.21 |
| G012152 | 4.80 | 0.14 | 0.80 | 0.14 | 4.10 | 0.00 |
| G012154 | 11.45 | 2.90 | 4.85 | 1.06 | 6.65 | 1.91 |
| G012156 | 7.85 | 1.34 | 3.70 | 0.42 | 4.30 | 0.85 |
| G012158 | 10.90 | 1.56 | 2.20 | 0.57 | 8.70 | 0.99 |
| G012159 | 11.35 | 0.49 | 2.35 | 0.07 | 9.10 | 0.57 |
| G012160 | 10.40 | 0.42 | 2.00 | 0.28 | 8.45 | 0.07 |
| G012162 | 3.95 | 0.49 | 1.75 | 0.35 | 2.30 | 0.14 |
| G012165 | 27.95 | 3.04 | 1.40 | 0.71 | 26.55 | 2.47 |
| G012167 | 27.95 | 1.06 | 18.70 | 0.57 | 9.35 | 0.49 |
| G012168 | 9.90 | 1.27 | 0.50 | 0.28 | 9.50 | 0.99 |
| G012169 | 20.20 | 2.97 | 4.05 | 0.78 | 16.30 | 2.12 |
| G012171 | 19.15 | 1.34 | 2.90 | 0.71 | 16.40 | 0.57 |
| G012172 | 15.85 | 2.47 | 2.15 | 0.35 | 13.85 | 2.19 |
| G012173 | 11.10 | 0.14 | 6.60 | 0.14 | 4.55 | 0.07 |

Table 5 shows the average and standard deviation for 00 Edit, 0% Insertion (Ins), and 0% Deletion (Del) for the tested LDHA sgRNAs electroporated with RNP in primary cynomolgus hepatocytes. N=2.

TABLE 5
LDHA editing data for sgRNAs delivered to primary
cynomolgus hepatocytes via RNP electroporation

| GUIDE | Avg | Std Dev | Avg | Std Dev | Avg | Std Dev |
|---|---|---|---|---|---|---|
| ID | % Edit | % Edit | % Ins | % Ins | % Del | % Del |
| G012090 | 11.40 | 8.34 | 0.20 | 0.14 | 11.30 | 8.20 |
| G012143 | 4.75 | 0.92 | 2.25 | 0.07 | 2.60 | 0.85 |
| G012145 | 4.10 | 1.70 | 0.15 | 0.07 | 3.95 | 1.63 |
| G012146 | 9.60 | 2.69 | 3.50 | 1.70 | 6.20 | 1.13 |
| G012147 | 0.20 | 0.00 | 0.00 | 0.00 | 0.15 | 0.07 |
| G012148 | 36.30 | 1.70 | 12.80 | 0.28 | 23.90 | 1.56 |
| G012149 | 31.00 | 3.82 | 1.30 | 0.00 | 29.65 | 3.75 |
| G012150 | 30.35 | 19.16 | 18.60 | 14.00 | 11.95 | 5.16 |
| G012151 | 65.05 | 4.45 | 36.60 | 2.26 | 28.50 | 2.12 |
| G012152 | 19.50 | 0.14 | 0.55 | 0.21 | 19.05 | 0.21 |
| G012153 | 0.90 | 0.42 | 0.05 | 0.07 | 0.85 | 0.35 |
| G012154 | 47.50 | 0.99 | 28.60 | 3.68 | 19.00 | 2.55 |
| G012155 | 65.55 | 3.32 | 2.25 | 0.21 | 63.65 | 3.18 |
| G012156 | 17.60 | 9.05 | 3.05 | 0.92 | 14.55 | 8.27 |
| G012157 | 42.80 | 6.36 | 7.70 | 0.28 | 35.10 | 6.65 |
| G012158 | 31.95 | 17.47 | 4.35 | 3.04 | 27.70 | 14.57 |
| G012159 | 44.70 | 1.41 | 3.60 | 0.28 | 41.10 | 1.13 |
| G012160 | 34.70 | 1.70 | 7.55 | 0.78 | 27.20 | 2.40 |
| G012161 | 25.75 | 6.58 | 5.75 | 3.18 | 20.20 | 3.39 |
| G012162 | 14.50 | 3.82 | 6.55 | 0.35 | 8.10 | 3.54 |
| G012163 | 28.30 | 4.53 | 0.40 | 0.00 | 28.00 | 4.53 |
| G012164 | 57.85 | 2.33 | 3.65 | 0.35 | 54.20 | 2.69 |
| G012165 | 42.75 | 14.07 | 1.30 | 0.14 | 41.45 | 13.93 |
| G012166 | 57.55 | 5.30 | 39.70 | 3.11 | 17.90 | 2.12 |
| G012167 | 47.95 | 12.94 | 23.50 | 6.65 | 24.70 | 6.08 |
| G012168 | 21.80 | N/A | 0.10 | N/A | 21.80 | N/A |
| G012169 | 58.25 | 5.73 | 2.50 | 0.57 | 55.85 | 5.30 |
| G012170 | 17.55 | 4.60 | 5.40 | 0.42 | 12.15 | 4.17 |
| G012171 | 49.25 | 9.83 | 6.75 | 3.04 | 42.55 | 6.86 |
| G012172 | 19.10 | 3.68 | 1.45 | 0.35 | 17.65 | 3.32 |
| G012173 | 21.35 | 8.27 | 7.75 | 3.18 | 13.65 | 5.16 |

Table 6 shows the average and standard deviation for 00 Edit across multiple chromosomal locations for the tested LDHA sgRNAs in primary cynomolgus hepatocytes using lipofection at 30 nM concentration of sgRNA. N=2.

TABLE 6
LDHA editing data for sgRNAs delivered
to primary cynomolgus hepatocytes

| | Chr12 | Chr12 | Chr14 | Chr14 | Chr17 | Chr17 |
|---|---|---|---|---|---|---|
| | Avg | Std Dev | Avg | Std Dev | Avg | Std Dev |
| GUIDE ID | % Edit | % Edit | % Edit | % Edit | % Edit | % Edit |
| G015538 | 0.0 | 0.0 | 0.0 | 0.0 | 0.0 | 0.0 |
| G015539 | 0.0 | 0.0 | 19.4 | 3.5 | 0.0 | 0.0 |
| G015540 | 0.0 | 0.0 | 34.6 | 0.5 | 0.0 | 0.0 |
| G015541 | 59.3 | 6.7 | 0.0 | 0.0 | 59.3 | 5.4 |
| G015542 | 0.0 | 0.0 | 0.0 | 0.0 | 31.7 | 1.1 |
| G015543 | 0.0 | 0.0 | 27.0 | 1.6 | 0.0 | 0.0 |
| G015544 | 0.0 | 0.0 | 7.6 | 0.8 | 0.0 | 0.0 |
| G015545 | 0.0 | 0.0 | 9.3 | 1.7 | 0.0 | 0.0 |
| G015546 | 0.0 | 0.0 | 0.0 | 0.0 | 0.0 | 0.0 |
| G015547 | 0.0 | 0.0 | 58.6 | 4.2 | 0.0 | 0.0 |
| G015548 | 0.0 | 0.0 | 32.5 | 4.0 | 0.0 | 0.0 |
| G015549 | 0.0 | 0.0 | 9.4 | 5.1 | 0.0 | 0.0 |
| G015550 | 15.0 | 4.2 | 0.0 | 0.0 | 15.9 | 4.3 |
| G015551 | 0.0 | 0.0 | 6.7 | 3.5 | 0.0 | 0.0 |
| G015552 | 25.7 | 16.6 | 0.0 | 0.0 | 26.7 | 16.0 |
| G015553 | 21.6 | 0.0 | 0.0 | 0.0 | 25.1 | 9.7 |
| G015554 | 0.0 | 0.0 | 20.4 | 7.4 | 0.0 | 0.0 |
| G015555 | 0.0 | 0.0 | 32.3 | 14.0 | 0.0 | 0.0 |
| G015556 | 0.0 | 0.0 | 0.0 | 0.0 | 0.0 | 0.0 |
| G015557 | 0.0 | 0.0 | 8.6 | 5.3 | 4.0 | 0.0 |
| G015558 | 0.0 | 0.0 | 15.9 | 11.2 | 0.0 | 0.0 |
| G015559 | 0.0 | 0.0 | 0.0 | 0.0 | 0.0 | 0.0 |
| G015560 | 0.0 | 0.0 | 36.7 | 0.0 | 0.0 | 0.0 |
| G015561 | 0.0 | 0.0 | 42.1 | 0.0 | 0.0 | 0.0 |
| G015562 | 51.6 | 0.0 | 0.0 | 0.0 | 43.8 | 0.0 |
| G015563 | 37.2 | 0.0 | 0.0 | 0.0 | 38.3 | 0.0 |
| G015564 | 44.9 | 0.0 | 0.0 | 0.0 | 40.2 | 0.0 |
| G015565 | 0.0 | 0.0 | 0.0 | 0.0 | 0.0 | 0.0 |
| G015566 | 35.6 | 0.0 | 0.0 | 0.0 | 36.5 | 0.0 |
| G015567 | 0.0 | 0.0 | 3.6 | 0.0 | 0.0 | 0.0 |
| G015568 | 0.0 | 0.0 | 10.3 | 3.0 | 0.0 | 0.0 |
| G015569 | 0.0 | 0.0 | 22.6 | 0.9 | 0.0 | 0.0 |
| G015570 | 0.0 | 0.0 | 17.4 | 1.0 | 0.0 | 0.0 |
| G015571 | 0.0 | 0.0 | 98.0 | 0.2 | 0.0 | 0.0 |
| G015572 | 0.0 | 0.0 | 14.7 | 0.7 | 0.0 | 0.0 |
| G015573 | 0.0 | 0.0 | 7.6 | 2.0 | 0.0 | 0.0 |
| G015574 | 0.0 | 0.0 | 15.8 | 3.8 | 0.0 | 0.0 |
| G015575 | 0.0 | 0.0 | 0.0 | 0.0 | 27.0 | 4.2 |
| G015576 | 0.0 | 0.0 | 0.0 | 0.0 | 16.5 | 2.5 |
| G015577 | 0.0 | 0.0 | 0.0 | 0.0 | 27.8 | 5.4 |
| G015578 | 0.0 | 0.0 | 0.0 | 0.0 | 0.0 | 0.0 |
| G015579 | 41.4 | 1.0 | 0.0 | 0.0 | 42.2 | 1.9 |
| G015580 | 17.4 | 0.0 | 0.0 | 0.0 | 24.2 | 1.6 |
| G015581 | 6.2 | 0.5 | 0.0 | 0.0 | 6.3 | 0.1 |
| G015582 | 0.0 | 0.0 | 0.0 | 0.0 | 0.0 | 0.0 |
| G015583 | 0.0 | 0.0 | 27.8 | 2.2 | 0.0 | 0.0 |
| G015584 | 0.0 | 0.0 | 6.5 | 0.0 | 0.0 | 0.0 |
| G015585 | 0.0 | 0.0 | 4.3 | 1.3 | 0.0 | 0.0 |
| G015586 | 0.0 | 0.0 | 20.5 | 0.8 | 15.0 | 1.1 |
| G015587 | 0.0 | 0.0 | 40.6 | 3.2 | 0.0 | 0.0 |
| G015588 | 0.0 | 0.0 | 21.2 | 1.2 | 0.0 | 0.0 |
| G015589 | 0.0 | 0.0 | 22.4 | 0.8 | 0.0 | 0.0 |
| G015590 | 0.0 | 0.0 | 29.3 | 4.3 | 0.0 | 0.0 |
| G015591 | 0.0 | 0.0 | 38.8 | 2.3 | 0.0 | 0.0 |
| G015592 | 0.0 | 0.0 | 0.0 | 0.0 | 0.0 | 0.0 |
| G015593 | 0.0 | 0.0 | 9.8 | 1.3 | 0.0 | 0.0 |
| G015594 | 0.0 | 0.0 | 41.4 | 6.4 | 0.0 | 0.0 |
| G015595 | 0.0 | 0.0 | 0.0 | 0.0 | 0.0 | 0.0 |
| G015596 | 0.0 | 0.0 | 5.1 | 2.7 | 0.0 | 0.0 |
| G015597 | 0.0 | 0.0 | 12.1 | 2.2 | 0.0 | 0.0 |
| G015598 | 0.0 | 0.0 | 25.6 | 3.5 | 0.0 | 0.0 |
| G015599 | 0.0 | 0.0 | 25.6 | 1.8 | 0.0 | 0.0 |
| G015600 | 35.9 | 4.7 | 0.0 | 0.0 | 38.4 | 7.6 |
| G015601 | 23.6 | 0.6 | 0.0 | 0.0 | 24.1 | 1.1 |
| G015602 | 0.0 | 0.0 | 37.6 | 1.7 | 0.0 | 0.0 |
| G015603 | 0.0 | 0.0 | 17.7 | 0.3 | 0.0 | 0.0 |
| G015604 | 53.5 | 7.2 | 0.0 | 0.0 | 72.6 | 2.5 |
| G015605 | 12.3 | 2.8 | 0.0 | 0.0 | 13.5 | 1.2 |
| G015606 | 30.5 | 0.8 | 0.0 | 0.0 | 27.3 | 1.6 |
| G015607 | 10.9 | 2.9 | 0.0 | 0.0 | 11.5 | 0.5 |
| G015608 | 0.0 | 0.0 | 0.0 | 0.0 | 20.3 | 1.5 |
| G015609 | 0.0 | 0.0 | 0.0 | 0.0 | 0.0 | 0.0 |
| G015610 | 0.0 | 0.0 | 29.5 | 0.3 | 0.0 | 0.0 |
| G015611 | 0.0 | 0.0 | 14.8 | 1.0 | 0.0 | 0.0 |
| G015612 | 0.00 | 0.00 | 2.00 | 0.00 | 22.90 | 0.00 |
| G015613 | 0.00 | 0.00 | 32.90 | 0.85 | 33.90 | 2.55 |
| G015614 | 0.00 | 0.00 | 0.00 | 0.00 | 12.25 | 0.64 |
| G015615 | 0.00 | 0.00 | 0.00 | 0.00 | 30.05 | 1.91 |
| G015616 | 0.00 | 0.00 | 0.00 | 0.00 | 5.25 | 0.21 |
| G015617 | 0.00 | 0.00 | 0.00 | 0.00 | 36.15 | 0.64 |
| G015618 | 0.00 | 0.00 | 0.00 | 0.00 | 8.75 | 0.92 |
| G015619 | 2.45 | 0.35 | 0.00 | 0.00 | 3.85 | 0.49 |
| G015620 | 0.00 | 0.00 | 0.00 | 0.00 | 18.25 | 2.90 |
| G015621 | 0.00 | 0.00 | 0.00 | 0.00 | 46.70 | 0.71 |
| G015622 | 41.60 | 2.83 | 3.05 | 1.06 | 0.00 | 0.00 |
| G015623 | 15.60 | 0.42 | 1.15 | 0.35 | 0.00 | 0.00 |
| G015624 | 0.00 | 0.00 | 1.70 | 0.57 | 0.00 | 0.00 |
| G015625 | 0.00 | 0.00 | 0.00 | 0.00 | 22.50 | 1.70 |
| G015626 | 0.00 | 0.00 | 0.00 | 0.00 | 50.45 | 1.48 |
| G015627 | 0.00 | 0.00 | 0.00 | 0.00 | 24.60 | 0.85 |
| G015628 | 0.00 | 0.00 | 0.00 | 0.00 | 8.70 | 1.27 |
| G015629 | 0.00 | 0.00 | 0.00 | 0.00 | 50.55 | 0.07 |
| G015630 | 17.10 | 0.28 | 0.00 | 0.00 | 0.00 | 0.00 |

Based on the primary human and primary cyno hepatocyte editing data, a subset of guide sequences were further evaluated. This subset is provided in Tables 7 and 8, with the corresponding editing data from primar hepatocyte screens reproduced.

TABLE 7
LDHA editing data for sgRNAs in primary human
hepatocytes chosen for further analysis in PHH

| GUIDE ID | % Edit (from Table 4 above) |
|---|---|
| G012089 | 38.15 |
| G012093 | 34.60 |
| G012095 | 28.45 |
| G012096 | 32.30 |
| G012103 | 34.90 |
| G012111 | 27.20 |
| G012115 | 31.10 |
| G012120 | 22.05 |
| G012133 | 23.45 |
| G012136 | 38.65 |

TABLE 8
LDHA editing data for sgRNAs in primary cynomolgus
hepatocytes chosen for further analysis in PCH

| GUIDE ID | % Edit (from Table 5 above) |
|---|---|
| G012151 | 65.05 |
| G012155 | 65.55 |
| G012157 | 42.8 |
| G012159 | 44.7 |
| G012162 | 14.5 |
| G012164 | 57.85 |
| G012165 | 42.75 |
| G012166 | 57.55 |
| G012167 | 47.95 |
| G012169 | 58.25 |

### Off-Target Analysis of LDHA Guides

A biochemical method (See, e.g., Cameron et al., Nature Methods. 6, 600-606; 2017) was used to determine potential off-target genomic sites cleaved by Cas9 targeting LDHA. In this experiment, 10 modified sgRNA targeting human LDHA (and two control guides with known off-target profiles) were screened using isolated HEK293 genomic DNA and the potential off-target results were plotted in FIG. 1. The assay identified potential off-target sites for the sgRNAs tested.

### Targeted Sequencing for Validating Potential Off-Target Sites

In known off-target detection assays such as the biochemical method used above, a large number of potential off-target sites are typically recovered, by design, so as to “cast a wide net” for potential sites that can be validated in other contexts, e.g., in a primary cell of interest. For example, the biochemical method typically overrepresents the number of potential off-target sites as the assay utilizes purified high molecular weight genomic DNA free of the cell environment and is dependent on the dose of Cas9 RNP used. Accordingly, potential off-target sites identified by these methods may be validated using targeted sequencing of the identified potential off-target sites.

In one approach, primary hepatocytes are treated with LNPs comprising Cas9 mRNA and a sgRNA of interest (e.g., a sgRNA having potential off-target sites for evaluation). The primary hepatocytes are then lysed and primers flanking the potential off-target site(s) are used to generate an amplicon for NGS analysis. Identification of indels at a certain level may validate potential off-target site, whereas the lack of indels found at the potential off-target site may indicate a false positive in the off-target assay that was utilized.

Cross Screening of Lipid Nanoparticle (LNP) Formulations Containing Spy Cas9 mRNA and sgRNA in Primary Human and Cynomolgus Hepatocytes

Lipid nanoparticle (LNP) formulations of modified sgRNAs targeting human LDHA and those homologous in cyno were tested on primary human hepatocytes and primary cynomolgus hepatocytes in a dose response assay. The LNPs were formulated as described in Example 1. Primary human and cynomolgus hepatocytes were plated as described in Example 1. Both cell lines were incubated at 37° C., 5% CO₂ for 48 hours prior to treatment with LNPs. LNPs were incubated in media containing 6% cynomolgus serum at 37° C. for 10 minutes. Post-incubation the LNPs were added to the human or cynomolgus hepatocytes in an 8 point 3-fold dose response curve starting at 300ng Cas9 mRNA. The cells were lysed 96 hours post-treatment for NGS analysis as described in Example 1. The dose response curve data for the guide sequences in both cell lines is shown inFIGS. 2 and 3. The percent editing at the 22 nM concentration are listed below in Tables 9 and 10.

Table 9 shows the average and standard deviation for 0% Edit, % Insertion (Ins), and % Deletion (Del) for the tested LDHA sgRNAs at 22 nM delivered with Spy Cas9 via LNP in primary human hepatocytes. These samples were generated in duplicate.

TABLE 9
LDHA editing data for sgRNAs/Cas9 mRNA delivered to
primary human hepatocytes via LNP at 22 nM (with
respect to the concentration of the sgRNA cargo)

| GUIDE | Avg | Std Dev | Avg | Std Dev | Avg | Std Dev | |
|---|---|---|---|---|---|---|---|
| ID | % Edit | % Edit | % Ins | % Ins | % Del | % Del | EC50 |
| G012089 | 69.10 | 4.95 | 22.65 | 3.04 | 46.50 | 1.98 | 90.93 |
| G012093 | 89.30 | 0.99 | 20.75 | 0.64 | 68.65 | 1.48 | 30.85 |
| G012095 | 76.75 | 2.19 | 8.70 | 0.14 | 68.20 | 2.40 | 71.83 |
| G012096 | 82.00 | 2.55 | 1.90 | 0.42 | 80.10 | 2.12 | 53.27 |
| G012103 | 84.30 | 0.00 | 5.65 | 1.20 | 78.75 | 1.20 | 8.73 |
| G012111 | 67.80 | 2.83 | 32.95 | 2.62 | 34.90 | 0.14 | 63.84 |
| G012115 | 80.05 | 3.46 | 34.65 | 1.91 | 45.55 | 1.48 | 50.98 |
| G012120 | 74.15 | 1.91 | 5.20 | 1.27 | 69.00 | 0.71 | 48.93 |
| G012133 | 75.25 | 1.20 | 24.55 | 1.20 | 50.75 | 2.33 | 55.54 |
| G012136 | 86.50 | 0.71 | 1.45 | 0.07 | 85.10 | 0.85 | 18.54 |

Table 10 shows the average and standard deviation for % Edit, % Insertion (Ins), and 00 Deletion (Del) for the tested LDHA sgRNAs at 22 nM delivered with Spy Cas9 via LNP in primary_cynomolgus hepatocytes. These samples were generated in triplicate.

TABLE 10
LDHA editing data for sgRNAs/Cas9 mRNA delivered to
primary cynomolgus hepatocytes via LNP at 22 nM (with
respect to the concentration of the sgRNA cargo)

| GUIDE | Avg | Std Dev | Avg | Std Dev | Avg | Std Dev | |
|---|---|---|---|---|---|---|---|
| ID | % Edit | % Edit | % Ins | % Ins | % Del | % Del | EC50 |
| G012151 | 94.87 | 0.12 | 78.50 | 1.39 | 16.77 | 1.33 | 0.599 |
| G012155 | 96.93 | 0.23 | 7.17 | 0.15 | 90.83 | 0.31 | 0.255 |
| G012157 | 77.43 | 3.33 | 31.77 | 1.76 | 46.80 | 2.17 | 1.111 |
| G012159 | 87.73 | 1.02 | 20.47 | 3.37 | 67.93 | 3.11 | 0.950 |
| G012162 | 95.17 | 0.64 | 28.77 | 0.25 | 67.17 | 0.99 | 0.801 |
| G012164 | 78.80 | 0.17 | 10.17 | 0.31 | 69.10 | 0.20 | 0.637 |
| G012165 | 83.40 | 2.20 | 14.87 | 0.83 | 69.27 | 2.72 | 0.953 |
| G012166 | 97.47 | 0.38 | 82.00 | 2.07 | 16.03 | 2.15 | 0.250 |
| G012167 | 96.63 | 0.29 | 70.37 | 0.90 | 27.87 | 1.29 | 0.297 |
| G012169 | 95.13 | 1.29 | 19.77 | 2.15 | 75.97 | 1.06 | 0.438 |

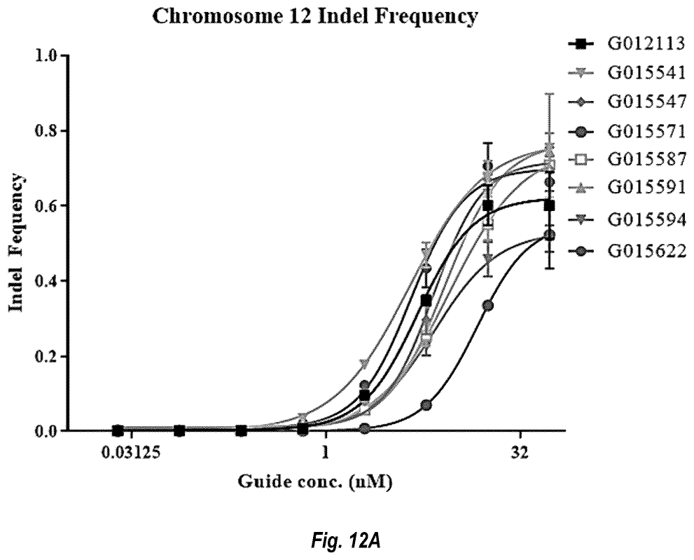

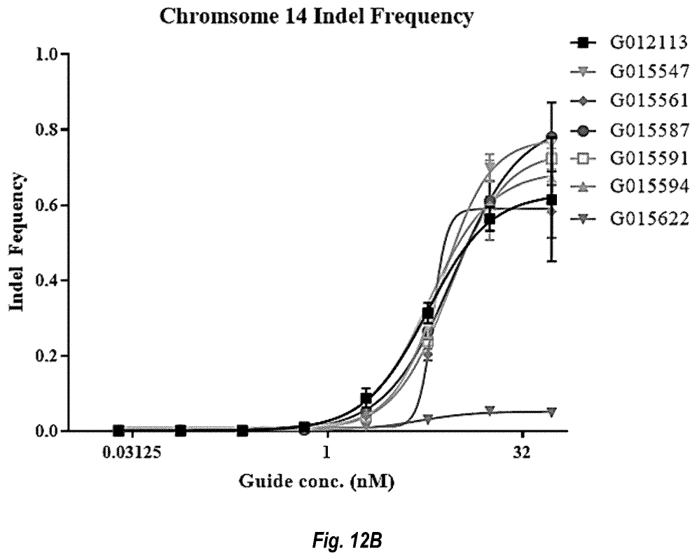

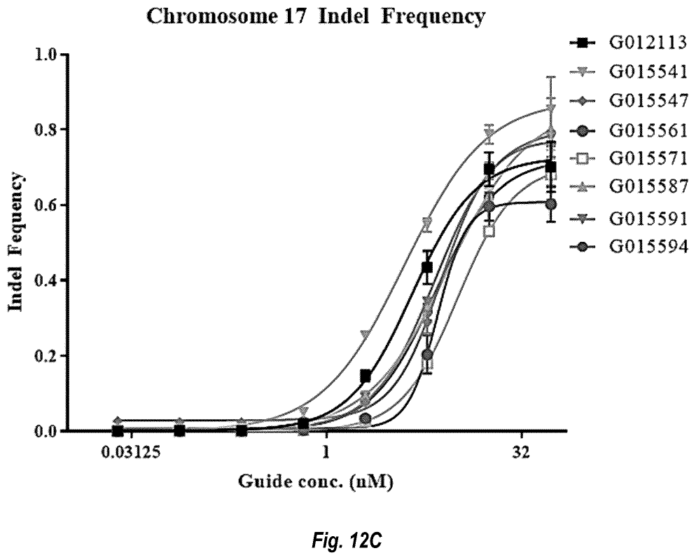

Cross screening of Spy Cas9 mRNA and sgRNA in primary cynomolgus hepatocytes using lipofection. Modified sgRNAs targeting LDHA were tested on primary cynomolgus hepatocytes in a dose response assay. Lipofection samples were prepared as described in Example 1. Primary cynomolgus hepatocytes were plated as described in Example 1. Cells were incubated at 37° C., 500 CO₂ for 48 hours prior to lipofection. Lipofection samples were incubated in media containing 600 cynomolgus serum at 37° C. for 10 minutes. Post-incubation the lipofection samples were added to the cynomolgus hepatocytes in an 8 point 3-fold dose response curve starting at 53 nM sgRNA (n=2). The cells were lysed 96 hours post-treatment for NGS analysis as described in Example 1. The dose response curve data for the guide sequences is shown in FIGS. 12A-12C. The 00 editing at the 53 nM concentration is listed below in Table 11.

TABLE 11
LDHA editing data for sgRNAs delivered to primary cynomolgus
hepatocytes via lipofection at 53 nM sgRNA

| | | Std Dev | | | Std Dev | | | Std Dev | |
|---|---|---|---|---|---|---|---|---|---|
| | Chr12 | Chr12 | | Chr14 | Avg | | Chr17 | Chr17 | |
| | Avg | Avg | Chr12 | Avg | Chr14 | Chr14 | Avg | Avg | Chr17 |
| Guide ID | % Edit | % Edit | EC50 | % Edit | % Edit | EC50 | % Edit | % Edit | EC50 |
| G012113 | 60.0 | 8.9 | 5.1 | 61.5 | 16.5 | 5.9 | 70.1 | 6.6 | 4.6 |
| G015541 | 75.4 | 14.4 | 4.4 | NA | NA | NA | 85.4 | 8.7 | 4.0 |
| G015547 | 69.8 | 5.7 | 6.8 | 76.0 | 1.1 | 7.5 | 76.2 | 3.5 | 7.6 |
| G015561 | NA | NA | NA | 58.3 | 7.0 | 6.5 | 60.3 | 4.7 | 7.1 |
| G015571 | 52.3 | 9.1 | 14.7 | NA | NA | NA | 68.1 | 6.6 | 10.2 |
| G015587 | 70.8 | 8.5 | 9.2 | 78.0 | 9.2 | 9.3 | 80.3 | 8.1 | 8.7 |
| G015591 | 74.6 | 1.1 | 8.3 | 72.3 | 2.8 | 8.6 | 77.9 | 1.1 | 6.9 |
| G015594 | 51.3 | 3.5 | 6.8 | 67.2 | 6.6 | 6.1 | 70.2 | 5.4 | 6.7 |
| G015622 | 66.3 | 6.9 | 4.5 | 4.9 | 0.3 | 5.0 | NA | NA | NA |

### Example 3. Phenotypic Analysis

### Western Blot Analysis of Intracellular Lactate Dehydrogenase A

Lipid nanoparticle (LNP) formulations of modified sgRNAs targeting human LDHA were administered to primary human hepatocytes to generate samples for Western Blotting. The LNPs were formulated as described in Example 1. Primary human hepatocytes were plated as described in Example 1. Cells were incubated at 37° C., 5% CO₂ for 48 hours prior to treatment with LNPs. LNPs were incubated in media containing 6% cynomolgus serum at 37° C. for 10 minutes. Post-incubation the LNPs were added to the human hepatocytes at a concentration of 25 nM of sgRNA per sample. At 96 hours post-transfection, a portion of the cells were collected and processed for NGS sequencing as described in Example 1. The remaining cells were harvested twenty-one days post-transfection and whole cell extracts (WCEs) were prepared and subjected to analysis by Western Blot as described in Example 1.

The editing data for these cells is provided in Table 12.

TABLE 12
LDHA editing data for sgRNA delivered
to primary human hepatocytes

| GUIDE ID | Edit frequency in PHH |
|---|---|
| G012089 | 0.871 |
| G012093 | 0.961 |
| G012095 | 0.926 |
| G012096 | 0.93 |
| G012103 | 0.882 |
| G012111 | 0.886 |
| G012115 | 0.933 |
| G012120 | 0.895 |
| G012133 | 0.915 |
| G012136 | 0.895 |

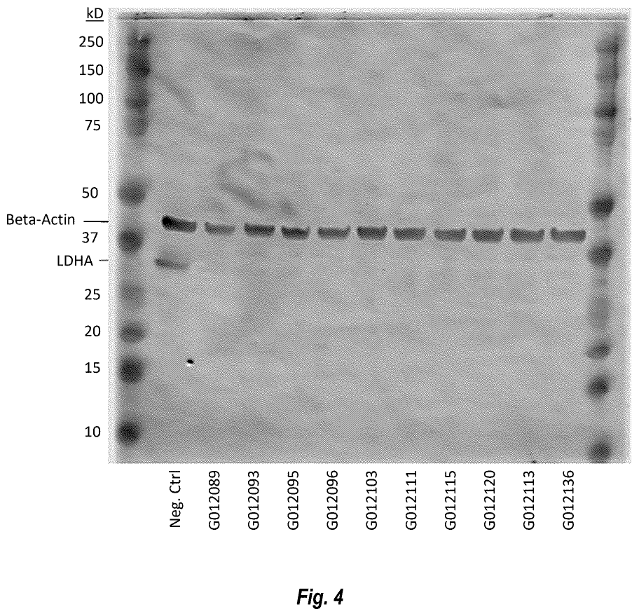

WCEs were analyzed by Western Blot for reduction of LDHA protein. Full length LDHA protein has 332 amino acids and a predicted molecular weight of 36.6 kD. A band at this molecular weight was observed in the control lane (untreated cells) but not in any of the treated lanes (FIG. 4).

### Transcript Analysis of Lactate Dehydrogenase A

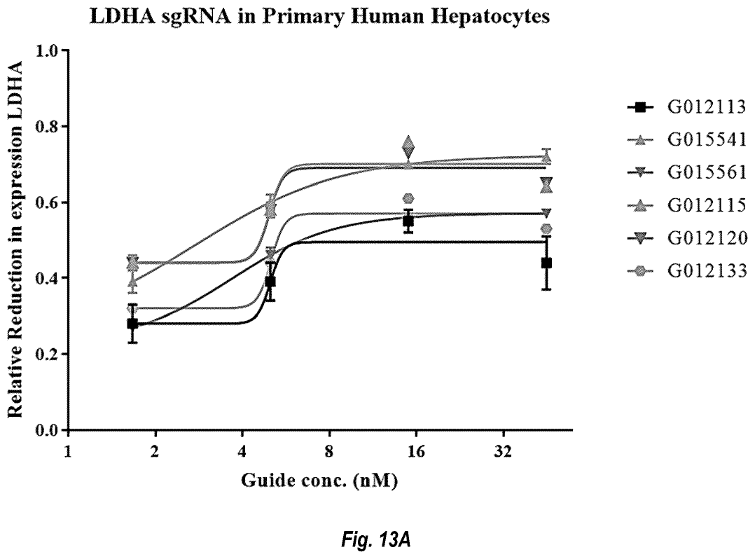

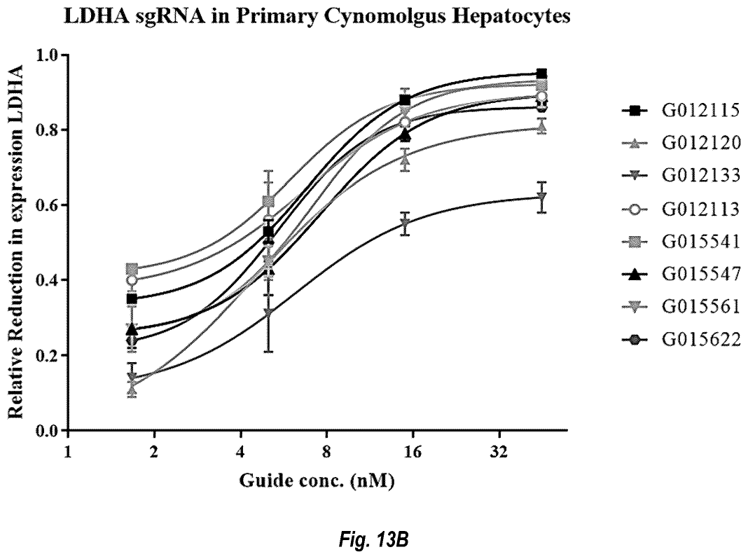

Select modified sgRNAs targetingLDHA were administered to primary human and cynomolgus hepatocytes by lipofection to generate samples for qPCR. The lipofection samples were formulated as described in Example 1. Primary hepatocytes were plated as described in Example 1. Cells were incubated at 37° C., 5 CO₂ for 48 hours prior to treatment with lipid packets. Lipofection samples were incubated in media containing 60 cynomolgus serum at 37° C. for 10 minutes. Post-incubation the lipid packets were added to the hepatocytes at multiples concentrations. At 96 hours post-lipofection, the cells were collected and processed for RNA as described in Example 1. Average LDHA transcript reduction in primary human and cynomolgus hepatocytes at 15 nM guide is contained within Table 13 below, with full dose-response data displayed in FIGS. 13A-13B.

TABLE 13
Average relatives LDHA reduction in primary human
and cynomolgus hepatocytes at 15 nM sgRNA

| | Primary Human | | Primary Cynomolgus | |
|---|---|---|---|---|
| | Hepatocytes | | Hepatocytes | |
| | | Std Dev Avg | | Std Dev Avg |
| | Avg Relative | Relative | Avg Relative | Relative |
| | Reduction in | Reduction in | Reduction in | Reduction in |
| | LDHA | LDHA | LDHA | LDHA |
| GuideID | Expression | Expression | Expression | Expression |
| G012113 | 0.55 | 0.03 | 0.82 | 0 |
| G012115 | 0.76 | 0.01 | 0.88 | 0.01 |
| G012120 | 0.73 | 0 | 0.72 | 0.03 |
| G012133 | 0.61 | 0.01 | 0.55 | 0.03 |
| G015541 | 0.7 | 0.01 | 0.88 | 0.03 |
| G015547 | NA | NA | 0.79 | 0.02 |
| G015561 | 0.56 | 0.01 | 0.85 | 0.01 |
| G015622 | NA | NA | 0.82 | 0.01 |

### Example 4. In Vivo Editing of Ldha in a Mouse Model of PH1

Both wildtype and AGT-deficient mice (Agxt1^{-/-}), e.g., null mutant mice lacking liver AGXT mRNA and protein were used in this study. The AGT-deficient mice exhibit hyperoxaluria and crystalluria and thus represent a phenotypic model of PH1, as previously described by Salido et al., Proc Natl Acad Sci USA. 2006 Nov. 28; 103(48):18249-54. The wildtype mice were used to determine which formulation to test in the AGT-deficient mice.

Prior to formulating LNPs, RNPs comprising dgRNAs targeting murine Ldha were screened for editing efficiency similarly as described in Example 2 for the human and cyno LDHA-targeting gRNAs. Having identified active gRNAs from the dgRNA screen, a smaller set of modified sgRNAs based on these gRNAs were synthesized for further evaluation in vivo.

Animals were weighed and grouped according to body weight for preparing dosing solutions based on group average weight. LNPs containing modified sgRNAs targeting murine Ldha (see Table 14 below) were dosed via the lateral tail vein in a volume of 0.2 mL per animal (approximately 10 mL per kilogram body weight). The LNPs were formulated as described in Example 1. One week post-treatment, wildtype mice were euthanized and liver tissue was collected for DNA extraction and analysis of editing of murine Ldha. As shown in Table 14 below, dose-dependent levels of editing were observed in treated mice.

TABLE 14
LDHA editing data for sgRNAs
targeting murine Ldha

| | sgRNA Sequence | | | | |
|---|---|---|---|---|---|
| | (* = PS linkage; | Dose | | | |
| | ‘m’ = 2′-O-Me | (mpk, total | Avg % | Std Dev | |
| Guide ID | nucleotide) | RNA cargo) | Edit | % Edit | n |
| G009438 | mG*mU*mU*CACGCGCUGAG | 0.3 | 19.20 | 7.01 | 5 |
| | CUGUCAGUUUUAGAmGmCmU | 1 | 59.08 | 9.83 | 5 |
| | mAmGmAmAmAmUmAmGmCAA | 3 | 74.54 | 0.74 | 5 |
| | GUUAAAAUAAGGCUAGUCCG | | | | |
| | UUAUCAmAmCmUmUmGmAmA | | | | |
| | mAmAmAmGmUmGmGmCmAmC | | | | |
| | mCmGmAmGmUmCmGmGmUmG | | | | |
| | mCmU*mU*mU*mU (SEQ | | | | |
| | ID NO: 86) | | | | |
| G009439 | mG*mG*mG*GGCCCGUCAGC | 0.3 | 9.40 | 2.75 | 5 |
| | AAGAGGGUUUUAGAmGmCmU | 1 | 37.56 | 9.30 | 5 |
| | mAmGmAmAmAmUmAmGmCAA | 3 | 65.94 | 5.37 | 5 |
| | GUUAAAAUAAGGCUAGUCCG | | | | |
| | UUAUCAmAmCmUmUmGmAmA | | | | |
| | mAmAmAmGmUmGmGmCmAmC | | | | |
| | mCmGmAmGmUmCmGmGmUmG | | | | |
| | mCmU*mU*mU*mU (SEQ | | | | |
| | ID NO: 87) | | | | |
| G009442 | mG*mU*mU*GCAAUCUGGAU | 0.3 | 15.90 | 1.74 | 5 |
| | UCAGCGGUUUUAGAmGmCmU | 1 | 49.98 | 7.41 | 5 |
| | mAmGmAmAmAmUmAmGmCAA | 3 | 68.40 | 3.85 | 5 |
| | GUUAAAAUAAGGCUAGUCCG | | | | |
| | UUAUCAmAmCmUmUmGmAmA | | | | |
| | mAmAmAmGmUmGmGmCmAmC | | | | |
| | mCmGmAmGmUmCmGmGmUmG | | | | |
| | mCmU*mU*mU*mU (SEQ | | | | |
| | ID NO: 88) | | | | |
| G009445 | mG*mU*mC*AUGGAAGACAA | 0.3 | 12.40 | 4.60 | 5 |
| | ACUCAAGUUUUAGAmGmCmU | 1 | 47.62 | 10.11 | 5 |
| | mAmGmAmAmAmUmAmGmCAA | 3 | 62.10 | 4.06 | 5 |
| | GUUAAAAUAAGGCUAGUCCG | | | | |
| | UUAUCAmAmCmUmUmGmAmA | | | | |
| | mAmAmAmGmUmGmGmCmAmC | | | | |
| | mCmGmAmGmUmCmGmGmUmG | | | | |
| | mCmU*mU*mU*mU (SEQ | | | | |
| | ID NO: 89) | | | | |
| G009447 | mA*mC*mU*GGGCACUGACG | 0.3 | 9.48 | 4.78 | 5 |
| | CAGACAGUUUUAGAmGmCmU | 1 | 40.88 | 11.07 | 5 |
| | mAmGmAmAmAmUmAmGmCAA | 3 | 66.10 | 4.69 | 5 |
| | GUUAAAAUAAGGCUAGUCCG | | | | |
| | UUAUCAmAmCmUmUmGmAmA | | | | |
| | mAmAmAmGmUmGmGmCmAmC | | | | |
| | mCmGmAmGmUmCmGmGmUmG | | | | |
| | mCmU*mU*mU*mU (SEQ | | | | |
| | ID NO: 90) | | | | |

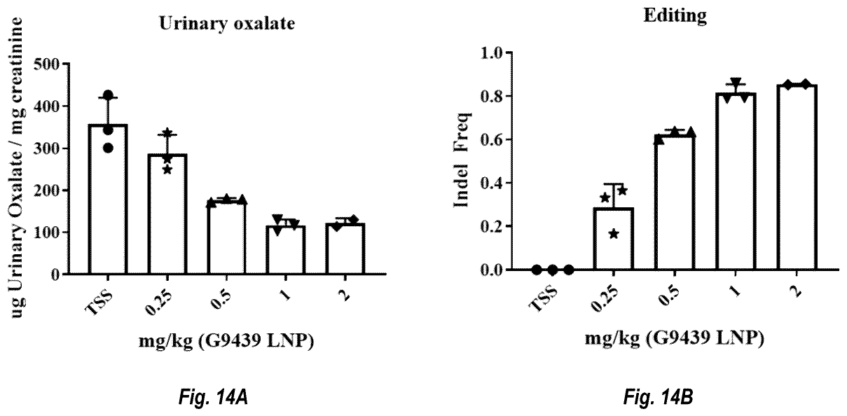

Having established the LNPs could edit the mouse Ldha gene in vivo, LNP containing G009439 was administered to the AGT-deficient mice in a dose response (0, 0.25, 0.5, 1, and 2 mpk) with respect to total mRNA cargo. These mice were housed in metabolic cages and urine was collected at various time points for oxalate levels, e.g., as described by Liebow et al., J Am Soc Nephrol. 2017 February; 28(2):494-503. Editing of the Ldha gene and secretion of oxalate were shown to increase and decrease, respectively, with increasing doses of LNP. The % editing and ug urinary oxalate/mg creatinine excreted are contained within Table 15 below and displayed in FIGS. 14A-14C.

TABLE 15
The % editing and ug urinary oxalate/mg creatinine
excreted after administration of LNP containing
G009439 to the AGT-deficient mice.

| | | | | Avg ug | Std Dev Avg | |
|---|---|---|---|---|---|---|
| | | Avg | Std Dev | Urinary | Urinary | |
| | | Editing | Avg | Oxalate/mg | Oxalate/mg | |
| Treatment | | % | Editing % | Creatinine | Creatinine | n |
| TSS | | 0.0 | 0.0 | 357.3 | 63.4 | 3 |
| 0.25 | mpk Ldha | 28.7 | 10.7 | 287.2 | 45.0 | 3 |
| 0.5 | mpk Ldha | 62.5 | 2.0 | 176.4 | 4.9 | 3 |
| 1 | mpk Ldha | 81.6 | 3.8 | 117.3 | 13.4 | 3 |
| 2 | mpk Ldha | 85.5 | 0.2 | 122.2 | 11.5 | 2 |

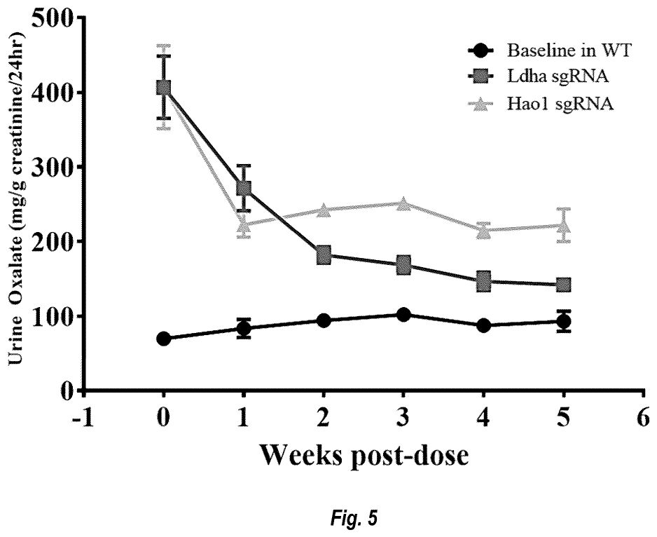

After establishing LNPs could reduce oxalate secretion in vivo, LNP containing G009439 was administered to the AGT-deficient mice at a dose of 2 mpk with respect to total mRNA cargo (n=4). As shown in FIG. 5, urine oxalate levels were reduced one week following treatment and this level of reduction was sustained out to at least 5 weeks post-dose at which point the study was terminated. No reduction was observed in control (PBS injected) animals (n=4). The percent editing in each treated animal is reported in Table 16, and the % reduction of urinary oxalate is shown at each week post-treatment in Table 18.

In the same study, AGT-deficient mice were also dosed with LNP (at a dose of 2 mpk (n=4)) containing a sgRNA (G000723) which targets murine Haol. As also shown in FIG. 5 and Table 17, oxalate levels were reduced one week following treatment with LNP comprising this gRNA and this level of reduction was sustained out to at least 5 weeks post-dose.

G000723:
(SEQ ID NO: 85)
mC*mA*mC*GUGAGCCAUGCACUGCAGUUUUAGAmGmCmUmAmGmAmAmA
mUmAmGmCAAGUUAAAAUAAGGCUAGUCCGUUAUCAmAmCmUmUmGmAmA
mAmAmAmGmUmGmGmCmAmCmCmGmAmGmUmCmGmGmUmGmCmU*mU*mU
*mU * = PS linkage; ‘m’ = 2′-O-Me nucleotide

TABLE 16
Editing results from AGXT −/− mice treated with LNP
comprising LDHA targeting gRNA (G009439) at 2 mpk

| Mouse # | % Edit | % Insertion | % Deletion |
|---|---|---|---|
| 1 | 90.8 | 1.1 | 89.7 |
| 2 | 86.1 | 1.3 | 84.8 |
| 3 | 90.5 | 1.1 | 89.4 |
| 4 | 90.3 | 1.2 | 89.2 |

TABLE 17
Editing results from AGXT −/− mice treated with LNP
comprising HAO1 targeting gRNA (G000723) at 2 mpk

| Mouse # | % Edit | % Insertion | % Deletion |
|---|---|---|---|
| 1 | 71.1 | 47.7 | 23.4 |
| 2 | 83.1 | 56 | 27.1 |
| 3 | 81.5 | 52.8 | 28.7 |
| 4 | 83.7 | 54.9 | 28.8 |

TABLE 18
Average oxalate levels and % reduction from baseline in
AGXT −/− mice treated with LNP comprising LDHA targeting
gRNA (G009439) at 2 mpk (of total RNA cargo) over 5 weeks. N = 4

| Collection | Avg ug Oxalate/mg | Avg % Reduction ug |
|---|---|---|
| date | creatinine | Oxalate/mg creatinine |
| Baseline | 407 | 0.00 |
| Week 1 | 272 | 33.18 |
| Week 2 | 182 | 55.25 |
| Week 3 | 168 | 58.59 |
| Week 4 | 146 | 64.11 |
| Week 5 | 142 | 65.11 |

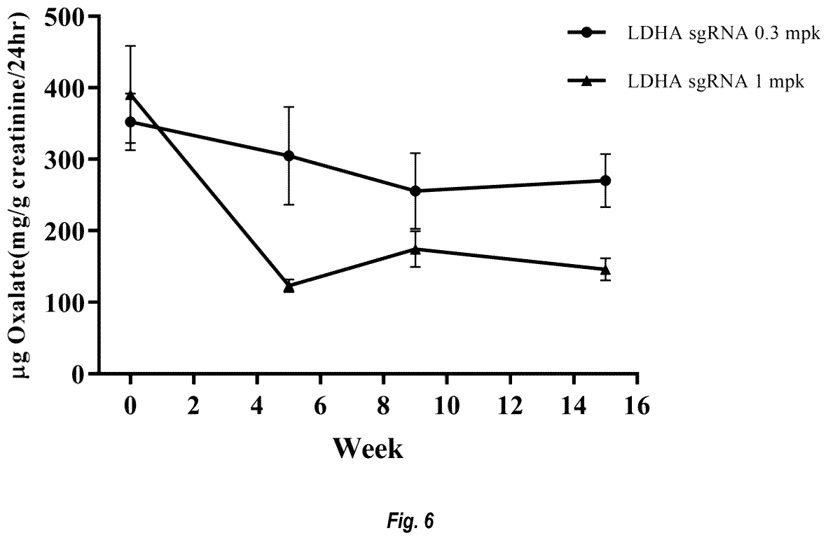

Having demonstrated sustained urine oxalate reduction in AGT-deficient mice up to 5 weeks after LNP treatment, an additional study was conducted to track urine oxalate up to 15 weeks post-dose. LNP containing G009439 was administered to AGT-deficient mice at doses of 0.3 mpk (n=4) and 1 mpk (n=4). These mice were housed in metabolic cages and urine was collected at various time points for oxalate levels, as described above. Table 19 shows the editing results for the AGT-deficient mice. The average % editing achieved at 0.3 mpk dose was 33.42, std. dev. 11.95. The average % editing achieved at 1 mpk dose was 75.68, std. dev. 7.35. As shown in FIG. 6, urine oxalate levels were reduced following treatment and this level of reduction was sustained to 15 weeks post-dose at which point the study was terminated. The data depicted in FIG. 6 are shown in Table 20. No reduction was observed (data not shown) in control (PBS injected) animals (n=3).

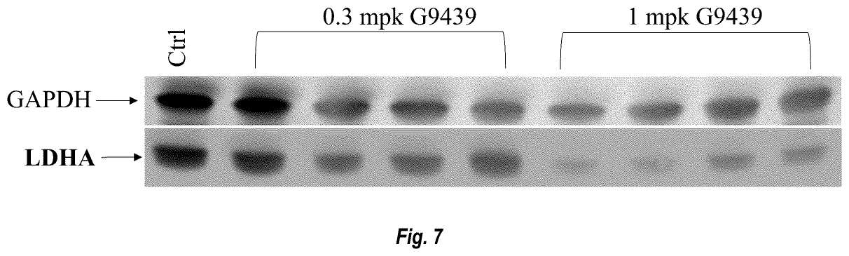

Liver samples from the treated mice were processed and run on Western Blots as described in Example 1. Percent reduction of LDHA protein was calculated using the Licor Odyssey Image Studio Ver 5.2 software. GAPDH was used as a loading control and probed simultaneously with LDHA. A ratio was calculated for the densitometry values for GAPDH within each sample compared to the total region encompassing the band for LDHA. Percent reduction of LDHA protein was determined after the ratios were normalized to negative control lanes. Results are shown in Table 19 and depicted in FIG. 7.

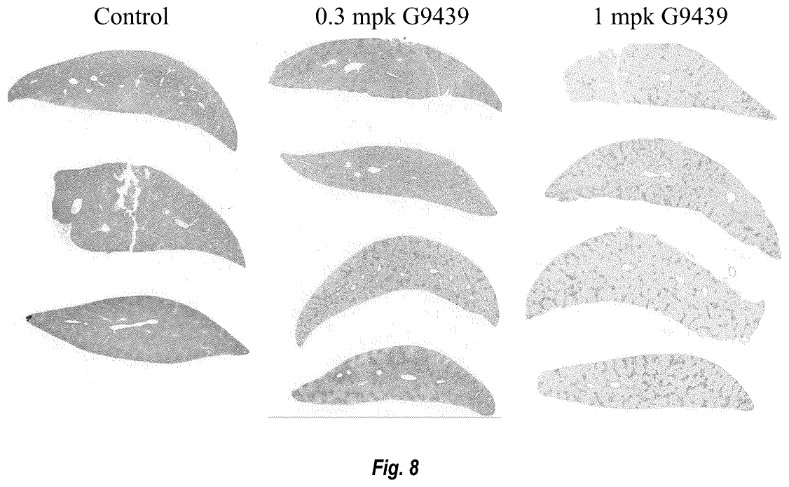

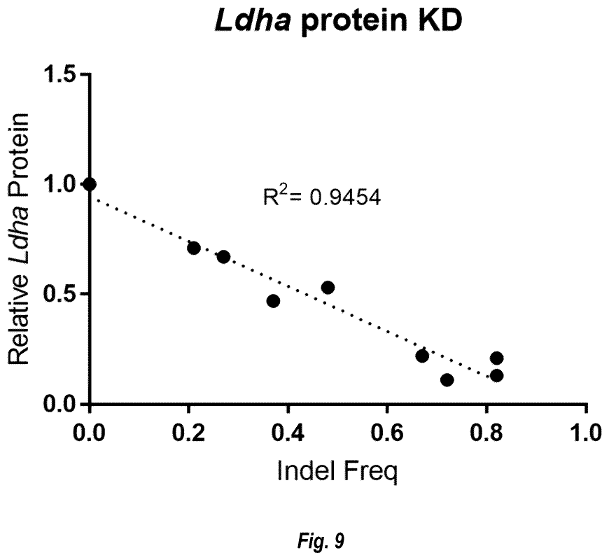

LDHA protein in treated and nontreated mice was additionally characterized through immunohistochemical staining as described in Example 1 and depicted in FIG. 8. A progressive reduction in LDHA staining was observed in 0.3 mpk-dosed mice and 1mpk-dosed mice compared to control mice. FIG. 9 shows a correlation with an R² value of 0.95 between the editing and protein levels in Table 19.

TABLE 19
Agxt1−/− Mouse Model Editing and Protein Data, 15 Week Study

| | | | | | LDHA Protein |
|---|---|---|---|---|---|
| | | | | | remaining |
| | mpk | % | % | % | (relative to |
| Mouse # | G009439 | Edit | Insertion | Deletion | negative control) |
| 1 | 0.3 | 27.2 | 0.3 | 26.9 | 0.67 |
| 2 | 0.3 | 37.2 | 0.5 | 36.7 | 0.47 |
| 3 | 0.3 | 48.3 | 0.7 | 47.7 | 0.53 |
| 4 | 0.3 | 21.0 | 0.5 | 20.6 | 0.71 |
| 5 | 1 | 81.6 | 1.2 | 80.5 | 0.13 |
| 6 | 1 | 72.0 | 1.0 | 71.1 | 0.11 |
| 7 | 1 | 67.1 | 0.8 | 66.3 | 0.22 |
| 8 | 1 | 82.0 | 1.2 | 80.8 | 0.21 |

TABLE 20
Agxt1−/− Mouse Model Average Urine
Oxalate (n = 4 for each dose)

| | Dose G009439 | Avg Urine Oxalate | Std Dev Avg |
|---|---|---|---|
| Week | (mpk) | (mg/g creatinine/24 hr) | Urine Oxalate |
| 0 | TSS | 377.47 | 58.22 |
| 5 | TSS | 413.72 | 77.33 |
| 9 | TSS | 354.77 | 43.75 |
| 15 | TSS | 345.95 | 88.18 |
| 0 | 0.3 | 352.09 | 39.77 |
| 5 | 0.3 | 304.78 | 68.34 |
| 9 | 0.3 | 255.69 | 53.17 |
| 15 | 0.3 | 270.24 | 37.08 |
| 0 | 1.0 | 390.46 | 68.06 |
| 5 | 1.0 | 123.26 | 8.94 |
| 9 | 1.0 | 174.33 | 25.01 |
| 15 | 1.0 | 145.91 | 15.46 |

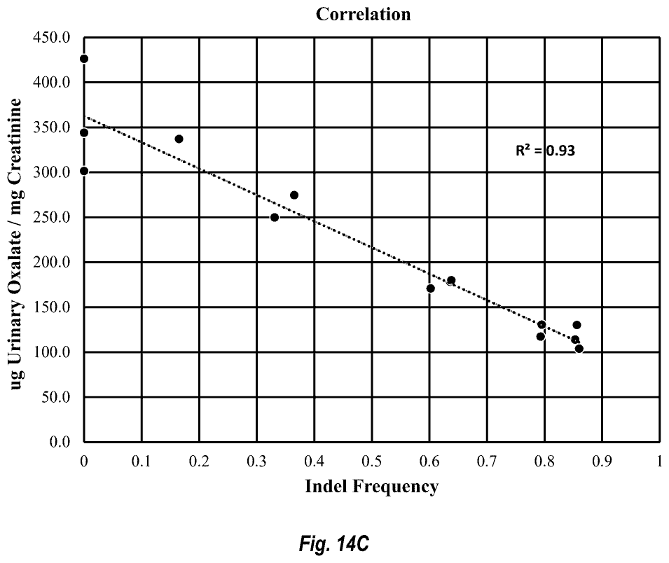

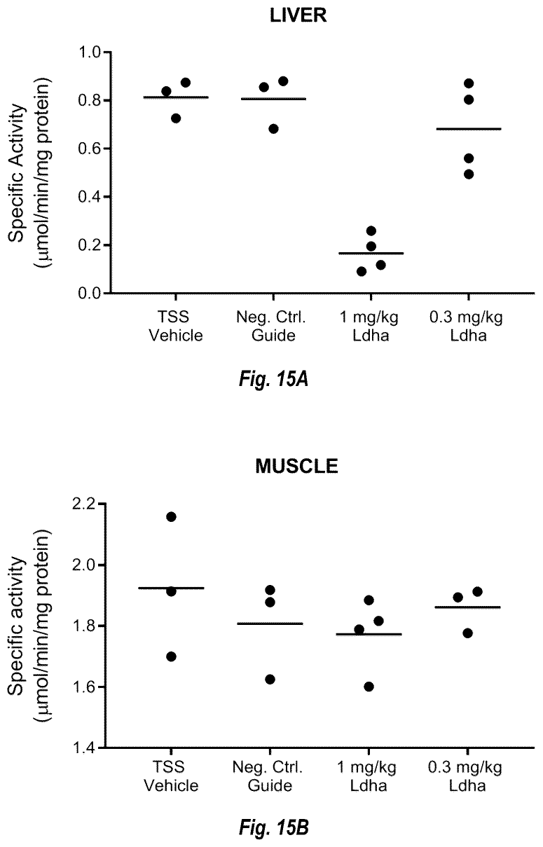

Liver and muscle samples from the treated mice were processed for LDH activity as described in Example 1. Reduction of LDH activity was observed in liver samples from mice treated with 1mpk of Ldha LNP. Specific activity (mol/min/mg protein) from the treated and control mice are contained in Table 21 below and data displayed in FIGS. 15A-15B.

TABLE 21
Liver and muscle specific LDH activity

| | | Std Dev Avg | | Std Dev Avg | |
|---|---|---|---|---|---|
| | Avg Specific | Specific | Avg Specific | Specific | |
| | Activity | Activity | Activity | Activity | |
| | (μmol/min/mg | (μmol/min/mg | (μmol/min/mg | (μmol/min/mg | |
| | protein) - | protein) - | protein) - | protein) - | |
| Treatment | Liver | Liver | Muscle | Muscle | n |
| TSS | 0.8 | 0.1 | 1.9 | 0.2 | 3.0 |
| Neg. Ctrl. Guide | 0.8 | 0.1 | 1.8 | 0.2 | 3.0 |
| 0.3 mpk Ldha Guide | 0.7 | 0.2 | 1.6 | 0.5 | 4.0 |
| 1 mpk Ldha Guide | 0.2 | 0.1 | 1.8 | 0.1 | 4.0 |

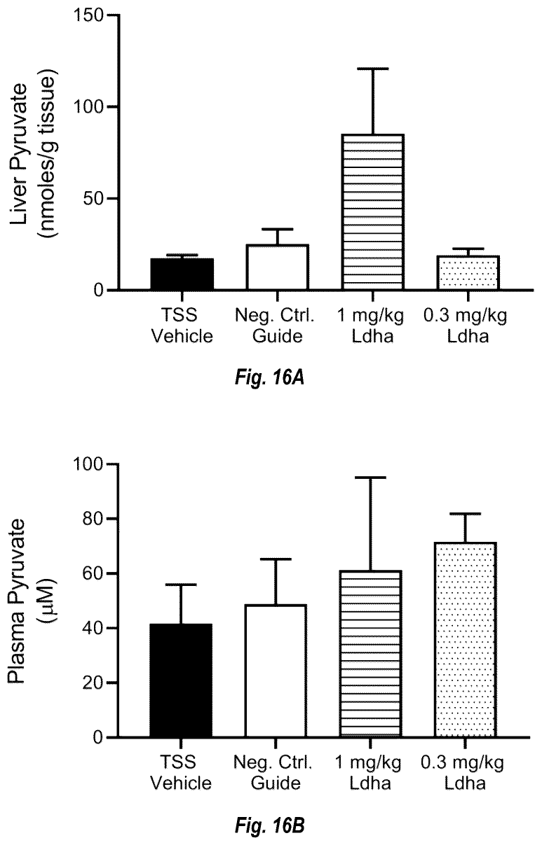

Liver and plasma samples from the treated mice were also analyzed for pyruvate, as described in Example 1. Pyruvate is a metabolite converted to lactate by lactate dehydrogenase (Urbanska K et al, nt Mol Sci. 2019 Apr. 27; 20(9)). Pyruvate concentrations proved to be elevated in liver samples from 1mpk-treated mice, but little differences in plasma pyruvate concentrations were observed between treated and control mice. These data are contained in Table 22 and shown in FIGS. 16A-16B.

TABLE 22
Liver and plasma pyruvate quantification

| | Avg Liver | Std Dev Avg | | Std Dev Avg | |
|---|---|---|---|---|---|
| | Pyruvate | Liver Pyruvate | Avg Plasma | Plasma | |
| | (nmols/g | (nmols/g | Pyruvate | Pyruvate | |
| Treatment | tissue) | tissue) | (μM) | (μM) | n |
| TSS | 17.40 | 1.76 | 41.64 | 14.29 | 3 |
| Neg. Ctrl. Guide | 25.12 | 8.17 | 48.76 | 16.47 | 3 |
| 0.3 mpk Ldha Guide | 19.11 | 3.58 | 71.64 | 10.20 | 4 |
| 1 mpk Ldha Guide | 85.46 | 35.30 | 61.32 | 33.82 | 4 |

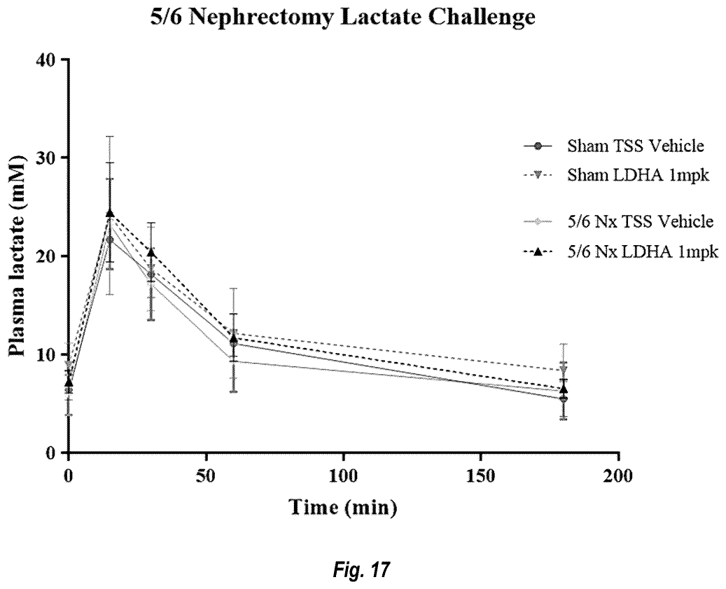

Having demonstrated sustained urine oxalate reduction in AGT-deficient mice up to 15 weeks after LNP treatment, an additional study was conducted to determine the ability of mice with compromised kidney function to clear lactate after LDHA knockdown. C51B16 male mice that had undergone either 5/6 nephrectomy or sham surgeries were obtained from the Jackson Laboratory (Bar Harbor, ME). One-week post-surgery, animals were bled for baseline lactate levels as described in Example 1. Animals were then dosed with LNP containing G009439 at a dose of 2mpk (n=6). Two weeks post-dose, animals were given a lactate challenge comprising of 2 g/kg of sodium lactate dissolved in phosphate buffered saline (concentration 200 mg/mL, ˜18 mM) pH 7.4, delivered intraperitoneally. Animals were tail-bled before the challenge and then 15, 30, 60, and 180 minutes post-challenge. Blood samples were analyzed for lactate levels as described in Example 1. No significant differences in lactate clearance were observed in mice that had received the nephrectomy surgeries and LDHA LNP, compared to sham surgery and vehicle treatment mice. Table 23 below details the average plasma pyruvate across animal groups, as also shown in FIG. 17.

TABLE 23
Nephrectomy study plasma lactate clearance

| | Sham Surgery - | | Sham Surgery - | | 5/6 Nephrectomy - | | 5/6 Nephrectomy - | |
|---|---|---|---|---|---|---|---|---|
| | TSS Vehicle Ctrl | | Ldha 1 mpk | | TSS Vehicle Ctrl | | Ldha 1 mpk | |
| | (n = 5) | | (n = 6) | | (n = 5) | | (n = 5) | |
| | | Std Dev | | Std Dev | | Std Dev | | Std Dev |
| | Avg | Avg | Avg | Avg | Avg | Avg | Avg | Avg |
| | Plasma | Plasma | Plasma | Plasma | Plasma | Plasma | Plasma | Plasma |
| Time | Lactate | Lactate | Lactate | Lactate | Lactate | Lactate | Lactate | Lactate |
| (min) | (mM) | (mM) | (mM) | (mM) | (mM) | (mM) | (mM) | (mM) |
| 0 | 7.2 | 2.9 | 5.9 | 2.0 | 9.0 | 2.3 | 6.5 | 2.8 |
| 15 | 24.5 | 10.3 | 23.3 | 4.6 | 24.2 | 8.1 | 21.7 | 9.1 |
| 30 | 20.4 | 8.2 | 17.2 | 3.7 | 18.7 | 4.3 | 18.1 | 7.7 |
| 60 | 11.7 | 4.9 | 9.4 | 3.1 | 12.2 | 4.5 | 11.1 | 4.7 |
| 180 | 6.6 | 2.6 | 6.3 | 2.9 | 8.4 | 2.7 | 5.5 | 2.8 |

1.-8. (canceled)

9. A composition comprising:
a) a guide RNA comprising
i) the guide sequence CCUAUCAUACAGUGCUUAUG (SEQ ID NO: 5); or
ii) at least 17 contiguous nucleotides of the guide sequence CCUAUCAUACAGUGCUUAUG (SEQ ID NO: 5); or
iii) a guide sequence that is at least 95% or 90% identical to CCUAUCAUACAGUGCUUAUG (SEQ ID NO: 5); or
iv) a guide sequence comprising the sequence

| (SEQ ID NO: 5) | |
|---|---|
| | CCUAUCAUACAGUGCUUAUG |

10.-216. (canceled)

217. The composition of claim 9, wherein the guide RNA is formulated in a lipid nanoparticle (LNP).

218. The composition of claim 9, wherein the composition further comprises an RNA-guided DNA binding agent or an mRNA that encodes an RNA-guided DNA binding agent.

219. The composition of claim 218, wherein the RNA-guided DNA binding agent is a Cas9.

220. The composition of claim 9, wherein the composition comprises a single guide RNA (sgRNA) comprising in 5′ to 3′ order:
1) the guide sequence; and
2) the nucleotide sequence
(SEQ ID NO: 201)
GUUUUAGAGCUAGAAAUAGCAAGUUAAAAUAAGGCUAGUCCGUUAUCAAC
UUGAAAAAGUGGCACCGAGUCGGUGCUUUU.

221. The composition of claim 220, wherein the sgRNA comprises (1) a 5′ end modification; (2) a 3′ end modification; or (3) a 5′ end modification and a 3′ end modification.

222. The composition of claim 221, wherein the composition further comprises an RNA-guided DNA binding agent or an mRNA that encodes an RNA-guided DNA binding agent.

223. The composition of claim 222, wherein the RNA-guided DNA binding agent is a Cas9.

224. The composition of claim 9, wherein the composition comprises a single guide RNA (sgRNA) comprising in 5′ to 3′ order:
1) the guide sequence; and
2) the nucleotide sequence GUUUUAGAmGmCmUmAmGmAmAmAmUmAmGmCAAGUUAAAAUAAGGCUA GUCCGUUAUCAmAmCmUmUmGmAmAmAmAmAmGmUmGmGmCmAmCmCm GmAmGmUmCmGmGmUmGmCmU*mU*mU*mU (SEQ ID NO: 405), wherein a lower case “m” indicates that the nucleotide is 2′-O-Me modified and a * denotes a phosphorothioate (PS) linkage.
